(19) United States
(12) Patent Application Publication
(10) Pub. No.: US 2026/0327624 A1
(43) Pub. Date: Oct. 22, 2026

(54) COMPOSITIONS AND METHODS FOR INHIBITING EXPRESSION OF COAGULATION FACTOR XI (FXI)

(71) Applicant: SHANGHAI ARGO BIOPHARMACEUTICAL CO., LTD., Shanghai (CN)

(72) Inventors: Dongxu SHU, Zhejiang (CN); Pengcheng Patrick SHAO, Warrington Township, PA (US); Shiwei XIA, Zhejiang (CN)

(21) Appl. No.: 19/484,003

(22) PCT Filed: May 17, 2024
(86) PCT No.: PCT/CN2024/093796

§ 371 Date: Nov. 13, 2025

(30) Foreign Application Priority Data
May 19, 2023 (WO) PCT/CN2023/095175

(51) Int. Cl. C12N 15/113

(52) U.S. Cl. CPC C12N 15/1137; C12Y 304/21027; C12N 2310/14; C12N 2310/321; C12N 2310/322; C12N 2310/3231; C12N 2310/351

(57) ABSTRACT

Compositions and methods useful to inhibiting the expression of Plasma Coagulation Factor XI (FXI) gene and for treatment of FXI-associated diseases and conditions are provided. Provided are FXI dsRNA agents, FXI antisense polynucleotide agents, compositions comprising FXI dsRNA agents, and compositions comprising FXI antisense polynucleotide agents that can be used to inhibiting FXI expression in cells and subjects.

### FIELD OF THE INVENTION

The invention relates, in part, to compositions and methods that can be used to inhibit coagulation factor XI (FXI) gene expression.

### BACKGROUND

The dynamic balance of the circulatory system requires mechanisms that prevent blood loss, as well as those that counteract inappropriate intravascular obstructions. Plasma Coagulation Factor XI (hereinafter referred to as “FXI”) synthesized in the liver, is a member of the coagulation cascade “intrinsic pathway”, an essential component of the contact activation pathway, is conducive to the production of thrombin, which in turn is an important component that is engaged in the fibrin formation and offers protection from fibrinolysis. High levels of FXI are one of the risk factors for venous thrombosis. Thromboembolism may cause conditions such as deep vein thrombosis, pulmonary embolism, myocardial infarction, and stroke. Factor XI deficiency (also known as plasma thromboplastin antecedent (PTA) deficiency, Rosenthal syndrome and hemophilia C) is an autosomal recessive disease associated with a tendency to bleed. Most patients with Factor XI deficiency do not bleed spontaneously but can bleed seriously after trauma. Low levels of Factor XI can also occur in other disease states, including Noonan syndrome.

The most commonly used anticoagulants, warfarin, heparin, low molecular weight heparin (LMWH), and newer direct oral anticoagulants (DOAC) for the treatment of thrombotic disease, all possess significant drawbacks. For example, direct oral anticoagulants (DOAC) Rivaroxaban, Dabigatran, and Edoxaban all exhibit increased bleeding, especially increased GI bleeding risk. Currently, there remains a need for therapies to treat thromboembolic conditions without the risk of increased bleeding.

Accordingly, there is a need to find alternative treatments for thrombotic diseases with more attractive clinical profiles. One of the crucial technologies for developing RNAi drugs targeting FXI represents a novel approach to reducing FXI levels and treating FXI-related diseases, such as thromboembolic conditions without increasing bleeding risk (thromboembolic conditions such as vein thrombosis, venous or arterial thrombosis, pulmonary embolism, myocardial infarction, stroke, thrombosis associated with chronic kidney disease or end-stage renal disease (ESRD), including thrombosis associated with dialysis, or other procoagulant condition). Also provided herein RNAi drugs targeting FXI to an animal at risk for inflammatory disease.

### SUMMARY OF THE INVENTION

In general, the present disclosure features novel FXI gene-specific RNAi agents, compositions that include FXI RNAi agents, and methods for inhibiting expression of a FXI gene in vitro and/or in vivo using the FXI RNAi agents and compositions that include FXI RNAi agents described herein. The FXI RNAi agents described herein can selectively and efficiently decrease, inhibit, or silence expression of a FXI gene in a subject, e.g., a human or animal subject.

According to an aspect of the invention, a double-stranded ribonucleic acid (dsRNA) agent for inhibiting expression of coagulation factor XI (FXI) is provided, wherein the dsRNA agent includes a sense strand and an antisense strand, nucleotide positions 2 to 18 in the antisense strand comprising a region of complementarity to a FXI RNA transcript, wherein the region of complementarity includes at least 15, 16, 17, 18, 19, 20 or 21 contiguous nucleotides that differ by 0, 1, 2, or 3 nucleotides from one of the antisense sequences listed in one of Tables 1-3, and optionally comprising a targeting ligand.

Another aspect of the invention, a double-stranded ribonucleic acid (dsRNA) agent for inhibiting expression of coagulation factor XI (FXI) is provided, wherein the dsRNA agent includes a sense strand and an antisense strand, wherein the sense strand comprises at least 15, 16, 17, 18, 19, 20 or 21 contiguous nucleotides that differ by 0, 1, 2, or 3 nucleotides from any one of the nucleotide sequences of nucleotides 143-173, 150-180, 380-410, 384-414,389-419, 402-432, 419-449, 536-566, 1141-1171, 1488-1518, 1561-1591, 2111-2141, 2112-2142, 2113-2143, 2115-2145, 2111-2145 of SEQ ID NO: 1, and the antisense strand comprises at least 15, 16, 17, 18, 19, 20 or 21 contiguous nucleotides differing by 0, 1, 2, or 3 nucleotides from the corresponding nucleotide sequence of SEQ ID NO:2, wherein the sense strand and the antisense strand can be partially, substantially, or fully complementary to each other.

In some embodiments, the dsRNA agent comprises a sense strand and an antisense strand forming a double stranded region, wherein the sense strand comprises at least 15, 16, 17, 18, 19, 20 or 21 contiguous nucleotides that differ by 0, 1, 2, or 3 nucleotides from any one of the nucleotide sequences of nucleotides 145-171, 152-178, 386-412, 382-408, 404-430, 1490-1516, 1563-1589, 2113-2139, 2114-2140, 2115-2141, 2117-2143, 2113-2143 of SEQ ID NO: 1, and the antisense strand comprises at least 15, 16, 17, 18, 19, 20 or 21 contiguous nucleotides differing by 0, 1, 2, or 3 nucleotides from the corresponding nucleotide sequence of SEQ ID NO:2.

In some embodiments, the dsRNA agent comprises a sense strand and an antisense strand forming a double stranded region, wherein the sense strand comprises at least 15, 16, 17, 18, 19, 20 or 21 contiguous nucleotides that differ by 0, 1, 2, or 3 nucleotides from any one of the nucleotide sequences of nucleotides 148-168, 155-175,385-405,389-409,394-414,407-427, 424-444,541-561, 1146-1166, 1493-1513, 1566-1586, 2116-2136, 2117-2137, 2118-2138, 2120-2140, 2116-2140 of SEQ ID NO: 1, and the antisense strand comprises at least 15, 16, 17, 18, 19, 20 or 21 contiguous nucleotides differing by 0, 1, 2, or 3 nucleotides from the corresponding nucleotide sequence of SEQ ID NO:2.

In some embodiments, the FXI RNA transcript is SEQ ID NO: 1.

In some embodiments, the sense strand and the antisense strand may be the same length or different lengths. In some embodiments, each strand is no more than 40 nucleotides in length. In some embodiments, each strand is no more than 30 nucleotides in length. In some embodiments, each strand is no more than 25 nucleotides in length. In some embodiments, each strand is no more than 23 nucleotides in length. In some embodiments, each strand is no more than 21 nucleotides in length. In some embodiments, the sense and antisense strands of the RNAi agents can each be 15 to 49 nucleotides in length. In some embodiments, the antisense strand is independently 15, 16, 17, 18, 19, 20, 21, 22, 23, 24, 25, 26, 27, 28, 29, or 30 nucleotides in length. In some embodiments, the length of the sense strand is independently 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides. In some embodiments, the sense strand and the antisense strand are both 21 nucleotides in length.

In some embodiments, the sense strand is complementary or substantially complementary to the antisense strand, and the region of complementarity is between 15 and 23 nucleotides in length. In some embodiments, the region of complementarity is 19-21 nucleotides in length. In some embodiments, the region of complementarity is 14, 15, 16, 17, 18, 19, 20, 21, 22, 23, 24, 25, 26, 27, 28, 29, or 30 nucleotides in length.

In some embodiments, the dsRNA agent includes a sense strand and an antisense strand forming a double stranded region, wherein said antisense strand comprises a region of complementarity to part of a FXI RNA transcript, which comprises at least 15, 16, 17, 18 or 19 contiguous nucleotides from any one of the antisense sequences listed in any one of Tables 1-3.

In some embodiments, the dsRNA agent includes a sense strand and an antisense strand forming a double stranded region, wherein said antisense strand comprises a region of complementarity to part of a FXI RNA transcript which comprises at least 15, 16, 17, 18 or 19 contiguous nucleotides with 0, 1, 2, 3, 4 or 5 mismatches. In some embodiments, the dsRNA agent includes a sense strand and an antisense strand forming a double stranded region, wherein said antisense strand comprises a region of complementarity to a target region of a FXI RNA transcript which comprises at least 15, 16, 17, 18 or 19 contiguous nucleotides with 0, 1, 2, 3, 4 or 5 mismatches.

In some embodiments, the antisense strand of the dsRNA agent is at least substantially complementary to any one of a target region of SEQ ID NO: 1 and is provided in any one of Tables 1-3. In some embodiments, the antisense strand of the dsRNA agent is fully complementary to any one of a target region of SEQ ID NO: 1 and is provided in any one of Tables 1-3. In some embodiments, the dsRNA agent includes a sense strand sequence set forth in any one of Tables 1-3, wherein the sense strand sequence is at least substantially complementary to the antisense strand sequence in the dsRNA agent. In certain embodiments, the dsRNA agent includes a sense strand sequence set forth in any one of Tables 1-3, wherein the sense strand sequence is fully complementary to the antisense strand sequence in the dsRNA agent. In some embodiments, the dsRNA agent includes an antisense strand sequence set forth in any one of Tables 1-3. In some embodiments, the dsRNA agent includes the sequences set forth as a duplex sequence in any of Tables 1-3.

In some embodiments, the dsRNA agent includes at least one modified nucleotide. In certain embodiments, all or substantially all of the nucleotides of the antisense strand are modified nucleotides. In some embodiments, at least one of the modified nucleotides comprises: 2′-O-methyl nucleotide, 2′-fluoro nucleotide, 2′-deoxy nucleotide, 2′3′-seco nucleotide mimic, locked nucleotide, unlocked nucleic acid nucleotide (UNA), glycol nucleic acid nucleotide (GNA), 2′-F-Arabino nucleotide, 2′-methoyxyethyl nucleotide, abasic nucleotide, ribitol, inverted nucleotide, inverted abasic nucleotide, inverted 2′-OMe nucleotide, inverted 2′-deoxy nucleotide, isomannide nucleotide, 2′-amino-modified nucleotide, 2′-alkyl-modified nucleotide, mopholino nucleotide, and 3′-OMe nucleotide, a nucleotide including a 5′-phosphorothioate group, or a terminal nucleotide linked to a cholesteryl derivative or dodecanoic acid bisdecylamide group, a 2′-amino-modified nucleotide, a phosphoramidite, or a non-natural base including nucleotide.

In some embodiments, the dsRNA agent includes an E-vinylphosphonate nucleotide at the 5′ end of the antisense strand.

In certain embodiments, the dsRNA agent includes at least one phosphorothioate internucleoside linkage. In certain embodiments, the sense strand includes at least one phosphorothioate internucleoside linkage. In some embodiments, the antisense strand includes at least one phosphorothioate internucleoside linkage. In some embodiments, the sense strand includes 1, 2, 3, 4, 5, or 6 phosphorothioate internucleoside linkages. In some embodiments, the antisense strand includes 1, 2, 3, 4, 5, or 6 phosphorothioate internucleoside linkages. In some embodiments, the sense strand and the antisense strand each independently comprise two phosphorothioate linkages at the 5′-terminus. In some embodiments, the sense strand and the antisense strand each independently comprise two phosphorothioate linkages at the 3′-terminus.

In some embodiments, all or substantially all of the nucleotides of the sense strand and the antisense strand are modified nucleotides.

In some embodiments, the antisense strand comprises 15 or more modified nucleotides independently selected from a 2′-O-methyl nucleotide, a 2′-fluoro nucleotide and an UNA modified nucleotide, wherein less than 6 modified nucleotides are 2′-fluoro nucleotides. In some embodiments, the antisense strand comprises 3 or 5 2′-fluoro nucleotides, preferably, the antisense strand comprises 5 2′-fluoro nucleotides. In some embodiments, the sense strand comprises 15 or more modified nucleotides independently selected from a 2′-O-methyl nucleotide and a 2′-fluoro nucleotide, wherein less than 4 modified nucleotides are 2′-fluoro nucleotides. In certain embodiments, the sense strand comprises 3 2′-fluoro nucleotides. In some embodiments, the antisense strand comprises at least one UNA modified nucleotide and 5 2′-fluoro nucleotides. In some embodiments, the antisense strand comprises 5 2′-fluoro nucleotides at positions 2, 5, 12, 14 and 18 counting from the first matching position of the 5′ end, and the rest 2′-O-methyl nucleotides. In some embodiments, the antisense strand comprises 5 2′-fluoro nucleotides at positions 2, 7, 12, 14 and 16 counting from the first matching position of the 5′ end, and the rest 2′-O-methyl nucleotides. In some embodiments, the antisense strand comprises one UNA modified nucleotide at position 7 and 5 2′-fluoro nucleotides at positions 2, 5, 12, 14 and 18 counting from the first matching position of the 5′ end, and the rest 2′-O-methyl nucleotides. In some embodiments, the antisense strand comprises one UNA modified nucleotide at position 7 and 5 2′-fluoro nucleotides at positions 2, 7, 12, 14 and 16 counting from the first matching position of the 5′ end, and the rest 2′-O-methyl nucleotides. In some embodiments, the sense strand comprises 3 2′-fluoro nucleotides at positions 8, 11 and 13 counting from the first matching position of the 3′ end, and the rest 2′-O-methyl nucleotides. In some embodiments, the sense strand comprises 3 2′-fluoro nucleotides at positions 9, 11 and 13 counting from the first matching position of the 3′ end, and the rest 2′-O-methyl nucleotides. In some embodiments, the modified sense strand is a modified sense strand sequence set forth in one of Tables 2-3. In some embodiments, the modified antisense strand is a modified antisense strand sequence set forth in one of Tables 2-3.

In some embodiments, the dsRNA agent includes at least one modified nucleotide and further includes one or more targeting groups or linking groups. In some embodiments, the one or more targeting groups or linking groups are conjugated to the sense strand. In some embodiments, the targeting group or linking group includes N-acetyl-galactosamine (GalNAc).

In some embodiments, the targeting group has a structure:

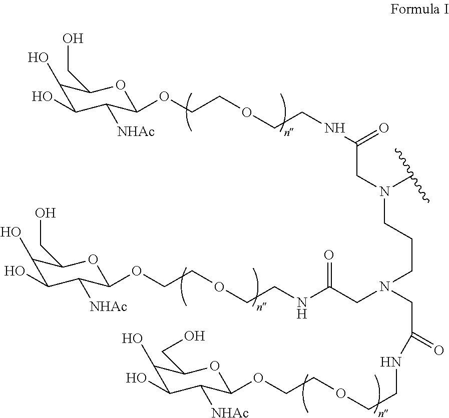

n″ are independently selected from 1 or 2.

In some embodiments, the targeting group has a structure:

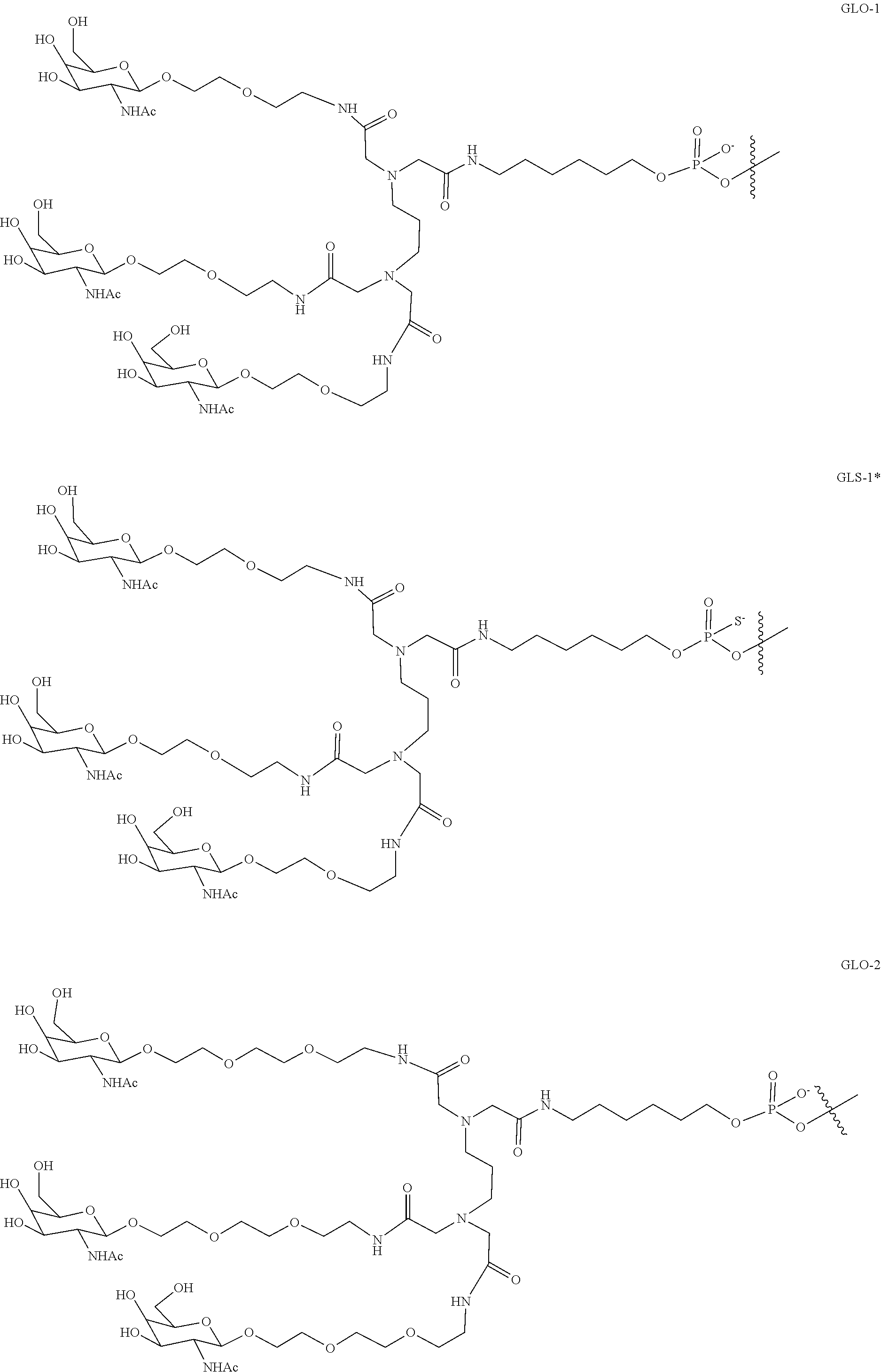 [SMILES: *OP(=O)([O-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCNC(=O)CN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

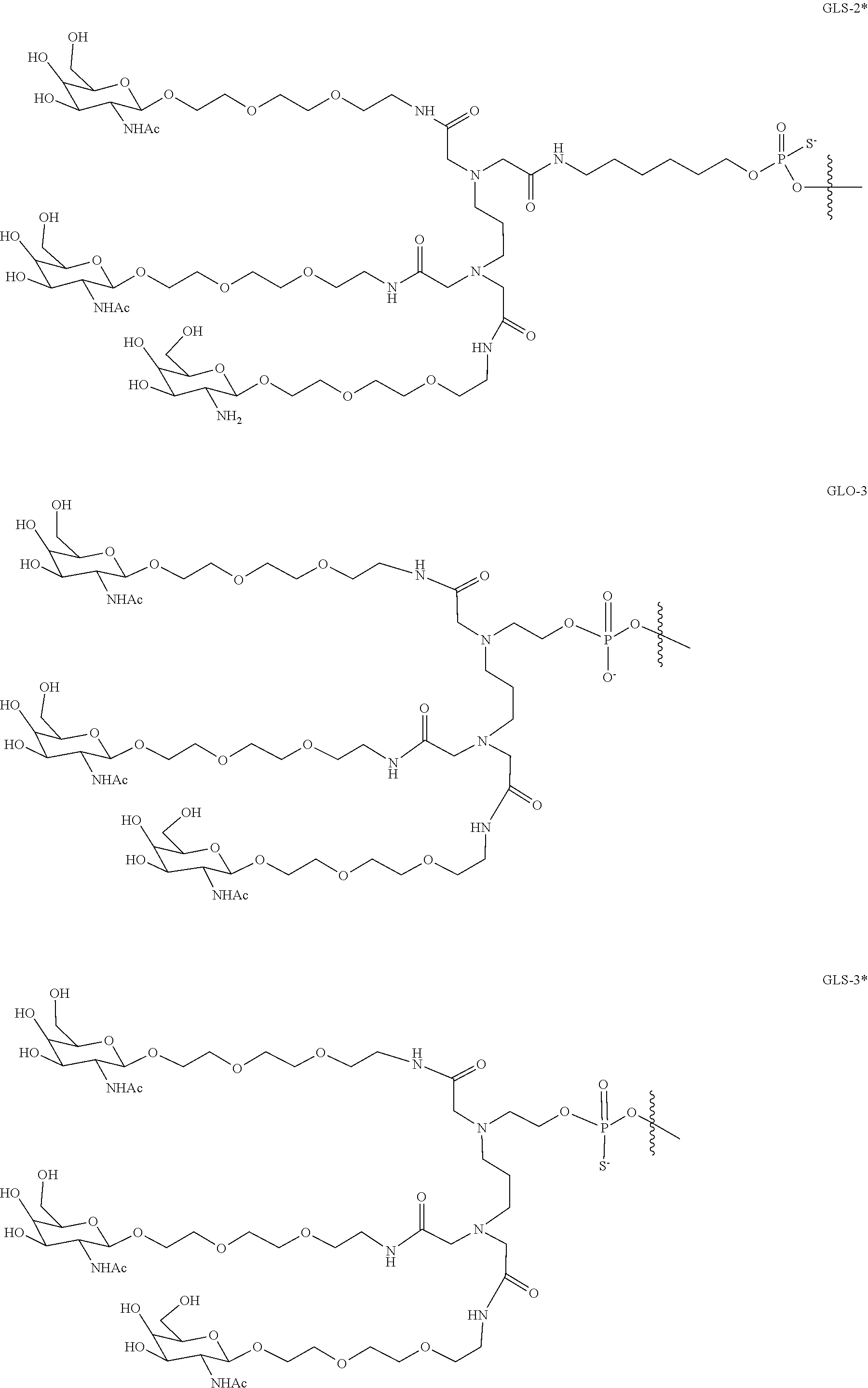

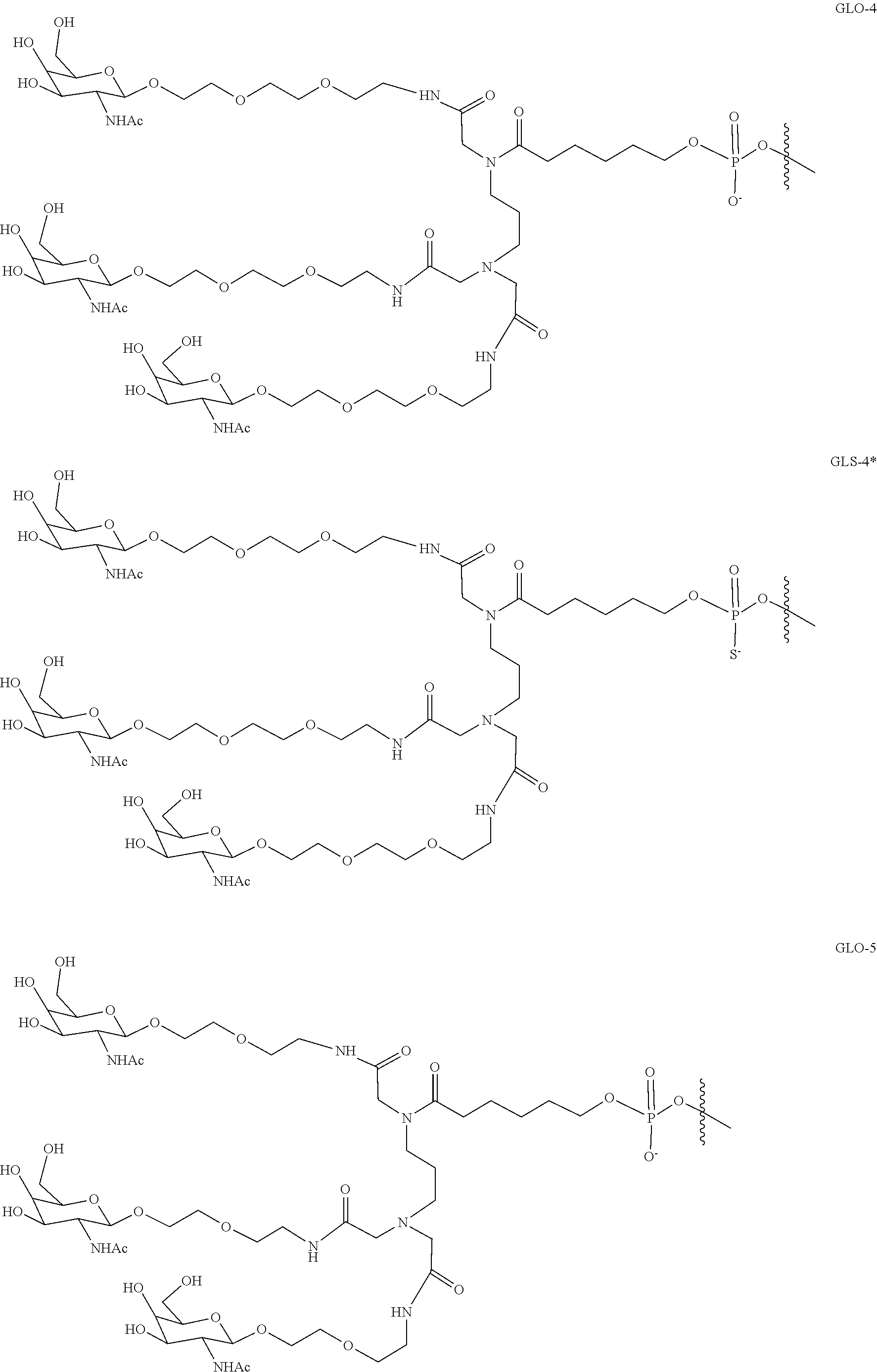 [SMILES: *OP(=O)([O-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

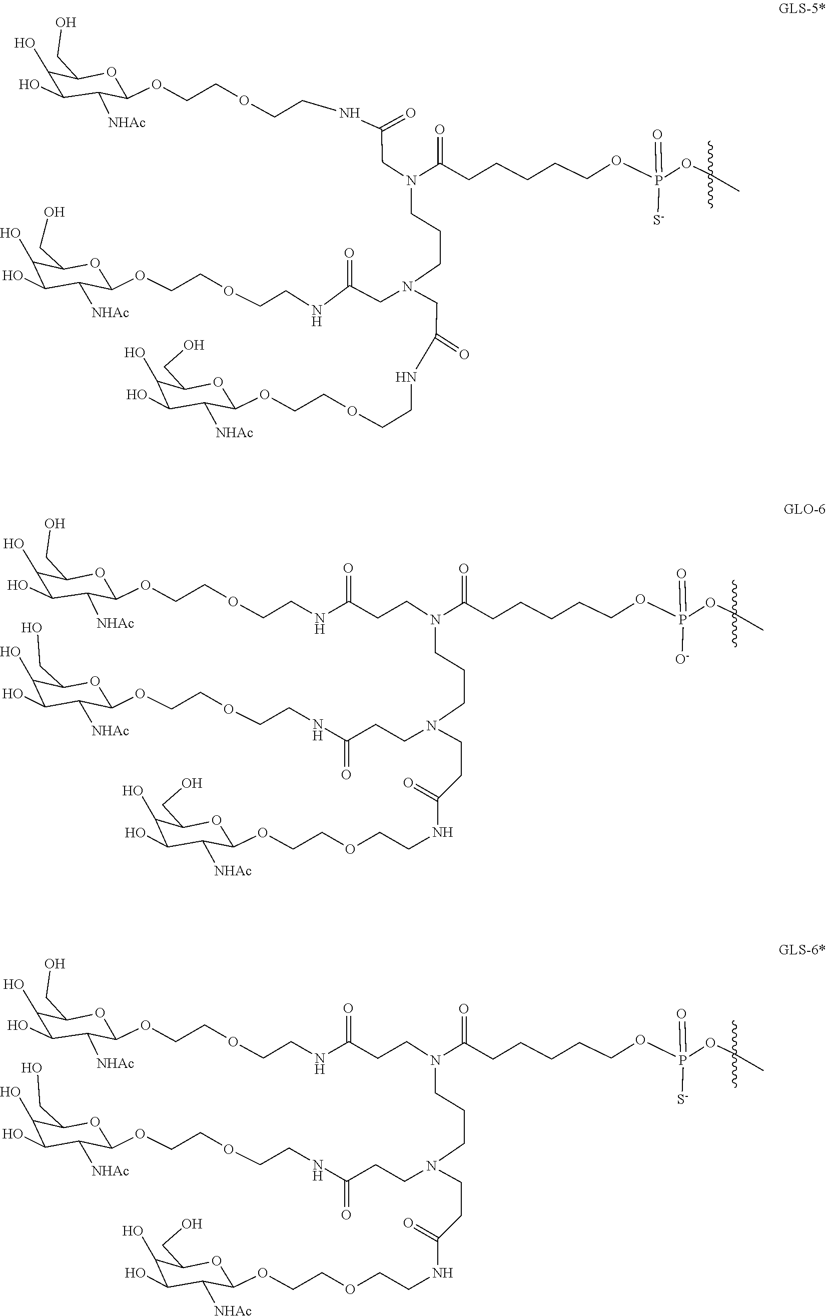 [SMILES: *OP(=O)([S-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O]

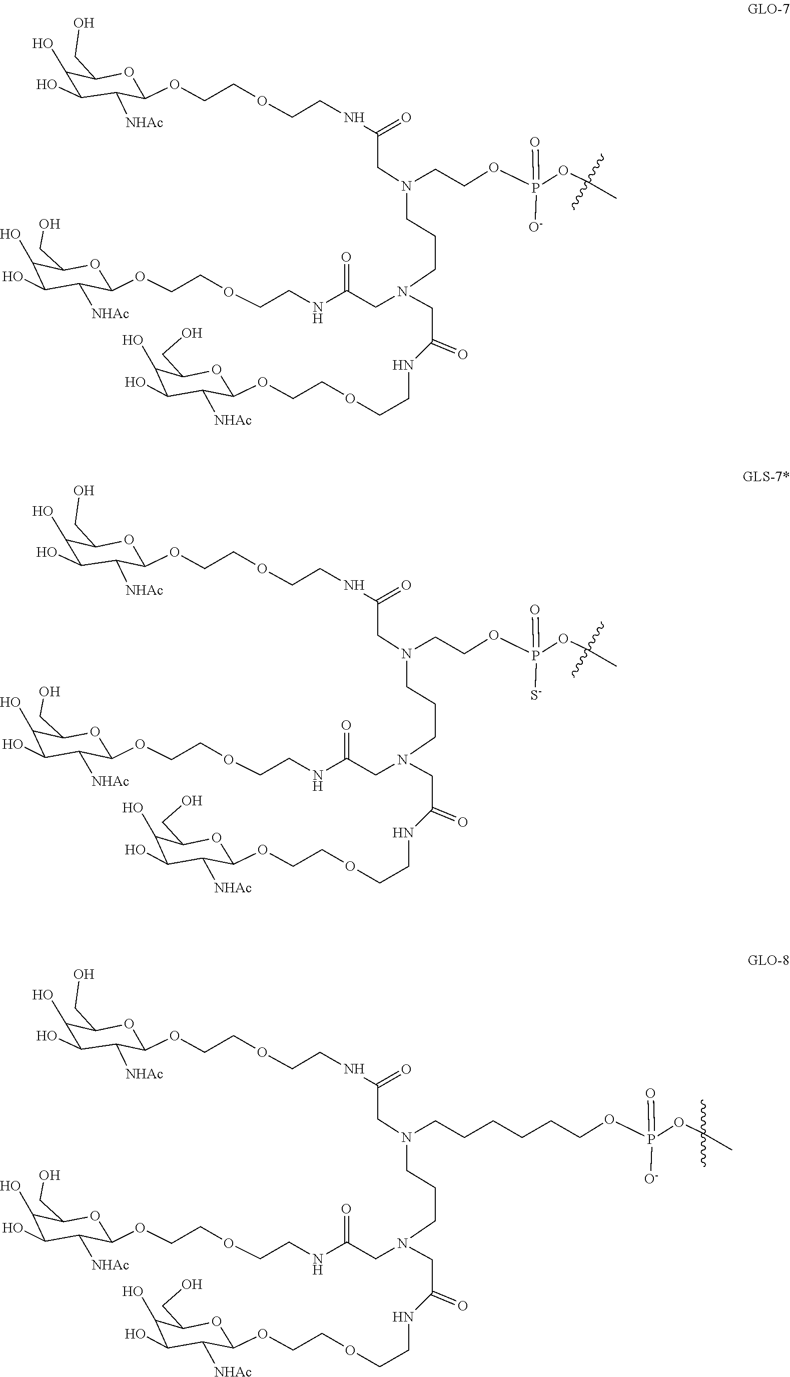 [SMILES: *OP(=O)([O-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

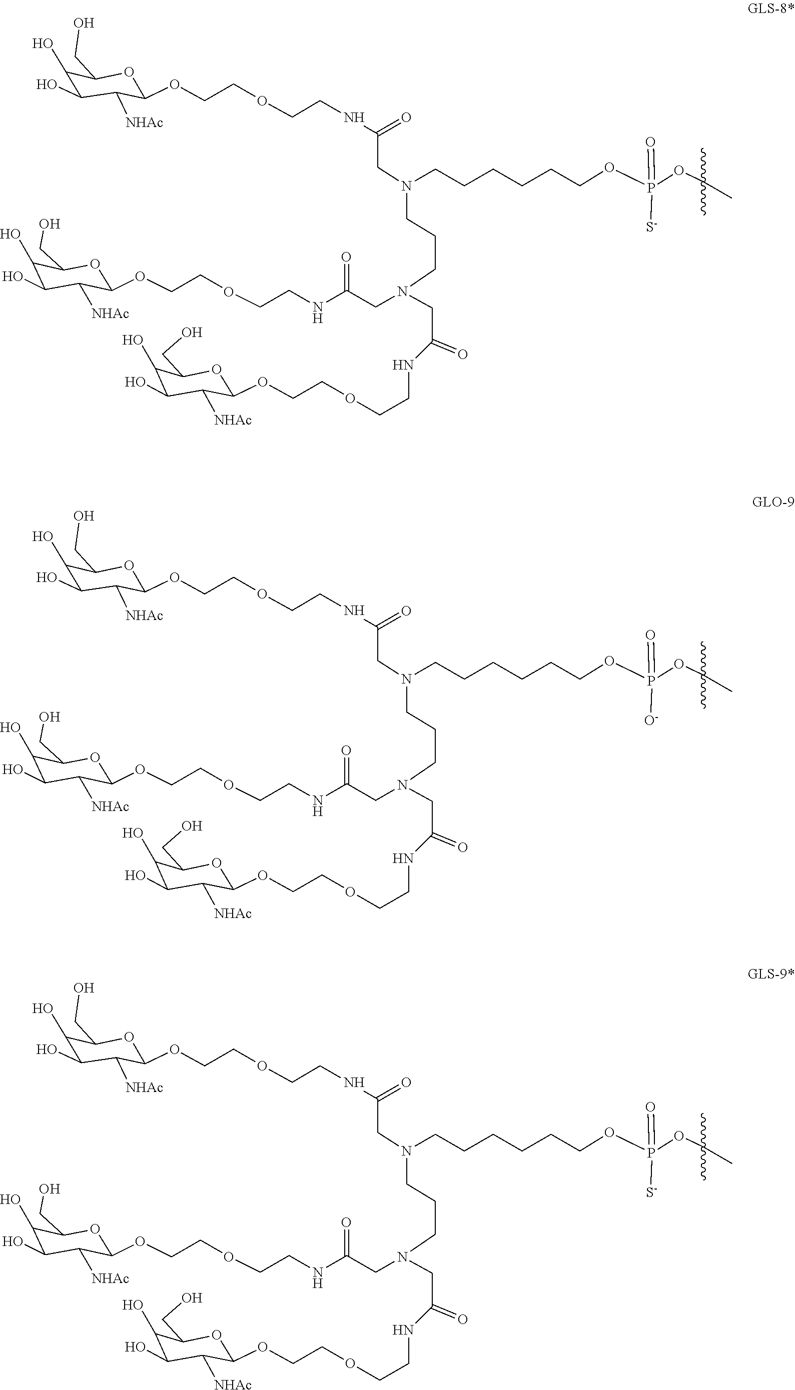 [SMILES: *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

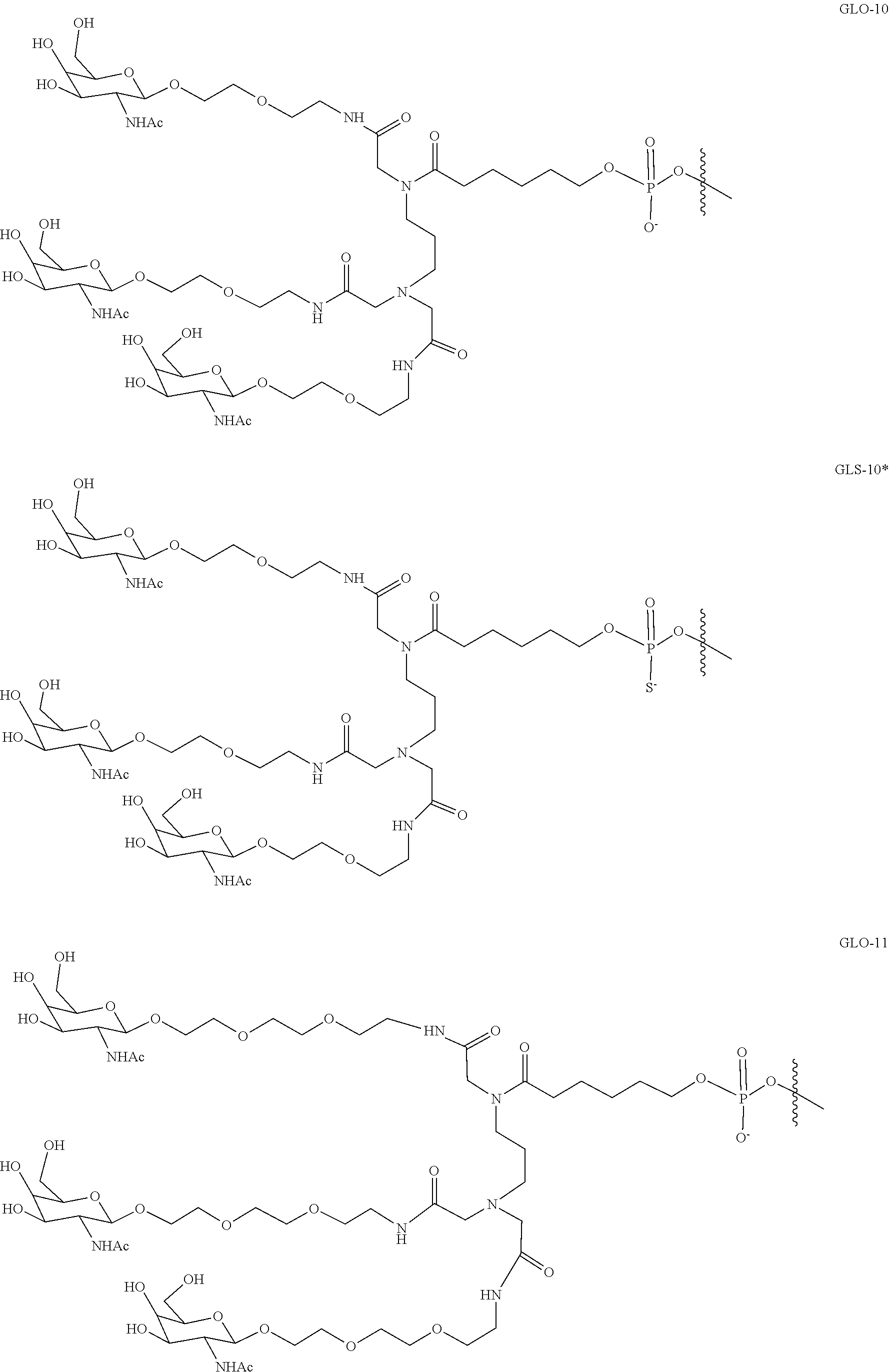 [SMILES: *OP(=O)([O-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

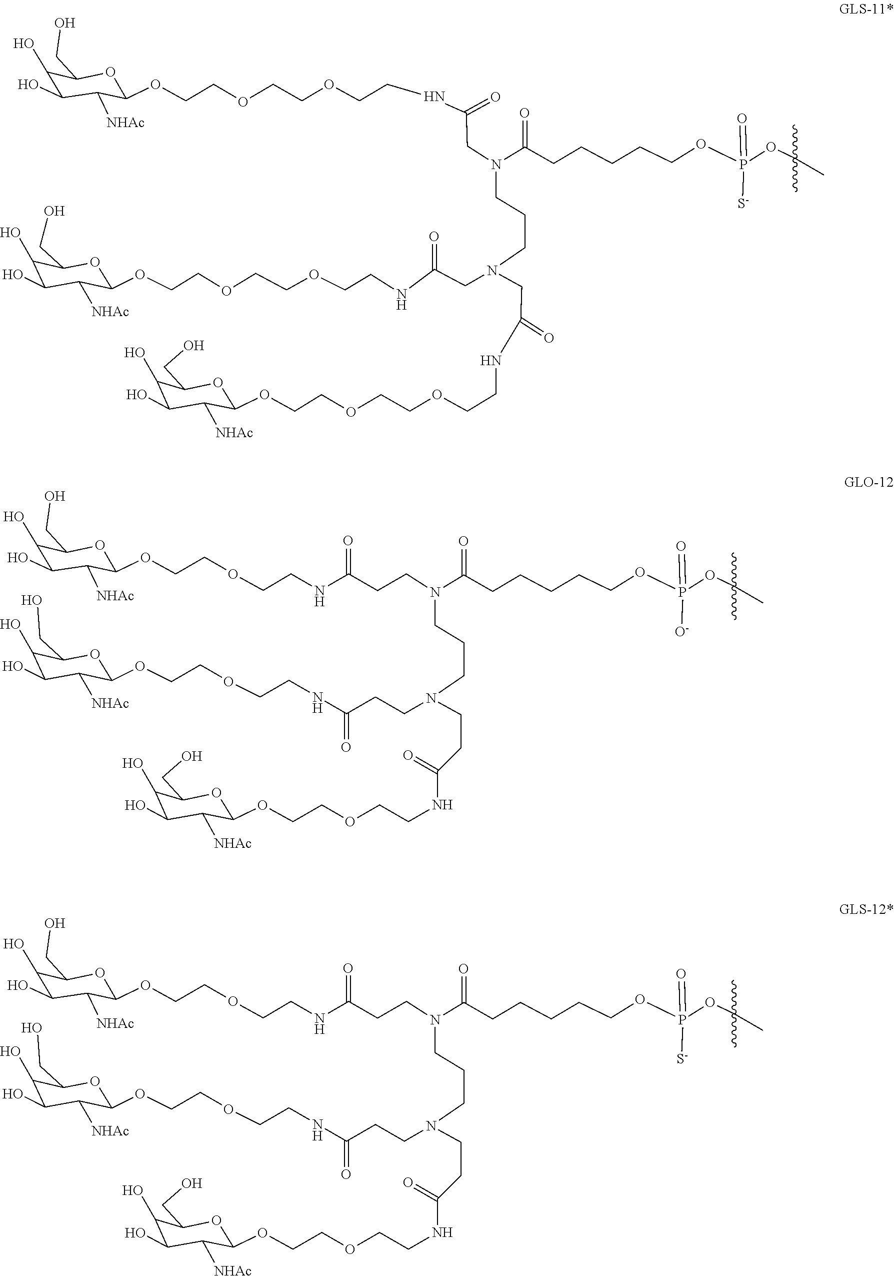 [SMILES: *OP(=O)([S-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O]

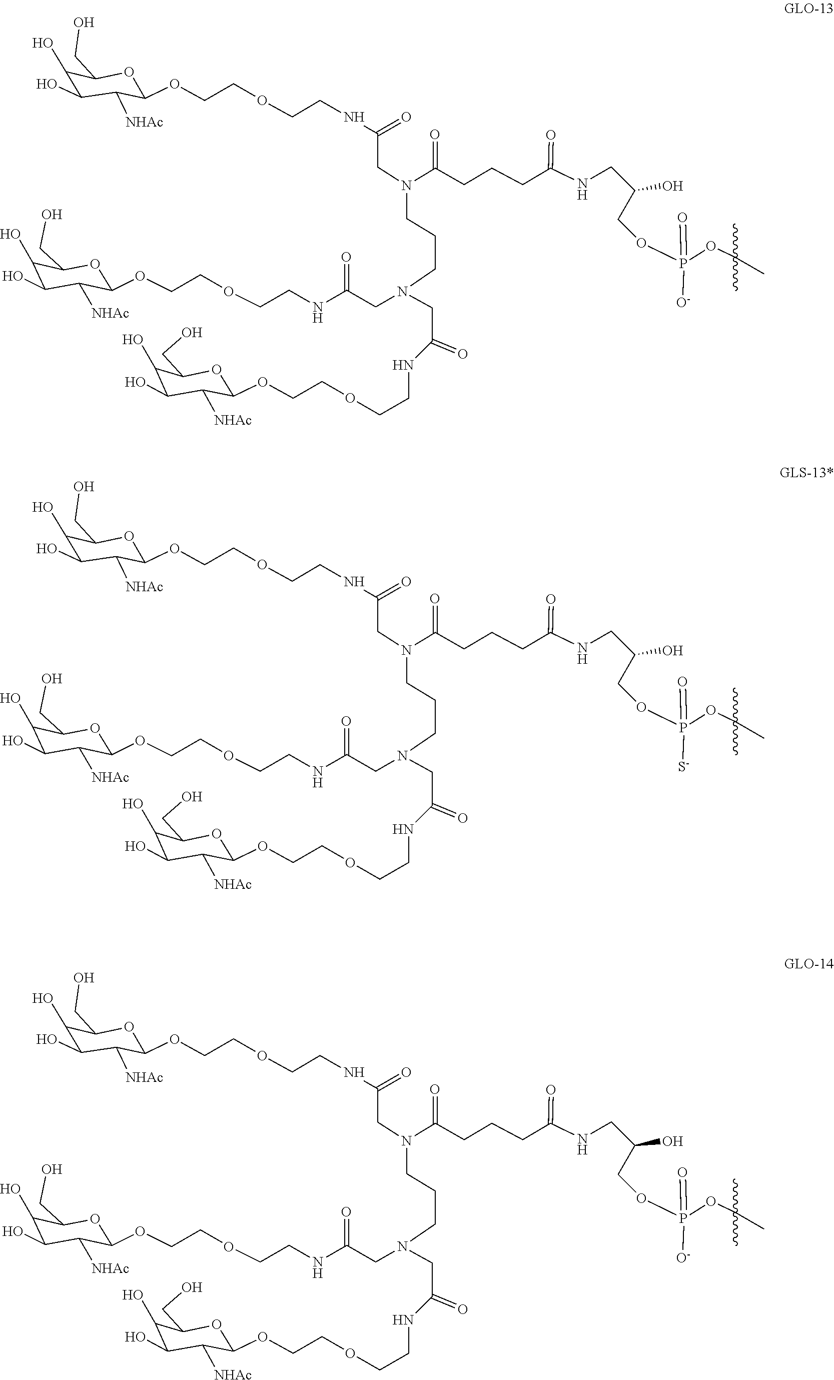 [SMILES: *OP(=O)([O-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

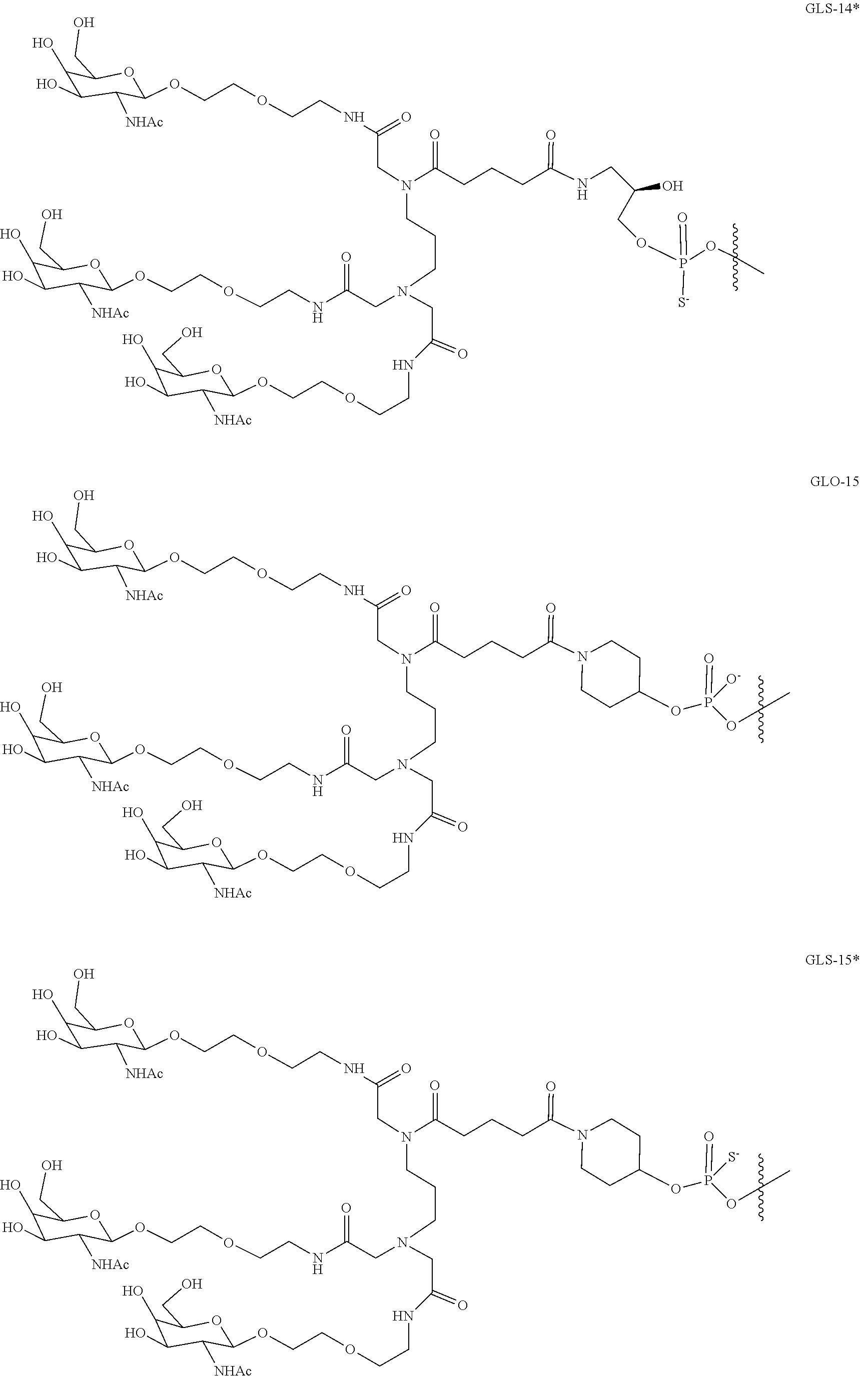 [SMILES: *OP(=O)([S-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1 ; *OP(=O)([S-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1]

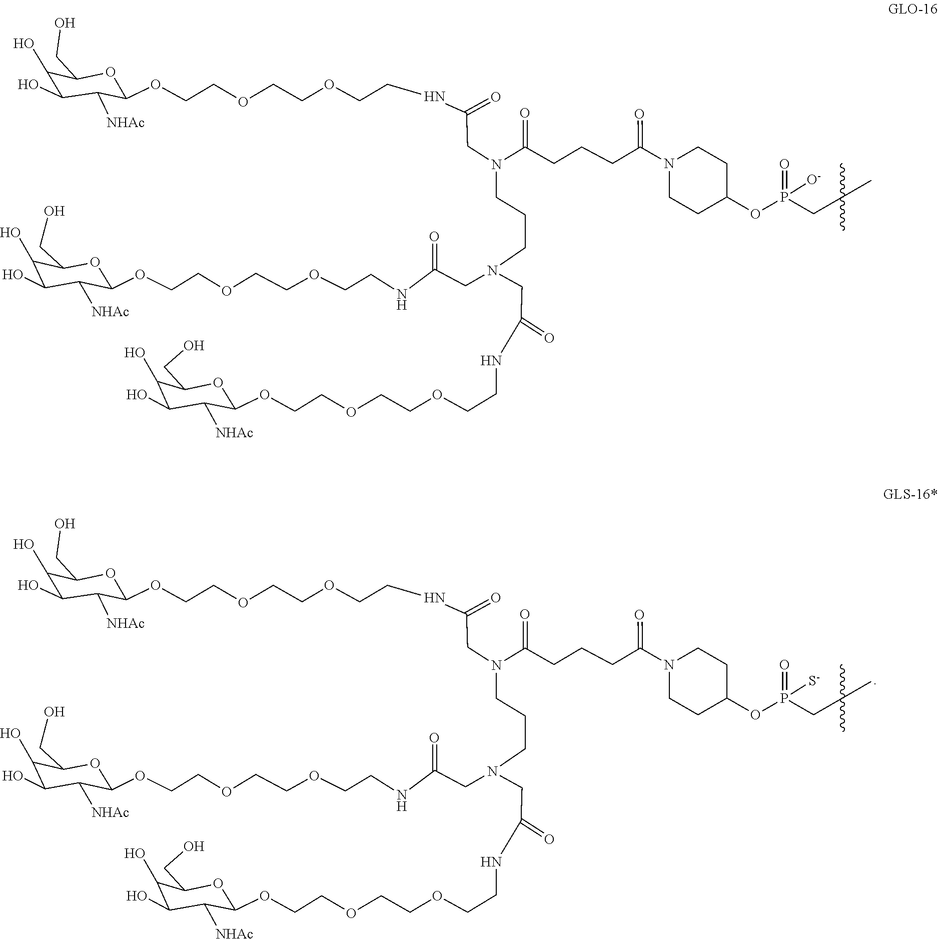 [SMILES: *CP(=O)([O-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1 ; *CP(=O)([S-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1]

In certain embodiments, the dsRNA agent includes a targeting group that is conjugated to the 5′-terminal end of the sense strand. In some embodiments, the dsRNA agent includes a targeting group that is conjugated to the 3′-terminal end of the sense strand.

In some embodiments, the antisense strand includes one inverted abasic residue at 3′-terminal end. In certain embodiments, the sense strand includes one or two inverted abasic residues and/or one or two imann residues at 3′ or/and 5′ terminal end. In certain embodiments, each end of the sense strand includes one inverted abasic residue. In certain embodiments, each end of the sense strand includes one imann residue.

In some embodiments, the dsRNA agent has two blunt ends. In some embodiments, at least one strand includes a 3′ overhang of at least 1 nucleotide. In some embodiments, at least one strand includes a 3′ overhang of at least 2 nucleotides.

In certain embodiments, the dsRNA comprises a duplex selected from the group consisting of AV01124.um, AV01126.um, AV01138.um, AV01141.um, AV01152.um, AV01180.um, AV01204.um, AV01124-19-Lum, AV01126-19-Lum, AV01138-19-1.um, AV01141-19-1.um, AV01152-19-1.um, AV01180-19-1.um, AV01204-19-1.um. In certain embodiments, the dsRNA comprises a duplex selected from the group consisting of AV01124, AV01126, AV01138, AV01141, AV01152, AV01180, AV01204. In certain embodiments, the dsRNA comprises a duplex selected from the group consisting of AD00990, AD00991, AD00997, AD00768, AD00999, AD01002, AD00773, AD00998, AD00776, AD01018, AD00990-1, AD00991-1, AD00997-1, AD00998-1, AD00999-1, AD01002-1, AD01016-1, AD00990-2, AD00997-2, AD02352, AD02362, AD02375-1, AD02399, AD02444, AD02445, AD02446, AD02447.

According to another aspect of the invention, a double-stranded ribonucleic acid (dsRNA) agent for inhibiting expression of coagulation factor XI (FXI) is provided, wherein the dsRNA agent including a sense strand and an antisense strand, wherein the sense strand is complementary to the antisense strand, wherein the antisense strand comprises a region complementary to a FXI RNA transcript, wherein each strand is about 15 to about 30 nucleotides in length, wherein the sense strand sequence may be represented by formula (II):

5′-(N′_{L})ₙ′N′_{L}N′_{L}N′_{L}N′_{L}N′_{F}N′L N′_{F}N′_{L}N′_{N1}N′_{N2}N′_{L}N′_{L}N′_{L}N′_{L}N′_{L}(N′_{L})_{m′}-3′ formula (II)
wherein:
each N′_{F} represents a 2′-fluoro-modified nucleotide; each N′_{N1} and N′_{N2} independently represents a modified or unmodified nucleotide, N′_{N1} and N′_{N2} include only one 2′-Fluorine modified nucleotides; each N′_{L} independently represents a modified or unmodified nucleotide but not a 2′-fluoro-modified nucleotide, and m′ and n′ are each independently an integer of 0 to 7.

In some embodiments, N′_{N1} independently represents a 2′-fluoro-modified nucleotide.

In some embodiments, N′_{N2} independently represents a 2′-fluoro-modified nucleotide.

In some embodiments, m′ is 2 and n′ is 4, or m′ is 2 and n′ is 2. In some embodiments, m′ is 1 and n′ is 4, or m′ is 1 and n′ is 2. In some embodiments, m′ is 0 and n′ is 4, or m′ is 0 and n′ is 2.

In some embodiments, the dsRNA agent includes a targeting group that is conjugated to the 5′-terminal end of the sense strand, preferably, the targeting group is any one selected from aforesaid GLO-1 through GLO-16 and GLS-1* through GLS-16*, more preferably, the targeting group is aforesaid GLS-15*. In certain embodiments, the dsRNA agent includes a targeting group that is conjugated to the 3′-terminal end of the sense strand. In certain embodiments, the antisense strand includes one inverted abasic residue at 3′-terminal end. In certain embodiments, the sense strand includes one or two inverted abasic residues and/or one or two imann residues at 3′ or/and 5′ terminal end. In certain embodiments, each 3′ and 5′ terminal end of the sense strand independently includes an inverted abasic residue. In certain embodiments, each 3′ and 5′ terminal end of the sense strand independently includes an imann residue. In certain embodiments, the sense strand includes two inverted abasic residues at 3′ and 5′ terminal end and either residue at 3′ or 5′ terminal end is further conjugated to a targeting group, which preferably is aforesaid GLS-15*. In certain embodiments, the sense strand includes two imann residues at 3′ and 5′ terminal end and either residue at 3′ or 5′ terminal end is further conjugated to a targeting group, which preferably is aforesaid GLS-15*.

According to another aspect of the invention, a double-stranded ribonucleic acid (dsRNA) agent for inhibiting expression of coagulation factor XI (FXI) is provided, wherein the dsRNA agent including a sense strand and an antisense strand, wherein the sense strand is complementary to the antisense strand, wherein the antisense strand comprises a region complementary to a FXI RNA transcript, wherein each strand is about 18 to about 30 nucleotides in length, wherein the antisense strand sequence may be represented by formula (III):

3′-(N_{L})ₙ N_{M1}N_{L} N_{M2}N_{L} N_{F} N_{L} N_{M3}N_{L} N_{L} N_{L} N_{L} N_{M4}N_{L} N_{M5}N_{L} N_{L} N_{F} N_{L}-5′ formula (III)
wherein:
each N_{F} represents a 2′-fluoro-modified nucleotide; each N_{M1}, N_{M2}, N_{M3}, N_{M4}, and N_{M5} independently represents a modified or unmodified nucleotide; each N_{L} independently represents a modified or unmodified nucleotide but not a 2′-fluoro-modified nucleotide, and n is an integer of 0 to 7.

In some embodiments, N_{M2}, N_{M3} and N_{M4} each independently represents a 2′-fluoro-modified nucleotide.

In some embodiments, N_{M1}, N_{M3} and N_{M5} each independently represents a 2′-fluoro-modified nucleotide.

In some embodiments, N_{M1}, N_{M2}, N_{M3}, N_{M4}, and N_{M5} have only three 2′-fluoro-modified nucleotides.

In some embodiments, N_{M1}, N_{M3} and N_{M5} each independently represents a 2′-fluoro-modified nucleotide and N_{M4} represents an UNA modified nucleotide.

In some embodiments, n is 1, or n is 2, or n is 3.

According to another aspect of the invention, a double-stranded ribonucleic acid (dsRNA) agent for inhibiting expression of coagulation factor XI (FXI) is provided, wherein the dsRNA agent including a sense strand and an antisense strand, wherein the sense strand and the antisense strand form a dsRNA duplex, wherein said sense strand is complementary to the antisense strand, wherein said antisense strand comprises a region of complementarity to a FXI RNA transcript, wherein the region of complementarity comprises at least 15 contiguous nucleotides, wherein the dsRNA duplex represented by formula (IV):

sense:5′-(N′_{L})_{n′}N′_{L} N′_{L} N′_{L} N′_{L} N′_{F}N′_{L} N′_{F}N′_{L} N′_{N1}N′_{N2}N′_{L} N′_{L} N′_{L} N′_{L} N′_{L}(N′_{L})_{m′}-3

antisense:3′-(N_{L})ₙ N_{M1}N_{L} N_{M2}N_{L} N_{F}N_{L} N_{M3}N_{L} N_{L} N_{L} N_{L} N_{M4}N_{L} N_{M5}N_{L} N_{L} N_{F}N_{L}-5′ formula (IV)
wherein:
each strand is about 18 to about 30 nucleotides in length;
each N_{F} and N′_{F} independently represents a 2′-fluoro-modified nucleotide; N_{M1}, N_{M2}, N_{M3}, N_{M4}, N_{M5}, N′_{N1}, and N′_{N2} each independently represents a modified or unmodified nucleotide; N′_{N1} and N′_{N2} include only one 2′-Fluorine modified nucleotides; each N_{L} and N′_{L} independently represents a modified or unmodified nucleotide but not a 2′-fluoro-modified nucleotide, and m′, n′ and n are each independently an integer of 0 to 7.

In some embodiments, N′_{N1} independently represents a 2′-fluoro-modified nucleotide.

In some embodiments, N′_{N2} independently represents a 2′-fluoro-modified nucleotide.

In some embodiments, m′ is 2 and n′ is 4, or m′ is 2 and n′ is 2. In some embodiments, m′ is 1 and n′ is 4, or m′ is 1 and n′ is 2.

In some embodiments, N_{M2}, N_{M3} and N_{M4} each independently represents a 2′-fluoro-modified nucleotide.

In some embodiments, N_{M1}, N_{M3} and N_{M5} each independently represents a 2′-fluoro-modified nucleotide.

In some embodiments, N_{M1}, N_{M2}, N_{M3}, N_{M4}, and N_{M5} have only three 2′-fluoro-modified nucleotides.

In some embodiments, N_{M1}, N_{M3} and N_{M5} each independently represents a 2′-fluoro-modified nucleotide and N_{M4} represents an UNA modified nucleotide.

In some embodiments, the dsRNA agent includes a targeting group that is conjugated to the 5′-terminal end of the sense strand, preferably, the targeting group is any one selected from aforesaid GLO-1 through GLO-16 and GLS-1* through GLS-16*, more preferably, the targeting group is aforesaid GLS-15*. In certain embodiments, the dsRNA agent includes a targeting group that is conjugated to the 5′-terminal end of the sense strand. In certain embodiments, the antisense strand includes one inverted abasic residue at 3′-terminal end. In certain embodiments, the sense strand includes one or two inverted abasic residues and/or one or two imann residues at 3′ or/and 5′ terminal end. In certain embodiments, each 3′ and 5′ terminal end of the sense strand independently includes an inverted abasic residue. In certain embodiments, each 3′ and 5′ terminal end of the sense strand independently includes an imann residue. In certain embodiments, the sense strand includes two inverted abasic residues at 3′ and 5′ terminal end and either residue at 3′ or 5′ terminal end is further conjugated to a targeting group, which preferably is aforesaid GLS-15. In certain embodiments, the sense strand includes two imann residues at 3′ and 5′ terminal end and either residue at 3′ or 5′ terminal end is further conjugated to a targeting group, which preferably is aforesaid GLS-15*. In certain embodiments, the dsRNA agent has two blunt ends. In certain embodiments, at least one strand includes a 3′ overhang of at least 1 nucleotide. In certain embodiments, at least one strand includes a 3′ overhang of at least 2 nucleotides.

According to an aspect of the invention, a composition is provided that includes any embodiment of the aforementioned dsRNA agent aspect of the invention. In certain embodiments, the composition also includes a pharmaceutically acceptable carrier. In some embodiments, the composition also includes one or more additional therapeutic agents. In certain embodiments, the composition is packaged in a kit, container, pack, dispenser, pre-filled syringe, or vial. In some embodiments, the composition is formulated for subcutaneous administration or is formulated for intravenous (IV) administration.

According to another aspect of the invention a cell is provided that includes any embodiment of an aforementioned dsRNA agent aspect of the invention. In some embodiments, the cell is a mammalian cell, optionally a human cell.

According to another aspect of the invention, a method of inhibiting the expression of a FXI gene in a cell, is provided, the method including: (i) preparing a cell including an effective amount of any embodiment of the aforementioned dsRNA agent aspect of the invention or any embodiment of an aforementioned composition of the invention. In certain embodiments, the method also includes: (ii) maintaining the prepared cell for a time sufficient to obtain degradation of the mRNA transcript of a FXI gene, thereby inhibiting expression of the FXI gene in the cell. In some embodiments, the cell is in a subject and the dsRNA agent is administered to the subject subcutaneously. In some embodiments, the cell is in a subject and the dsRNA agent is administered to the subject by IV administration. In certain embodiments, the method also includes assessing inhibition of the FXI gene, following the administration of the dsRNA agent to the subject, wherein a means for the assessing comprises: (i) determining one or more physiological characteristics of a FXI-associated disease or condition in the subject and (ii) comparing the determined physiological characteristic(s) to a baseline pre-treatment physiological characteristic of the FXI-associated disease or condition and/or to a control physiological characteristic of the FXI-associated disease or condition, wherein the comparison indicates one or more of a presence or absence of inhibition of expression of the FXI gene in the subject. In some embodiments, the physiological characteristic is one or more of: the FXI mRNA level and the FXI protein level. A reduction in the expression of FXI may also be assessed indirectly by measuring a decrease in biological activity of FXI, e.g., In some embodiments, the FXI protein may be an active form (FXIa). The active form of FXI may promote converting FIX from its inactive form (FIX) to its active form (FIXa). FXI activity may be assessed with a blood test that characterizes coagulation of blood. Non-limiting examples of such blood tests are a partial thromboplastin time (PTT) test or activated partial thromboplastin time test (aPTT or APTT). FXI activity in a test plasma sample may be assayed by adding the test plasma sample to a plasma sample that is immunodepleted of FXI and comparing clotting of the resulting combined sample to a reference sample with a reference amount of FXI.

According to another aspect of the invention, a method of treating a disease or condition associated with the presence of FXI protein is provided, the method including: administering to a subject an effective amount of an embodiment of any aforementioned dsRNA agent aspect of the invention or an embodiment of any aforementioned composition of the invention, to inhibit FXI gene expression. In some embodiments, the disease, disorder or condition associated with FXI is thromboembolic condition. In certain embodiments, the thromboembolic condition is deep vein thrombosis, venous or arterial thrombosis, pulmonary embolism, myocardial infarction, stroke, thrombosis associated with chronic kidney disease or end-stage renal disease (ESRD), including thrombosis associated with dialysis, or other procoagulant condition. Also provided are methods useful treating, preventing, or ameliorating a thromboembolic condition without increasing bleeding risk in an individual. In certain embodiments, the individual is at risk for a thromboembolic condition, including, but not limited to infarct, thrombosis, embolism, thromboembolism such as deep vein thrombosis, pulmonary embolism, myocardial infarction, and stroke. Such diseases, disorders, and conditions can have one or more risk factors, causes, or outcomes in common. Certain risk factors and causes for development of a thromboembolic condition include immobility, surgery (particularly orthopedic surgery), dialysis, malignancy, pregnancy, older age, use of oral contraceptives, atrial fibrillation, previous thromboembolic condition, chronic inflammatory disease, inherited or acquired prothrombotic clotting disorders and thrombosis associated with chronic kidney disease or end-stage renal disease (ESRD). Certain outcomes associated with development of athromboembolic condition include decreased blood flow through an affected vessel, death of tissue, and death.

In some embodiments, the method also includes: administering an additional therapeutic regimen to the subject. In some embodiments, the additional therapeutic regimen includes a treatment for the FXI-associated disease or condition. In certain embodiments, methods comprise co-administering one or more pharmaceutical compositions described herein with one or more other pharmaceutical agents at the same time. In certain embodiments, methods comprise co-administering one or more pharmaceutical compositions described herein with one or more other pharmaceutical agents at different times. In certain embodiments, one or more pharmaceutical compositions described herein and one or more other pharmaceutical agents are prepared together in a single formulation. In certain embodiments, one or more pharmaceutical compositions described herein and one or more other pharmaceutical agents are prepared separately. In certain embodiments, pharmaceutical agents that may be co-administered with a pharmaceutical composition described herein include anticoagulant or antiplatelet agents. In certain embodiments, pharmaceutical agents that may be co-administered with a pharmaceutical composition described herein include NSAID/Cyclooxygenase inhibitors, such as, aspirin. In certain embodiments, pharmaceutical agents that may be co-administered with a pharmaceutical composition described herein include adenosine diphosphate (ADP) receptor inhibitors, such as, clopidogrel (PLAVIX) and ticlopidine (TICLID). In certain embodiments, pharmaceutical agents that may be co-administered with a pharmaceutical composition described herein include phosphodiesterase inhibitors, such as, cilostazol (PLETAL). In certain embodiments, pharmaceutical agents that may be co-administered with a pharmaceutical composition described herein include, glycoprotein IIB/IIIA inhibitors, such as, abciximab (REOPRO), eptifibatide (INTEGRILIN), tirofiban (AGGRASTAT), and defibrotide. In certain embodiments, pharmaceutical agents that may be co-administered with a pharmaceutical composition described herein include, adenosine reuptake inhibitors, such as, dipyridamole (PERSANTINE). In certain embodiments, pharmaceutical agents that may be co-administered with a pharmaceutical composition described herein include, but are not limited to, warfarin (and related coumarins), heparin, direct thrombin inhibitors (such as lepirudin, bivalirudin), apixaban, LOVENOX (enoxaparin), and small molecular compounds that interfere directly with the enzymatic action of particular coagulation factors (e.g. rivaroxaban, which interferes with Factor Xa). In certain embodiments, the anticoagulant or antiplatelet agent is administered prior to administration of a pharmaceutical composition described herein. In certain embodiments, the anticoagulant or antiplatelet agent is administered following administration of a pharmaceutical composition described herein. In certain embodiments the anticoagulant or antiplatelet agent is administered at the same time as a pharmaceutical composition described herein. In certain embodiments, the dosage unit of a co-administered anticoagulant or antiplatelet agent is the same as the dosage unit that would be administered if the anticoagulant or antiplateletagent was administered alone. In certain embodiments the dosage unit of a co-administered anticoagulant or antiplatelet agent is lower than the dosage unit that would be administered if the anticoagulant or antiplatelet agent was administered alone. In certain embodiments the dosage unit of a co-administered anticoagulant or antiplatelet agent is greater than the dosage unit that would be administered if the anticoagulant or antiplatelet agent was administered alone. In certain embodiments, methods may comprise co-administering a pharmaceutical composition described herein with an anti-inflammatory agent. In certain embodiments, the anti-inflammatory agent modifies a symptom of the inflammatory disease or condition. In certain embodiments, the anti-inflammatory agent modifies progression of the inflammatory disease or condition. Non-limiting examples of inflammatory disesases and conditions are autoimmune diseases (e.g. arthritis, colitis or diabetes), trauma, surgery, sepsis, allergic inflammation, and asthma. Non-limiting examples of anti-inflammatory agents include, but are not limited to, methotrexate, abatacept, infliximab, cyclophosphamide, azathioprine, corticosteroids, cyclosporin A, aminosalicylates, sulfasalazine, hydroxychloroquine, leflunomide, etanercept, efalizumab, 6-mercapto-purine (6-MP), and tumor necrosis factor-alpha (TNFalpha), and other cytokine blockers or antagonists. In certain embodiments, the anti-inflammatory agent comprises a non-steroidal anti-inflammatory drug (NSAID). In certain embodiments, NSAIDs reduce inflammatory reactions in an animal. NSAIDS include, but are not limited to, acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, meloxicam and tramadol. In certain embodiments, methods comprise administering a pharmaceutical composition disclosed herein and an anti-inflammatory agent concomitantly or sequentially. In certain embodiments, methods comprise administering the anti inflammatory agent prior to administering the pharmaceutical composition described herein. In certain embodiments, methods comprise administering the anti-inflammatory agent after administering a pharmaceutical composition described herein. In certain embodiments, methods comprise administering the anti-inflammatory agent and a pharmaceutical composition described herein at the same time. In certain embodiments, the dosage unit of a co-administered anti-inflammatory agent is the same as the dosage unit that would be administered if the anti-inflammatory agent was administered alone. In certain embodiments the dosage unit of a co-administered anti-inflammatory agent is lower than the dosage unit that would be administered if the anti-inflammatory agent was administered alone. In certain embodiments the dosage unit of a co-administered anti-inflammatory agent is greater than the dosage unit that would be administered if the anticoagulant or antiplatelet agent was administered alone. In certain embodiments, the co-administration of a second pharmaceutical agent enhances the anticoagulant or anti-inflammatory effect of a pharmaceutical composition described herein, such that co administration of the two results in an anticoagulant or anti-inflammatory effect that is greater than the effect of administering the pharmaceutical composition described herein. In certain embodiments, the co-administration results in anticoagulant or anti-inflammatory effects that are additive of the effects of the two when administered alone. In certain embodiments, the co-administration results in anticoagulant or anti inflammatory effects that are supra-additive of the effects of the two when administered alone. In certain embodiments, co-administration of a second pharmaceutical agent increases an antithrombotic activity or anti-inflammatory activity of the pharmaceutical composition relative to that provided by the pharmaceutical composition alone, without increased bleeding risk. In certain embodiments, methods comprise co-administering a pharmaceutical composition described herein and an antiplatelet therapy to an animal in need thereof. In certain embodiments, the animal in need thereof may be an animal having a condition selected from thromboembolism, atrial fibrillation, a heart valve disorder, valvular heart disease, stroke, coronary artery disease (CAD), and a mechanical heart valve, and a combination thereof. In certain embodiments, administering a pharmaceutical composition described herein in combination with an antiplatelet therapy results in little to no detectable increase in risk of bleeding as compared to antiplatelet therapy alone. In certain embodiments, the risk profile or risk indications are unchanged over antiplatelet therapy alone. In certain embodiments, methods comprise administering a pharmaceutical composition described herein to a dialysis patient, including but not limited to an animal with end stage renal disease (ESRD) or chronic kidney disease (CKD), wherein the animal is co-administered at least one pharmaceutical agent selected from heparin, erythropoietin, darbopoetin, iron, Vitamin D or analogues thereof, phosphate binders, and a combination thereof. In certain embodiments, the animal receives dialysis. In certain embodiments, the pharmaceutical agent is administered at a dose that does not differ from a comparative dose that would be prescribed in the absence of administering the pharmaceutical composition.

In some embodiments, the dsRNA agent is administered to the subject subcutaneously. In certain embodiments, the dsRNA agent is administered to the subject by IV administration. In some embodiments, the method also includes determining an efficacy of the administered double-stranded ribonucleic acid (dsRNA) agent in the subject. In some embodiments, a means of determining an efficacy of the treatment in the subject comprises: (i) determining one or more physiological characteristics of the FXI-associated disease or condition in the subject and (ii) comparing the determined physiological characteristic(s) to a baseline pre-treatment physiological characteristic of the FXI-associated disease or condition wherein the comparison indicates one or more of a presence, absence, and level of efficacy of the administration of the double-stranded ribonucleic acid (dsRNA) agent to the subject. In some embodiments, expression of the FXI gene can be assessed based on the level or change in level of any variable associated with FXI gene expression, such as FXI mRNA level, FXI protein level in the subject, or. the FXI protein may be an active form (FXIa). FXI activity may be assessed with a blood test that characterizes coagulation of blood. blood tests are a partial thromboplastin time (PTT) test or activated partial thromboplastin time test (aPTT or APTT).

According to another aspect of the invention, a method of decreasing a level of FXI protein in a subject compared to a baseline pre-treatment level of FXI protein in the subject, is provided, the method including administering to the subject an effective amount of an embodiment of any aforementioned dsRNA agent of the invention or an embodiment of any aforementioned composition of the invention, to decrease the level of FXI gene expression. In some embodiments, the dsRNA agent is administered to the subject subcutaneously or is administered to the subject by IV administration.

According to another aspect of the invention, a method of altering a physiological characteristic of a FXI-associated disease or condition in a subject compared to a baseline pre-treatment physiological characteristic of the FXI-associated disease or condition in the subject is provided, the method including administering to the subject an effective amount of an embodiment of any aforementioned dsRNA agent of the invention or an embodiment of any aforementioned composition of the invention, to alter the physiological characteristic of the FXI-associated disease or condition in the subject. In some embodiments, the dsRNA agent is administered to the subject subcutaneously or is administered to the subject by IV administration. In certain embodiments, the physiological characteristic is one or more of: FXI mRNA level, FXI protein level in the subject, or FXI activity. The FXI activity may cause a reduction in blood clotting activity in the animal. Blood clotting may be measured by a standard test, for example, but not limited to, activated partial thromboplastin time (aPTT) test, prothrombin time (PT) test, thrombin time (TCT), bleeding time, or presence of D-dimer in a blood sample of the animal.

According to another aspect of the invention, an antisense polynucleotide agent for inhibiting expression of FXI protein is provided, the agent including from 10 to 30 contiguous nucleotides, wherein at least one of the contiguous nucleotides is a modified nucleotide, and wherein the nucleotide sequence of the agent is about 80% complementary over its entire length to the equivalent region of the nucleotide sequence of SEQ ID NO: 1. In some embodiments, the equivalent region is any one of the target regions of SEQ ID NO: 1 and the complementary sequence is one provided in one of Tables 1-3. In certain embodiments, the antisense polynucleotide agent includes one of the antisense sequences provided in one of Tables 1-3.

According to another aspect of the invention, a composition including an embodiment of any aforementioned antisense polynucleotide agents is provided. In some embodiments, the composition also includes a pharmaceutically acceptable carrier. In some embodiments, the composition also includes one or more additional therapeutic agents for treatment of a FXI-associated disease or condition. In certain embodiments, the composition is packaged in a kit, container, pack, dispenser, pre-filled syringe, or vial. In certain embodiments, the composition is formulated for subcutaneous or IV administration.

According to another aspect of the invention a cell that includes an embodiment of any of the aforementioned antisense polynucleotide agents is provided. In some embodiments, the cell is a mammalian cell, optionally a human cell.

According to another aspect of the invention, a method of inhibiting the expression of a FXI gene in a cell is provided, the method including: (i) preparing a cell including an effective amount of an embodiment of any aforementioned antisense polynucleotide agents. In some embodiments, the method also includes (ii) maintaining the cell prepared in (i) for a time sufficient to obtain degradation of the mRNA transcript of a FXI gene, thereby inhibiting expression of the FXI gene in the cell.

According to another aspect of the invention, a method of inhibiting expression of a FXI gene in a subject is provided, the method including administering to the subject an effective amount of an embodiment of any of the aforementioned antisense polynucleotide agent.

According to another aspect of the invention, a method of treating a disease or condition associated with the presence of FXI protein, the method including administering to a subject an effective amount of an embodiment of any of the aforementioned antisense polynucleotide agents or an embodiment of any aforementioned composition of the invention, to inhibit FXI gene expression. In certain embodiments, the disease or condition is thromboembolic condition. In certain embodiments, the thromboembolic condition is deep vein thrombosis, venous or arterial thrombosis, pulmonary embolism, myocardial infarction, stroke, thrombosis associated with chronic kidney disease or end-stage renal disease (ESRD), including thrombosis associated with dialysis, or other procoagulant condition. Also provided are methods useful treating, preventing, or ameliorating a thromboembolic condition without increasing bleeding risk in an individual. In certain embodiments, the individual is at risk for a thromboembolic condition, including, but not limited to infarct, thrombosis, embolism, thromboembolism such as deep vein thrombosis, pulmonary embolism, myocardial infarction, and stroke. Such diseases, disorders, and conditions can have one or more risk factors, causes, or outcomes in common. Certain risk factors and causes for development of a thromboembolic condition include immobility, surgery (particularly orthopedic surgery), dialysis, malignancy, pregnancy, older age, use of oral contraceptives, atrial fibrillation, previous thromboembolic condition, chronic inflammatory disease, inherited or acquired prothrombotic clotting disorders and thrombosis associated with chronic kidney disease or end-stage renal disease (ESRD). Certain outcomes associated with development of a thromboembolic condition include decreased blood flow through an affected vessel, death of tissue, and death.

According to another aspect of the invention, a method of decreasing a level of FXI protein in a subject compared to a baseline pre-treatment level of FXI protein in the subject is provided, the method including administering to the subject an effective amount of an embodiment of any of the aforementioned antisense polynucleotide agents or an embodiment of any aforementioned composition of the invention, to decrease the level of FXI gene expression. In certain embodiments, the antisense polynucleotide agent is administered to the subject subcutaneously or by IV administration.

### BRIEF DESCRIPTION OF THE SEQUENCES

SEQ ID NO: 1 and SEQ ID NO: 2 (reverse complement) are Homo sapiens coagulation factor XI mRNA [NCBI Reference Sequence: NM_000128.4].

SEQ ID NO: 3 and SEQ ID NO: 4 (reverse complement) are Mus musculus (house mouse) coagulation factor XI mRNA [NCBI Reference Sequence: NM_001411665.1].

SEQ ID NOs: 5-198, 715-1000 are shown in Table 1 and are sense strand sequences.

SEQ ID NOs: 199-392, 1001-1286 are shown in Table 1 and are antisense strand sequences.

SEQ ID NOs: 393-586, 1287-1568 are shown in Table 2 with chemical modifications.

SEQ ID NOs: 587-714, 1567-1728 are shown in Table 3. A delivery molecule is indicated as “GLX-” at the 3′ end or 5′ end of each sense strand.

### DETAILED DESCRIPTION

The invention in part, includes RNAi agents, for example, though not limited to double stranded (ds) RNAi agents, which are capable of inhibiting coagulation factor XI (FXI) gene expression. The invention, in part also includes compositions comprising FXI RNAi agents and methods of use of the compositions. FXI RNAi agents disclosed herein may be attached to delivery compounds for delivery to cells, including to hepatocytes. Pharmaceutical compositions of the invention may include at least one dsRNA FXI agent and a delivery compound. In some embodiments of compositions and methods of the invention, the delivery compound is a GalNAc-containing delivery compound. FXI RNAi agents delivered to cells are capable of inhibiting FXI gene expression, thereby reducing activity in the cell of the FXI protein product of the gene. dsRNAi agents of the invention can be used to treat FXI-associated diseases and conditions.

In some embodiments of the invention reducing FXI expression in a cell or subject treats a disease or condition associated with FXI expression in the cell or subject, respectively. Non-limiting examples of diseases and conditions that may be treated by reducing FXI activity are: thromboembolic condition, such as deep vein thrombosis, venous or arterial thrombosis, pulmonary embolism, myocardial infarction, stroke, thrombosis associated with chronic kidney disease or end-stage renal disease (ESRD), including thrombosis associated with dialysis, or other procoagulant condition, or other diseases for which reducing a level and activity of FXI protein is medically beneficial.

As used herein, “G,” “C,” “A” and “U” each generally stand for a nucleotide that contains guanine, cytosine, adenine, and uracil as a base, respectively. However, it will be understood that the term “ribonucleotide” or “nucleotide” can also refer to a modified nucleotide, as further detailed below, or a surrogate replacement moiety. The skilled person understands that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the invention by a nucleotide containing, for example, inosine. Sequences comprising such replacement moieties are embodiments of the invention.

As used herein, “coagulation factor XI gene expression” used interchangeably with the term “FXI” refers to the naturally occurring gene that encodes a cell inducing coagulation factor XI protein from any vertebrate or mammalian source, including, but not limited to, human, bovine, chicken, rodent, mouse, rat, porcine, ovine, primate, monkey, and guinea pig, unless specified otherwise. The term also refers to fragments and variants of native FXI that maintain at least one in vivo or in vitro activity of a native FXI. As used herein, the term “inhibiting coagulation factor XI gene expression” includes inhibiting the expression of the FXI gene as well as variants (e. g., naturally occurring variants) or mutants of the factor XI gene, inhibiting the expression of FXI mRNA, inhibiting the expression of FXI RNA transcript, and/or inhibiting the expression of FXI protein. The FXI gene may be a wild-type human FXI gene, a mutant human FXI gene, or a transgenic human FXI gene in the case of genetically manipulated cells, cell groups, or organisms. The amino acid and complete coding sequences of the reference sequence of the human FXI gene may be found in, for example, GenBank Ref Seq Accession No. NM_000128.4 (SEQ ID NO: 1 and SEQ ID NO:2). Mus musculus (house mouse) coagulation FXI mRNA GenBank Ref Seq Accession No. NM_001411665.1 (SEQ ID NO:3 and SEQ ID NO:4); Additional examples of FXI mRNA sequences are readily available using publicly available databases, e.g., GenBank, UniProt, Ensembl and OMIM, for example, NM_001354804.2 (coagulation factor XI isoform 3 precursor).

“Factor XI”, “FXI”, “Factor 11” and “F11” is used interchangeably herein. “Factor XI nucleic acid” or “Factor XI nucleic acid” means any nucleic acid encoding FXI. For example, in certain embodiments, a Factor XI nucleic acid includes a DNA sequence encoding Factor XI, an RNA sequence transcribed from DNA encoding Factor XI (including genomic DNA comprising introns and exons), and an mRNA sequence encoding Factor XI. “Factor XI mRNA” means an mRNA encoding a Factor XI protein.

“FXI specific inhibitor” refers to any agent capable of specifically inhibiting the expression of FXI mRNA and/or FXI protein at the molecular level. For example, FXI specific inhibitors include nucleic acids (including antisense compounds), peptides, antibodies, small molecules, and other agents capable of inhibiting the expression of FXI mRNA and/or FXI protein. In certain embodiments, by specifically modulating FXI mRNA level and/or FXI protein expression, FXI specific inhibitors may affect components of the inflammatory pathway. Similarly, in certain embodiments, FXI specific inhibitors may affect other molecular processes in an animal.

The following describes how to make and use compositions comprising FXI single-stranded (ssRNA) and dsRNA agents to inhibit FXI gene expression, as well as compositions and methods for treating diseases and conditions caused by or modulated by FXI gene expression. The term “RNAi” is also known in the art, and may be referred to as “siRNA”.

As used herein, the term “RNAi” refers to an agent that comprises RNA and mediates targeted cleavage of an RNA transcript via an RNA-induced silencing complex (RISC) pathway. As is known in the art, an RNAi a target region refers to a contiguous portion of the nucleotide sequence of an mRNA molecule formed during the transcription of a gene, including messenger RNA (mRNA) that is a product of RNA processing of a primary transcription product. The target portion of the sequence will be at least long enough to serve as a substrate for RNAi-directed cleavage at or near that portion. A target sequence may be from 8-30 nucleotides long (inclusive), from 10-30 nucleotides long (inclusive), from 12-25 nucleotides long (inclusive), from 15-23 nucleotides long (inclusive), from 16-23 nucleotides long (inclusive), or from 18-23 nucleotides long (inclusive), including all shorter lengths within each stated range. In some embodiments of the invention, a target sequence is 9, 10, 11, 12, 13, 14, 15, 16, 17, 18, 19, 20, 21, 22, 23, 24, 25, or 26 nucleotides long. In certain embodiment a target sequence is between 9 and 26 nucleotides long (inclusive), including all sub-ranges and integers there between. For example, though not intended to be limiting, in certain embodiments of the invention a target sequence is 8, 9, 10, 11, 12, 13, 14, 15, 16, 17, 18, 19, 20, 21, 22, 23, 24, 25, 26, 27, 28, 29, or 30 nucleotides long, with the sequence fully or at least substantially complementary to at least part of an RNA transcript of a FXI gene. Some aspects of the invention include pharmaceutical compositions comprising one or more FXI dsRNA agents and a pharmaceutically acceptable carrier. In certain embodiments of the invention, a FXI RNAi as described herein inhibits expression of FXI protein.

As used herein, a “dsRNA agent” means a composition that contains an RNA or RNA-like (e.g., chemically modified RNA) oligonucleotide molecule that is capable of degrading or inhibiting translation of messenger RNA (mRNA) transcripts of a target mRNA in a sequence specific manner. Although not wishing to be limited to a particular theory, dsRNA agents of the invention may operate through the RNA interference mechanism (i.e., inducing RNA interference through interaction with the RNA interference pathway machinery (RNA-induced silencing complex or RISC) of mammalian cells), or by any alternative mechanism(s) or pathway(s). Methods for silencing genes in plant, invertebrate, and vertebrate cells are well known in the art [see, for example, (Sharp et al., Genes Dev. 2001, 15:485; Bernstein, et al., (2001) Nature 409:363; Nykanen, et al., (2001) Cell 107:309; and Elbashir, et al., (2001) Genes Dev. 15:188)], the disclosure of each of which is incorporated herein by reference in its entirety.]. Art-known gene silencing procedures can be used in conjunction with the disclosure provided herein to inhibit expression of FXI.

DsRNA agents disclosed herein are comprised of a sense strand and an antisense strand, and include, but are not limited to short interfering RNAs (siRNAs), RNAi agents, micro RNAs (miRNAs), short hairpin RNAs (shRNA), and dicer substrates. The antisense strand of the dsRNA agents described herein is at least partially complementary to the mRNA being targeted. It is understood in the art that different lengths of dsRNA duplex structure can be used to inhibit target gene expression. For example, dsRNAs having a duplex structure of 19, 20, 21, 22, and 23 base pairs are known to be effective to induce RNA interference (Elbashir et al., EMBO 2001, 20:6877-6888). It is also known in the art that shorter or longer RNA duplex structures are also effective to induce RNA interference. FXI dsRNAs in certain embodiments of the invention can include at least one strand of a length of minimally 21 nt or may have shorter duplexes based on one of the sequences set forth in any one of Tables 1-3, but minus 1, 2, 3, or 4 nucleotides on one or both ends may also be effective as compared to the dsRNAs set forth in Tables 1-3, respectively. In some embodiments of the invention, FXI dsRNA agents may have a partial sequence of at least 15, 16, 17, 18, 19, 20, or more contiguous nucleotides from one or more sequences of Tables 1-3, and differ in their ability to inhibit the expression of a FXI gene by not more than 5%, 10%, 15%, 20%, 25%, or 30% from the level of inhibition resulting from a dsRNA comprising the full sequence. A sense sequence, an antisense sequence and a duplex disclosed in Tables 1-3 may be referred to herein as a “parent” sequence, meaning that the sequences disclosed in Tables 1-3 may be modified, shorten, lengthened, include substitutions, etc. as set forth herein, with the resulting sequences retaining all or at least a portion of the efficacy of their parent sequences in methods and compositions of the invention. Sense and antisense strands included in a dsRNA of the invention are independently selected. As used herein the term “independently selected” means each of two or more like elements can be selected independent of the selection of the other elements. For example, though not intended to be limiting, in preparing a dsRNA of the invention, one may select the “elements” of the two strands to include in the duplex. One selected element, the sense sequence may be SEQ ID NO: 394 (shown in Table 2) and the other selected element, the antisense sequence, may be SEQ ID NO:491, or may be SEQ ID NO: 491 that is modified, shortened, lengthened, and/or includes 1, 2, or 3 substitutions as compared to its parent sequence SEQ ID NO: 491. It will be understood that a duplex of the invention need not include both sense and antisense sequences shown as paired in duplexes in Tables 1-3. Each sense and antisense strand sequence in the tables is immediately followed by its SEQ ID NO.

Certain embodiments of compositions and methods of the invention comprise a single-strand RNA in a composition and/or administered to a subject. For example, an antisense strand such as one listed in any one of Tables 1-3 may be a composition or in a composition administered to a subject to reduce FXI polypeptide activity and/or expression of FXI gene in the subject. Tables 1-3 show certain FXI dsRNA agent antisense strand and sense strand core stretch base sequences. A single-strand antisense molecule that may be included in certain compositions and/or administered in certain methods of the invention are referred to herein as a “single-strand antisense agent” or an “antisense polynucleotide agent”. A single-strand sense molecule that may be included in certain compositions and/or administered in certain methods of the invention are referred to herein as a “single-strand sense agent” or a “sense polynucleotide agent”. The term “base sequence” is used herein in reference to a polynucleotide sequence without chemical modifications or delivery compounds. For example, the sense strand GCAAGGUCUUUUCAGGAUGAA (SEQ ID NO: 6) shown in Table 1 is the base sequence for SEQ ID NO: 394 in Table 2 and for SEQ ID NO: 587 in Table 3, with SEQ ID NO: 394 and SEQ ID NO: 587 shown with their chemical modifications and a delivery compound. Sequences disclosed herein may be assigned identifiers. For example, a single-stranded sense sequence may be identified with a “Sense strand SS #”; a single stranded antisense sequence may be identified with an “Antisense strand AS #” and a duplex that includes a sense strand and an antisense strand may be identified with a “Duplex AD #/AV #”.

Table 1 includes sense and antisense strands and provides the identification number of duplexes formed from the sense and antisense strand on the same line in Table 1. In certain embodiments of the invention an antisense sequence includes nucleobase u or nucleobase a in position 1 of the antisense sequence. In certain embodiments of the invention an antisense sequence includes nucleobase u in position 1 of the antisense sequence. As used herein, the term “matching position” in a sense and an antisense strand are the positions in each strand that “pair” when the two strands are duplexed strands. For example, in a 21 nucleobases sense strand and a 21 nucleobases antisense strand, nucleobase in position 1 of the sense strand and position 21 in the antisense strand are in “matching positions”. In yet another non-limiting example in a 23 nucleobases sense strand and a 23 nucleobases antisense strand, nucleobase 2 of the sense strand and position 22 of the antisense strand are in matching positions. In another non-limiting example, in an 18 nucleobases sense strand and an 18 nucleobases antisense strand, nucleobase in position 1 of the sense strand and nucleobase 18 in the antisense strand are in matching positions, and nucleobase 4 in the sense strand and nucleobase 15 in the antisense strand are in matching positions. A skilled artisan will understand how to identify matching positions in sense and antisense strands that are or will be duplexed strands and paired strands.

The first column in Table 1 indicates a Duplex AV #for a duplex that includes the sense and antisense sequences in the same table row. For example, Table 1 discloses the duplex assigned Duplex AV #AV01118.um, which includes sense strand SEQ ID NO: 6 and antisense strand SEQ ID NO: 200. Thus, each row in Table 1 identifies a duplex of the invention, each comprising the sense and antisense sequences shown in the same row, with the assigned identifier for each duplex shown in the first column in the row.

In some embodiments of methods of the invention, an RNAi agent comprising a polynucleotide sequence shown in any one of Tables 1-3 is administered to a subject. In some embodiments of the invention an RNAi agent administered to a subject comprises is a duplex comprising at least one of the base sequences set forth in Table 1, including 0, 1, 2, 3, 4, 5, 6, 7, 8, 9, 10, 11, 12, 13, 14, 15, 16, 17, 18, 19, 20, 21, 22, 23, or 24 sequence modifications. In some embodiments of methods of the invention an RNAi agent comprising a polynucleotide sequence shown in any one of Tables 1-3 is attached to a delivery molecule, a non-limiting example of which is a delivery compound comprising a GalNAc compound, or a GLS-15* compound.

Table 1: Unmodified FXI RNAi agent antisense strand and sense strand sequences. All sequences shown 5′ to 3′ direction. Duplex AV #/AD #s are the number assigned to the duplex of the two strands in the same row in the table.

TABLE 1
Unmodified FXI RNAi agent antisense strand and sense strand sequences.
All sequences shown 5′ to 3′ direction.
Duplex AV#/AD#s are the number assigned to the duplex of the
two strands in the same row in the table.

| | | SEQ ID | | SEQ ID |
|---|---|---|---|---|
| Duplex AV# | Sense Strand | NO | Antisense Strand | NO |
| AV01117.um | GAACAAGGUCUUUUCAGGAUA | 5 | UAUCCUGAAAAGACCUUGUUC | 199 |
| AV01118.um | GCAAGGUCUUUUCAGGAUGAA | 6 | UUCAUCCUGAAAAGACCUUGC | 200 |
| AV01119.um | GAGGUCUUUUCAGGAUGAUUA | 7 | UAAUCAUCCUGAAAAGACCUC | 201 |
| AV01120.um | CUCUUUUCAGGAUGAUUUUCA | 8 | UGAAAAUCAUCCUGAAAAGAG | 202 |
| AV01121.um | GUUUCAGGAUGAUUUUCUUAA | 9 | UUAAGAAAAUCAUCCUGAAAC | 203 |
| AV01122.um | GAGGAUGAUUUUCUUAUAUCA | 10 | UGAUAUAAGAAAAUCAUCCUC | 204 |
| AV01123.um | CGAUGAUUUUCUUAUAUCAAA | 11 | UUUGAUAUAAGAAAAUCAUCG | 205 |
| AV01124.um | GCUUCAGUUUCUGGUGAAUGA | 12 | UCAUUCACCAGAAACUGAAGC | 206 |
| AV01125.um | GUCAGUUUCUGGUGAAUGUGA | 13 | UCACAUUCACCAGAAACUGAC | 207 |
| AV01126.um | GUUCUGGUGAAUGUGUGACUA | 14 | UAGUCACACAUUCACCAGAAC | 208 |
| AV01127.um | GUGGUUUACUUGUGUCCUGAA | 15 | UUCAGGACACAAGUAAACCAC | 209 |
| AV01128.um | GGGUUUACUUGUGUCCUGAAA | 16 | UUUCAGGACACAAGUAAACCC | 210 |
| AV01129.um | CAAAGACAGUGUUACAGAAAA | 17 | UUUUCUGUAACACUGUCUUUG | 211 |
| AV01130.um | GAAGACAGUGUUACAGAAACA | 18 | UGUUUCUGUAACACUGUCUUC | 212 |
| AV01131.um | GGACAGUGUUACAGAAACACA | 19 | UGUGUUUCUGUAACACUGUCC | 213 |
| AV01132.um | GUACAGAAACACUGCCAAGAA | 20 | UUCUUGGCAGUGUUUCUGUAC | 214 |
| AV01133.um | GACAGAAACACUGCCAAGAGA | 21 | UCUCUUGGCAGUGUUUCUGUC | 215 |
| AV01134.um | CAAACACUGCCAAGAGUGAAA | 22 | UUUCACUCUUGGCAGUGUUUG | 216 |
| AV01135.um | GAACACUGCCAAGAGUGAAUA | 23 | UAUUCACUCUUGGCAGUGUUC | 217 |
| AV01136.um | GACUGCCAAGAGUGAAUAGGA | 24 | UCCUAUUCACUCUUGGCAGUC | 218 |
| AV01137.um | GAGCAGCGAUUUCUGGGUAUA | 25 | UAUACCCAGAAAUCGCUGCUC | 219 |
| AV01138.um | CCAGCGAUUUCUGGGUAUUCA | 26 | UGAAUACCCAGAAAUCGCUGG | 220 |
| AV01139.um | GGAUUUCUGGGUAUUCUUUCA | 27 | UGAAAGAAUACCCAGAAAUCC | 221 |
| AV01140.um | GUCUGGGUAUUCUUUCAAGCA | 28 | UGCUUGAAAGAAUACCCAGAC | 222 |
| AV01141.um | GCUGGGUAUUCUUUCAAGCAA | 29 | UUGCUUGAAAGAAUACCCAGC | 223 |
| AV01142.um | GUGGGUAUUCUUUCAAGCAAA | 30 | UUUGCUUGAAAGAAUACCCAC | 224 |
| AV01143.um | GGGGUAUUCUUUCAAGCAAUA | 31 | UAUUGCUUGAAAGAAUACCCC | 225 |
| AV01144.um | CGUAUUCUUUCAAGCAAUGCA | 32 | UGCAUUGCUUGAAAGAAUACG | 226 |
| AV01145.um | GUUCUUUCAAGCAAUGCUCAA | 33 | UUGAGCAUUGCUUGAAAGAAC | 227 |
| AV01146.um | GUCUUUCAAGCAAUGCUCACA | 34 | UGUGAGCAUUGCUUGAAAGAC | 228 |
| AV01147.um | CCAAUGCUCACACCAAAUAAA | 35 | UUUAUUUGGUGUGAGCAUUGG | 229 |
| AV01148.um | GAAUGCUCACACCAAAUAAGA | 36 | UCUUAUUUGGUGUGAGCAUUC | 230 |
| AV01149.um | GAUGCUCACACCAAAUAAGCA | 37 | UGCUUAUUUGGUGUGAGCAUC | 231 |
| AV01150.um | GCACCAAAUAAGCGCUUGCAA | 38 | UUGCAAGCGCUUAUUUGGUGC | 232 |
| AV01151.um | GACCAAAUAAGCGCUUGCAAA | 39 | UUUGCAAGCGCUUAUUUGGUC | 233 |
| AV01152.um | GAAAUAAGCGCUUGCAACAAA | 40 | UUUGUUGCAAGCGCUUAUUUC | 234 |
| AV01153.um | GAAUAAGCGCUUGCAACAAAA | 41 | UUUUGUUGCAAGCGCUUAUUC | 235 |
| AV01154.um | CCUUGCAACAAAGACAUUUAA | 42 | UUAAAUGUCUUUGUUGCAAGG | 236 |
| AV01155.um | GGCAACAAAGACAUUUAUGUA | 43 | UACAUAAAUGUCUUUGUUGCC | 237 |
| AV01156.um | CCAACAAAGACAUUUAUGUGA | 44 | UCACAUAAAUGUCUUUGUUGG | 238 |
| AV01157.um | GAACAAAGACAUUUAUGUGGA | 45 | UCCACAUAAAUGUCUUUGUUC | 239 |
| AV01158.um | GACAAAGACAUUUAUGUGGAA | 46 | UUCCACAUAAAUGUCUUUGUC | 240 |
| AV01159.um | GAAGACAUUUAUGUGGACCUA | 47 | UAGGUCCACAUAAAUGUCUUC | 241 |
| AV01160.um | GAGACAUUUAUGUGGACCUAA | 48 | UUAGGUCCACAUAAAUGUCUC | 242 |
| AV01161.um | GGACAUUUAUGUGGACCUAGA | 49 | UCUAGGUCCACAUAAAUGUCC | 243 |
| AV01162.um | CACAUUUAUGUGGACCUAGAA | 50 | UUCUAGGUCCACAUAAAUGUG | 244 |
| AV01163.um | GAUGUGGACCUAGACAUGAAA | 51 | UUUCAUGUCUAGGUCCACAUC | 245 |
| AV01164.um | GUAGACAUGAAGGGCAUAAAA | 52 | UUUUAUGCCCUUCAUGUCUAC | 246 |
| AV01165.um | GAGACAUGAAGGGCAUAAACA | 53 | UGUUUAUGCCCUUCAUGUCUC | 247 |
| AV01166.um | GGACAUGAAGGGCAUAAACUA | 54 | UAGUUUAUGCCCUUCAUGUCC | 248 |
| AV01167.um | GCAUGAAGGGCAUAAACUAUA | 55 | UAUAGUUUAUGCCCUUCAUGC | 249 |
| AV01168.um | GAUGAAGGGCAUAAACUAUAA | 56 | UUAUAGUUUAUGCCCUUCAUC | 250 |
| AV01169.um | GUGAAGGGCAUAAACUAUAAA | 57 | UUUAUAGUUUAUGCCCUUCAC | 251 |
| AV01170.um | GGAAGGGCAUAAACUAUAACA | 58 | UGUUAUAGUUUAUGCCCUUCC | 252 |
| AV01171.um | CAAGGGCAUAAACUAUAACAA | 59 | UUGUUAUAGUUUAUGCCCUUG | 253 |
| AV01172.um | GAGGGCAUAAACUAUAACAGA | 60 | UCUGUUAUAGUUUAUGCCCUC | 254 |
| AV01173.um | CGGCAUAAACUAUAACAGCUA | 61 | UAGCUGUUAUAGUUUAUGCCG | 255 |
| AV01174.um | CCAUAAACUAUAACAGCUCAA | 62 | UUGAGCUGUUAUAGUUUAUGG | 256 |
| AV01175.um | CCCAAGAGUGCUCAAGAAUGA | 63 | UCAUUCUUGAGCACUCUUGGG | 257 |
| AV01176.um | GCAAGAAUGCCAAGAAAGAUA | 64 | UAUCUUUCUUGGCAUUCUUGC | 258 |
| AV01177.um | GAAGAAUGCCAAGAAAGAUGA | 65 | UCAUCUUUCUUGGCAUUCUUC | 259 |
| AV01178.um | GCCACUGCCACUUUUUCACGA | 66 | UCGUGAAAAAGUGGCAGUGGC | 260 |
| AV01179.um | GCACUGCCACUUUUUCACGUA | 67 | UACGUGAAAAAGUGGCAGUGC | 261 |
| AV01180.um | GACUGCCACUUUUUCACGUAA | 68 | UUACGUGAAAAAGUGGCAGUC | 262 |
| AV01181.um | GAGAAAUCUUUGUCUCCUUAA | 69 | UUAAGGAGACAAAGAUUUCUC | 263 |
| AV01182.um | GAUCUUUGUCUCCUUAAAACA | 70 | UGUUUUAAGGAGACAAAGAUC | 264 |
| AV01183.um | GUUUGUCUCCUUAAAACAUCA | 71 | UGAUGUUUUAAGGAGACAAAC | 265 |
| AV01184.um | GCUCCUUAAAACAUCUGAGAA | 72 | UUCUCAGAUGUUUUAAGGAGC | 266 |
| AV01185.um | GCCUUAAAACAUCUGAGAGUA | 73 | UACUCUCAGAUGUUUUAAGGC | 267 |
| AV01186.um | GAAACAUCUGAGAGUGGAUUA | 74 | UAAUCCACUCUCAGAUGUUUC | 268 |
| AV01187.um | GCACGCAUUAAAAAGAGCAAA | 75 | UUUGCUCUUUUUAAUGCGUGC | 269 |
| AV01188.um | GCGCAUUAAAAAGAGCAAAGA | 76 | UCUUUGCUCUUUUUAAUGCGC | 270 |
| AV01189.um | GUAAAAAGAGCAAAGCUCUUA | 77 | UAAGAGCUUUGCUCUUUUUAC | 271 |
| AV01190.um | GAAGAGCAAAGCUCUUUCUGA | 78 | UCAGAAAGAGCUUUGCUCUUC | 272 |
| AV01191.um | GGAGCAAAGCUCUUUCUGGUA | 79 | UACCAGAAAGAGCUUUGCUCC | 273 |
| AV01192.um | CAGCAAAGCUCUUUCUGGUUA | 80 | UAACCAGAAAGAGCUUUGCUG | 274 |
| AV01193.um | CCAAAGCUCUUUCUGGUUUCA | 81 | UGAAACCAGAAAGAGCUUUGG | 275 |
| AV01194.um | GAAAGCUCUUUCUGGUUUCAA | 82 | UUGAAACCAGAAAGAGCUUUC | 276 |
| AV01195.um | CAAGAACUGGAUAUUGUUGCA | 83 | UGCAACAAUAUCCAGUUCUUG | 277 |
| AV01196.um | GAGAACUGGAUAUUGUUGCUA | 84 | UAGCAACAAUAUCCAGUUCUC | 278 |
| AV01197.um | GUGCCAGAAACUGUGCACCAA | 85 | UUGGUGCACAGUUUCUGGCAC | 279 |
| AV01198.um | CCCAGAAACUGUGCACCAAUA | 86 | UAUUGGUGCACAGUUUCUGGG | 280 |
| AV01199.um | GCGCUGCCAGUUUUUUACCUA | 87 | UAGGUAAAAAACUGGCAGCGC | 281 |
| AV01200.um | CCUGCCAGUUUUUUACCUAUA | 88 | UAUAGGUAAAAAACUGGCAGG | 282 |
| AV01201.um | GAGUGUUACUUAAAGCUUUCA | 89 | UGAAAGCUUUAAGUAACACUC | 283 |
| AV01202.um | GGUGUUACUUAAAGCUUUCUA | 90 | UAGAAAGCUUUAAGUAACACC | 284 |
| AV01203.um | CUGUUACUUAAAGCUUUCUUA | 91 | UAAGAAAGCUUUAAGUAACAG | 285 |
| AV01204.um | GUACUUAAAGCUUUCUUCAAA | 92 | UUUGAAGAAAGCUUUAAGUAC | 286 |
| AV01205.um | GACUUAAAGCUUUCUUCAAAA | 93 | UUUUGAAGAAAGCUUUAAGUC | 287 |
| AV01206.um | GGGAUCUCCAACUAAAAUACA | 94 | UGUAUUUUAGUUGGAGAUCCC | 288 |
| AV01207.um | CGAUCUCCAACUAAAAUACUA | 95 | UAGUAUUUUAGUUGGAGAUCG | 289 |
| AV01208.um | CAUCUCCAACUAAAAUACUUA | 96 | UAAGUAUUUUAGUUGGAGAUG | 290 |
| AV01209.um | CCAUCUCUGGAUACACAUUAA | 97 | UUAAUGUGUAUCCAGAGAUGG | 291 |
| AV01210.um | GAUCUCUGGAUACACAUUAAA | 98 | UUUAAUGUGUAUCCAGAGAUC | 292 |
| AV01211.um | GUCUCUGGAUACACAUUAAGA | 99 | UCUUAAUGUGUAUCCAGAGAC | 293 |
| AV01212.um | GCUCUGGAUACACAUUAAGGA | 100 | UCCUUAAUGUGUAUCCAGAGC | 294 |
| AV01213.um | GUGUGUAAAAUGGAUAAUGAA | 101 | UUCAUUAUCCAUUUUACACAC | 295 |
| AV01117-19-1.um | ACAAGGUCUUUUCAGGAUA | 102 | UAUCCUGAAAAGACCUUGU | 296 |
| AV01118-19-1.um | AAGGUCUUUUCAGGAUGAA | 103 | UUCAUCCUGAAAAGACCUU | 297 |
| AV01119-19-1.um | GGUCUUUUCAGGAUGAUUA | 104 | UAAUCAUCCUGAAAAGACC | 298 |
| AV01120-19-1.um | CUUUUCAGGAUGAUUUUCA | 105 | UGAAAAUCAUCCUGAAAAG | 299 |
| AV01121-19-1.um | UUCAGGAUGAUUUUCUUAA | 106 | UUAAGAAAAUCAUCCUGAA | 300 |
| AV01122-19-1.um | GGAUGAUUUUCUUAUAUCA | 107 | UGAUAUAAGAAAAUCAUCC | 301 |
| AV01123-19-1.um | AUGAUUUUCUUAUAUCAAA | 108 | UUUGAUAUAAGAAAAUCAU | 302 |
| AV01124-19-1.um | UUCAGUUUCUGGUGAAUGA | 109 | UCAUUCACCAGAAACUGAA | 303 |
| AV01125-19-1.um | CAGUUUCUGGUGAAUGUGA | 110 | UCACAUUCACCAGAAACUG | 304 |
| AV01126-19-1.um | UCUGGUGAAUGUGUGACUA | 111 | UAGUCACACAUUCACCAGA | 305 |
| AV01127-19-1.um | GGUUUACUUGUGUCCUGAA | 112 | UUCAGGACACAAGUAAACC | 306 |
| AV01128-19-1.um | GUUUACUUGUGUCCUGAAA | 113 | UUUCAGGACACAAGUAAAC | 307 |
| AV01129-19-1.um | AAGACAGUGUUACAGAAAA | 114 | UUUUCUGUAACACUGUCUU | 308 |
| AV01130-19-1.um | AGACAGUGUUACAGAAACA | 115 | UGUUUCUGUAACACUGUCU | 309 |
| AV01131-19-1.um | ACAGUGUUACAGAAACACA | 116 | UGUGUUUCUGUAACACUGU | 310 |
| AV01132-19-1.um | ACAGAAACACUGCCAAGAA | 117 | UUCUUGGCAGUGUUUCUGU | 311 |
| AV01133-19-1.um | CAGAAACACUGCCAAGAGA | 118 | UCUCUUGGCAGUGUUUCUG | 312 |
| AV01134-19-1.um | AACACUGCCAAGAGUGAAA | 119 | UUUCACUCUUGGCAGUGUU | 313 |
| AV01135-19-1.um | ACACUGCCAAGAGUGAAUA | 120 | UAUUCACUCUUGGCAGUGU | 314 |
| AV01136-19-1.um | CUGCCAAGAGUGAAUAGGA | 121 | UCCUAUUCACUCUUGGCAG | 315 |
| AV01137-19-1.um | GCAGCGAUUUCUGGGUAUA | 122 | UAUACCCAGAAAUCGCUGC | 316 |
| AV01138-19-1.um | AGCGAUUUCUGGGUAUUCA | 123 | UGAAUACCCAGAAAUCGCU | 317 |
| AV01139-19-1.um | AUUUCUGGGUAUUCUUUCA | 124 | UGAAAGAAUACCCAGAAAU | 318 |
| AV01140-19-1.um | CUGGGUAUUCUUUCAAGCA | 125 | UGCUUGAAAGAAUACCCAG | 319 |
| AV01141-19-1.um | UGGGUAUUCUUUCAAGCAA | 126 | UUGCUUGAAAGAAUACCCA | 320 |
| AV01142-19-1.um | GGGUAUUCUUUCAAGCAAA | 127 | UUUGCUUGAAAGAAUACCC | 321 |
| AV01143-19-1.um | GGUAUUCUUUCAAGCAAUA | 128 | UAUUGCUUGAAAGAAUACC | 322 |
| AV01144-19-1.um | UAUUCUUUCAAGCAAUGCA | 129 | UGCAUUGCUUGAAAGAAUA | 323 |
| AV01145-19-1.um | UCUUUCAAGCAAUGCUCAA | 130 | UUGAGCAUUGCUUGAAAGA | 324 |
| AV01146-19-1.um | CUUUCAAGCAAUGCUCACA | 131 | UGUGAGCAUUGCUUGAAAG | 325 |
| AV01147-19-1.um | AAUGCUCACACCAAAUAAA | 132 | UUUAUUUGGUGUGAGCAUU | 326 |
| AV01148-19-1.um | AUGCUCACACCAAAUAAGA | 133 | UCUUAUUUGGUGUGAGCAU | 327 |
| AV01149-19-1.um | UGCUCACACCAAAUAAGCA | 134 | UGCUUAUUUGGUGUGAGCA | 328 |
| AV01150-19-1.um | ACCAAAUAAGCGCUUGCAA | 135 | UUGCAAGCGCUUAUUUGGU | 329 |
| AV01151-19-1.um | CCAAAUAAGCGCUUGCAAA | 136 | UUUGCAAGCGCUUAUUUGG | 330 |
| AV01152-19-1.um | AAUAAGCGCUUGCAACAAA | 137 | UUUGUUGCAAGCGCUUAUU | 331 |
| AV01153-19-1.um | AUAAGCGCUUGCAACAAAA | 138 | UUUUGUUGCAAGCGCUUAU | 332 |
| AV01154-19-1.um | UUGCAACAAAGACAUUUAA | 139 | UUAAAUGUCUUUGUUGCAA | 333 |
| AV01155-19-1.um | CAACAAAGACAUUUAUGUA | 140 | UACAUAAAUGUCUUUGUUG | 334 |
| AV01156-19-1.um | AACAAAGACAUUUAUGUGA | 141 | UCACAUAAAUGUCUUUGUU | 335 |
| AV01157-19-1.um | ACAAAGACAUUUAUGUGGA | 142 | UCCACAUAAAUGUCUUUGU | 336 |
| AV01158-19-1.um | CAAAGACAUUUAUGUGGAA | 143 | UUCCACAUAAAUGUCUUUG | 337 |
| AV01159-19-1.um | AGACAUUUAUGUGGACCUA | 144 | UAGGUCCACAUAAAUGUCU | 338 |
| AV01160-19-1.um | GACAUUUAUGUGGACCUAA | 145 | UUAGGUCCACAUAAAUGUC | 339 |
| AV01161-19-1.um | ACAUUUAUGUGGACCUAGA | 146 | UCUAGGUCCACAUAAAUGU | 340 |
| AV01162-19-1.um | CAUUUAUGUGGACCUAGAA | 147 | UUCUAGGUCCACAUAAAUG | 341 |
| AV01163-19-1.um | UGUGGACCUAGACAUGAAA | 148 | UUUCAUGUCUAGGUCCACA | 342 |
| AV01164-19-1.um | AGACAUGAAGGGCAUAAAA | 149 | UUUUAUGCCCUUCAUGUCU | 343 |
| AV01165-19-1.um | GACAUGAAGGGCAUAAACA | 150 | UGUUUAUGCCCUUCAUGUC | 344 |
| AV01166-19-1.um | ACAUGAAGGGCAUAAACUA | 151 | UAGUUUAUGCCCUUCAUGU | 345 |
| AV01167-19-1.um | AUGAAGGGCAUAAACUAUA | 152 | UAUAGUUUAUGOCCUUCAU | 346 |
| AV01168-19-1.um | UGAAGGGCAUAAACUAUAA | 153 | UUAUAGUUUAUGCCCUUCA | 347 |
| AV01169-19-1.um | GAAGGGCAUAAACUAUAAA | 154 | UUUAUAGUUUAUGCCCUUC | 348 |
| AV01170-19-1.um | AAGGGCAUAAACUAUAACA | 155 | UGUUAUAGUUUAUGCCCUU | 349 |
| AV01171-19-1.um | AGGGCAUAAACUAUAACAA | 156 | UUGUUAUAGUUUAUGCOCU | 350 |
| AV01172-19-1.um | GGGCAUAAACUAUAACAGA | 157 | UCUGUUAUAGUUUAUGCCC | 351 |
| AV01173-19-1.um | GCAUAAACUAUAACAGCUA | 158 | UAGCUGUUAUAGUUUAUGC | 352 |
| AV01174-19-1.um | AUAAACUAUAACAGCUCAA | 159 | UUGAGCUGUUAUAGUUUAU | 353 |
| AV01175-19-1.um | CAAGAGUGCUCAAGAAUGA | 160 | UCAUUCUUGAGCACUCUUG | 354 |
| AV01176-19-1.um | AAGAAUGCCAAGAAAGAUA | 161 | UAUCUUUCUUGGCAUUCUU | 355 |
| AV01177-19-1.um | AGAAUGCCAAGAAAGAUGA | 162 | UCAUCUUUCUUGGCAUUCU | 356 |
| AV01178-19-1.um | CACUGCCACUUUUUCACGA | 163 | UCGUGAAAAAGUGGCAGUG | 357 |
| AV01179-19-1.um | ACUGCCACUUUUUCACGUA | 164 | UACGUGAAAAAGUGGCAGU | 358 |
| AV01180-19-1.um | CUGCCACUUUUUCACGUAA | 165 | UUACGUGAAAAAGUGGCAG | 359 |
| AV01181-19-1.um | GAAAUCUUUGUCUCCUUAA | 166 | UUAAGGAGACAAAGAUUUC | 360 |
| AV01182-19-1.um | UCUUUGUCUCCUUAAAACA | 167 | UGUUUUAAGGAGACAAAGA | 361 |
| AV01183-19-1.um | UUGUCUCCUUAAAACAUCA | 168 | UGAUGUUUUAAGGAGACAA | 362 |
| AV01184-19-1.um | UCCUUAAAACAUCUGAGAA | 169 | UUCUCAGAUGUUUUAAGGA | 363 |
| AV01185-19-1.um | CUUAAAACAUCUGAGAGUA | 170 | UACUCUCAGAUGUUUUAAG | 364 |
| AV01186-19-1.um | AACAUCUGAGAGUGGAUUA | 171 | UAAUCCACUCUCAGAUGUU | 365 |
| AV01187-19-1.um | ACGCAUUAAAAAGAGCAAA | 172 | UUUGCUCUUUUUAAUGCGU | 366 |
| AV01188-19-1.um | GCAUUAAAAAGAGCAAAGA | 173 | UCUUUGCUCUUUUUAAUGC | 367 |
| AV01189-19-1.um | AAAAAGAGCAAAGCUCUUA | 174 | UAAGAGCUUUGCUCUUUUU | 368 |
| AV01190-19-1.um | AGAGCAAAGCUCUUUCUGA | 175 | UCAGAAAGAGCUUUGCUCU | 369 |
| AV01191-19-1.um | AGCAAAGCUCUUUCUGGUA | 176 | UACCAGAAAGAGCUUUGCU | 370 |
| AV01192-19-1.um | GCAAAGCUCUUUCUGGUUA | 177 | UAACCAGAAAGAGCUUUGC | 371 |
| AV01193-19-1.um | AAAGCUCUUUCUGGUUUCA | 178 | UGAAACCAGAAAGAGCUUU | 372 |
| AV01194-19-1.um | AAGCUCUUUCUGGUUUCAA | 179 | UUGAAACCAGAAAGAGCUU | 373 |
| AV01195-19-1.um | AGAACUGGAUAUUGUUGCA | 180 | UGCAACAAUAUCCAGUUCU | 374 |
| AV01196-19-1.um | GAACUGGAUAUUGUUGCUA | 181 | UAGCAACAAUAUCCAGUUC | 375 |
| AV01197-19-1.um | GCCAGAAACUGUGCACCAA | 182 | UUGGUGCACAGUUUCUGGC | 376 |
| AV01198-19-1.um | CAGAAACUGUGCACCAAUA | 183 | UAUUGGUGCACAGUUUCUG | 377 |
| AV01199-19-1.um | GCUGCCAGUUUUUUACCUA | 184 | UAGGUAAAAAACUGGCAGC | 378 |
| AV01200-19-1.um | UGCCAGUUUUUUACCUAUA | 185 | UAUAGGUAAAAAACUGGCA | 379 |
| AV01201-19-1.um | GUGUUACUUAAAGCUUUCA | 186 | UGAAAGCUUUAAGUAACAC | 380 |
| AV01202-19-1.um | UGUUACUUAAAGCUUUCUA | 187 | UAGAAAGCUUUAAGUAACA | 381 |
| AV01203-19-1.um | GUUACUUAAAGCUUUCUUA | 188 | UAAGAAAGCUUUAAGUAAC | 382 |
| AV01204-19-1.um | ACUUAAAGCUUUCUUCAAA | 189 | UUUGAAGAAAGCUUUAAGU | 383 |
| AV01205-19-1.um | CUUAAAGCUUUCUUCAAAA | 190 | UUUUGAAGAAAGCUUUAAG | 384 |
| AV01206-19-1.um | GAUCUCCAACUAAAAUACA | 191 | UGUAUUUUAGUUGGAGAUC | 385 |
| AV01207-19-1.um | AUCUCCAACUAAAAUACUA | 192 | UAGUAUUUUAGUUGGAGAU | 386 |
| AV01208-19-1.um | UCUCCAACUAAAAUACUUA | 193 | UAAGUAUUUUAGUUGGAGA | 387 |
| AV01209-19-1.um | AUCUCUGGAUACACAUUAA | 194 | UUAAUGUGUAUCCAGAGAU | 388 |
| AV01210-19-1.um | UCUCUGGAUACACAUUAAA | 195 | UUUAAUGUGUAUCCAGAGA | 389 |
| AV01211-19-1.um | CUCUGGAUACACAUUAAGA | 196 | UCUUAAUGUGUAUCCAGAG | 390 |
| AV01212-19-1.um | UCUGGAUACACAUUAAGGA | 197 | UCCUUAAUGUGUAUCCAGA | 391 |
| AV01213-19-1.um | GUGUAAAAUGGAUAAUGAA | 198 | UUCAUUAUCCAUUUUACAC | 392 |
| AV05205.um | GUUCAGGAUGAUUUUCUUAUA | 715 | UAUAAGAAAAUCAUCCUGAAC | 1001 |
| AV05206.um | GUCAGGAUGAUUUUCUUAUAA | 716 | UUAUAAGAAAAUCAUCCUGAC | 1002 |
| AV05207.um | GAGUUUCUGGUGAAUGUGUGA | 717 | UCACACAUUCACCAGAAACUC | 1003 |
| AV05208.um | CGGUAUUCUUUCAAGCAAUGA | 718 | UCAUUGCUUGAAAGAAUACCG | 1004 |
| AV05209.um | GCAAGCAAUGCUCACACCAAA | 719 | UUUGGUGUGAGCAUUGCUUGC | 1005 |
| AV05210.um | GAAGCAAUGCUCACACCAAAA | 720 | UUUUGGUGUGAGCAUUGCUUC | 1006 |
| AV05211.um | GUGCAACAAAGACAUUUAUGA | 721 | UCAUAAAUGUCUUUGUUGCAC | 1007 |
| AV05212.um | CACAUGAAGGGCAUAAACUAA | 722 | UUAGUUUAUGCCCUUCAUGUG | 1008 |
| AV05213.um | GGGGCAUAAACUAUAACAGCA | 723 | UGCUGUUAUAGUUUAUGCCCC | 1009 |
| AV05214.um | GUGAGUGUACCACCAAAAUCA | 724 | UGAUUUUGGUGGUACACUCAC | 1010 |
| AV05215.um | GGAGUGUACCACCAAAAUCAA | 725 | UUGAUUUUGGUGGUACACUCC | 1011 |
| AV05216.um | CAGUGUACCACCAAAAUCAAA | 726 | UUUGAUUUUGGUGGUACACUG | 1012 |
| AV05217.um | CGGUAGAGUCACCUAAGAUUA | 727 | UAAUCUUAGGUGACUCUACCG | 1013 |
| AV05218.um | CUCACCUAAGAUUUUGCGUGA | 728 | UCACGCAAAAUCUUAGGUGAG | 1014 |
| AV05219.um | GCACCUAAGAUUUUGCGUGUA | 729 | UACACGCAAAAUCUUAGGUGC | 1015 |
| AV05220.um | GCCUAAGAUUUUGCGUGUCUA | 730 | UAGACACGCAAAAUCUUAGGC | 1016 |
| AV05221.um | GCUAAGAUUUUGCGUGUCUAA | 731 | UUAGACACGCAAAAUCUUAGC | 1017 |
| AV05222.um | GAAGAUUUUGCGUGUCUACAA | 732 | UUGUAGACACGCAAAAUCUUC | 1018 |
| AV05223.um | GCAGUGGCAUUUUAAAUCAAA | 733 | UUUGAUUUAAAAUGCCACUGC | 1019 |
| AV05224.um | GAGUGGCAUUUUAAAUCAAUA | 734 | UAUUGAUUUAAAAUGCCACUC | 1020 |
| AV05225.um | GUUUAAAUCAAUCUGAAAUAA | 735 | UUAUUUCAGAUUGAUUUAAAC | 1021 |
| AV05226.um | GUAAAUCAAUCUGAAAUAAAA | 736 | UUUUAUUUCAGAUUGAUUUAC | 1022 |
| AV05227.um | GCACAUCUUUCUUUGGGGUUA | 737 | UAACCCCAAAGAAAGAUGUGC | 1023 |
| AV05228.um | GCAUCUUUCUUUGGGGUUCAA | 738 | UUGAACCCCAAAGAAAGAUGC | 1024 |
| AV05229.um | GAUCUUUCUUUGGGGUUCAAA | 739 | UUUGAACCCCAAAGAAAGAUC | 1025 |
| AV05230.um | GUUCUUUGGGGUUCAAGAAAA | 740 | UUUUCUUGAACCCCAAAGAAC | 1026 |
| AV05231.um | GUCUUUGGGGUUCAAGAAAUA | 741 | UAUUUCUUGAACCCCAAAGAC | 1027 |
| AV05232.um | GCUUUGGGGUUCAAGAAAUAA | 742 | UUAUUUCUUGAACCOCAAAGC | 1028 |
| AV05233.um | CGGUUCAAGAAAUAAUAAUCA | 743 | UGAUUAUUAUUUCUUGAACCG | 1029 |
| AV05234.um | GGAAAUAAUAAUCCAUGAUCA | 744 | UGAUCAUGGAUUAUUAUUUCC | 1030 |
| AV05235.um | GCAGUAUAAAAUGGCAGAAAA | 745 | UUUUCUGCCAUUUUAUACUGC | 1031 |
| AV05236.um | GAGUAUAAAAUGGCAGAAAGA | 746 | UCUUUCUGCCAUUUUAUACUC | 1032 |
| AV05237.um | CGGUAUGAUAUUGCCUUGUUA | 747 | UAACAAGGCAAUAUCAUACCG | 1033 |
| AV05238.um | GGAUAUUGCCUUGUUGAAACA | 748 | UGUUUCAACAAGGCAAUAUCC | 1034 |
| AV05239.um | CAUAUUGCCUUGUUGAAACUA | 749 | UAGUUUCAACAAGGCAAUAUG | 1035 |
| AV05240.um | GAUUGCCUUGUUGAAACUGGA | 750 | UCCAGUUUCAACAAGGCAAUC | 1036 |
| AV05241.um | CCCUUGUUGAAACUGGAAACA | 751 | UGUUUCCAGUUUCAACAAGGG | 1037 |
| AV05242.um | GGUUGAAACUGGAAACCACAA | 752 | UUGUGGUUUCCAGUUUCAACC | 1038 |
| AV05243.um | GGGAAACCACAGUGAAUUACA | 753 | UGUAAUUCACUGUGGUUUCCC | 1039 |
| AV05244.um | GAACCACAGUGAAUUACACAA | 754 | UUGUGUAAUUCACUGUGGUUC | 1040 |
| AV05245.um | GCCACAGUGAAUUACACAGAA | 755 | UUCUGUGUAAUUCACUGUGGC | 1041 |
| AV05246.um | GCAAAAUACUCUCCAGAAAGA | 756 | UCUUUCUGGAGAGUAUUUUGC | 1042 |
| AV05247.um | GCUCUCCAGAAAGCCAAGAUA | 757 | UAUCUUGGCUUUCUGGAGAGC | 1043 |
| AV05248.um | GAAGCCAAGAUACCCUUAGUA | 758 | UACUAAGGGUAUCUUGGCUUC | 1044 |
| AV05249.um | CAAGAGAUACAGAGGACAUAA | 759 | UUAUGUCCUCUGUAUCUCUUG | 1045 |
| AV05250.um | CAGAUACAGAGGACAUAAAAA | 760 | UUUUUAUGUCCUCUGUAUCUG | 1046 |
| AV05251.um | GACAGAGGACAUAAAAUAACA | 761 | UGUUAUUUUAUGUCCUCUGUC | 1047 |
| AV05252.um | GCAGAGGACAUAAAAUAACCA | 762 | UGGUUAUUUUAUGUCCUCUGC | 1048 |
| AV05253.um | CAGGACAUAAAAUAACCCAUA | 763 | UAUGGGUUAUUUUAUGUCCUG | 1049 |
| AV05254.um | CACAUAAAAUAACCCAUAAGA | 764 | UCUUAUGGGUUAUUUUAUGUG | 1050 |
| AV05255.um | GCAUAAAAUAACCCAUAAGAA | 765 | UUCUUAUGGGUUAUUUUAUGC | 1051 |
| AV05256.um | GACCCAUAAGAUGAUCUGUGA | 766 | UCACAGAUCAUCUUAUGGGUC | 1052 |
| AV05257.um | CGACUGGAUUCUGGAGAAAAA | 767 | UUUUUCUCCAGAAUCCAGUCG | 1053 |
| AV05258.um | CACUGGAUUCUGGAGAAAACA | 768 | UGUUUUCUCCAGAAUCCAGUG | 1054 |
| AV05499.um | GUCAUUCAGAGGAGGAUGAAA | 769 | UUUCAUCCUCCUCUGAAUGAC | 1055 |
| AV05500.um | GAAGGUCUUUUCAGGAUGAUA | 770 | UAUCAUCCUGAAAAGACCUUC | 1056 |
| AV05501.um | GCUUUUCAGGAUGAUUUUCUA | 771 | UAGAAAAUCAUCCUGAAAAGC | 1057 |
| AV05502.um | GGGAUGAUUUUCUUAUAUCAA | 772 | UUGAUAUAAGAAAAUCAUCCC | 1058 |
| AV05503.um | GGAUGGUUUACUUGUGUCCUA | 773 | UAGGACACAAGUAAACCAUCC | 1059 |
| AV05504.um | GGCCAAGAGUGAAUAGGACAA | 774 | UUGUCCUAUUCACUCUUGGCC | 1060 |
| AV05505.um | GUUUCUGGGUAUUCUUUCAAA | 775 | UUUGAAAGAAUACCCAGAAAC | 1061 |
| AV05506.um | GUUCUGGGUAUUCUUUCAAGA | 776 | UCUUGAAAGAAUACCCAGAAC | 1062 |
| AV05507.um | GCAAAUAAGCGCUUGCAACAA | 777 | UUGUUGCAAGCGCUUAUUUGC | 1063 |
| AV05508.um | GGCUUGCAACAAAGACAUUUA | 778 | UAAAUGUCUUUGUUGCAAGCC | 1064 |
| AV05509.um | GCAAAGACAUUUAUGUGGACA | 779 | UGUCCACAUAAAUGUCUUUGC | 1065 |
| AV05510.um | GUAUGUGGACCUAGACAUGAA | 780 | UUCAUGUCUAGGUCCACAUAC | 1066 |
| AV05511.un | CUUGCCAAGAGUGCUCAAGAA | 781 | UUCUUGAGCACUCUUGGCAAG | 1067 |
| AV05512.um | CCCAAGAAAGAUGCACGGAUA | 782 | UAUCCGUGCAUCUUUCUUGGG | 1068 |
| AV05513.um | GGAAAUCUUUGUCUCCUUAAA | 783 | UUUAAGGAGACAAAGAUUUCC | 1069 |
| AV05514.um | CAAAUCUUUGUCUCCUUAAAA | 784 | UUUUAAGGAGACAAAGAUUUG | 1070 |
| AV05515.um | GAAUCUUUGUCUCCUUAAAAA | 785 | UUUUUAAGGAGACAAAGAUUC | 1071 |
| AV05516.um | GCUUUGUCUCCUUAAAACAUA | 786 | UAUGUUUUAAGGAGACAAAGC | 1072 |
| AV05517.um | GUUGUCUCCUUAAAACAUCUA | 787 | UAGAUGUUUUAAGGAGACAAC | 1073 |
| AV05518.um | GUGUCUCCUUAAAACAUCUGA | 788 | UCAGAUGUUUUAAGGAGACAC | 1074 |
| AV05519.um | GGUCUCCUUAAAACAUCUGAA | 789 | UUCAGAUGUUUUAAGGAGACC | 1075 |
| AV05520.um | GUCCUUAAAACAUCUGAGAGA | 790 | UCUCUCAGAUGUUUUAAGGAC | 1076 |
| AV05521.um | GUUAAAACAUCUGAGAGUGGA | 791 | UCCACUCUCAGAUGUUUUAAC | 1077 |
| AV05522.um | GAGCUCUUUCUGGUUUCAGUA | 792 | UACUGAAACCAGAAAGAGCUC | 1078 |
| AV05523.um | GGAACUGGAUAUUGUUGCUGA | 793 | UCAGCAACAAUAUCCAGUUCC | 1079 |
| AV05524.um | CAGGAGGCAUCUCUGGAUACA | 794 | UGUAUCCAGAGAUGCCUCCUG | 1080 |
| AV05525.um | CGCAUCUCUGGAUACACAUUA | 795 | UAAUGUGUAUCCAGAGAUGCG | 1081 |
| AV05526.um | CUUGUGUAAAAUGGAUAAUGA | 796 | UCAUUAUCCAUUUUACACAAG | 1082 |
| AV05527.um | GGUGUAAAAUGGAUAAUGAGA | 797 | UCUCAUUAUCCAUUUUACACC | 1083 |
| AV05528.um | CUGUACCACCAAAAUCAAGCA | 798 | UGCUUGAUUUUGGUGGUACAG | 1084 |
| AV05529.um | GCCAGGAUCGUUGGAGGAACA | 799 | UGUUCCUCCAACGAUCCUGGC | 1085 |
| AV05530.um | CUAGAGUCACCUAAGAUUUUA | 800 | UAAAAUCUUAGGUGACUCUAG | 1086 |
| AV05531.um | GGGCAUUUUAAAUCAAUCUGA | 801 | UCAGAUUGAUUUAAAAUGCCC | 1087 |
| AV05532.um | CGCAUUUUAAAUCAAUCUGAA | 802 | UUCAGAUUGAUUUAAAAUGCG | 1088 |
| AV05533.um | CCAUUUUAAAUCAAUCUGAAA | 803 | UUUCAGAUUGAUUUAAAAUGG | 1089 |
| AV05534.um | CGACACAUCUUUCUUUGGGGA | 804 | UCCCCAAAGAAAGAUGUGUCG | 1090 |
| AV05535.um | GCUUUCUUUGGGGUUCAAGAA | 805 | UUCUUGAACCCCAAAGAAAGC | 1091 |
| AV05536.um | GUUUGGGGUUCAAGAAAUAAA | 806 | UUUAUUUCUUGAACCCCAAAC | 1092 |
| AV05537.um | CUUCAAGAAAUAAUAAUCCAA | 807 | UUGGAUUAUUAUUUCUUGAAG | 1093 |
| AV05538.um | GUCAAGAAAUAAUAAUCCAUA | 808 | UAUGGAUUAUUAUUUCUUGAC | 1094 |
| AV05539.um | GGGGUAUGAUAUUGCCUUGUA | 809 | UACAAGGCAAUAUCAUACCCC | 1095 |
| AV05540.um | GUAUUGCCUUGUUGAAACUGA | 810 | UCAGUUUCAACAAGGCAAUAC | 1096 |
| AV05541.um | GCUUGUUGAAACUGGAAACCA | 811 | UGGUUUCCAGUUUCAACAAGC | 1097 |
| AV05542.um | CGAAACCACAGUGAAUUACAA | 812 | UUGUAAUUCACUGUGGUUUCG | 1098 |
| AV05543.um | GUCUCCAGAAAGCCAAGAUAA | 813 | UUAUCUUGGCUUUCUGGAGAC | 1099 |
| AV05544.um | GUCCAGAAAGCCAAGAUACCA | 814 | UGGUAUCUUGGCUUUCUGGAC | 1100 |
| AV05545.um | CAAAGCCAAGAUACCCUUAGA | 815 | UCUAAGGGUAUCUUGGCUUUG | 1101 |
| AV05546.um | GGCCAAGAUACCCUUAGUGAA | 816 | UUCACUAAGGGUAUCUUGGCC | 1102 |
| AV05547.um | GCAAGAUACCCUUAGUGACCA | 817 | UGGUCACUAAGGGUAUCUUGC | 1103 |
| AV05548.um | GAAGAUACCCUUAGUGACCAA | 818 | UUGGUCACUAAGGGUAUCUUC | 1104 |
| AV05549.um | GGAAGAGUGCCAGAAGAGAUA | 819 | UAUCUCUUCUGGCACUCUUCC | 1105 |
| AV05550.um | CUGCCAGAAGAGAUACAGAGA | 820 | UCUCUGUAUCUCUUCUGGCAG | 1106 |
| AV05551.um | GGAAGAGAUACAGAGGACAUA | 821 | UAUGUCCUCUGUAUCUCUUCC | 1107 |
| AV05552.um | GAGAGAUACAGAGGACAUAAA | 822 | UUUAUGUCCUCUGUAUCUCUC | 1108 |
| AV05553.um | GGAUACAGAGGACAUAAAAUA | 823 | UAUUUUAUGUCCUCUGUAUCC | 1109 |
| AV05554.um | CAUACAGAGGACAUAAAAUAA | 824 | UUAUUUUAUGUCCUCUGUAUG | 1110 |
| AV05555.um | GGGACAUAAAAUAACCCAUAA | 825 | UUAUGGGUUAUUUUAUGUCCC | 1111 |
| AV05556.um | GAAAAUAACCCAUAAGAUGAA | 826 | UUCAUCUUAUGGGUUAUUUUC | 1112 |
| AV05557.um | GAAAUAACCCAUAAGAUGAUA | 827 | UAUCAUCUUAUGGGUUAUUUC | 1113 |
| AV05558.um | GUAACCCAUAAGAUGAUCUGA | 828 | UCAGAUCAUCUUAUGGGUUAC | 1114 |
| AV05559.um | GAACCCAUAAGAUGAUCUGUA | 829 | UACAGAUCAUCUUAUGGGUUC | 1115 |
| AV05560.um | GGUACGUGGACUGGAUUCUGA | 830 | UCAGAAUCCAGUCCACGUACC | 1116 |
| AV05561.um | CUGGACUGGAUUCUGGAGAAA | 831 | UUUCUCCAGAAUCCAGUCCAG | 1117 |
| AV05562.um | GGGAUUCUGGAGAAAACUCAA | 832 | UUGAGUUUUCUCCAGAAUCCC | 1118 |
| AV05563.um | CGAUUCUGGAGAAAACUCAAA | 833 | UUUGAGUUUUCUCCAGAAUCG | 1119 |
| AV05564.um | CAUUCUGGAGAAAACUCAAGA | 834 | UCUUGAGUUUUCUCCAGAAUG | 1120 |
| AV05565.um | GGCAUGCUUCCUCCACAGUAA | 835 | UUACUGUGGAGGAAGCAUGCC | 1121 |
| AV05566.um | GGCUAGAAGAAAACAAACUGA | 836 | UCAGUUUGUUUUCUUCUAGCC | 1122 |
| AV05567.um | CCUAGAAGAAAACAAACUGUA | 837 | UACAGUUUGUUUUCUUCUAGG | 1123 |
| AV05568.um | GUAGAAGAAAACAAACUGUCA | 838 | UGACAGUUUGUUUUCUUCUAC | 1124 |
| AV05569.um | GAGAAGAAAACAAACUGUCAA | 839 | UUGACAGUUUGUUUUCUUCUC | 1125 |
| AV05570.um | CAAGAAAACAAACUGUCACAA | 840 | UUGUGACAGUUUGUUUUCUUG | 1126 |
| AV06227.um | AAAAUGCUAGAAGAAAACAAA | 841 | UUUGUUUUCUUCUAGCAUUUU | 1127 |
| AV06228.um | AAAUGCUAGAAGAAAACAAAA | 842 | UUUUGUUUUCUUCUAGCAUUU | 1128 |
| AV06229.um | AAUGCUAGAAGAAAACAAACA | 843 | UGUUUGUUUUCUUCUAGCAUU | 1129 |
| AV06230.um | AUGCUAGAAGAAAACAAACUA | 844 | UAGUUUGUUUUCUUCUAGCAU | 1130 |
| AV06231.um | UGCUAGAAGAAAACAAACUGA | 845 | UCAGUUUGUUUUCUUCUAGCA | 1131 |
| AV06232.um | GCUAGAAGAAAACAAACUGUA | 846 | UACAGUUUGUUUUCUUCUAGC | 1132 |
| AV06233.um | CUAGAAGAAAACAAACUGUCA | 847 | UGACAGUUUGUUUUCUUCUAG | 1133 |
| AV06234.um | UAGAAGAAAACAAACUGUCAA | 848 | UUGACAGUUUGUUUUCUUCUA | 1134 |
| AV06235.um | AGAAGAAAACAAACUGUCACA | 849 | UGUGACAGUUUGUUUUCUUCU | 1135 |
| AV06236.um | GAAGAAAACAAACUGUCACAA | 850 | UUGUGACAGUUUGUUUUCUUC | 1136 |
| AV06237.um | AAGAAAACAAACUGUCACAAA | 851 | UUUGUGACAGUUUGUUUUCUU | 1137 |
| AV06238.um | AGAAAACAAACUGUCACAAGA | 852 | UCUUGUGACAGUUUGUUUUCU | 1138 |
| AV06239.um | GAAAACAAACUGUCACAAGUA | 853 | UACUUGUGACAGUUUGUUUUC | 1139 |
| AV06240.um | AAAACAAACUGUCACAAGUUA | 854 | UAACUUGUGACAGUUUGUUUU | 1140 |
| AV06241.um | AAACAAACUGUCACAAGUUGA | 855 | UCAACUUGUGACAGUUUGUUU | 1141 |
| AV01126-1.um | GCGUCAGUUUCUGGUGAAUGA | 856 | UCAUUCACCAGAAACUGACGC | 1142 |
| AV01138-1.um | CGAGCGAUUUCUGGGUAUUCA | 857 | UGAAUACCCAGAAAUCGCUCG | 1143 |
| AV05205-19-1.um | UCAGGAUGAUUUUCUUAUA | 858 | UAUAAGAAAAUCAUCCUGA | 1144 |
| AV05206-19-1.um | CAGGAUGAUUUUCUUAUAA | 859 | UUAUAAGAAAAUCAUCCUG | 1145 |
| AV05207-19-1.um | GUUUCUGGUGAAUGUGUGA | 860 | UCACACAUUCACCAGAAAC | 1146 |
| AV05208-19-1.um | GUAUUCUUUCAAGCAAUGA | 861 | UCAUUGCUUGAAAGAAUAC | 1147 |
| AV05209-19-1.um | AAGCAAUGCUCACACCAAA | 862 | UUUGGUGUGAGCAUUGCUU | 1148 |
| AV05210-19-1.um | AGCAAUGCUCACACCAAAA | 863 | UUUUGGUGUGAGCAUUGCU | 1149 |
| AV05211-19-1.um | GCAACAAAGACAUUUAUGA | 864 | UCAUAAAUGUCUUUGUUGC | 1150 |
| AV05212-19-1.um | CAUGAAGGGCAUAAACUAA | 865 | UUAGUUUAUGCCCUUCAUG | 1151 |
| AV05213-19-1.um | GGCAUAAACUAUAACAGCA | 866 | UGCUGUUAUAGUUUAUGCC | 1152 |
| AV05214-19-1.um | GAGUGUACCACCAAAAUCA | 867 | UGAUUUUGGUGGUACACUC | 1153 |
| AV05215-19-1.um | AGUGUACCACCAAAAUCAA | 868 | UUGAUUUUGGUGGUACACU | 1154 |
| AV05216-19-1.um | GUGUACCACCAAAAUCAAA | 869 | UUUGAUUUUGGUGGUACAC | 1155 |
| AV05217-19-1.um | GUAGAGUCACCUAAGAUUA | 870 | UAAUCUUAGGUGACUCUAC | 1156 |
| AV05218-19-1.um | CACCUAAGAUUUUGCGUGA | 871 | UCACGCAAAAUCUUAGGUG | 1157 |
| AV05219-19-1.um | ACCUAAGAUUUUGCGUGUA | 872 | UACACGCAAAAUCUUAGGU | 1158 |
| AV05220-19-1.um | CUAAGAUUUUGCGUGUCUA | 873 | UAGACACGCAAAAUCUUAG | 1159 |
| AV05221-19-1.um | UAAGAUUUUGCGUGUCUAA | 874 | UUAGACACGCAAAAUCUUA | 1160 |
| AV05222-19-1.um | AGAUUUUGCGUGUCUACAA | 875 | UUGUAGACACGCAAAAUCU | 1161 |
| AV05223-19-1.um | AGUGGCAUUUUAAAUCAAA | 876 | UUUGAUUUAAAAUGCCACU | 1162 |
| AV05224-19-1.um | GUGGCAUUUUAAAUCAAUA | 877 | UAUUGAUUUAAAAUGCCAC | 1163 |
| AV05225-19-1.um | UUAAAUCAAUCUGAAAUAA | 878 | UUAUUUCAGAUUGAUUUAA | 1164 |
| AV05226-19-1.um | AAAUCAAUCUGAAAUAAAA | 879 | UUUUAUUUCAGAUUGAUUU | 1165 |
| AV05227-19-1.um | ACAUCUUUCUUUGGGGUUA | 880 | UAACCCCAAAGAAAGAUGU | 1166 |
| AV05228-19-1.um | AUCUUUCUUUGGGGUUCAA | 881 | UUGAACCCCAAAGAAAGAU | 1167 |
| AV05229-19-1.um | UCUUUCUUUGGGGUUCAAA | 882 | UUUGAACCCCAAAGAAAGA | 1168 |
| AV05230-19-1.um | UCUUUGGGGUUCAAGAAAA | 883 | UUUUCUUGAACCCCAAAGA | 1169 |
| AV05231-19-1.um | CUUUGGGGUUCAAGAAAUA | 884 | UAUUUCUUGAACCCCAAAG | 1170 |
| AV05232-19-1.um | UUUGGGGUUCAAGAAAUAA | 885 | UUAUUUCUUGAACCCCAAA | 1171 |
| AV05233-19-1.um | GUUCAAGAAAUAAUAAUCA | 886 | UGAUUAUUAUUUCUUGAAC | 1172 |
| AV05234-19-1.um | AAAUAAUAAUCCAUGAUCA | 887 | UGAUCAUGGAUUAUUAUUU | 1173 |
| AV05235-19-1.um | AGUAUAAAAUGGCAGAAAA | 888 | UUUUCUGCCAUUUUAUACU | 1174 |
| AV05236-19-1.um | GUAUAAAAUGGCAGAAAGA | 889 | UCUUUCUGCCAUUUUAUAC | 1175 |
| AV05237-19-1.um | GUAUGAUAUUGCCUUGUUA | 890 | UAACAAGGCAAUAUCAUAC | 1176 |
| AV05238-19-1.um | AUAUUGCCUUGUUGAAACA | 891 | UGUUUCAACAAGGCAAUAU | 1177 |
| AV05239-19-1.um | UAUUGCCUUGUUGAAACUA | 892 | UAGUUUCAACAAGGCAAUA | 1178 |
| AV05240-19-1.um | UUGCCUUGUUGAAACUGGA | 893 | UCCAGUUUCAACAAGGCAA | 1179 |
| AV05241-19-1.um | CUUGUUGAAACUGGAAACA | 894 | UGUUUCCAGUUUCAACAAG | 1180 |
| AV05242-19-1.um | UUGAAACUGGAAACCACAA | 895 | UUGUGGUUUCCAGUUUCAA | 1181 |
| AV05243-19-1.um | GAAACCACAGUGAAUUACA | 896 | UGUAAUUCACUGUGGUUUC | 1182 |
| AV05244-19-1.um | ACCACAGUGAAUUACACAA | 897 | UUGUGUAAUUCACUGUGGU | 1183 |
| AV05245-19-1.um | CACAGUGAAUUACACAGAA | 898 | UUCUGUGUAAUUCACUGUG | 1184 |
| AV05246-19-1.um | AAAAUACUCUCCAGAAAGA | 899 | UCUUUCUGGAGAGUAUUUU | 1185 |
| AV05247-19-1.um | UCUCCAGAAAGCCAAGAUA | 900 | UAUCUUGGCUUUCUGGAGA | 1186 |
| AV05248-19-1.um | AGCCAAGAUACCCUUAGUA | 901 | UACUAAGGGUAUCUUGGCU | 1187 |
| AV05249-19-1.um | AGAGAUACAGAGGACAUAA | 902 | UUAUGUCCUCUGUAUCUCU | 1188 |
| AV05250-19-1.um | GAUACAGAGGACAUAAAAA | 903 | UUUUUAUGUCCUCUGUAUC | 1189 |
| AV05251-19-1.um | CAGAGGACAUAAAAUAACA | 904 | UGUUAUUUUAUGUCCUCUG | 1190 |
| AV05252-19-1.um | AGAGGACAUAAAAUAACCA | 905 | UGGUUAUUUUAUGUCCUCU | 1191 |
| AV05253-19-1.um | GGACAUAAAAUAACCCAUA | 906 | UAUGGGUUAUUUUAUGUCC | 1192 |
| AV05254-19-1.um | CAUAAAAUAACCCAUAAGA | 907 | UCUUAUGGGUUAUUUUAUG | 1193 |
| AV05255-19-1.um | AUAAAAUAACCCAUAAGAA | 908 | UUCUUAUGGGUUAUUUUAU | 1194 |
| AV05256-19-1.um | CCCAUAAGAUGAUCUGUGA | 909 | UCACAGAUCAUCUUAUGGG | 1195 |
| AV05257-19-1.um | ACUGGAUUCUGGAGAAAAA | 910 | UUUUUCUCCAGAAUCCAGU | 1196 |
| AV05258-19-1.um | CUGGAUUCUGGAGAAAACA | 911 | UGUUUUCUCCAGAAUCCAG | 1197 |
| AV05499-19-1.um | CAUUCAGAGGAGGAUGAAA | 912 | UUUCAUCCUCCUCUGAAUG | 1198 |
| AV05500-19-1.um | AGGUCUUUUCAGGAUGAUA | 913 | UAUCAUCCUGAAAAGACCU | 1199 |
| AV05501-19-1.um | UUUUCAGGAUGAUUUUCUA | 914 | UAGAAAAUCAUCCUGAAAA | 1200 |
| AV05502-19-1.um | GAUGAUUUUCUUAUAUCAA | 915 | UUGAUAUAAGAAAAUCAUC | 1201 |
| AV05503-19-1.um | AUGGUUUACUUGUGUCCUA | 916 | UAGGACACAAGUAAACCAU | 1202 |
| AV05504-19-1.um | CCAAGAGUGAAUAGGACAA | 917 | UUGUCCUAUUCACUCUUGG | 1203 |
| AV05505-19-1.um | UUCUGGGUAUUCUUUCAAA | 918 | UUUGAAAGAAUACCCAGAA | 1204 |
| AV05506-19-1.um | UCUGGGUAUUCUUUCAAGA | 919 | UCUUGAAAGAAUACCCAGA | 1205 |
| AV05507-19-1.um | AAAUAAGCGCUUGCAACAA | 920 | UUGUUGCAAGCGCUUAUUU | 1206 |
| AV05508-19-1.um | CUUGCAACAAAGACAUUUA | 921 | UAAAUGUCUUUGUUGCAAG | 1207 |
| AV05509-19-1.um | AAAGACAUUUAUGUGGACA | 922 | UGUCCACAUAAAUGUCUUU | 1208 |
| AV05510-19-1.um | AUGUGGACCUAGACAUGAA | 923 | UUCAUGUCUAGGUCCACAU | 1209 |
| AV05511-19-1.um | UGCCAAGAGUGCUCAAGAA | 924 | UUCUUGAGCACUCUUGGCA | 1210 |
| AV05512-19-1.um | CAAGAAAGAUGCACGGAUA | 925 | UAUCCGUGCAUCUUUCUUG | 1211 |
| AV05513-19-1.um | AAAUCUUUGUCUCCUUAAA | 926 | UUUAAGGAGACAAAGAUUU | 1212 |
| AV05514-19-1.um | AAUCUUUGUCUCCUUAAAA | 927 | UUUUAAGGAGACAAAGAUU | 1213 |
| AV05515-19-1.um | AUCUUUGUCUCCUUAAAAA | 928 | UUUUUAAGGAGACAAAGAU | 1214 |
| AV05516-19-1.um | UUUGUCUCCUUAAAACAUA | 929 | UAUGUUUUAAGGAGACAAA | 1215 |
| AV05517-19-1.um | UGUCUCCUUAAAACAUCUA | 930 | UAGAUGUUUUAAGGAGACA | 1216 |
| AV05518-19-1.um | GUCUCCUUAAAACAUCUGA | 931 | UCAGAUGUUUUAAGGAGAC | 1217 |
| AV05519-19-1.um | UCUCCUUAAAACAUCUGAA | 932 | UUCAGAUGUUUUAAGGAGA | 1218 |
| AV05520-19-1.um | CCUUAAAACAUCUGAGAGA | 933 | UCUCUCAGAUGUUUUAAGG | 1219 |
| AV05521-19-1.um | UAAAACAUCUGAGAGUGGA | 934 | UCCACUCUCAGAUGUUUUA | 1220 |
| AV05522-19-1.um | GCUCUUUCUGGUUUCAGUA | 935 | UACUGAAACCAGAAAGAGC | 1221 |
| AV05523-19-1.um | AACUGGAUAUUGUUGCUGA | 936 | UCAGCAACAAUAUCCAGUU | 1222 |
| AV05524-19-1.um | GGAGGCAUCUCUGGAUACA | 937 | UGUAUCCAGAGAUGCCUCC | 1223 |
| AV05525-19-1.um | CAUCUCUGGAUACACAUUA | 938 | UAAUGUGUAUCCAGAGAUG | 1224 |
| AV05526-19-1.um | UGUGUAAAAUGGAUAAUGA | 939 | UCAUUAUCCAUUUUACACA | 1225 |
| AV05527-19-1.um | UGUAAAAUGGAUAAUGAGA | 940 | UCUCAUUAUCCAUUUUACA | 1226 |
| AV05528-19-1.um | GUACCACCAAAAUCAAGCA | 941 | UGCUUGAUUUUGGUGGUAC | 1227 |
| AV05529-19-1.um | CAGGAUCGUUGGAGGAACA | 942 | UGUUCCUCCAACGAUCCUG | 1228 |
| AV05530-19-1.um | AGAGUCACCUAAGAUUUUA | 943 | UAAAAUCUUAGGUGACUCU | 1229 |
| AV05531-19-1.um | GCAUUUUAAAUCAAUCUGA | 944 | UCAGAUUGAUUUAAAAUGC | 1230 |
| AV05532-19-1.um | CAUUUUAAAUCAAUCUGAA | 945 | UUCAGAUUGAUUUAAAAUG | 1231 |
| AV05533-19-1.um | AUUUUAAAUCAAUCUGAAA | 946 | UUUCAGAUUGAUUUAAAAU | 1232 |
| AV05534-19-1.um | ACACAUCUUUCUUUGGGGA | 947 | UCCCCAAAGAAAGAUGUGU | 1233 |
| AV05535-19-1.um | UUUCUUUGGGGUUCAAGAA | 948 | UUCUUGAACCCCAAAGAAA | 1234 |
| AV05536-19-1.um | UUGGGGUUCAAGAAAUAAA | 949 | UUUAUUUCUUGAACCCCAA | 1235 |
| AV05537-19-1.um | UCAAGAAAUAAUAAUCCAA | 950 | UUGGAUUAUUAUUUCUUGA | 1236 |
| AV05538-19-1.um | CAAGAAAUAAUAAUCCAUA | 951 | UAUGGAUUAUUAUUUCUUG | 1237 |
| AV05539-19-1.um | GGUAUGAUAUUGCCUUGUA | 952 | UACAAGGCAAUAUCAUACC | 1238 |
| AV05540-19-1.um | AUUGCCUUGUUGAAACUGA | 953 | UCAGUUUCAACAAGGCAAU | 1239 |
| AV05541-19-1.um | UUGUUGAAACUGGAAACCA | 954 | UGGUUUCCAGUUUCAACAA | 1240 |
| AV05542-19-1.um | AAACCACAGUGAAUUACAA | 955 | UUGUAAUUCACUGUGGUUU | 1241 |
| AV05543-19-1.um | CUCCAGAAAGCCAAGAUAA | 956 | UUAUCUUGGCUUUCUGGAG | 1242 |
| AV05544-19-1.um | CCAGAAAGCCAAGAUACCA | 957 | UGGUAUCUUGGCUUUCUGG | 1243 |
| AV05545-19-1.um | AAGCCAAGAUACCCUUAGA | 958 | UCUAAGGGUAUCUUGGCUU | 1244 |
| AV05546-19-1.um | CCAAGAUACCCUUAGUGAA | 959 | UUCACUAAGGGUAUCUUGG | 1245 |
| AV05547-19-1.um | AAGAUACCCUUAGUGACCA | 960 | UGGUCACUAAGGGUAUCUU | 1246 |
| AV05548-19-1.um | AGAUACCCUUAGUGACCAA | 961 | UUGGUCACUAAGGGUAUCU | 1247 |
| AV05549-19-1.um | AAGAGUGCCAGAAGAGAUA | 962 | UAUCUCUUCUGGCACUCUU | 1248 |
| AV05550-19-1.um | GCCAGAAGAGAUACAGAGA | 963 | UCUCUGUAUCUCUUCUGGC | 1249 |
| AV05551-19-1.um | AAGAGAUACAGAGGACAUA | 964 | UAUGUCCUCUGUAUCUCUU | 1250 |
| AV05552-19-1.um | GAGAUACAGAGGACAUAAA | 965 | UUUAUGUCCUCUGUAUCUC | 1251 |
| AV05553-19-1.um | AUACAGAGGACAUAAAAUA | 966 | UAUUUUAUGUCCUCUGUAU | 1252 |
| AV05554-19-1.um | UACAGAGGACAUAAAAUAA | 967 | UUAUUUUAUGUCCUCUGUA | 1253 |
| AV05555-19-1.um | GACAUAAAAUAACCCAUAA | 968 | UUAUGGGUUAUUUUAUGUC | 1254 |
| AV05556-19-1.um | AAAUAACCCAUAAGAUGAA | 969 | UUCAUCUUAUGGGUUAUUU | 1255 |
| AV05557-19-1.um | AAUAACCCAUAAGAUGAUA | 970 | UAUCAUCUUAUGGGUUAUU | 1256 |
| AV05558-19-1.um | AACCCAUAAGAUGAUCUGA | 971 | UCAGAUCAUCUUAUGGGUU | 1257 |
| AV05559-19-1.um | ACCCAUAAGAUGAUCUGUA | 972 | UACAGAUCAUCUUAUGGGU | 1258 |
| AV05560-19-1.um | UACGUGGACUGGAUUCUGA | 973 | UCAGAAUCCAGUCCACGUA | 1259 |
| AV05561-19-1.um | GGACUGGAUUCUGGAGAAA | 974 | UUUCUCCAGAAUCCAGUCC | 1260 |
| AV05562-19-1.um | GAUUCUGGAGAAAACUCAA | 975 | UUGAGUUUUCUCCAGAAUC | 1261 |
| AV05563-19-1.um | AUUCUGGAGAAAACUCAAA | 976 | UUUGAGUUUUCUCCAGAAU | 1262 |
| AV05564-19-1.um | UUCUGGAGAAAACUCAAGA | 977 | UCUUGAGUUUUCUCCAGAA | 1263 |
| AV05565-19-1.um | CAUGCUUCCUCCACAGUAA | 978 | UUACUGUGGAGGAAGCAUG | 1264 |
| AV05566-19-1.um | CUAGAAGAAAACAAACUGA | 979 | UCAGUUUGUUUUCUUCUAG | 1265 |
| AV05567-19-1.um | UAGAAGAAAACAAACUGUA | 980 | UACAGUUUGUUUUCUUCUA | 1266 |
| AV05568-19-1.um | AGAAGAAAACAAACUGUCA | 981 | UGACAGUUUGUUUUCUUCU | 1267 |
| AV05569-19-1.um | GAAGAAAACAAACUGUCAA | 982 | UUGACAGUUUGUUUUCUUC | 1268 |
| AV05570-19-1.um | AGAAAACAAACUGUCACAA | 983 | UUGUGACAGUUUGUUUUCU | 1269 |
| AV06227-19-1.um | AAUGCUAGAAGAAAACAAA | 984 | UUUGUUUUCUUCUAGCAUU | 1270 |
| AV06228-19-1.um | AUGCUAGAAGAAAACAAAA | 985 | UUUUGUUUUCUUCUAGCAU | 1271 |
| AV06229-19-1.um | UGCUAGAAGAAAACAAACA | 986 | UGUUUGUUUUCUUCUAGCA | 1272 |
| AV06230-19-1.um | GCUAGAAGAAAACAAACUA | 987 | UAGUUUGUUUUCUUCUAGC | 1273 |
| AV06231-19-1.um | CUAGAAGAAAACAAACUGA | 988 | UCAGUUUGUUUUCUUCUAG | 1274 |
| AV06232-19-1.um | UAGAAGAAAACAAACUGUA | 989 | UACAGUUUGUUUUCUUCUA | 1275 |
| AV06233-19-1.um | AGAAGAAAACAAACUGUCA | 990 | UGACAGUUUGUUUUCUUCU | 1276 |
| AV06234-19-1.um | GAAGAAAACAAACUGUCAA | 991 | UUGACAGUUUGUUUUCUUC | 1277 |
| AV06235-19-1.um | AAGAAAACAAACUGUCACA | 992 | UGUGACAGUUUGUUUUCUU | 1278 |
| AV06236-19-1.um | AGAAAACAAACUGUCACAA | 993 | UUGUGACAGUUUGUUUUCU | 1279 |
| AV06237-19-1.um | GAAAACAAACUGUCACAAA | 994 | UUUGUGACAGUUUGUUUUC | 1280 |
| AV06238-19-1.um | AAAACAAACUGUCACAAGA | 995 | UCUUGUGACAGUUUGUUUU | 1281 |
| AV06239-19-1.um | AAACAAACUGUCACAAGUA | 996 | UACUUGUGACAGUUUGUUU | 1282 |
| AV06240-19-1.um | AACAAACUGUCACAAGUUA | 997 | UAACUUGUGACAGUUUGUU | 1283 |
| AV06241-19-1.um | ACAAACUGUCACAAGUUGA | 998 | UCAACUUGUGACAGUUUGU | 1284 |
| AV01126-1-19-1.um | GUCAGUUUCUGGUGAAUGA | 999 | UCAUUCACCAGAAACUGAC | 1285 |
| AV01138-1-19-1.um | AGCGAUUUCUGGGUAUUCA | 1000 | UGAAUACCCAGAAAUCGCU | 1286 |
| AV01139-1.um | GGAUUUCUGGGUAUUCUUUCU | 1729 | AGAAAGAAUACCCAGAAAUCC | 1730 |
| AV01139-1-19-1.um | AUUUCUGGGUAUUCUUUCU | 1733 | AGAAAGAAUACCCAGAAAU | 1734 |

Sense strand sequences of AV01124, AV01126, AV01138, AV01141, AV01152, AV01180, and AV01204 are shown in table 1, corresponding respectively position of 148-168, 155-175, 385-405, 394-414, 424-444, 541-561, 1146-1166 of SEQ ID NO: 1.

Table 2 shows certain chemically modified FXI RNAi agent antisense strand and sense strand sequences of the invention. In some embodiments of methods of the invention, an RNAi agent with a polynucleotide sequence shown in Table 2 is administered to a cell and/or subject. In some embodiments of methods of the invention, an RNAi agent with a polynucleotide sequence shown in Table 2 is administered to a subject. In some embodiments of the invention an RNAi agent administered to a subject comprises is a duplex identified in a row in Table 2, column one and includes the sequence modifications show in the sense and antisense strand sequences in the same row in Table 2, columns four and seven, respectively. In some embodiments of methods of the invention, a sequence shown in Table 2 may be attached to (also referred to herein as “conjugated to”) a compound capable of delivering the RNAi agent to a cell and/or tissue in a subject. A non-limiting example of a delivery compound that may be used in certain embodiments of the invention is a GalNAc-containing compound or a GLS-15-containing compound. In Table 2, the first column indicates the Duplex AV #of the base sequences as shown in Table 1. Table 2 discloses Duplex AV #and also shows chemical modifications included in sense and antisense sequence of the duplexes. For example, Table 1 shows base single-strand sequences SEQ ID NO: 6 (sense) and SEQ ID NO: 200 (antisense), which together are the double-stranded duplex identified as: Duplex AV #AV01118.um and Table 2 lists Duplex AV #AV01118, which indicates that the duplex of SEQ ID NO: 394 and SEQ ID NO:491 includes base sequences of SEQ ID NO: 6 and SEQ ID NO: 200, respectively, but with the chemical modifications shown in the sense and antisense sequences shown in columns four and seven, respectively. The “Sense strand SS #” in Table 2 column two is the assigned identifier for the Sense Sequence (including modifications) shown column 4 in the same row. The “Antisense strand AS #” in Table 2 column 5 is the assigned identifier for the Antisense sequence (including modifications) shown in column seven.

TABLE 2
provides chemically modified FXI RNAi agent antisense strand and sense strand
sequences. All sequences shown 5′ to 3′. These sequences were used in certain in vitro testing
studies described herein.

| | Sense | SEQ | | Antisense | SEQ | |
|---|---|---|---|---|---|---|
| Duplex | strand | ID | | strand | ID | |
| AV# | SS# | NO | Sense Strand | AS# | NO | Antisense Strand |
| AV01117 | AV01117-SS | 393 | g*a*acaaggUfcUfuuUfcagga*u*a | AV01117-AS | 490 | u*Af*ucCfugaaaaGfaCfcuuGfu*u*c |
| AV01118 | AV01118-SS | 394 | gfc*aaggucUfuUfucAfggaug*a*a | AV01118-AS | 491 | u*Uf*caUfccugaaAfaGfaccUfu*g*c |
| AV01119 | AV01119-SS | 395 | g*a*ggucuuUfuCfagGfaugau*u*a | AV01119-AS | 492 | u*Af*auCfauccugAfaAfagaCfc*u*c |
| AV01120 | AV01120-SS | 396 | c*u*cuuuucAfgGfauGfauuuu*c*a | AV01120-AS | 493 | u*Gf*aaAfaucaucCfuGfaaaAfg*a*g |
| AV01121 | AV01121-SS | 397 | g*u*uucaggAfuGfauUfuucuu*a*a | AV01121-AS | 494 | u*Uf*aaGfaaaaucAfuCfcugAfa*avc |
| AV01122 | AV01122-SS | 398 | g*a*ggaugaUfuUfucUfuauau*c*a | AV01122-AS | 495 | u*Gf*auAfuaagaaAfaUfcauCfc*u*c |
| AV01123 | AV01123-SS | 399 | c*g*augauuUfuCfuuAfuauca*a*a | AV01123-AS | 496 | u*Uf*ugAfuauaagAfaAfaucAfu*c*g |
| AV01124 | AV01124-SS | 400 | g*c*uucaguUfuCfugGfugaau*g*a | AV01124-AS | 497 | u*Cf*auUfcaccagAfaAfcugAfa*g*c |
| AV01125 | AV01125-SS | 401 | g*u*caguuuCfuGfguGfaaugu*g*a | AV01125-AS | 498 | u*Cf*acAfuucaccAfgAfaacUfg*a*c |
| AV01126 | AV01126-SS | 402 | g*u*ucugguGfaAfugUfgugac*u*a | AV01126-AS | 499 | u*Af*guCfacacauUfcAfccaGfa*a*c |
| AV01127 | AV01127-SS | 403 | g*u*gguuuaCfuUfguGfuccug*a*a | AV01127-AS | 500 | u*Uf*caGfg(aUNA)cacaAfgUfaaaCf |
| | | | | | | c*a*c |
| AV01128 | AV01128-SS | 404 | g*g*guuuacUfuGfugUfccuga*a*a | AV01128-AS | 501 | u*Uf*ucAfggacacAfaGfuaaAfc*c*c |
| AV01129 | AV01129-SS | 405 | c*a*aagacaGfuGfuuAfcagaa*a*a | AV01129-AS | 502 | u*Uf*uuCfaguaacAfcUfgucUfu*u*g |
| AV01130 | AV01130-SS | 406 | g*a*agacagUfgUfuaCfagaaa*c*a | AV01130-AS | 503 | u*Gf*uuUfcuguaaCfaCfuguCfu*u*c |
| AV01131 | AV01131-SS | 407 | g*g*acagugUfuAfcaGfaaaca*c*a | AV01131-AS | 504 | u*Gf*ugUfuucuguAfaCfacuGfu*c*c |
| AV01132 | AV01132-SS | 408 | g*u*acagaaAfcAfcuGfccaag*a*a | AV01132-AS | 505 | u*Uf*cuUfg(gUNA)caguGfuUfucuGf |
| | | | | | | u*a*c |
| AV01133 | AV01133-SS | 409 | g*a*cagaaaCfaCfugCfcaaga*g*a | AV01133-AS | 506 | u*Cf*ucUfu(gUNA)gcagUfgUfuucUf |
| | | | | | | g*u*c |
| AV01134 | AV01134-SS | 410 | c*a*aacacuGfcCfaaGfaguga*a*a | AV01134-AS | 507 | u*Uf*ucAfcucuugGfcAfgugUfu*ukg |
| AV01135 | AV01135-SS | 411 | g*a*acacugCfcAfagAfgugaa*u*a | AV01135-AS | 508 | u*Af*uuCfacucuuGfgCfaguGfu*ufc |
| AV01136 | AV01136-SS | 412 | g*a*cugccaAfgAfguGfaauag*g*a | AV01136-AS | 509 | u*Cf*cuAfuucacuCfuUfggcAfg*u*c |
| AV01137 | AV01137-SS | 413 | g*a*gcagcgAfuUfucUfgggua*u*a | AV01137-AS | 510 | u*Af*uaCfccagaaAfuCfgcuGfc*u*c |
| AV01138 | AV01138-SS | 414 | c*c*agcgauUfuCfugGfguauu*c*a | AV01138-AS | 511 | u*Gf*aaUfacccagAfaAfucgCfu*g*g |
| AV01139 | AV01139-SS | 415 | g*g*auuucuGfgGfuaUfucuuu*c*a | AV01139-AS | 512 | u*Gf*aaAfgaauacCfcAfgaaAfu*c*c |
| AV01140 | AV01140-SS | 416 | g*u*cuggouAfuUfcuUfucaag*c*a | AV01140-AS | 513 | u*Gf*cuUfgaaagaAfuAfcccAfg*a*c |
| AV01141 | AV01141-SS | 417 | g*c*uggguaUfuCfuuUfcaagc*a*a | AV01141-AS | 514 | u*Uf*gcUfugaaagAfaUfaccCfa*g*c |
| AV01142 | AV01142-SS | 418 | g*u*gcguauUfcUfuuCfaagca*a*a | AV01142-AS | 515 | u*Uf*ugCfuugaaaGfaAfuacCfc*a*c |
| AV01143 | AV01143-SS | 419 | g*g*gguauuCfuUfucAfagcaa*u*a | AV01143-AS | 516 | u*Af*uuGfcuugaaAfgAfauaCfc*c*c |
| AV01144 | AV01144-SS | 420 | c*g*uauucuUfuCfaaGfcaaug*c*a | AV01144-AS | 517 | u*Gf*caUfu(gUNA)cuugAfaAfgaaUf |
| | | | | | | a*c*g |
| AV01145 | AV01145-SS | 421 | g*u*ucuuucAfaGfcaAfugcuc*a*a | AV01145-AS | 518 | u*Uf*gaGfcauugcUfuGfaaaGfa*a*c |
| AV01146 | AV01146-SS | 422 | g*u*cuuucaAfgCfaaUfgcuca*c*a | AV01146-AS | 519 | u*Gf*ugAfg(cUNA)auugCfuUfgaaAf |
| | | | | | | g*a*c |
| AV01147 | AV01147-SS | 423 | c*c*aaugcuCfaCfacCfaaaua*a*a | AV01147-AS | 520 | u*Uf*uaUfuuggugUfgAfgcaUfu*g*g |
| AV01148 | AV01148-SS | 424 | g*a*augcucAfcAfccAfaauaa*g*a | AV01148-AS | 521 | u*Cf*uuAfuuugguGfaGfagcAfu*u*c |
| AV01149 | AV01149-SS | 425 | g*a*ugcucaCfaCfcaAfauaag*c*a | AV01149-AS | 522 | u*Gf*cuUfauuuggUfgUfgagCfa*u*c |
| AV01150 | AV01150-SS | 426 | g*c*accaaaUfaAfgcGfcuugc*a*a | AV01150-AS | 523 | u*Uf*gcAfa(gUNA)cgcuUfaUfuugGf |
| | | | | | | u*g*c |
| AV01151 | AV01151-SS | 427 | g*a*ccaaauAfaGfcgCfuugca*a*a | AV01151-AS | 524 | u*Uf*ugCfaagcgcUfuAfuuuGfg*u*c |
| AV01152 | AV01152-SS | 428 | g*a*aauaagCfgCfuuGfcaaca*a*a | AV01152-AS | 525 | u*Uf*ugUfugcaagCfgCfuuaUfu*u*c |
| AV01153 | AV01153-SS | 429 | g*a*auaagcGfcUfugCfaacaa*a*a | AV01153-AS | 526 | u*Uf*uuGfuugcaaGfcGfcuuAfu*u*c |
| AV01154 | AV01154-SS | 430 | c*c*uugcaaCfaAfagAfcauuu*a*a | AV01154-AS | 527 | u*Uf*aaAfugucuuUfgUfugcAfa*g*g |
| AV01155 | AV01155-SS | 431 | g*g*caacaaAfgAfcaUfuuaug*u*a | AV01155-AS | 528 | u*Af*caUfaaauguCfuUfuguUfg*c*c |
| AV01156 | AV01156-SS | 432 | c*c*aacaaaGfaCfauUfuaugu*g*a | AV01156-AS | 529 | u*Cf*acAfuaaaugUfcUfuugUfu*g*g |
| AV01157 | AV01157-SS | 433 | g*a*acaaagAfcAfuuUfaugug*g*a | AV01157-AS | 530 | u*Cf*caCfauaaauGfuCfuuuGfu*u*c |
| AV01158 | AV01158-SS | 434 | g*a*caaagaCfaUfuuAfugugg*a*a | AV01158-AS | 531 | u*Uf*ccAfcauaaaUfgUfcuuUfg*u*c |
| AV01159 | AV01159-SS | 435 | g*a*agacauUfuAfugUfggacc*u*a | AV01159-AS | 532 | u*Af*ggUfc(cUNA)acauAfaAfuguCf |
| | | | | | | u*u*c |
| AV01160 | AV01160-SS | 436 | g*a*gacauuUfaUfguGfgaccu*a*a | AV01160-AS | 533 | u*Uf*agGfu(cUNA)cacaUfaAfaugUf |
| | | | | | | c*u*c |
| AV01161 | AV01161-SS | 437 | g*g*acauuuAfuGfugGfaccua*g*a | AV01161-AS | 534 | u*Cf*uaGfg(uUNA)ccacAfuAfaauGf |
| | | | | | | u*c*c |
| AV01162 | AV01162-SS | 438 | c*a*cauuuaUfgUfggAfccuag*a*a | AV01162-AS | 535 | u*Uf*cuAfgguccaCfaUfaaaUfg*u*g |
| AV01163 | AV01163-SS | 439 | g*a*uguggaCfcUfagAfcauga*a*a | AV01163-AS | 536 | u*Uf*ucAfugucuaGfgUfccaCfa*u*c |
| AV01164 | AV01164-SS | 440 | g*u*agacauGfaAfggGfcauaa*a*a | AV01164-AS | 537 | u*Uf*uuAfugcccuUfcAfuguCfu*a*c |
| AV01165 | AV01165-SS | 441 | g*a*gacaugAfaGfggCfauaaa*c*a | AV01165-AS | 538 | u*Gf*uuUfaugcccUfuCfaugUfc*u*c |
| AV01166 | AV01166-SS | 442 | g*g*acaugaAf@GfgcAfuaaac*u*a | AV01166-AS | 539 | u*Af*guUfuaugccCfuUfcauGfu*c*c |
| AV01167 | AV01167-SS | 443 | g*c*augaagGfgCfauAfaacua*u*a | AV01167-AS | 540 | u*Af*uaGfuuuaugCfcCfuucAfu*g*c |
| AV01168 | AV01168-SS | 444 | g*a*ugaaggGfcAfuaAfacuau*a*a | AV01168-AS | 541 | u*Uf*auAfguuuauGfcCfcuuCfa*u*c |
| AV01169 | AV01169-SS | 445 | g*u*gaagggCfaUfaaAfcuaua*a*a | AV01169-AS | 542 | u*Uf*uaUfaguuuaUfgCfccuUfc*a*c |
| AV01170 | AV01170-SS | 446 | g*g*aagggcAfuAfaaCfuauaa*c*a | AV01170-AS | 543 | u*Gf*uuAfuaguuuAfuGfcccUfu*c*c |
| AV01171 | AV01171-SS | 447 | c*a*agggcaUfaAfacUfauaac*a*a | AV01171-AS | 544 | u*Uf*guUfauaguuUfaUfgccCfu*u*g |
| AV01172 | AV01172-SS | 448 | g*a*gggcauAfaAfcuAfuaaca*g*a | AV01172-AS | 545 | u*Cf*ugUfuauaguUfuAfugcCfc*u*c |
| AV01173 | AV01173-SS | 449 | c*g*gcauaaAfcUfauAfacagc*u*a | AV01173-AS | 546 | u*Af*gcUfguuauaGfuUfuauGfc*c*g |
| AV01174 | AV01174-SS | 450 | c*c*auaaacUfaUfaaCfagcuc*a*a | AV01174-AS | 547 | u*Uf*gaGfc(uUNA)guuaUfaGfuuuAf |
| | | | | | | u*g*g |
| AV01175 | AV01175-SS | 451 | c*c*caagagUfgCfucAfagaau*g*a | AV01175-AS | 548 | u*Cf*auUfcuugagCfaCfucuUfg*g*g |
| AV01176 | AV01176-SS | 452 | g*c*aagaauGfcCfaaGfaaaga*u*a | AV01176-AS | 549 | u*Af*ucUfuucuugGfcAfuucUfu*g*c |
| AV01177 | AV01177-SS | 453 | g*a*agaaugCfcAfagAfaagau*g*a | AV01177-AS | 550 | u*Cf*auCfuuucuuGfgCfauuCfu*u*c |
| AV01178 | AV01178-SS | 454 | g*c*cacugcCfaCfuuUfuucac*g*a | AV01178-AS | 551 | u*Cf*guGfaaaaagUfgGfcagUfg*g*c |
| AV01179 | AV01179-SS | 455 | g*c*acugccAfcUfuuUfucacg*u*a | AV01179-AS | 552 | u*Af*cgUfgaaaaaGfuGfgcaGfu*g*c |
| AV01180 | AV01180-SS | 456 | g*a*cugccaCfuUfuuUfcacgu*a*a | AV01180-AS | 553 | u*Uf*acGfugaaaaAfgUfggcAfg*u*c |
| AV01181 | AV01181-SS | 457 | g*a*gaaaucUfuUfguCfuccuu*a*a | AV01181-AS | 554 | u*Uf*aaGfgagacaAfaGfauuUfc*u*c |
| AV01182 | AV01182-SS | 458 | g*a*ucuuugUfcUfccUfuaaaa*c*a | AV01182-AS | 555 | u*Gf*uuUfuaaggaGfaCfaaaGfa*u*c |
| AV01183 | AV01183-SS | 459 | g*u*uugucuCfcUfuaAfaacau*c*a | AV01183-AS | 556 | u*Gf*auGfuuuuaaGfgAfgacAfa*a*c |
| AV01184 | AV01184-SS | 460 | g*c*uccuuaAfaAfcaUfcugag*a*a | AV01184-AS | 557 | u*Uf*cuCfagauguUfuUfaagGfa*g*c |
| AV01185 | AV01185-SS | 461 | g*c*cuuaaaAfcAfucUfgagag*u*a | AV01185-AS | 558 | u*Af*cuCfucagauGfuUfuuaAfg*g*c |
| AV01186 | AV01186-SS | 462 | g*a*aacaucUfgAfgaGfuggau*u*a | AV01186-AS | 559 | u*Af*auCfcacucuCfaGfaugUfu*u*c |
| AV01187 | AV01187-SS | 463 | g*c*acgcauUfaAfaaAfgagca*a*a | AV01187-AS | 560 | u*Uf*ugCfucuuuuUfaAfugcGfu*g*c |
| AV01188 | AV01188-SS | 464 | g*c*gcauuaAfaAfagAfccaaa*g*a | AV01188-AS | 561 | u*Cf*uuUfccucuuUfuUfaauGfc*g*c |
| AV01189 | AV01189-SS | 465 | g*u*aaaaagAfgCfaaAfgcucu*u*a | AV01189-AS | 562 | u*Af*agAfccuuugCfuCfuuuUfu*a*c |
| AV01190 | AV01190-SS | 466 | g*a*agagcaAfaGfcuCfuuucu*g*a | AV01190-AS | 563 | u*Cf*agAfaagagcUfuUfgcuCfu*u*c |
| AV01191 | AV01191-SS | 467 | g*g*agcaaaGfcUfcuUfucugg*u*a | AV01191-AS | 564 | u*Af*ccAfgaaagaGfcUfuugCfu*c*c |
| AV01192 | AV01192-SS | 468 | c*a*gcaaagCfuCfuuUfcuggu*u*a | AV01192-AS | 565 | u*Af*acCfagaaagAfgCfuuuGfc*u*g |
| AV01193 | AV01193-SS | 469 | c*c*aaagcuCfuUfucUfgguuu*c*a | AV01193-AS | 566 | u*Gf*aaAfccagaaAfgAfgcuUfu*g*g |
| AV01194 | AV01194-SS | 470 | g*a*aagcucUfuUfcuGfguuuc*a*a | AV01194-AS | 567 | u*Uf*gaAfaccagaAfaGfagcUfu*u*c |
| AV01195 | AV01195-SS | 471 | c*a*agaacuGfgAfuaUfuguug*c*a | AV01195-AS | 568 | u*Gf*caAfcaauauCfcAfguuCfu*u*g |
| AV01196 | AV01196-SS | 472 | g*a*gaacugGfaUfauUfguugc*u*a | AV01196-AS | 569 | u*Af*gcAfacaauaUfcCfaguUfc*u*c |
| AV01197 | AV01197-SS | 473 | g*u*gccagaAfaCfugUfgcacc*a*a | AV01197-AS | 570 | u*Uf*ggUfg(cUNA)acagUfuUfcugGf |
| | | | | | | c*a*c |
| AV01198 | AV01198-SS | 474 | c*c*cagaaaCfuGfugCfaccaa*u*a | AV01198-AS | 571 | u*Af*uuGfgugcacAfgUfuucUfg*g*g |
| AV01199 | AV01199-SS | 475 | g*c*gcugccAfgUfuuUfuuacc*u*a | AV01199-AS | 572 | u*Af*ggUfaaaaaaCfuGfgcaGfc*g*c |
| AV01200 | AV01200-SS | 476 | c*c*ugccagUfuUfuuUfaccua*u*a | AV01200-AS | 573 | u*Af*uaGfguaaaaAfaCfuggCfa*g*g |
| AV01201 | AV01201-SS | 477 | g*a*guguuaCfuUfaaAfgcuuu*c*a | AV01201-AS | 574 | u*Gf*aaAfgcuuuaAfgUfaacAfc*u*c |
| AV01202 | AV01202-SS | 478 | g*g*uguuacUfuAfaaGfcuuuc*u*a | AV01202-AS | 575 | u*Af*gaAfagcuuuAfaGfuaaCfa*c*c |
| AV01203 | AV01203-SS | 479 | c*u*guuacuUfaAfagCfuuucu*u*a | AV01203-AS | 576 | u*Af*agAfaagcuuUfaAfguaAfc*a*g |
| AV01204 | AV01204-SS | 480 | g*u*acuuaaAfgCfuuUfcuuca*a*a | AV01204-AS | 577 | u*Uf*ugAfagaaagCfuUfuaaGfu*a*c |
| AV01205 | AV01205-SS | 481 | g*a*cuuaaaGfcUfuuCfuucaa*a*a | AV01205-AS | 578 | u*Uf*uuGfaagaaaGfcUfuuaAfg*u*c |
| AV01206 | AV01206-SS | 482 | g*g*gaucucCfaAfcuAfaaaua*c*a | AV01206-AS | 579 | u*Gf*uaUfuuuaguUfgGfagaUfc*c*c |
| AV01207 | AV01207-SS | 483 | c*g*aucuccAfaCfuaAfaauac*u*a | AV01207-AS | 580 | u*Af*guAfuuuuagUfuGfgagAfu*c*g |
| AV01208 | AV01208-SS | 484 | c*a*ucuccaAfcUfaaAfauacu*u*a | AV01208-AS | 581 | u*Af*agUfauuuuaGfuUfggaGfa*u*g |
| AV01209 | AV01209-SS | 485 | c*c*aucucuGfgAfuaCfacauu*a*a | AV01209-AS | 582 | u*Uf*aaUfguguauCfcAfgagAfu*g*g |
| AV01210 | AV01210-SS | 486 | g*a*ucucugGfaUfacAfcauua*a*a | AV01210-AS | 583 | u*Uf*uaAfuguguaUfcCfagaGfa*u*c |
| AV01211 | AV01211-SS | 487 | g*u*cucuggAfuAfcaCfauuaa*g*a | AV01211-AS | 584 | u*Cf*uuAfauguguAfuCfcagAfg*a*c |
| AV01212 | AV01212-SS | 488 | g*c*ucuggaUfaCfacAfuuaag*g*a | AV01212-AS | 585 | u*Cf*cuUfaaugugUfaUfccaGfa*g*c |
| AV01213 | AV01213-SS | 489 | g*u*guguaaAfaUfggAfuaaug*a*a | AV01213-AS | 586 | u*Uf*caUfuauccaUfuUfuacAfc*a*c |
| AV05205 | AV05205-SS | 1287 | g*u*ucaggaUfgAfuUfuucuua*u*a | AV05205-AS | 1428 | u*Af*uaagAfaaauCfaUfcCfuga*a*c |
| AV05206 | AV05206-SS | 1288 | g*u*caggauGfaUfuUfucuuau*a*a | AV05206-AS | 1429 | u*Uf*auaaGfaaaaUfcAfuCfcug*a*c |
| AV05207 | AV05207-SS | 1289 | g*a*guuucuGfgUfgAfaugugu*g*a | AV05207-AS | 1430 | u*Cf*acacAfuucaCfcAfgAfaac*u*c |
| AV05208 | AV05208-SS | 1290 | c*g*quauucUfuUfcAfagcaau*g*a | AV05208-AS | 1431 | u*Cf*auugCfuugaAfaGfaAfuac*c*g |
| AV05209 | AV05209-SS | 1291 | g*c*aagcaaUfgCfuCfacacca*a*a | AV05209-AS | 1432 | u*Uf*ugguGfugagCfaUfuGfcuu*g*c |
| AV05210 | AV05210-SS | 1292 | g*a*agcaauGfcUfcAfcaccaa*a*a | AV05210-AS | 1433 | u*Uf*uuggUfgugaGfcAfuUfgcu*u*c |
| AV05211 | AV05211-SS | 1293 | g*u*gcaacaAfaGfaCfauuuau*g*a | AV05211-AS | 1434 | u*Cf*auaaAfugucUfuUfgUfugc*a*c |
| AV05212 | AV05212-SS | 1294 | c*a*caugaaGfgGfcAfuaaacu*a*a | AV05212-AS | 1435 | u*Uf*aguuUfaugcCfcUfuCfaug*u*g |
| AV05213 | AV05213-SS | 1295 | g*g*ggcauaAfaCfuAfuaacag*c*a | AV05213-AS | 1436 | u*Gf*cuguUfauagUfuUfaUfgcc*c*c |
| AV05214 | AV05214-SS | 1296 | g*u*gaguguAfcCfaCfcaaaau*c*a | AV05214-AS | 1437 | u*Gf*auuuUfggugGfuAfcAfcuc*a*c |
| AV05215 | AV05215-SS | 1297 | g*g*aguguaCfcAfcCfaaaauc*a*a | AV05215-AS | 1438 | u*Uf*gauuUfugguGfgUfaCfacu*c*c |
| AV05216 | AV05216-SS | 1298 | c*a*guguacCfaCfcAfaaauca*a*a | AV05216-AS | 1439 | u*Uf*ugauUfuuggUfgGfuAfcac*u*g |
| AV05217 | AV05217-SS | 1299 | c*g*guagagUfcAfcCfuaagau*u*a | AV05217-AS | 1440 | u*Af*aucuUfagguGfaCfuCfuac*c*g |
| AV05218 | AV05218-SS | 1300 | c*u*caccuaAfgAfuUfuugcgu*g*a | AV05218-AS | 1441 | u*Cf*acGfc(aUNA)aaauCfuUfaGfgu |
| | | | | | | g*a*g |
| AV05219 | AV05219-SS | 1301 | g*c*accuaaGfaUfuUfugcgug*u*a | AV05219-AS | 1442 | u*Af*caCfg(cUNA)aaaaUfcUfuAfgg |
| | | | | | | u*g*c |
| AV05220 | AV05220-SS | 1302 | g*c*cuaagaUfuUfuGfcguguc*u*a | AV05220-AS | 1443 | u*Af*gaCfa(cUNA)gcaaAfaUfcUfua |
| | | | | | | g*g*c |
| AV05221 | AV05221-SS | 1303 | g*c*uaagauUfuUfgCfgugucu*a*a | AV05221-AS | 1444 | u*Uf*agacAfcgcaAfaAfuCfuua*g*c |
| AV05222 | AV05222-SS | 1304 | g*a*agauuuUfgCfgUfgucuac*a*a | AV05222-AS | 1445 | u*Uf*guagAfcacgCfaAfaAfucu*u*c |
| AV05223 | AV05223-SS | 1305 | g*c*aguggcAfuUfuUfaaauca*a*a | AV05223-AS | 1446 | u*Uf*ugauUfuaaaAfuGfcCfacu*g*c |
| AV05224 | AV05224-SS | 1306 | g*a*guggcaUfuUfuAfaaucaa*u*a | AV05224-AS | 1447 | u*Af*uugaUfuuaaAfaUfgCfcac*u*c |
| AV05225 | AV05225-SS | 1307 | g*u*uuaaauCfaAfuCfugaaau*a*a | AV05225-AS | 1448 | u*Uf*auuuCfagauUfgAfuUfuaa*a*c |
| AV05226 | AV05226-SS | 1308 | g*u*aaaucaAfuCfuGfaaauaa*a*a | AV05226-AS | 1449 | u*Uf*uuauUfucagAfuUfgAfuuu*a*c |
| AV05227 | AV05227-SS | 1309 | g*c*acaucuUfuCfuUfuggggu*u*a | AV05227-AS | 1450 | u*Af*acCfc(cUNA)aaagAfaAfgAfug |
| | | | | | | u*g*c |
| AV05228 | AV05228-SS | 1310 | g*c*aucuuuCfuUfuGfggguuc*a*a | AV05228-AS | 1451 | u*Uf*gaAfc(cUNA)ccaaAfgAfaAfga |
| | | | | | | u*g*c |
| AV05229 | AV05229-SS | 1311 | g*a*ucuuucUfuUfgGfgguuca*a*a | AV05229-AS | 1452 | u*Uf*ugaaCfcccaAfaGfaAfaga*u*c |
| AV05230 | AV05230-SS | 1312 | g*u*ucuuugGfgGfuUfcaagaa*a*a | AV05230-AS | 1453 | u*Uf*uucuUfgaacCfcCfaAfaga*a*c |
| AV05231 | AV05231-SS | 1313 | g*u*cuuugoGfoUfuCfaagaaa*u*a | AV05231-AS | 1454 | u*Af*uuucUfugaaCfcCfcAfaag*a*c |
| AV05232 | AV05232-SS | 1314 | g*c*uuugggGfuUfcAfagaaau*a*a | AV05232-AS | 1455 | u*Uf*auuuCfuugaAfcCfcCfaaa*g*c |
| AV05233 | AV05233-SS | 1315 | c*g*guucaaGfaAfaUfaauaau*c*a | AV05233-AS | 1456 | u*Gf*auuaUfuauuUfcUfuGfaac*c*g |
| AV05234 | AV05234-SS | 1316 | g*g*aaauaaUfaAfuCfcaugau*c*a | AV05234-AS | 1457 | u*Gf*aucaUfggauUfaUfuAfuuu*c*c |
| AV05235 | AV05235-SS | 1317 | g*c*aguauaAfaAfuGfgcagaa*a*a | AV05235-AS | 1458 | u*Uf*uucuGfccauUfuUfaUfacu*g*c |
| AV05236 | AV05236-SS | 1318 | g*a*guauaaAfaUfgGfcagaaa*g*a | AV05236-AS | 1459 | u*Cf*uuucUfgccaUfuUfuAfuac*u*c |
| AV05237 | AV05237-SS | 1319 | c*g*guaugaUfaUfuGfccuugu*u*a | AV05237-AS | 1460 | u*Af*acaaGfgcaaUfaUfcAfuac*c*g |
| AV05238 | AV05238-SS | 1320 | g*g*auauugCfcUfuGfuugaaa*c*a | AV05238-AS | 1461 | u*Gf*uuucAfacaaGfgCfaAfuau*c*c |
| AV05239 | AV05239-SS | 1321 | c*a*uauugcCfuUfgUfugaaac*u*a | AV05239-AS | 1462 | u*Af*guuuCfaacaAfgGfcAfaua*u*g |
| AV05240 | AV05240-SS | 1322 | g*a*uugccuUfgUfuGfaaacug*g*a | AV05240-AS | 1463 | u*Cf*caguUfucaaCfaAfgGfcaa*u*c |
| AV05241 | AV05241-SS | 1323 | c*c*cuuguuGfaAfaCfuggaaa*c*a | AV05241-AS | 1464 | u*Gf*uuucCfaguuUfcAfaCfaag*g*g |
| AV05242 | AV05242-SS | 1324 | g*g*uugaaaCfuGfgAfaaccac*a*a | AV05242-AS | 1465 | u*Uf*guggUfuuccAfgUfuUfcaa*c*c |
| AV05243 | AV05243-SS | 1325 | g*g*gaaaccAfcAfgUfgaauua*c*a | AV05243-AS | 1466 | u*Gf*uaauUfcacuGfuGfgUfuuc*c*c |
| AV05244 | AV05244-SS | 1326 | g*a*accacaGfuGfaAfuuacac*a*a | AV05244-AS | 1467 | u*Uf*guguAfauucAfcUfgUfggu*u*c |
| AV05245 | AV05245-SS | 1327 | g*c*cacaguGfaAfuUfacacag*a*a | AV05245-AS | 1468 | u*Uf*cuguGfuaauUfcAfcUfgug*g*c |
| AV05246 | AV05246-SS | 1328 | g*c*aaaauaCfuCfuCfcagaaa*g*a | AV05246-AS | 1469 | u*Cf*uuucUfggagAfgUfaUfuuu*g*c |
| AV05247 | AV05247-SS | 1329 | g*c*ucuccaGfaAfaGfccaaga*u*a | AV05247-AS | 1470 | u*Af*ucuuGfgcuuUfcUfgGfaga*g*c |
| AV05248 | AV05248-SS | 1330 | g*a*agccaaGfaUfaCfccuuag*u*a | AV05248-AS | 1471 | u*Af*cuaaGfgguaUfcUfuGfgcu*u*c |
| AV05249 | AV05249-SS | 1331 | c*a*agagauAfcAfgAfggacau*a*a | AV05249-AS | 1472 | u*Uf*auguCfcucuGfuAfuCfucu*u*g |
| AV05250 | AV05250-SS | 1332 | c*a*gauacaGfaGfgAfcauaaa*a*a | AV05250-AS | 1473 | u*Uf*uuuaUfguccUfcUfgUfauc*u*g |
| AV05251 | AV05251-SS | 1333 | g*a*cagaggAfcAfuAfaaauaa*c*a | AV05251-AS | 1474 | u*Gf*uuauUfuuauGfuCfcUfcug*u*c |
| AV05252 | AV05252-SS | 1334 | g*c*agaggaCfaUfaAfaauaac*c*a | AV05252-AS | 1475 | u*Gf*guuaUfuuuaUfgUfcCfucu*g*c |
| AV05253 | AV05253-SS | 1335 | c*a*ggacauAfaAfaUfaaccca*u*a | AV05253-AS | 1476 | u*Af*ugggUfuauuUfuAfuGfucc*u*g |
| AV05254 | AV05254-SS | 1336 | c*a*cauaaaAfuAfaCfccauaa*g*a | AV05254-AS | 1477 | u*Cf*uuauGfgguuAfuUfuUfaug*u*g |
| AV05255 | AV05255-SS | 1337 | g*c*auaaaaUfaAfcCfcauaag*a*a | AV05255-AS | 1478 | u*Uf*cuuaUfggguUfaUfuUfuau*g*c |
| AV05256 | AV05256-SS | 1338 | g*a*cccauaAfgAfuGfaucugu*g*a | AV05256-AS | 1479 | u*Cf*acagAfucauCfuUfaUfggg*u*c |
| AV05257 | AV05257-SS | 1339 | c*g*acuggaUfuCfuGfgagaaa*a*a | AV05257-AS | 1480 | u*Uf*uuucUfccagAfaUfcCfagu*c*g |
| AV05258 | AV05258-SS | 1340 | c*a*cuggauUfcUf@Gfagaaaa*c*a | AV05258-AS | 1481 | u*Gf*uuuuCfuccaGfaAfuCfcag*u*g |
| AV05499 | AV05499-SS | 1341 | g*u*cauucaGfaGfgAfggauga*a*a | AV05499-AS | 1482 | u*Uf*ucauCfcuccUfcUfgAfaug*a*c |
| AV05500 | AV05500-SS | 1342 | g*a*aggucuUfuUfcAfggauga*u*a | AV05500-AS | 1483 | u*Af*ucauCfcugaAfaAfgAfccu*u*c |
| AV05501 | AV05501-SS | 1343 | g*c*uuuucaGfgAfuGfauuuuc*ufa | AV05501-AS | 1484 | u*Af*gaaaAfucauCfcUfgAfaaa*g*c |
| AV05502 | AV05502-SS | 1344 | g*g*gaugauUfuUfcUfuauauc*a*a | AV05502-AS | 1485 | u*Uf*gauaUfaagaAfaAfuCfauc*c*c |
| AV05503 | AV05503-SS | 1345 | g*g*augguuUfaCfuUfgugucc*u*a | AV05503-AS | 1486 | u*Af*ggAfc(aUNA)caagUfaAfaCfca |
| | | | | | | u*c*c |
| AV05504 | AV05504-SS | 1346 | g*g*ccaagaGfuGfaAfuaggac*a*a | AV05504-AS | 1487 | u*Uf*guccUfauucAfcUfcUfugg*c*c |
| AV05505 | AV05505-SS | 1347 | g*u*uucuggGfuAfuUfcuuuca*a*a | AV05505-AS | 1488 | u*Uf*ugaaAfgaauAfcCfcAfgaa*a*c |
| AV05506 | AV05506-SS | 1348 | g*u*ucugggUfaUfuCfuuucaa*g*a | AV05506-AS | 1489 | u*Cf*uugaAfagaaUfaCfcCfaga*a*c |
| AV05507 | AV05507-SS | 1349 | g*c*aaauaaGfcGfcUfugcaac*a*a | AV05507-AS | 1490 | u*Uf*guugCfaagcGfcUfuAfuuu*g*c |
| AV05508 | AV05508-SS | 1350 | g*g*cuugcaAfcAfaAfgacauu*u*a | AV05508-AS | 1491 | u*Af*aaugUfcuuuGfuUfgCfaag*c*c |
| AV05509 | AV05509-SS | 1351 | g*c*aaagacAfuUfuAfugugga*c*a | AV05509-AS | 1492 | u*Gf*ucCfa(cUNA)auaaAfuGfuCfuu |
| | | | | | | u*g*c |
| AV05510 | AV05510-SS | 1352 | g*u*auguggAfcCfuAfgacaug*a*a | AV05510-AS | 1493 | u*Uf*caugUfcuagGfuCfcAfcau*a*c |
| AV05511 | AV05511-SS | 1353 | c*u*ugccaaGfaGfuGfcucaag*a*a | AV05511-AS | 1494 | u*Uf*cuugAfgcacUfcUfuGfgca*a*g |
| AV05512 | AV05512-SS | 1354 | c*c*caagaaAfgAfuGfcacgga*u*a | AV05512-AS | 1495 | u*Af*ucCfg(uUNA)gcauCfuUfuCfuu |
| | | | | | | g*g*g |
| AV05513 | AV05513-SS | 1355 | g*g*aaaucuUfuGfuCfuccuua*a*a | AV05513-AS | 1496 | u*Uf*uaagGfagacAfaAfgAfuuu*c*c |
| AV05514 | AV05514-SS | 1356 | c*a*aaucuuUfoUfcUfccuuaa*a*a | AV05514-AS | 1497 | u*Uf*uuaaGfgagaCfaAfaGfauu*u*g |
| AV05515 | AV05515-SS | 1357 | g*a*aucuuuGfuCfuCfcuuaaa*a*a | AV05515-AS | 1498 | u*Uf*uuuaAfggagAfcAfaAfgau*u*c |
| AV05516 | AV05516-SS | 1358 | g*c*uuugucUfcCfuUfaaaaca*u*a | AV05516-AS | 1499 | u*Af*uguuUfuaagGfaGfaCfaaa*g*c |
| AV05517 | AV05517-SS | 1359 | g*u*ugucucCfuUfaAfaacauc*u*a | AV05517-AS | 1500 | u*Af*gaugUfuuuaAfgGfaGfaca*a*c |
| AV05518 | AV05518-SS | 1360 | g*u*gucuccUfuAfaAfacaucu*g*a | AV05518-AS | 1501 | u*Cf*agauGfuuuuAfaGfgAfgac*a*c |
| AV05519 | AV05519-SS | 1361 | g*g*ucuccuUfaAfaAfcaucug*a*a | AV05519-AS | 1502 | u*Uf*cagaUfguuuUfaAfgGfaga*c*c |
| AV05520 | AV05520-SS | 1362 | g*u*ccuuaaAfaCfaUfcugaga*g*a | AV05520-AS | 1503 | u*Cf*ucUfc(aUNA)gaugUfuUfuAfag |
| | | | | | | g*a*c |
| AV05521 | AV05521-SS | 1363 | g*u*uaaaacAfuCfuGfagagug*g*a | AV05521-AS | 1504 | u*Cf*caCfu(cUNA)ucagAfuGfuUfuu |
| | | | | | | a*a*c |
| AV05522 | AV05522-SS | 1364 | g*a*gcucuuUfcUf&Gfuuucag*u*a | AV05522-AS | 1505 | u*Af*cugaAfaccaGfaAfaGfagc*u*c |
| AV05523 | AV05523-SS | 1365 | g*g*aacuggAfuAfuUfguugcu*g*a | AV05523-AS | 1506 | u*Cf*agCfa(aUNA)caauAfuCfcAfgu |
| | | | | | | u*c*c |
| AV05524 | AV05524-SS | 1366 | c*a*ggagocAfuCfuCfuggaua*c*a | AV05524-AS | 1507 | u*Gf*uaucCfagagAfuGfcCfucc*u*g |
| AV05525 | AV05525-SS | 1367 | c*g*caucucUfgGfaUfacacau*u*a | AV05525-AS | 1508 | u*Af*auguGfuaucCfaGfaGfaug*c*g |
| AV05526 | AV05526-SS | 1368 | c*u*uguguaAfaAfuGfgauaau*g*a | AV05526-AS | 1509 | u*Cf*auuaUfccauUfuUfaCfaca*a*g |
| AV05527 | AV05527-SS | 1369 | g*g*uguaaaAfuGfgAfuaauga*g*a | AV05527-AS | 1510 | u*Cf*ucauUfauccAfuUfuUfaca*c*c |
| AV05528 | AV05528-SS | 1370 | c*u*guaccaCfcAfaAfaucaag*c*a | AV05528-AS | 1511 | u*Gf*cuugAfuuuuGfgUfgGfuac*a*g |
| AV05529 | AV05529-SS | 1371 | g*c*caggauCfgUfuGfgagzaa*c*a | AV05529-AS | 1512 | u*Gf*uuCfc(uUNA)ccaaCfgAfuCfcu |
| | | | | | | g*g*c |
| AV05530 | AV05530-SS | 1372 | c*u*agagucAfcCfuAfagauuu*u*a | AV05530-AS | 1513 | u*Af*aaauCfuuagGfuGfaCfucu*a*g |
| AV05531 | AV05531-SS | 1373 | g*g*gcauuuUfaAfaUfcaaucu*g*a | AV05531-AS | 1514 | u*Cf*agauUfgauuUfaAfaAfugc*c*c |
| AV05532 | AV05532-SS | 1374 | c*g*cauuuuAfaAfuCfaaucug*a*a | AV05532-AS | 1515 | u*Uf*cagaUfugauUfuAfaAfaug*c*g |
| AV05533 | AV05533-SS | 1375 | c*c*auuuuaAfaUfcAfaucuga*a*a | AV05533-AS | 1516 | u*Uf*ucagAfuugaUfuUfaAfaau*g*g |
| AV05534 | AV05534-SS | 1376 | c*g*acacauCfuUfuCfuuuggg*g*a | AV05534-AS | 1517 | u*Cf*ccCfa(aUNA)agaaAfgAfuGfug |
| | | | | | | u*c*g |
| AV05535 | AV05535-SS | 1377 | g*c*uuucuuUfgGfgGfuucaag*a*a | AV05535-AS | 1518 | u*Uf*cuugAfacccCfaAfaGfaaa*g*c |
| AV05536 | AV05536-SS | 1378 | g*u*uuggggUfuCfaAfgaaaua*a*a | AV05536-AS | 1519 | u*Uf*uauuUfcuugAfaCfcCfcaa*a*c |
| AV05537 | AV05537-SS | 1379 | c*u*ucaagaAfaUfaAfuaaucc*a*a | AV05537-AS | 1520 | u*Uf*ggauUfauuaUfuUfcUfuga*a*g |
| AV05538 | AV05538-SS | 1380 | g*u*caagaaAfuAfaUfaaucca*u*a | AV05538-AS | 1521 | u*Af*uggaUfuauuAfaUfuCfuug*a*c |
| AV05539 | AV05539-SS | 1381 | g*g*gguaugAfuAfuUfgccuug*u*a | AV05539-AS | 1522 | u*Af*caAfg(uUNA)caauAfuCfaUfac |
| | | | | | | c*c*c |
| AV05540 | AV05540-SS | 1382 | g*u*auugccUfuGfuUfgaaacu*g*a | AV05540-AS | 1523 | u*Cf*aguuUfcaacAfaGfgCfaau*a*c |
| AV05541 | AV05541-SS | 1383 | g*c*uuguugAfaAfcUfggaaac*c*a | AV05541-AS | 1524 | u*Gf*guUfu(cUNA)caguUfuCfaAfca |
| | | | | | | a*g*c |
| AV05542 | AV05542-SS | 1384 | c*g*aaaccaCfaGfuGfaauuac*a*a | AV05542-AS | 1525 | u*Uf*guaaUfucacUfgUfgGfuuu*c*g |
| AV05543 | AV05543-SS | 1385 | g*u*cuccagAfaAfgCfcaagau*a*a | AV05543-AS | 1526 | u*Uf*aucuUfggcuUfuCfuGfgag*a*c |
| AV05544 | AV05544-SS | 1386 | g*u*ccagaaAfgCfcAfagauac*c*a | AV05544-AS | 1527 | u*Gf*guauCfuuggCfuUfuCfugg*a*c |
| AV05545 | AV05545-SS | 1387 | c*a*aagccaAfgAfuAfcccuua*g*a | AV05545-AS | 1528 | u*Cf*uaAfg(gUNA)guauCfuUfgGfcu |
| | | | | | | u*u*g |
| AV05546 | AV05546-SS | 1388 | g*g*ccaagaUfaCfcCfuuagug*a*a | AV05546-AS | 1529 | u*Uf*cacuAfagggUfaUfcUfugg*c*c |
| AV05547 | AV05547-SS | 1389 | g*c*aagauaCfcCfuUfagugac*c*a | AV05547-AS | 1530 | u*Gf*guCfa(cUNA)uaagGfgUfaUfcu |
| | | | | | | u*g*c |
| AV05548 | AV05548-SS | 1390 | g*a*agauacCfcUfuAfgugacc*a*a | AV05548-AS | 1531 | u*Uf*ggUfc(aUNA)cuaaGfgGfuAfuc |
| | | | | | | u*u*c |
| AV05549 | AV05549-SS | 1391 | g*g*aagaguGfcCfaGfaagaga*u*a | AV05549-AS | 1532 | u*Af*ucucUfucugGfcAfcUfcuu*c*c |
| AV05550 | AV05550-SS | 1392 | c*u*gccagaAfgAfgAfuacaga*g*a | AV05550-AS | 1533 | u*Cf*ucugUfaucuCfuUfcUfggc*a*g |
| AV05551 | AV05551-SS | 1393 | g*g*aagagaUfaCfaGfaggaca*u*a | AV05551-AS | 1534 | u*Af*ugucCfucugUfaUfcUfcuu*c*c |
| AV05552 | AV05552-SS | 1394 | g*a*gagauaCfaGfaGfgacaua*a*a | AV05552-AS | 1535 | u*Uf*uaugUfccucUfgUfaUfcuc*u*c |
| AV05553 | AV05553-SS | 1395 | g*g*auacagAfgGfaCfauaaaa*u*a | AV05553-AS | 1536 | u*Af*uuuuAfugucCfuCfuGfuau*c*c |
| AV05554 | AV05554-SS | 1396 | c*a*uacagaGfgAfcAfuaaaau*a*a | AV05554-AS | 1537 | u*Uf*auuuUfauguCfcUfcUfgua*u*g |
| AV05555 | AV05555-SS | 1397 | g*g*gacauaAfaAfuAfacccau*a*a | AV05555-AS | 1538 | u*Uf*auggGfuuauUfuUfaUfguc*c*c |
| AV05556 | AV05556-SS | 1398 | g*a*aaauaaCfcCfaUfaagaug*a*a | AV05556-AS | 1539 | u*Uf*caucUfuaugGfgUfuAfuuu*u*c |
| AV05557 | AV05557-SS | 1399 | g*a*aauaacCfcAfuAfagauga*u*a | AV05557-AS | 1540 | u*Af*ucauCfuuauGfgGfuUfauu*u*c |
| AV05558 | AV05558-SS | 1400 | g*u*aacccaUfaAfgAfugaucu*g*a | AV05558-AS | 1541 | u*Cf*agauCfaucuUfaUfgGfguu*a*c |
| AV05559 | AV05559-SS | 1401 | g*a*acccauAfaGfaUfgaucug*u*a | AV05559-AS | 1542 | u*Af*cagaUfcaucUfuAfuGfggu*u*c |
| AV05560 | AV05560-SS | 1402 | g*g*uacgugGfaCfuGfgauucu*g*a | AV05560-AS | 1543 | u*Cf*agaaUfccagUfcCfaCfgua*c*c |
| AV05561 | AV05561-SS | 1403 | c*u*ggacugGfaUfuCfuggaga*a*a | AV05561-AS | 1544 | u*Uf*ucucCfagaaUfcCfaGfucc*a*g |
| AV05562 | AV05562-SS | 1404 | g*g*gauucuGfgAfgAfaaacuc*a*a | AV05562-AS | 1545 | u*Uf*gaguUfuucuCfcAfgAfauc*c*c |
| AV05563 | AV05563-SS | 1405 | c*g*auucugGfaGfaAfaacuca*a*a | AV05563-AS | 1546 | u*Uf*ugagUfuuucUfcCfaGfaau*c*g |
| AV05564 | AV05564-SS | 1406 | c*a*uucuggAfgAfaAfacucaa*g*a | AV05564-AS | 1547 | u*Cf*uugaGfuuuuCfuCfcAfgaa*u*g |
| AV05565 | AV05565-SS | 1407 | g*g*caugcuUfcCfuCfcacagu*a*a | AV05565-AS | 1548 | u*Uf*acugUfggagGfaAfgCfaug*c*c |
| AV05566 | AV05566-SS | 1408 | g*g*cuagaaGfaAfaAfcaaacu*g*a | AV05566-AS | 1549 | u*Cf*aguuUfguuuUfcUfuCfuag*c*c |
| AV05567 | AV05567-SS | 1409 | c*c*uagaagAfaAfaCfaaacug*u*a | AV05567-AS | 1550 | u*Af*caguUfuguuUfuCfuUfcua*g*g |
| AV05568 | AV05568-SS | 1410 | g*u*agaagaAfaAfcAfaacugu*c*a | AV05568-AS | 1551 | u*Gf*acagUfuuguUfuUfcUfucu*a*c |
| AV05569 | AV05569-SS | 1411 | g*a*gaagaaAfaCfaAfacuguc*a*a | AV05569-AS | 1552 | u*Uf*gacaGfuuugUfuUfuCfuuc*u*c |
| AV05570 | AV05570-SS | 1412 | c*a*agaaaaCfaAfaCfugucac*a*a | AV05570-AS | 1553 | u*Uf*gugaCfaguuUfgUfuUfucu*u*g |
| AV06227 | AV06227-SS | 1413 | a*a*aaugcuAfgAfaGfaaaaca*a*a | AV06227-AS | 1554 | u*Uf uguuUfucuuCfuAfgCfauu*u*u |
| AV06228 | AV06228-SS | 1414 | a*a*augcuaGfaAfgAfaaacaa*a*a | AV06228-AS | 1555 | u*Uf*uuguUfuucuUfcUfaGfcau*u*u |
| AV06229 | AV06229-SS | 1415 | a*a*ugcuagAfaGfaAfaacaaa*c*a | AV06229-AS | 1556 | u*Gf*uuugUfuuucUfuCfuAfgca*u*u |
| AV06230 | AV06230-SS | 1416 | a*u*gcuagaAfgAfaAfacaaac*u*a | AV06230-AS | 1557 | u*Af*guuuGfuuuuCfuUfcUfagc*a*u |
| AV06231 | AV06231-SS | 1417 | u*g*cuagaaGfaAfaAfcaaacu*g*a | AV06231-AS | 1558 | u*Cf*aguuUfguuuUfcUfuCfuag*c*a |
| AV06232 | AV06232-SS | 1418 | g*c*uagaagAfaAfaCfaaacug*u*a | AV06232-AS | 1559 | u*Af*caguUfuguuUfuCfuUfcua*g*c |
| AV06233 | AV06233-SS | 1419 | c*u*agaagaAfaAfcAfaacugu*c*a | AV06233-AS | 1560 | u*Gf*acagUfuuguUfuUfcUfucu*a*g |
| AV06234 | AV06234-SS | 1420 | u*a*gaagaaAfaCfaAfacuguc*a*a | AV06234-AS | 1561 | u*Uf*gacaGfuuugUfuUfuCfuuc*u*a |
| AV06235 | AV06235-SS | 1421 | a*g*aagaaaAfcAfaAfcuguca*c*a | AV06235-AS | 1562 | u*Gf*ugacAfguuuGfuUfuUfcuu*c*u |
| AV06236 | AV06236-SS | 1422 | g*a*agaaaaCfaAfaCfugucac*a*a | AV06236-AS | 1563 | u*Uf*gugaCfaguuUfgUfuUfucu*u*c |
| AV06237 | AV06237-SS | 1423 | a*a*gaaaacAfaAfcUfgucaca*a*a | AV06237-AS | 1564 | u*Uf*ugugAfcaguUfuGfuUfuuc*u*u |
| AV06238 | AV06238-SS | 1424 | a*g*aaaacaAfaCfuGfucacaa*g*a | AV06238-AS | 1565 | u*Cf*uuguGfacagUfuUfgUfuuu*c*u |
| AV06239 | AV06239-SS | 1425 | g*a*aaacaaAfcUfgUfcacaag*u*a | AV06239-AS | 1566 | u*Af*cuugUfgacaGfuUfuGfuuu*u*c |
| AV06240 | AV06240-SS | 1426 | a*a*aacaaaCfuGfuCfacaagu*u*a | AV06240-AS | 1567 | u*Af*acuuGfugacAfgUfuUfguu*u*u |
| AV06241 | AV06241-SS | 1427 | a*a*acaaacUfgUfcAfcaaguu*g*a | AV06241-AS | 1568 | u*Cf*aacuUfgugaCfaGfuUfugu*u*u |

Table 3 shows certain chemically modified FXI RNAi agent antisense strand and sense strand sequences of the invention. In some embodiments of methods of the invention, RNAi agents shown in Table 3 are administered to a cell and/or subject. In some embodiments of methods of the invention, an RNAi agent with a polynucleotide sequence shown in Table 3 is administered to a subject. In some embodiments of the invention an RNAi agent administered to a subject comprises is a duplex identified in a row in Table 3, column one and includes the sequence modifications and/or delivery compound show in the sense and antisense strand sequences in the same row in Table 3, columns four and seven, respectively. The sequences were used in certain in vivo testing studies described elsewhere herein. In some embodiments of methods of the invention, a sequence shown in Table 3 may be attached to (also referred to herein as “conjugated to”) a compound for delivery, a non-limiting example of which is a GalNAc-containing compound, with a delivery compound identified in Table 3 as “GLX-n” on sense strands in column four. As used herein, “GLX-n” is used to represent either a “GLS-n*” or a GLO-n” delivery compound (“X” can be either “S” or “O”) and GLX-0 can be any of the “GLS-n*” and “GLO-n” delivery compounds that can be attached to 3′-end of oligonucleotide during synthesis. As used herein and shown in Table 3, “GLX-n” is used to indicate the attached GalNAc-containing compound is any one of compounds GLS-1*, GLS-2*, GLS-3*, GLS-4*, GLS-5*, GLS-6*, GLS-7*, GLS-8*, GLS-9*, GLS-10*, GLS-11*, GLS-12*, GLS-13*, GLS-14*, GLS-15*, GLS-16*, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16, The structure of each of which is provided elsewhere herein. One skilled in the art will be able to prepare and use a dsRNA compound of the invention in which the attached delivery compound is one of GLS-1*, GLS-2*, GLS-3*, GLS-4*, GLS-5*, GLS-6*, GLS-7*, GLS-8*, GLS-9*, GLS-10*, GLS-11*, GLS-12*, GLS-13*, GLS-14*, GLS-15*, GLS-16*, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16. Column one of Table 3 provides a Duplex AD #assigned to the duplex of the sense and antisense sequences in that row of the table. For example, Duplex AD #AD00764 is the duplex of sense strand SEQ ID NO: 587 and antisense strand SEQ ID NO: 651. Each line in Table 3 provides a sense strand and an antisense strand, and discloses the duplex of the sense and antisense strands shown. The “Sense strand SS #” in Table 3 column two is the assigned identifier for the Sense Sequence (including modifications) shown column 4 in the same row. The “Antisense strand AS #” in Table 3 column 5 is the assigned identifier for the Antisense sequence (including modifications) shown in column seven. An identifier for certain attached GalNAc-containing “GLO-n” or “GLS-n*” compounds is shown as GLS-5*, GLS-15* or GLX-0, and it will be understood that another of the “GLO-n” or “GLS-n*” compounds may be substituted for the compound shown as GLO-0, with the resulting compound included in an embodiment of a method and/or a composition of the invention.

Table 3 provides chemically modified FXI RNAi agent antisense strand and sense strand sequences. All sequences shown 5′ to 3′. The sequences were used in certain in vivo testing studies described elsewhere herein. A delivery molecule used in the in vivo studies is indicated as “GLO-n” or “GLS-n*” at the 3′-end or 5′-end of each sense strand.

| | Sense | SEQ | | Antisense | SEQ | |
|---|---|---|---|---|---|---|
| Duplex | strand | ID | | strand | ID | |
| AD# | SS# | NO | Sense Strand | AS# | NO | Antisense Strand |
| AD00764 | AD00764-SS | 587 | (GLS- | AD00764-AS | 651 | u*Uf*caUfccugaaAfaGfaccUf |
| | | | 15*)(invab*)gcaaggucUfuUfuc | | | u*g*c |
| | | | Afggauga*a*(invab) | | | |
| AD00765 | AD00765-SS | 588 | (GLS- | AD00765-AS | 652 | u*Gf*auAfuaagaaAfaUfcauCf |
| | | | 15*)(invab*)gaggaugaUfuUfuc | | | c*u*c |
| | | | Ufuauauc*a*(invab) | | | |
| AD00766 | AD00766-SS | 589 | (GLS- | AD00766-AS | 653 | u*Uf*ucAfggacacAfaGfuaaAf |
| | | | 15*)(invab*)ggguuuacUfuGfug | | | c*c*c |
| | | | Ufccugaa*a*(invab) | | | |
| AD00767 | AD00767-SS | 590 | (GLS- | AD00767-AS | 654 | u*Uf*unCfaguaacAfcUfgucUf |
| | | | 15*)(invab*)caaagacaGfuGfuu | | | u*u*g |
| | | | Afcagaaa*a*(invab) | | | |
| AD00768 | AD00768-SS | 591 | (GLS- | AD00768-AS | 655 | u*Uf*gcUfugaaagAfaUfaccCf |
| | | | 15*)(invab*)gcuggguaUfuCfuu | | | a*g*c |
| | | | Ufcaagca*a*(invab) | | | |
| AD00769 | AD00769-SS | 592 | (GLS- | AD00769-AS | 656 | u*Uf*uuGfuugcaaGfcGfcuuAf |
| | | | 15*)(invab*)gaauaagcGfcUfug | | | u*u*c |
| | | | Cfaacaaa*a*(invab) | | | |
| AD00770 | AD00770-SS | 593 | (GLS- | AD00770-AS | 657 | u*Uf*ccAfcauaaaUfgUfcuuUf |
| | | | 15*)(invab*)gacaaagaCfaUfuu | | | g*u*c |
| | | | Afugugga*a*(invab) | | | |
| AD00771 | AD00771-SS | 594 | (GLS- | AD00771-AS | 658 | u*Af*ggUfc(cUNA)acauAfaAf |
| | | | 15*)(invab*)gaagacauUfuAfug | | | uguCfu*u*c |
| | | | Ufggaccu*a*(invab) | | | |
| AD00772 | AD00772-SS | 595 | (GLS- | AD00772-AS | 659 | u*Uf*auAfguuuauGfcCfcuuCf |
| | | | 15*)(invab*)gaugaaggGfcAfua | | | a*u*c |
| | | | Afacuaua*a*(invab) | | | |
| AD00773 | AD00773-SS | 596 | (GLS- | AD00773-AS | 660 | u*Uf*acGfugaaaaAfgUfggcAf |
| | | | 15*)(invab*)gacugccaCfuUfuu | | | g*u*c |
| | | | Ufcacqua*a*(invab) | | | |
| AD00774 | AD00774-SS | 597 | (GLS- | AD00774-AS | 661 | u*Af*cuCfucagauGfuUfunaAf |
| | | | 15*)(invab*)gccuuaaaAfcAfuc | | | g*g*c |
| | | | Ufgagagu*a*(invab) | | | |
| AD00775 | AD00775-SS | 598 | (GLS- | AD00775-AS | 662 | u*Gf*aaAfccagaaAfgAfgcuUf |
| | | | 15*)(invab*)ccaaagcuCfuUfuc | | | u*g*g |
| | | | Ufgguuuc*a*(invab) | | | |
| AD00776 | AD00776-SS | 599 | (GLS- | AD00776-AS | 663 | u*Uf*ugAfagaaagCfuUfuaaGf |
| | | | 15*)(invab*)guacuuaaAfgCfuu | | | u*a*c |
| | | | Ufcuucaa*a*(invab) | | | |
| AD00988 | AD00988-SS | 600 | (GLS- | AD00988-AS | 664 | u*Gf*auauAfagaaAfaUfcAfuc |
| | | | 15*)(invab*)gaggaugaUfuUfuC | | | c*u*c |
| | | | fuuauauc*a*(invab) | | | |
| AD00989 | AD00989-SS | 601 | (GLS- | AD00989-AS | 665 | u*Uf*ugauAfuaagAfaAfaUfca |
| | | | 15*)(invab*)cgaugauuUfuCfuU | | | u*c*g |
| | | | fauaucaa*a*(invab) | | | |
| AD00990 | AD00990-SS | 602 | (GLS- | AD00990-AS | 666 | u*Cf*auucAfccagAfaAfcUfga |
| | | | 15*)(invab*)gcuucaguUfuCfuG | | | a*g*c |
| | | | fgugaaug*a*(invab) | | | |
| AD00991 | AD00991-SS | 603 | (GLS- | AD00991-AS | 667 | u*Af*gucaCfacauUfcAfcCfag |
| | | | 15*)(invab*)guucugguGfaAfuG | | | a*a*c |
| | | | fugugacu*a*(invab) | | | |
| AD00992 | AD00992-SS | 604 | GLS- | AD00992-AS | 668 | ufUf*caGfg(aUNA)cacaAfgUf |
| | | | 15*)(invab*)gugguuuaCfuUfgU | | | aAfacc*a*c |
| | | | fguccuga*a*(invab) | | | |
| AD00993 | AD00993-SS | 605 | (GLS- | AD00993-AS | 669 | u*Gf*uuucUfguaaCfaCfuGfuc |
| | | | 15*)(invab*)gaagacagUfgUfuA | | | u*u*c |
| | | | fcagaaac*a*(invab) | | | |
| AD00994 | AD00994-SS | 606 | (GLS- | AD00994-AS | 670 | u*G*fuguuUfcuguAfaCfaCfug |
| | | | 15*)(invab*)ggacagugUfuAfcA | | | u*c*c |
| | | | fgaaacac*a*(invab) | | | |
| AD00995 | AD00995-SS | 607 | GLS- | AD00995-AS | 671 | u*Uf*ucacUfcuugGfcAfgUfgu |
| | | | 15*)(invab*)caaacacuGfcCfaA | | | u*u*g |
| | | | fgagugaa*a*(invab) | | | |
| AD00996 | AD00996-SS | 608 | (GLS- | AD00996-AS | 672 | u*Af*uaccCfagaaAfuCfgCfug |
| | | | 15*)(invab*)gagcagcgAfuUfuC | | | c*u*c |
| | | | fuggguau*a*(invab) | | | |
| AD00997 | AD00997-SS | 609 | (GLS- | AD00997-AS | 673 | u*Gf*aauaCfccagAfaAfuCfgc |
| | | | 15*)(invab*)ccagcgauUfuCfuG | | | u*g*g |
| | | | fgguauuc*a*(invab) | | | |
| AD00998 | AD00998-SS | 610 | (GLS- | AD00998-AS | 674 | u*Uf*acguGfaaaaAfgUfeGfca |
| | | | 15*)(invab*)gacugccaCfuUfuU | | | g*u*c |
| | | | fucacgua*a*(invab) | | | |
| AD00999 | AD00999-SS | 611 | (GLS- | AD00999-AS | 675 | u*Uf*gcuuGfaaagAfaUfaCfcc |
| | | | 15*)(invab*)gcuggguaUfuCfuU | | | a*g*c |
| | | | fucaagca*a*(invab) | | | |
| AD01000 | AD01000-SS | 612 | (GLS- | AD01000-AS | 676 | u*Af*uugcUfugaaAfgAfaUfac |
| | | | 15*)(invab*)gggguauuCfuUfuC | | | c*c*c |
| | | | faagcaau*a*(invab) | | | |
| AD01001 | AD01001-SS | 613 | (GLS- | AD01001-AS | 677 | u*Gf*ugAfg(cUNA)auugCfuUf |
| | | | 15*)(invab*)gucuuucaAfgCfaA | | | gAfaag*a*c |
| | | | fugcucac*a*(invab) | | | |
| AD01002 | AD01002-SS | 614 | (GLS- | AD01002-AS | 678 | u*Uf*uguuGfcaagCfgCfuUfau |
| | | | 15*)(invab*)gaaauaagCfgCfuU | | | u*u*c |
| | | | fgcaacaa*a*(invab) | | | |
| AD01003 | AD01003-SS | 615 | (GLS- | AD01003-AS | 679 | u*Uf*aaauGfucuuUfgUfuGfca |
| | | | 15*)(invab*)ccuugcaaCfaAfaG | | | a*g*g |
| | | | facauuua*a*(invab) | | | |
| AD01004 | AD01004-SS | 616 | GLS- | AD01004-AS | 680 | u*Cf*cacaUfaaauGfuCfuUfug |
| | | | 15*)(invab*)gaacaaagAfcAfuU | | | u*u*c |
| | | | fuaugugg*a*(invab) | | | |
| AD01005 | AD01005-SS | 617 | (GLS- | AD01005-AS | 681 | u*Cf*uaGfg(uUNA)ccacAfuAf |
| | | | 15*)(invab*)ggacauuuAfuGfuG | | | aAfugu*c*c |
| | | | fgaccuag*a*(invab) | | | |
| AD01006 | AD01006-SS | 618 | GLS- | AD01006-AS | 682 | u*Uf*ucauGfucuaGfgUfcCfac |
| | | | 15*)(invab*)gauguggaCfcUfaG | | | a*u*c |
| | | | facaugaa*a*(invab) | | | |
| AD01007 | AD01007-SS | 619 | (GLS- | AD01007-AS | 683 | u*Af*guuuAfugccCfuUfcAfug |
| | | | 15*)(invab*)ggacaugaAfgGfgC | | | u*c*c |
| | | | fauaaacu*a*(invab) | | | |
| AD01008 | AD01008-SS | 620 | (GLS- | AD01008-AS | 684 | u*Af*uaguUfuaugCfcCfuUfca |
| | | | 15*)(invab*)gcaugaagGfgCfaU | | | u*g*c |
| | | | faaacuau*a*(invab) | | | |
| AD01009 | AD01009-SS | 621 | GLS- | AD01009-AS | 685 | u*Uf*guuaUfaguuUfaUfgCfcc |
| | | | 15*)(invab*)caaggecaUfaAfaC | | | u*u*g |
| | | | fuauaaca*a*(invab) | | | |
| AD01010 | AD01010-SS | 622 | (GLS- | AD01010-AS | 686 | u*Cf*auucUfugagCfaCfuCfuu |
| | | | 15*)(invab*)cccaagagUfgCfuC | | | g*g*g |
| | | | faagaaug*a*(invab) | | | |
| AD01011 | AD01011-SS | 623 | (GLS- | AD01011-AS | 687 | u*Uf*aaggAfgacaAfaGfaUfuu |
| | | | 15*)(invab*)gagaaaucUfuUfgU | | | c*u*c |
| | | | fcuccuua*a*(invab) | | | |
| AD01012 | AD01012-SS | 624 | (GLS- | AD01012-AS | 688 | u*Gf*auguUfuuaaGfgAfgAfca |
| | | | 15*)(invab*)guuugucuCfcUfuA | | | a*a*c |
| | | | faaacauc*a*(invab) | | | |
| AD01013 | AD01013-SS | 625 | (GLS- | AD01013-AS | 689 | u*Af*agagCfuuugCfuCfuUfuu |
| | | | 15*)(invab*)guaaaaagAfpCfaA | | | u*a*c |
| | | | fagcucuu*a*(invab) | | | |
| AD01014 | AD01014-SS | 626 | (GLS- | AD01014-AS | 690 | u*Gf*caacAfauauCfcAfgUfuc |
| | | | 15*)(invab*)caagaacuGfgAfuA | | | u*u*g |
| | | | fuuguugc*a*(invab) | | | |
| AD01015 | AD01015-SS | 627 | (GLS- | AD01015-AS | 691 | u*Af*uaggUfaaaaAfaCfuGfgc |
| | | | 15*)(invab*)ccugccagUfuUfuU | | | a*g*g |
| | | | fuaccuau*a*(invab) | | | |
| AD01016 | AD01016-SS | 628 | (GLS- | AD01016-AS | 692 | u*Gf*aaagCfuuuaAfgUfaAfca |
| | | | 15*)(invab*)gaguguuaCfuUfaA | | | c*u*c |
| | | | fagcunuc*a*(invab) | | | |
| AD01017 | AD01017-SS | 629 | GLS | AD01017-AS | 693 | u*Af*agaaAfgcuuUfaAfgUfaa |
| | | | 15*)(invab*)cuguuacuUfaAfaG | | | c*a*g |
| | | | fcuuucuu*a*(invab) | | | |
| AD01018 | AD01018-SS | 630 | (GLS- | AD01018-AS | 694 | u*Uf*ugaaGfaaagCfuUfuAfag |
| | | | 15*)(invab*)guacuuaaAfgCfuU | | | u*a*c |
| | | | fucuucaa*a*(invab) | | | |
| AD01019 | AD01019-SS | 631 | (GLS- | AD01019-AS | 695 | u*Uf*uugaAfgaaaGfcUfuUfaa |
| | | | 15*)(invab*)gacuuaaaGfcUfuU | | | g*u*c |
| | | | fcuucaaa*a*(invab) | | | |
| AD01020 | AD01020-SS | 632 | (GLS- | AD01020-AS | 696 | u*Uf*aaugUfguauCfcAfgAfga |
| | | | 15*)(invab*)ccaucucuGfgAfuA | | | u*g*g |
| | | | fcacauna*a*(invab) | | | |
| AD01477 | AD01477-SS | 633 | GLS | AD01477-AS | 697 | u*Cf*acauUfcaccAfgAfaAfcu |
| | | | 15*)(imann*)gucaguuuCfuGfgU | | | g*a*c |
| | | | fgaaugug*a*(imann) | | | |
| AD01478 | AD01478-SS | 634 | (GLS- | AD01478-AS | 698 | u*Gf*cuugAfaagaAfuAfcCfca |
| | | | 15*)(imann*)gucuggguAfuUfcU | | | g*a*c |
| | | | fuucaagc*a*(imann) | | | |
| AD01479 | AD01479-SS | 635 | GLS- | AD01479-AS | 699 | u*Uf*gagcAfuugcUfuGfaAfag |
| | | | 15*)(imann*)guucuuncAfaGfcA | | | a*a*c |
| | | | faugcuca*a*(imann) | | | |
| AD01480 | AD01480-SS | 636 | (GLS- | AD01480-AS | 700 | u*Uf*uauuUfggugUfgAfgCfau |
| | | | 15*)(imann*)ccaaugcuCfaCfaC | | | u*g*g |
| | | | fcaaauaa*a*(imann) | | | |
| AD01481 | AD01481-SS | 637 | (GLS- | AD01481-AS | 701 | u*Cf*uuauUfugguGfuGfaGfca |
| | | | 15*)(imann*)gaaugcucAfcAfcC | | | u*u*c |
| | | | faaauaag*a*(imann) | | | |
| AD01482 | AD01482-SS | 638 | (GLS- | AD01482-AS | 702 | u*Gf*cuuaUfuuggUfgUfgAfgc |
| | | | 15*)(imann*)gaugcucaCfaCfcA | | | a*u*c |
| | | | faauaagc*a*(imann) | | | |
| AD01483 | AD01483-SS | 639 | (GLS- | AD01483-AS | 703 | u*Uf*gcAfa(gUNA)cgcuUfaUf |
| | | | 15*)(imann*)gcaccaaaUfaAfgC | | | uUfggu*g*c |
| | | | fgcuugca*a*(imann) | | | |
| AD01484 | AD01484-SS | 640 | (GLS- | AD01484-AS | 704 | u*Uf*ugcaAfgcgcUfuAfuUfug |
| | | | 15*)(imann*)gaccaaauAfaGfcG | | | g*u*c |
| | | | fcuugcaa*a*(imann) | | | |
| AD01485 | AD01485-SS | 641 | (GLS- | AD01485-AS | 705 | u*Cf*gugaAfaaagUfgGfcAfgu |
| | | | 15*)(imann*)gccacugcCfaCfuU | | | g*g*c |
| | | | fuuucacg*a*(imann) | | | |
| AD01486 | AD01486-SS | 642 | (GLS- | AD01486-AS | 706 | u*Af*cgugAfaaaaGfuGfgCfag |
| | | | 15*)(imann*)gcacugccAfcUfuU | | | u*g*c |
| | | | fuucacgu*a*(imann) | | | |
| AD01487 | AD01487-SS | 643 | GLS- | AD01487-AS | 707 | u*Af*gaaaGfcuunAfaGfuAfac |
| | | | 15*)(imann*)gguguuacUfuAfaA | | | a*c*c |
| | | | fgcuuucu*a*(imann) | | | |
| AD00990-1 | AD00990-SS | 644 | (GLS- | AD00990-AS | 708 | u*Cf*auucAfccagAfaAfcUfga |
| | | | 15*)(imann*)gcuucaguUfuCfuG | | | a*g*c |
| | | | fgugaaug*a*(imann) | | | |
| AD00991-1 | AD00991-SS | 645 | (GLS- | AD00991-AS | 709 | u*Af*gucaCfacauUfcAfcCfag |
| | | | 15*)(imann*)guucugguGfaAfuG | | | a*a*c |
| | | | fugugacu*a*(imann) | | | |
| AD00997-1 | AD00997-SS | 646 | GLS- | AD00997-AS | 710 | u*Gf*aauaCfccagAfaAfuCfgc |
| | | | 15*)(imann*)ccagcgauUfuCfu | | | u*g*g |
| | | | Gfgguauuc*a*(imam) | | | |
| AD00998-1 | AD00998-SS | 647 | GLS- | AD00998-AS | 711 | u*Uf*acguGfaaaaAfgUfgGfca |
| | | | 15*)(imann*)gacugccaCfuUfuU | | | g*u*c |
| | | | fucacgua*a*(imann) | | | |
| AD00999-1 | AD00999-SS | 648 | (GLS- | AD00999-AS | 712 | u*Uf*gcuuGfaaagAfaUfaCfcc |
| | | | 15*)(imann*)gcuggguaUfuCfuU | | | a*g*c |
| | | | fucaagca*a*(imann) | | | |
| AD01002-1 | AD01002-SS | 649 | (GLS- | AD01002-AS | 713 | u*Uf*uguuGfcaagCfgCfuUfau |
| | | | 15*)(imann*)gaaauaagCfgCfuU | | | u*u*c |
| | | | fgcaacaa*a*(imann) | | | |
| AD01016-1 | AD01016-SS | 650 | GLS- | AD01016-AS | 714 | u*Gf*aaagCfuuuaAfgUfaAfca |
| | | | 15*)(imann*)gaguguuaCfuUfaA | | | c*u*c |
| | | | fagcuuuc*a*(imann) | | | |
| AD02349 | AD02349-SS | 1731 | (GLS- | AD02349-AS | 1648 | u*Af*uaagAfaaauCfaUfcCfug |
| | | | 15*)(invab*)guucaggaUfgAfuU | | | a*a*c |
| | | | fuucuuau*a*(invab) | | | |
| AD02350 | AD02350-SS | 1732 | (GLS- | AD02350-AS | 1649 | u*Uf*auaaGfaaaaUfcAfuCfcu |
| | | | 15*)(invab*)gucaggauGfaUfuU | | | g*a*c |
| | | | fucuuaua*a*(invab) | | | |
| AD02351 | AD02351-SS | 1569 | (GLS- | AD02351-AS | 1650 | u*Cf*acacAfuucaCfcAfgAfaa |
| | | | 15*)(invab*)gaguuucuGfgUfgA | | | c*u*c |
| | | | faugugug*a*(invab) | | | |
| AD02352 | AD02352-SS | 1570 | (GLS- | AD02352-AS | 1651 | u*Uf*ugguGfugagCfaUfuGfcu |
| | | | 15*)(invab*)gcaagcaaUfgCfuC | | | u*g*c |
| | | | facaccaa*a*(invab) | | | |
| AD02353 | AD02353-SS | 1571 | (GLS- | AD02353-AS | 1652 | u*Uf*uuggUfgugaGfcAfuUfgc |
| | | | 15*)(invab*)gaagcaanGfcUfcA | | | u*u*c |
| | | | fcaccaaa*a*(invab) | | | |
| AD02354 | AD02354-SS | 1572 | (GLS- | AD02354-AS | 1653 | u*Uf*aguuUfaugcCfcUfuCfau |
| | | | 15*)(invab*)cacaugaaGfgGfcA | | | g*u*g |
| | | | fuaaacua*a*(invab) | | | |
| AD02355 | AD02355-SS | 1573 | (GLS- | AD02355-AS | 1654 | u*Gf(cuguUfauagUfuUfaUfgc |
| | | | 15*)(invab*)ggggcauaAfaCfuA | | | c*c*c |
| | | | fuaacagc*a*(invab) | | | |
| AD02356 | AD02356-SS | 1574 | (GLS- | AD02356-AS | 1655 | u*Af*aucuUfagguGfaCfuCfua |
| | | | 15*)(invab*)cgguagagUfcAfcC | | | c*c*g |
| | | | fuaagauu*a*(invab) | | | |
| AD02357 | AD02357-SS | 1575 | GLS- | AD02357-AS | 1656 | u*Uf*guagAfcacgCfaAfaAfuc |
| | | | 15*)(invab*)gaagauuuUfgCfgU | | | u*u*c |
| | | | fgucuaca*a*(invab) | | | |
| AD02358 | AD02358-SS | 1576 | (GLS- | AD02358-AS | 1657 | u*Uf*ugauUfuaaaAfuGfcCfac |
| | | | 15*)(invab*)gcaguggcAfuUfuU | | | u*g*c |
| | | | faaaucaa*a*(invab) | | | |
| AD02359 | AD02359-SS | 1577 | (GLS- | AD02359-AS | 1658 | u*Af*uugaUfuuaaAfaUfgCfca |
| | | | 15*)(invab*)gaguggcaUfuUfuA | | | c*u*c |
| | | | faaucaau*a*(invab) | | | |
| AD02360 | AD02360-SS | 1578 | (GLS- | AD02360-AS | 1659 | u*Uf*uuauUfucagAfuUfgAfuu |
| | | | 15*)(invab*)guaaaucaAfuCfuG | | | u*a*c |
| | | | faaauaaa*a*(invab) | | | |
| AD02361 | AD02361-SS | 1579 | (GLS- | AD02361-AS | 1660 | u*Af*uuucUfugaaCfcCfcAfaa |
| | | | 15*)(invab*)gucuuuggGfgUfuC | | | g*a*c |
| | | | faagaaau*a*(invab) | | | |
| AD02362 | AD02362-SS | 1580 | (GLS- | AD02362-AS | 1661 | u*Gf*aunaUfuaunUfcUfuGfaa |
| | | | 15*)(invab*)cgguucaaGfaAfaU | | | c*c*g |
| | | | faauaauc*a*(invab) | | | |
| AD02363 | AD02363-SS | 1581 | (GLS- | AD02363-AS | 1662 | u*Uf*uucuGfccauUfuUfaUfac |
| | | | 15*)(invab*)gcaguauaAfaAfuG | | | u*g*c |
| | | | fgcagaaa*a*(invab) | | | |
| AD02364 | AD02364-SS | 1582 | (GLS- | AD02364-AS | 1663 | u*Uf*guguAfauucAfcUfgUfgg |
| | | | 15*)(invab*)gaaccacaGfuGfaA | | | u*u*c |
| | | | fuuacaca*a*(invab) | | | |
| AD02365 | AD02365-SS | 1583 | (GLS- | AD02365-AS | 1664 | u*Uf*cuguGfuaauUfcAfcUfgu |
| | | | 15*)(invab*)gccacaguGfaAfuU | | | g*g*c |
| | | | facacaga*a*(invab) | | | |
| AD02366 | AD02366-SS | 1584 | (GLS- | AD02366-AS | 1665 | u*Af*ugggUfuauuUfuAfuGfuc |
| | | | 15*)(invab*)caggacauAfaAfaU | | | c*u*g |
| | | | faacccau*a*(invab) | | | |
| AD02369 | AD02369-SS | 1585 | (GLS- | AD02369-AS | 1666 | u*Af*ucauCfcugaAfaAfgAfcc |
| | | | 15*)(invab*)gaaggucuUfuUfcA | | | u*u*c |
| | | | fggaugau*a*(invab) | | | |
| AD02370 | AD02370-SS | 1586 | (GLS- | AD02370-AS | 1667 | u*Af*gaaaAfucauCfcUfgAfaa |
| | | | 15*)(invab*)gcuuuucaGfgAfuG | | | a*g*c |
| | | | fauuuucu*a*(invab) | | | |
| AD02371 | AD02371-SS | 1587 | (GLS- | AD02371-AS | 1668 | u*Uf*gauaUfaagaAfaAfuCfau |
| | | | 15*)(invab*)gggaugauUfuUfcU | | | c*c*c |
| | | | fuauauca*a*(invab) | | | |
| AD02372 | AD02372-SS | 1588 | (GLS- | AD02372-AS | 1669 | u*Af*ggAfc(aUNA)caagUfaAf |
| | | | 15*)(invab*)ggaugguuUfaCfuU | | | aCfcau*c*c |
| | | | fguguccu*a*(invab) | | | |
| AD02373 | AD02373-SS | 1589 | (GLS- | AD02373-AS | 1670 | u*Cf*cuauUfcacuCfuUfgGfca |
| | | | 15*)(invab*)gacugccaAfgAfgU | | | g*u*c |
| | | | fgaauagg*a*(invab) | | | |
| AD02374 | AD02374-SS | 1590 | (GLS- | AD02374-AS | 1671 | u*Uf*guccUfauucAfcUfcUfug |
| | | | 15*)(invab*)ggccaagaGfuGfaA | | | g*c*c |
| | | | fuaggaca*a*(invab) | | | |
| AD02375 | AD02375-SS | 1591 | (GLS- | AD02375-AS | 1672 | u*Gf*aaagAfauacCfcAfgAfaa |
| | | | 15*)(invab*)ggauuucuGfgGfuA | | | u*c*c |
| | | | fuucuuuc*a*(invab) | | | |
| AD02376 | AD02376-SS | 1592 | (GLS- | AD02376-AS | 1673 | u*Uf*ugaaAfgaauAfcCfcAfga |
| | | | 158)(invab*)guuucuggGfuAfuU | | | a*a*c |
| | | | fcuuucaa*a*(invab) | | | |
| AD02377 | AD02377-SS | 1593 | (GLS- | AD02377-AS | 1674 | u*Cf*ungaAfagaaUfaCfcCfag |
| | | | 15*)(invab*)guucugggUfaUfuC | | | a*a*c |
| | | | fuuucaag*a*(invab) | | | |
| AD02378 | AD02378-SS | 1594 | (GLS- | AD02378-AS | 1675 | u*Gf*caUfu(gUNA)cuugAfaAf |
| | | | 15*)(invab*)cguauucuUfuCfaÅ | | | gAfaua*c*g |
| | | | fgcaaugc*a*(invab) | | | |
| AD02379 | AD02379-SS | 1595 | (GLS- | AD02379-AS | 1676 | u*Uf*guugCfaagcGfcUfuAfuu |
| | | | 15*)(invab*)gcaaauaaGfcGfcU | | | u*g*c |
| | | | fugcaaca*a*(invab) | | | |
| AD02380 | AD02380-SS | 1596 | (GLS- | AD02380-AS | 1677 | u*Af*aaugUfcuunGfuUfgCfaa |
| | | | 15*)(invab*)ggcuugcaAfcAfaA | | | g*c*c |
| | | | fgacauuu*a*(invab) | | | |
| AD02381 | AD02381-SS | 1597 | (GLS- | AD02381-AS | 1678 | u*Gf*ucCfa(cUNA)auaaAfuGf |
| | | | 15*)(invab*)gcaaagacAfuUfuA | | | uCfuuu*g*c |
| | | | fuguggac*a*(invab) | | | |
| AD02382 | AD02382-SS | 1598 | (GLS- | AD02382-AS | 1679 | u*Uf*caugUfcuagGfuCfcAfca |
| | | | 15*)(invab*)guauguggAfcCfuA | | | u*a*c |
| | | | fgacauga*a*(invab) | | | |
| AD02383 | AD02383-SS | 1599 | (GLS- | AD02383-AS | 1680 | u*Uf*cuugAfgcacUfcUfuGfgc |
| | | | 15*)(invab*)cuugccaaGfaGfuG | | | a*a*g |
| | | | fcucaaga*a*(invab) | | | |
| AD02384 | AD02384-SS | 1600 | (GLS- | AD02384-AS | 1681 | u*Af+ucCfg(uUNA)gcauCfuUf |
| | | | 15*)(invab*)cccaagaaAfgAfuG | | | uCfuug*g*g |
| | | | fcacggau*a*(invab) | | | |
| AD02385 | AD02385-SS | 1601 | (GLS- | AD02385-AS | 1682 | u*Uf*uaagGfagacAfaAfgAfuu |
| | | | 15%)(invab*)ggaaaucuUfuGfuC | | | u*c*c |
| | | | fuccuuaa*a*(invab) | | | |
| AD02386 | AD02386-SS | 1602 | (GLS- | AD02386-AS | 1683 | u*Af*accaGfaaagAfgCfuUfug |
| | | | 15*)(invab*)cagcaaagCfuCfuU | | | c*u*g |
| | | | fucugguu*a*(invab) | | | |
| AD02387 | AD02387-SS | 1603 | (GLS- | AD02387-AS | 1684 | u*Af*guauUfuuagUfuGfgAfga |
| | | | 15*)(invab*)cgaucuccAfaCfuA | | | u*c*g |
| | | | faaauacu*a*(invab) | | | |
| AD02388 | AD02388-SS | 1604 | (GLS- | AD02388-AS | 1685 | u*Cf*aunaUfccauUfuUfaCfac |
| | | | 15*)(invab*)cuuguguaAfaAfuG | | | a*a*g |
| | | | fgauaaug*a*(invab) | | | |
| AD02389 | AD02389-SS | 1605 | (GLS- | AD02389-AS | 1686 | u*Cf*acGfc(aUNA)aaauCfuUf |
| | | | 15*)(invab*)cucaccuaAfgAfuU | | | aGfgug*a*g |
| | | | fuugcgug*a*(invab) | | | |
| AD02390 | AD02390-SS | 1606 | (GLS- | AD02390-AS | 1687 | u*Af*caCfg(cUNA)aaaaUfcUf |
| | | | 15*)(invab*)gcaccuaaGfaUfuU | | | uAfggu*g*C |
| | | | fugcgugu*a*(invab) | | | |
| AD02391 | AD02391-SS | 1607 | (GLS- | AD02391-AS | 1688 | u*Af*gaCfa(cUNA)gcaaAfaUf |
| | | | 15*)(invab*)gccuaagaUfuUfuG | | | cUfuag*g*c |
| | | | fcgugucu*a*(invab) | | | |
| AD02392 | AD02392-SS | 1608 | (GLS- | AD02392-AS | 1689 | u*Uf*cagaUfugauUfuAfaAfau |
| | | | 15*)(invab*)cgcauuuuAfaAfuC | | | g*c*g |
| | | | faaucuga*a*(invab) | | | |
| AD02393 | AD02393-SS | 1609 | (GLS- | AD02393-AS | 1690 | u*Af*acCfc(cUNA)aaagAfaAf |
| | | | 15*)(invab*)gcacaucuUfuCfuU | | | gAfugu*g*c |
| | | | fugggguu*a*(invab) | | | |
| AD02394 | AD02394-SS | 1610 | (GLS- | AD02394-AS | 1691 | u*Uf*gaAfc(cUNA)ccaaAfgAf |
| | | | 15*)(invab*)gcaucuuuCfuUfuG | | | aAfgau*g*c |
| | | | fggguuca*a*(invab) | | | |
| AD02395 | AD02395-SS | 1611 | (GLS- | AD02395-AS | 1692 | u*Uf*uucuUfgaacCfcCfaAfag |
| | | | 15*)(invab*)guucuuugGfgGfuU | | | a*a*c |
| | | | fcaagaaa*a*(invab) | | | |
| AD02396 | AD02396-SS | 1612 | (GLS- | AD02396-AS | 1693 | u*Cf*uuucUfgccaUfuUfuAfua |
| | | | 15*)(invab*)gaguauaaAfaUfgG | | | c*u*c |
| | | | fcagaaag*a*(invab) | | | |
| AD02397 | AD02397-SS | 1613 | (GLS- | AD02397-AS | 1694 | u*Af*acaaGfgcaaUfaUfcAfua |
| | | | 15*)(invab*)cgguaugaUfaUfuG | | | c*c*g |
| | | | fccuuguu*a*(invab) | | | |
| AD02398 | AD02398-SS | 1614 | (GLS- | AD02398-AS | 1695 | u*Gf*uuucAfacaaGfgCfaAfua |
| | | | 15*)(invab*)ggauauugCfcUfuG | | | u*c*c |
| | | | fuugaaac*a*(invab) | | | |
| AD02399 | AD02399-SS | 1615 | (GLS- | AD02399-AS | 1696 | u*Uf*guaaUfucacUfgUfgGfuu |
| | | | 15*)(invab*)cgaaaccaCfaGfuG | | | u*c*g |
| | | | faauuaca*a*(invab) | | | |
| AD02400 | AD02400-SS | 1616 | (GLS- | AD02400-AS | 1697 | u*Cf*uuucUfggagAfgUfaUfuu |
| | | | 15*)(invab*)gcaaaauaCfuCfuC | | | u*g*c |
| | | | fcagaaag*a*(invab) | | | |
| AD02401 | AD02401-SS | 1617 | GLS- | AD02401-AS | 1698 | u*Cf*uaAfg(gUNA)guauCfuUf |
| | | | 15*)(invab*)caaagccaAfgAfuA | | | gGfcuu*u*g |
| | | | fcccuuag*a*(invab) | | | |
| AD02402 | AD02402-SS | 1618 | (GLS- | AD02402-AS | 1699 | u*Gf*uuauUfuuauGfuCfcUfcu |
| | | | 15*)(invab*)gacagaggAfcAfuA | | | g*u*c |
| | | | faaauaac*a*(invab) | | | |
| AD02403 | AD02403-SS | 1619 | GLS- | AD02403-AS | 1700 | u*Cf*acagAfucauCfuUfaUfgg |
| | | | 15*)(invab*)gacccauaAfgAfuG | | | g*u*c |
| | | | faucugug*a*(invab) | | | |
| AD02404 | AD02404-SS | 1620 | GLS- | AD02404-AS | 1701 | u*Uf*uuucUfccagAfaUfcCfag |
| | | | 15*)(invab*)cgacuggaUfuCfuG | | | u*c*g |
| | | | fgagaaaa*a*(invab) | | | |
| AD00990-2 | AD00990-2- | 1621 | (GLS- | AD00990-2-AS | 1702 | u*Cf*auucAfccagAfaAfcUfga |
| | SS | | 15*)(invab*)gcgucaguUfuCfuG | | | c*g*c |
| | | | fgugaaug*a*(invab) | | | |
| AD00990 | AD00990-SS | 1622 | (GLS- | AD00990-AS | 1703 | u*Cf*auucAfccagAfaAfcUfga |
| | | | 15*)(invab*)gcuucaguUfuCfuG | | | a*g*c |
| | | | fgugaaug*a*(invab) | | | |
| AD00991 | AD00991-SS | 1623 | GLS- | AD00991-AS | 1704 | u*Af*gucaCfacauUfcAfcCfag |
| | | | 15*)(invab*)guucugguGfaAfuG | | | a*a*c |
| | | | fugugacu*a*(invab) | | | |
| AD00997-2 | AD00997-2- | 1624 | (GLS- | AD00997-2-AS | 1705 | u*Gf*aauaCfccagAfaAfuCfgc |
| | SS | | 15*)(invab*)cgagcgauUfuCfuG | | | u*c*g |
| | | | fgguauuc*a*(invab) | | | |
| AD00997 | AD00997-SS | 1625 | (GLS- | AD00997-AS | 1706 | u*Gf*aauaCfccagAfaAfuCfgc |
| | | | 15*)(invab*)ccagcgauUfuCfuG | | | u*g*g |
| | | | fgguauuc*a*(invab) | | | |
| AD02432 | AD02432-SS | 1626 | (GLS- | AD02432-AS | 1707 | u*Uf*uuuaAfggagAfcAfaAfga |
| | | | 15*)(invab*)gaaucunuGfuCfuC | | | u*u*c |
| | | | fcuuaaaa*a*(invab) | | | |
| AD02433 | AD02433-SS | 1627 | (GLS- | AD02433-AS | 1708 | u*Af*uguuUfuaagGfaGfaCfaa |
| | | | 15*)(invab*)gcuuugucUfcCfuU | | | a*g*c |
| | | | faaaacau*a*(invab) | | | |
| AD02434 | AD02434-SS | 1628 | (GLS- | AD02434-AS | 1709 | u*Uf*cagaUfguuuUfaAfgGfag |
| | | | 15*)(invab*)ggucuccuUfaAfaA | | | a*c*c |
| | | | fcaucuga*a*(invab) | | | |
| AD02435 | AD02435-SS | 1629 | (GLS- | AD02435-AS | 1710 | u*Gf*uuCfc(uUNA)ccaaCfgAf |
| | | | 15*)(invab*)gccaggauCfgUfuG | | | uCfcug*g*c |
| | | | fgaggaac*a*(invab) | | | |
| AD02436 | AD02436-SS | 1630 | (GLS- | AD02436-AS | 1711 | u*Uf*cuugAfacccCfaAfaGfaa |
| | | | 15*)(invab*)gcuuucuuUfgGfgG | | | a*g*c |
| | | | fuucaaga*a*(invab) | | | |
| AD02437 | AD02437-SS | 1631 | (GLS- | AD02437-AS | 1712 | u*Af*uggaUfuauuAfuUfuCfuu |
| | | | 15*)(invab*)gucaagaaAfuAfaU | | | g*a*c |
| | | | faauccau*a*(invab) | | | |
| AD02438 | AD02438-SS | 1632 | (GLS- | AD02438-AS | 1713 | u*Af*caAfg(gUNA)caauAfuCf |
| | | | 15*)(invab*)gggguaugAfuAfuU | | | aUfacc*c*c |
| | | | fgccuugu*a*(invab) | | | |
| AD02439 | AD02439-SS | 1633 | (GLS- | AD02439-AS | 1714 | u*Af*uuuuAfugucCfuCfuGfua |
| | | | 15*)(invab*)ggauacagAfgGfaC | | | u*cac |
| | | | fauaaaau*a*(invab) | | | |
| AD02440 | AD02440-SS | 1634 | (GLS- | AD02440-AS | 1715 | u*Uf*auuuUfauguCfcUfcUfgu |
| | | | 15*)(invab*)cauacagaGfgAfcA | | | a*u*g |
| | | | fuaaaaua*a*(invab) | | | |
| AD02441 | AD02441-SS | 1635 | (GLS- | AD02441-AS | 1716 | u*Uf*auggGfuuauUfuUfaUfgu |
| | | | 15*)(invab*)gggacquaAfaAfuA | | | c*c*c |
| | | | facccaua*a*(invab) | | | |
| AD02442 | AD02442-SS | 1636 | (GLS- | AD02442-AS | 1717 | u*Uf*ucucCfagaaUfcCfaGfuc |
| | | | 15*)(invab*)cuggacugGfaUfuC | | | c*a*g |
| | | | fuggagaa*a*(invab) | | | |
| AD02443 | AD02443-SS | 1637 | (GLS- | AD02443-AS | 1718 | u*Cf*aguuUfguuuUfcUfuCfua |
| | | | 15*)(invab*)ggcuagaaGfaAfaA | | | g*c*c |
| | | | fcaaacug*a*(invab) | | | |
| AD02444 | AD02444-SS | 1638 | (GLS- | AD02444-AS | 1719 | u*Af*caguUfuguuUfuCfuUfcu |
| | | | 15*)(invab>)ccuagaagAfaAfaC | | | a*g*g |
| | | | faaacugu*a*(invab) | | | |
| AD02445 | AD02445-SS | 1639 | (GLS- | AD02445-AS | 1720 | u*Gf*acagUfuuguUfuUfcUfuc |
| | | | 15*)(invab*)guagaagaAfaAfcA | | | u*a*c |
| | | | faacuguc*a*(invab) | | | |
| AD02446 | AD02446-SS | 1640 | (GLS- | AD02446-AS | 1721 | u*Uf*gacaGfuuugUfuUfuCfuu |
| | | | 15*)(invab*)gagaagaaAfaCfaA | | | c*u*c |
| | | | facuguca*a*(invab) | | | |
| AD02447 | AD02447-SS | 1641 | GLS- | AD02447-AS | 1722 | u*Uf*gugaCfaguuUfgUfuUfuc |
| | | | 15*)(invab*)caagaaaaCfaAfaC | | | u*u*g |
| | | | fugucaca*a*(invab) | | | |
| AD02375-1 | AD02375-1- | 1642 | (GLS- | AD02375-1-AS | 1723 | a*Gf*aaagAfauacCfcAfgAfaa |
| | SS | | 15*)(invab*)ggauuucuGfeGfuA | | | u*c*c |
| | | | fuucuuuc*u*(invab) | | | |
| AD01606 | AD01606-SS | 1643 | g*g*guauUfCfUfuucaagcaau | AD01606-AS | 1724 | (phos)a*Uf*ugcUfugaaagaAf |
| | | | (L96) | | | uAfccc*a*g |
| AD01607 | AD01607-SS | 1644 | a*u*uucuGfGfGfuauucuuuca | AD01607-AS | 1725 | (phos)u*Gf*aaaGfaauacccAf |
| | | | (L96) | | | gAfaau*c*g |
| AD00726 | AD00726-SS | 1645 | u*c*agGfaUfGfAfuuuucuuau*u* | AD00726-AS | 1726 | u*Af*uAfaGfaaaAfuCfaUfcCf |
| | | | (L96) | | | uGfa*a*a |
| AD00727 | AD00727-SS | 1646 | g*g*guauUfCfUfuucaagcaau | AD00727-AS | 1727 | a*Uf*ugcUfugaaagaAfuAfccc |
| | | | (L96) | | | *a*g |
| AD00728 | AD00728-SS | 1647 | a*u*uucuGfGfGfuauucuuuca | AD00728-AS | 1728 | u*Gf*aaaGfaauacccAfgAfaau |
| | | | (L96) | | | *c*g |

L96 is refers to the compound GalNAc3 in Jayaprakash, et al., (2014) J. Am. Chem. Soc., 136, 16958-16961.

In certain embodiments of the invention a dsRNA (also referred to herein as a “duplex”) is one disclosed in one of Tables 1-3. Each row in Tables 1-3 discloses a duplex comprising the sequence of the sense strand and the sequence of the antisense strand in that table row. In addition to the duplexes disclosed in Tables 1-3, it will be understood that in some embodiments, a duplex of the invention may include sense and antisense sequences shown in Tables 1-3, that differ by zero, one, two, or three nucleotides shown in a sequence shown in Tables 1-3. It will be understood that the sequence of the sense strand and the sequence of the antisense strand in a duplex of the invention may be independently selected. Thus, a dsRNA of the invention may comprise a sense strand and an antisense strand of a duplex disclosed in a row in Tables 1-3. Alternatively, in a dsRNA of the invention, one or both of the selected sense and antisense strand in the dsRNA may include sequences shown in Tables 1-3 but with one or both of the sense and antisense sequences including 1, 2, 3, or more nucleobase substitutions from the parent sequence. The selected sequences may in some embodiments be longer or shorter than their parent sequence. As non-limiting examples, in some embodiments, a sense strand in a duplex of the invention may be SEQ ID NO: 6, 7, 9 longer than SEQ ID NO: 102, 103, 105, respectively. Thus, dsRNA agents included in the invention can but need not include exact sequences of the sense and antisense pairs disclosed as duplexes in Tables 1-3.

In some embodiments, a dsRNA agent comprises a sense strand and an antisense strand, nucleotide positions 2 to 18 in the antisense strand comprising a region of complementarity to a FXI RNA transcript, wherein the region of complementarity comprises at least 15 contiguous nucleotides that differ by 0, 1, 2, or 3 nucleotides from one of the antisense sequences listed in one of Tables 1-3, and optionally comprising a targeting ligand. In some instances, the region of complementarity to the FXI RNA transcript comprises at least 15, 16, 17, 18, or 19 contiguous nucleotides that differ by no more than 3 nucleotides from one of the antisense sequences listed in one of Tables 1-3. In some embodiments of a dsRNA agent of the invention, the antisense strand of the dsRNA is at least substantially complementary to any one of a target region of SEQ ID NO: 1 and is provided in any one of Tables 1-3. In some embodiments, an antisense strand of a dsRNA agent of the invention is fully complementary to any one of a target region of SEQ ID NO: 1 and is provided in any one of Tables 1-3. In some embodiments a dsRNA agent includes a sense strand sequence set forth in any one of Tables 1-3, and the sense strand sequence is at least substantially complementary to the antisense strand sequence in the dsRNA agent. In other embodiments, a dsRNA agent of the invention comprises a sense strand sequence set forth in any one of Tables 1-3, and the sense strand sequence is fully complementary to the antisense strand sequence in the dsRNA agent. In some instances, a dsRNA agent of the invention comprises an antisense strand sequence set forth in anyone of Tables 1-3. Some embodiments of a dsRNA agent of the invention comprises the sense and antisense sequences disclosed as duplex in any of Tables 1-3. As described herein, it will be understood that the sense and antisense strands in a duplex of the invention may be independently selected.

### Mismatches

It is known to skilled in art, mismatches are tolerated for efficacy in dsRNA, especially the mismatches are within terminal region of dsRNA. Certain mismatches tolerate better, for example mismatches with wobble base pairs G:U and A:C are tolerated better for efficacy (Du et el., A systematic analysis of the silencing effects of an active siRNA at all single-nucleotide mismatched target sites. Nucleic Acids Res. 2005 Mar. 21; 33(5):1671-7. Doi: 10.1093/nar/gki312. Nucleic Acids Res. 2005; 33(11):3698). Some embodiments of methods and compounds of the invention a FXI dsRNA agent may contain one or more mismatches to the FXI target sequence. In some embodiments, FXI dsRNA agent of the invention includes no mismatches. In certain embodiments, FXI dsRNA agent of the invention includes no more than 1 mismatch. In some embodiments, FXI dsRNA agent of the invention includes no more than 2 mismatches. In certain embodiments, FXI dsRNA agent of the invention includes no more than 3 mismatches. In some embodiments of the invention, an antisense strand of a FXI dsRNA agent contains mismatches to a FXI target sequence that are not located in the center of the region of complementarity. In some embodiments, the antisense strand of the FXI dsRNA agent includes 1, 2, 3, 4, or more mismatches that are within the last 5, 4, 3, 2, or 1 nucleotide from one or both of the 5′ or 3′ end of the region of complementarity. Methods described herein and/or methods known in the art can be used to determine whether a FXI dsRNA agent containing a mismatch to a FXI target sequence is effective in inhibiting the expression of the FXI gene.

### Complementarity

As used herein, unless otherwise indicated, the term “complementary,” when used to describe a first nucleotide sequence (e.g., FXI dsRNA agent sense strand or targeted FXI mRNA) in relation to a second nucleotide sequence (e.g., FXI dsRNA agent antisense strand or a single-stranded antisense polynucleotide), means the ability of an oligonucleotide or polynucleotide including the first nucleotide sequence to hybridize [form base pair hydrogen bonds under mammalian physiological conditions (or similar conditions in vitro)] and form a duplex or double helical structure under certain conditions with an oligonucleotide or polynucleotide including the second nucleotide sequence. Other conditions, such as physiologically relevant conditions as can be encountered inside an organism, can apply. A skilled artisan will be able to determine the set of conditions most appropriate for a test of complementarity of two sequences in accordance with the ultimate application of the hybridized nucleotides. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification.

Complementary sequences, for example, within a FXI dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. It will be understood that in embodiments when two oligonucleotides are designed to form, upon hybridization, one or more single stranded overhangs, such overhangs are not regarded herein as mismatches with regard to the determination of complementarity. For example, a FXI dsRNA agent comprising one oligonucleotide 19 nucleotides in length and another oligonucleotide 20 nucleotides in length, wherein the longer oligonucleotide comprises a sequence of 19 nucleotides that is fully complementary to the shorter oligonucleotide, can yet be referred to as “fully complementary” for the purposes described herein. Thus, as used herein, “fully complementary” means that all (100%) of the bases in a contiguous sequence of a first polynucleotide will hybridize with the same number of bases in a contiguous sequence of a second polynucleotide. The contiguous sequence may comprise all or a part of a first or second nucleotide sequence.

The term “substantially complementary” as used herein means that in a hybridized pair of nucleobase sequences, at least about 85%, 86%, 87%, 88%, 89%, 90%, 91%, 92%, 93%, 94%, 95%, 96%, 97%, 98%, or 99%, but not all, of the bases in a contiguous sequence of a first polynucleotide will hybridize with the same number of bases in a contiguous sequence of a second polynucleotide. The term “substantially complementary” can be used in reference to a first sequence with respect to a second sequence if the two sequences include one or more, for example at least 1, 2, 3, 4, or 5 mismatched base pairs upon hybridization for a duplex up to 15, 16, 17, 18, 19, 20, 21, 22, 23, 24, 25, 26, 27, 28, 29 or 30 base pairs (bp), while retaining the ability to hybridize under the conditions most relevant to their ultimate application, e.g., inhibition of FXI gene expression via a RISC pathway.

The term, “partially complementary” may be used herein in reference to a hybridized pair of nucleobase sequences, in which at least 75%, but not all, of the bases in a contiguous sequence of a first polynucleotide will hybridize with the same number of bases in a contiguous sequence of a second polynucleotide. In some embodiments, “partially complementary” means at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the bases in a contiguous sequence of a first polynucleotide will hybridize with the same number of bases in a contiguous sequence of a second polynucleotide.

The terms “complementary,” “fully complementary,” “substantially complementary,” and “partially complimentary” are used herein in reference to the base matching between the sense strand and the antisense strand of a FXI dsRNA agent, between the antisense strand of a FXI dsRNA agent and a sequence of a target FXI mRNA, or between a single-stranded antisense oligonucleotide and a sequence of a target FXI mRNA. It will be understood that the term “antisense strand of a FXI dsRNA agent” may refer to the same sequence of an “FXI antisense polynucleotide agent”.

As used herein, the term “substantially identical” or “substantial identity” used in reference to a nucleic acid sequence means a nucleic acid sequence comprising a sequence with at least about 85% sequence identity or more, preferably at least 90%, at least 91%, at least 92%, at least 93%, at least 94%, at least 95%, at least 96%, at least 97%, at least 98%, or at least 99%, compared to a reference sequence. Percentage of sequence identity is determined by comparing two optimally aligned sequences over a comparison window. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity. The inventions disclosed herein encompasses nucleotide sequences substantially identical to those disclosed herein. e.g., in Tables 1-3. In some embodiments, the sequences disclosed herein are exactly identical, or at least about 85%, 86%, 87%, 88%, 89%, 90%, 91%, 92%, 93%, 94%, 95%, 96%, 97%, 98%, or 99% percent identical to those disclosed herein, e.g., in Tables 1-3.

As used herein, the term “strand comprising a sequence” means an oligonucleotide comprising a chain of nucleotides that is described by the sequence referred to using the standard nucleotide nomenclature. The term “double-stranded RNA” or “dsRNA,” as used herein, refers to an RNAi that includes an RNA molecule or complex of molecules having a hybridized duplex region comprising two anti-parallel and substantially or fully complementary nucleic acid strands, which are referred to as having “sense” and “antisense” orientations with respect to a target FXI RNA. The duplex region can be of any length that permits specific degradation of a desired target FXI RNA through a RISC pathway but will typically range from 9 to 30 base pairs in length, e.g., 15-30 base pairs in length. Considering a duplex between 9 and 30 base pairs, the duplex can be any length in this range, for example, 9, 10, 11, 12, 13, 14, 15, 16, 17, 18, 19, 20, 21, 22, 23, 24, 25, 26, 27, 28, 29, or 30, and any sub-range therein between, including, but not limited to 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, or 21-22 base pairs. FXI dsRNA agents generated in the cell by processing with Dicer and similar enzymes are generally in the range of 19-22 base pairs in length. One strand of the duplex region of a FXI dsDNA agent comprises a sequence that is substantially complementary to a region of a target FXI RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (herein referred to as a “hairpin loop”) between the 3′-end of one strand and the 5′-end of the respective other strand forming the duplex structure. In some embodiments of the invention, a hairpin look comprises at least 1, 2, 3, 4, 5, 6, 7, 8, 9, 10, 11, 12, 13, 14, 15, 16, 17, 18, 19, 20, or more unpaired nucleotides. Where the two substantially complementary strands of a FXI dsRNA agent are comprised by separate RNA molecules, those molecules need not, but can be covalently connected. Where the two strands are connected covalently by means other than a hairpin loop, the connecting structure is referred to as a “linker.” The term “siRNA” is also used herein to refer to a dsRNA agent as described herein.

In some embodiments of the invention a FXI dsRNA agent may include a sense and antisense sequence that have no-unpaired nucleotides or nucleotide analogs at one or both terminal ends of the dsRNA agent. An end with no unpaired nucleotides is referred to as a “blunt end” and as having no nucleotide overhang. If both ends of a dsRNA agent are blunt, the dsRNA is referred to as “blunt ended.” In some embodiments of the invention, a first end of a dsRNA agent is blunt, in some embodiments a second end of a dsRNA agent is blunt, and in certain embodiments of the invention, both ends of a FXI dsRNA agent are blunt.

In some embodiments of dsRNA agents of the invention, the dsRNA does not have one or two blunt ends. In such instances there is at least one unpaired nucleotide at the end of a strand of a dsRNA agent. For example, when a 3′-end of one strand of a dsRNA extends beyond the 5′-end of the other strand, or vice versa, there is a nucleotide overhang. A dsRNA can comprise an overhang of at least e1, 2, 3, 4, 5, 6, or more nucleotides. A nucleotide overhang can comprise or consist of a nucleotide/nucleoside analog, including a deoxynucleotide/nucleoside. It will be understood that in some embodiments a nucleotide overhang is on a sense strand of a dsRNA agent, on an antisense strand of a dsRNA agent, or on both ends of a dsRNA agent and nucleotide(s) of an overhang can be present on the 5′ end, 3′ end or both ends of either an antisense or sense strand of a dsRNA. In certain embodiments of the invention, one or more of the nucleotides in an overhang is replaced with a nucleoside thiophosphate.

As used herein, the term “antisense strand” or “guide strand” refers to the strand of a FXI dsRNA agent that includes a region that is substantially complementary to a FXI target sequence. As used herein the term “sense strand,” or “passenger strand” refers to the strand of a FXI dsRNA agent that includes a region that is substantially complementary to a region of the antisense strand of the FXI dsRNA agent.

### Modifications

In some embodiments of the invention the RNA of a FXI RNAi agent is chemically modified to enhance stability and/or one or more other beneficial characteristics. Nucleic acids in certain embodiments of the invention may be synthesized and/or modified by methods well established in the art, for example, those described in “Current protocols in Nucleic Acid Chemistry,” Beaucage, S. L. et al. (Eds.), John Wiley & Sons, Inc., New York, N.Y., USA, which is incorporated herein by reference. Modifications that can be present in certain embodiments of FXI dsRNA agents of the invention include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in certain embodiments of FXI dsRNA agents, FXI antisense polynucleotides, and FXI sense polynucleotides of the invention include, but are not limited to RNAs comprising modified backbones or no natural internucleoside linkages. As a non-limiting example, an RNA having a modified backbone may not have a phosphorus atom in the backbone. RNAs that do not have a phosphorus atom in their internucleoside backbone may be referred to as oligonucleosides. In certain embodiments of the invention, a modified RNA has a phosphorus atom in its internucleoside backbone.

It will be understood that the term “RNA molecule” or “RNA” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. The terms “ribonucleoside” and “ribonucleotide” may be used interchangeably herein. An RNA molecule can be modified in the nucleobase structure or in the ribose-phosphate backbone structure, e.g., as described herein below, and molecules comprising ribonucleoside analogs or derivatives must retain the ability to form a duplex. As non-limiting examples, an RNA molecule can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. In some embodiments of the invention, an RNA molecule comprises at least 1, 2, 3, 4, 5, 6, 7, 8, 9, 10, 11, 12, 13, 14, 15, 16, 17, 18, 19, 20, or up to the full length of the FXI dsRNA agent molecule's ribonucleosides that are modified ribonucleosides. The modifications need not be the same for each of such a plurality of modified ribonucleosides in an RNA molecule.

DsRNA agents, FXI antisense polynucleotides, and/or FXI sense polynucleotides of the invention may, in some embodiments comprise one or more independently selected modified nucleotide and/or one or more independently selected non-phosphodiester linkage. As used herein the term “independently selected” used in reference to a selected element, such as a modified nucleotide, non-phosphodiester linkage, etc., means that two or more selected elements can but need not be the same as each other.

As used herein, a “nucleotide base,” “nucleotide,” or “nucleobase” is a heterocyclic pyrimidine or purine compound, which is a standard constituent of all nucleic acids, and includes the bases that form the nucleotides adenine, guanine, cytosine, thymine, and uracil. A nucleobase may further be modified to include, though not intended to be limiting: universal bases, hydrophobic bases, promiscuous bases, size-expanded bases, and fluorinated bases. The term “ribonucleotide” or “nucleotide” may be used herein to refer to an unmodified nucleotide, a modified nucleotide, or a surrogate replacement moiety. Those in the art will recognize that guanine, cytosine, adenine, and uracil can be replaced by other moieties without substantially altering the base pairing properties of an oligonucleotide comprising a nucleotide bearing such replacement moiety.

In one embodiment, modified RNAs contemplated for use in methods and compositions described herein are peptide nucleic acids (PNAs) that have the ability to form the required duplex structure and that permit or mediate the specific degradation of a target RNA via a RISC pathway. In certain embodiments of the invention, a FXI RNA interference agent includes a single stranded RNA that interacts with a target FXI RNA sequence to direct the cleavage of the target FXI RNA.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Means of preparing phosphorus-containing linkages are routinely practiced in the art and such methods can be used to prepare certain modified FXI dsRNA agents, certain modified FXI antisense polynucleotides, and/or certain modified FXI sense polynucleotides of the invention.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Means of preparing modified RNA backbones that do not include a phosphorus atom are routinely practiced in the art and such methods can be used to prepare certain modified FXI dsRNA agents, certain modified FXI antisense polynucleotides, and/or certain modified FXI sense polynucleotides of the invention.

In certain embodiments of the invention, RNA mimetics are included in FXI dsRNAs, FXI antisense polynucleotides, and/or FXI sense polynucleotides, such as, but not limited to: replacement of the sugar and the internucleoside linkage, i.e., the backbone, of the nucleotide units with novel groups. In such embodiments, base units are maintained for hybridization with an appropriate FXI nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Means of preparing RNA mimetics are routinely practiced in the art and such methods can be used to prepare certain modified FXI dsRNA agents of the invention.

Some embodiments of the invention include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂— [wherein the native phosphodiester backbone is represented as —O—PO—CH₂—]. Means of preparing RNAs with phosphorothioate backbones and oligonucleosides with heteroatom backbones are routinely practiced in the art and such methods can be used to prepare certain modified FXI dsRNA agents, certain FXI antisense polynucleotides, and/or certain FXI sense polynucleotides of the invention.

Modified RNAs can also contain one or more substituted sugar moieties. FXI dsRNAs, FXI antisense polynucleotides, and/or FXI sense polynucleotides of the invention may comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)·OCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a FXI dsRNA agent, or a group for improving the pharmacodynamic properties of a FXI dsRNA agent, FXI antisense polynucleotide, and/or FXI sense polynucleotide, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂. Means of preparing modified RNAs such as those described are routinely practiced in the art and such methods can be used to prepare certain modified FXI dsRNA agents of the invention.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of a FXI dsRNA agent, FXI antisense polynucleotide, and/or FXI sense polynucleotide of the invention, particularly the 3′ position of the sugar on the 3′ terminal nucleotide or in 2′-5′ linked FXI dsRNAs, FXI antisense polynucleotides, or FXI sense polynucleotides, and the 5′ position of 5′ terminal nucleotide. FXI dsRNA agents, FXI antisense polynucleotides, and/or FXI sense polynucleotides may also have sugar mimetics such as cyclobutyl moieties in place of the pentofuranosyl sugar. Means of preparing modified RNAs such as those described are routinely practiced in the art and such methods can be used to prepare certain modified FXI dsRNA agents, FXI antisense polynucleotides, and/or FXI sense polynucleotides of the invention.

A FXI dsRNA agent, FXI antisense polynucleotide, and/or FXI sense polynucleotide may, in some embodiments, include nucleobase (often referred to in the art simply as “base”) modifications or substitutions. As used herein, “unmodified” or “natural” nucleobases include the purine bases adenine and guanine, and the pyrimidine bases thymine, cytosine and uracil. Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Additional nucleobases that may be included in certain embodiments of FXI dsRNA agents of the invention are known in the art, see for example: Modified Nucleosides in Biochemistry, Biotechnology and Medicine, Herdewijn, P. Ed. Wiley-VCH, 2008; The Concise Encyclopedia Of Polymer Science And Engineering, pages 858-859, Kroschwitz, J. L, Ed. John Wiley & Sons, 1990, English et al., Angewandte Chemie, International Edition, 1991, 30, 613, Sanghvi, Y S., Chapter 15, dsRNA Research and Applications, pages 289-302, Crooke, S. T. and Lebleu, B., Ed., CRC Press, 1993. Means of preparing dsRNAs, FXI antisense strand polynucleotides and/or FXI sense strand polynucleotides that comprise nucleobase modifications and/or substitutions such as those described herein are routinely practiced in the art and such methods can be used to prepare certain modified FXI dsRNA agents, FXI sense polynucleotides, and/or FXI antisense polynucleotides of the invention.

Certain embodiments of FXI dsRNA agents, FXI antisense polynucleotides, and/or FXI sense polynucleotides of the invention include RNA modified to include one or more locked nucleic acids (LNA). A locked nucleic acid is a nucleotide with a modified ribose moiety comprising an extra bridge connecting the 2′ and 4′ carbons. This structure effectively “locks” the ribose in the 3′-endo structural conformation. The addition of locked nucleic acids in a FXI dsRNA agent, FXI antisense polynucleotides, and/or FXI sense polynucleotides of the invention may increase stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Means of preparing dsRNA agents, FXI antisense polynucleotides, and/or FXI sense polynucleotides that comprise locked nucleic acid(s) are routinely practiced in the art and such methods can be used to prepare certain modified FXI dsRNA agents of the invention.

Certain embodiments of FXI dsRNA compounds, sense polynucleotides, and/or antisense polynucleotides of the invention, include at least one modified nucleotide, wherein the at least one modified nucleotide comprises: a 2′-O-methyl nucleotide, 2′-Fluoro nucleotide, 2′-deoxy nucleotide, 2′3′-seco nucleotide mimic, locked nucleotide, 2′-F-Arabino nucleotide, 2′-methoyxyethyl nucleotide, 2′-amino-modified nucleotide, 2′-alkyl-modified nucleotide, mopholino nucleotide, and 3′-Ome nucleotide, a nucleotide comprising a 5′-phosphorothioate group, or a terminal nucleotide linked to a cholesteryl derivative or dodecanoic acid bisdecylamide group, a 2′-amino-modified nucleotide, a phosphoramidate, or a non-natural base comprising nucleotide. In some embodiments, a FXI dsRNA compound includes an E-vinylphosphonate nucleotide at the 5′ end of the antisense strand, also referred to herein as the guide strand.

Certain embodiments of FXI dsRNA compounds, 3′ and 5′ end of sense polynucleotides, and/or 3′ end of antisense polynucleotides of the invention, include at least one modified nucleotide, wherein the at least one modified nucleotide comprises: abasic nucleotide, ribitol, inverted nucleotide, inverted abasic nucleotide, inverted 2′-OMe nucleotide, inverted 2′-deoxy nucleotide. It is known to skilled in art, including an abasic or inverted abasic nucleotide at the end of oligonucleotide enhances stability (Czauderna et al. Structural variations and stabilizing modifications of synthetic siRNAs in mammalian cells. Nucleic Acids Res. 2003; 31(11):2705-2716. doi:10.1093/nar/gkg393). In some embodiments, a FXI dsRNA compound includes one or more inverted abasic residues (invab) at either 3′-end or 5′-end, or both 3′-end and 5′-end. Exemplified inverted abasic residues (invab) include, but are not limited to the following:

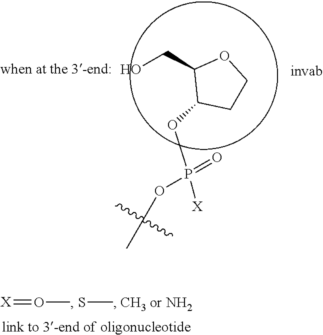

Certain embodiments of FXI dsRNA compounds, 3′ and 5′ end of sense polynucleotides, and/or 3′ end of antisense polynucleotides of the invention, include at least one modified nucleotide, wherein the at least one modified nucleotide comprises: isomannide nucleotide or stereoisomer of said isomannide nucleotide. Specific examples of isomannide nucleotides or stereoisomers of said isomannide nucleotides include, but are not limited to:

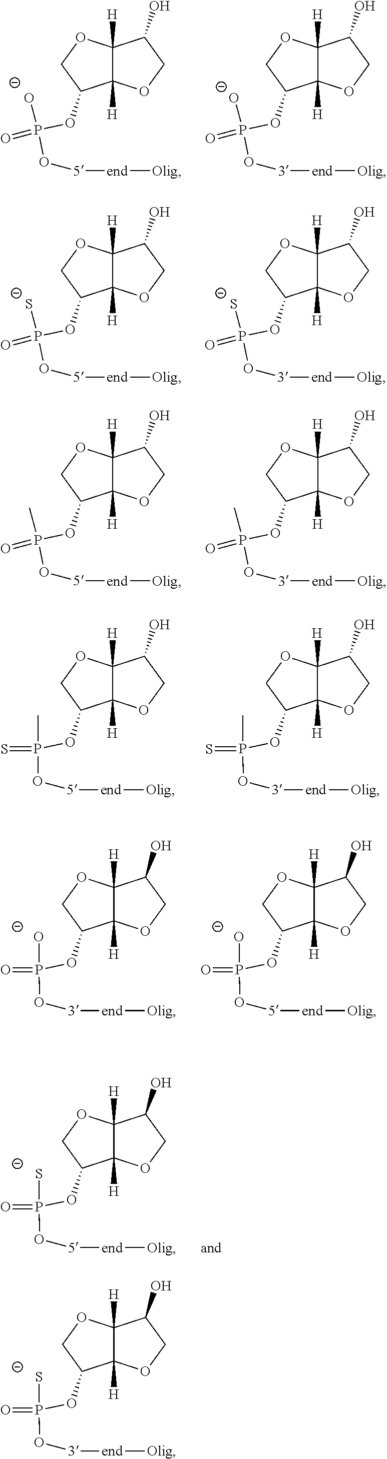

wherein the phrase “Olig” each independently represents a polynucleotide moiety. Exemplified isomannide residues (imann) include, but are not limited to, the following:

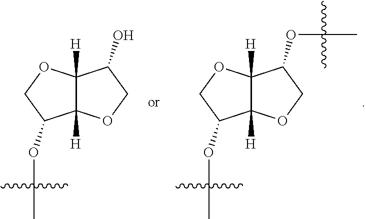 [SMILES: *O[C@@H]1CO[C@H]2[C@@H]1OC[C@H]2O ; *O[C@@H]1CO[C@H]2[C@@H]1OC[C@H]2O*]

In certain embodiments, the isomannide nucleotides may further conjugate to one or more targeting groups or delivery molecules, such as GalNAc moieties.

Certain embodiments of FXI dsRNA compounds, antisense polynucleotides of the invention, include at least one modified nucleotide, wherein the at least one modified nucleotide comprises unlocked nucleic acid nucleotide (UNA) or/and glycol nucleic acid nucleotide (GNA). It is known to skilled in art, UNA and GNA are thermally destabilizing chemical modifications, can significantly improves the off-target profile of a siRNA compound (Janas, et al., Selection of GalNAc-conjugated siRNAs with limited off-target-driven rat hepatotoxicity. Nat Commun. 2018; 9(1):723. doi:10.1038/s41467-018-02989-4; Laursen et al., Utilization of unlocked nucleic acid (UNA) to enhance siRNA performance in vitro and in vivo. Mol BioSyst. 2010; 6:862-70).

Another modification that may be included in the RNA of certain embodiments of FXI dsRNA agents, FXI antisense polynucleotides, and/or FXI sense polynucleotides of the invention, comprises chemically linking to the RNA one or more ligands, moieties or conjugates that enhance one or more characteristics of the FXI dsRNA agent, FXI antisense polynucleotide, and/or FXI sense polynucleotide, respectively. Non-limiting examples of characteristics that may be enhanced are: FXI dsRNA agent, FXI antisense polynucleotide, and/or FXI sense polynucleotide activity, cellular distribution, delivery of a FXI dsRNA agent, pharmacokinetic properties of a FXI dsRNA agent, and cellular uptake of the FXI dsRNA agent. In some embodiments of the invention, a FXI dsRNA agent comprises one or more targeting groups or linking groups, which in certain embodiments of FXI dsRNA agents of the invention are conjugated to the sense strand. A non-limiting example of a targeting group is a compound comprising N-acetyl-galactosamine (GalNAc). The terms “targeting group”, “targeting agent”, “linking agent”, “targeting compound”, “delivery molecule”, “delivery compound” and “targeting ligand” may be used interchangeably herein. In certain embodiments of the invention a FXI dsRNA agent comprises a targeting compound that is conjugated to the 5′-terminal end of the sense strand. In certain embodiments of the invention a FXI dsRNA agent comprises a targeting compound that is conjugated to the 3′-terminal end of the sense strand. In some embodiments of the invention, a FXI dsRNA agent comprises a targeting group that comprises GalNAc. In certain embodiments of the invention a FXI dsRNA agent does not include a targeting compound conjugated to one or both of the 3′-terminal end and the 5′-terminal end of the sense strand. In certain embodiments of the invention a FXI dsRNA agent does not include a GalNAc containing targeting compound conjugated to one or both of the 5′-terminal end and the 3′-terminal end of the sense strand.

Additional targeting and linking agents are well known in the art, for example, targeting and linking agents that may be used in certain embodiments of the invention include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

Certain embodiments of a composition comprising a FXI dsRNA agent, FXI antisense polynucleotide, and/or FXI sense polynucleotide may comprise a ligand that alters distribution, targeting, or etc. of the FXI dsRNA agent. In some embodiments of a composition comprising a FXI dsRNA agent of the invention, the ligand increases affinity for a selected target, e.g., molecule, cell or cell type, compartment, e.g., a cellular or organ compartment, tissue, organ or region of the body, as, e.g., compared to a species absent such a ligand. A ligand useful in a composition and/or method of the invention may be a naturally occurring substance, such as a protein (e.g., human serum albumin (HSA), low-density lipoprotein (LDL), or globulin); a carbohydrate (e.g., a dextran, pullulan, chitin, chitosan, inulin, cyclodextrin or hyaluronic acid); or a lipid. A ligand may also be a recombinant or synthetic molecule, such as a synthetic polymer, e.g., a synthetic polyamino acid or polyamine. Examples of polyamino acids are a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

A ligand included in a composition and/or method of the invention may comprise a targeting group, non-limiting examples of which are a cell or tissue targeting agent, e.g., a lectin, glycoprotein, lipid or protein, e.g., an antibody that binds to a specified cell type such as a kidney cell or a liver cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

A ligand included in a composition and/or method of the invention may be a protein, e.g., glycoprotein, or peptide, for example a molecule with a specific affinity for a co-ligand, or an antibody, for example an antibody, that binds to a specified cell type such as a cancer cell, endothelial cell, cardiac cell, or bone cell. A ligand useful in an embodiment of a composition and/or method of the invention can be a hormone or hormone receptor. A ligand useful in an embodiment of a composition and/or method of the invention can be a lipid, lectin, carbohydrates, vitamin, cofactos, multivalent lactose, multivalent galactose, N-acetyl-galactosamine, N-acetyl-gulucosamine multivalent mannose, or multivalent fucose. A ligand useful in an embodiment of a composition and/or method of the invention can be a substance that can increase uptake of the FXI dsRNA agent into the cell, for example, by disrupting the cell's cytoskeleton, e.g., by disrupting the cell's microtubules, microfilaments, and/or intermediate filaments. Non-limiting examples of this type of agent are: taxon, vincristine, vinblastine, cytochalasin, nocodazole, japlakinolide, latrunculin A, phalloidin, swinholide A, indanocine, and myoservin.

In some embodiments, a ligand attached to a FXI dsRNA agent of the invention functions as a pharmacokinetic (PK) modulator. An example of a PK modulator that may be used in compositions and methods of the invention includes but is not limited to: a lipophiles, a bile acid, a steroid, a phospholipid analogue, a peptide, a protein binding agent, PEG, a vitamin, cholesterol, a fatty acid, cholic acid, lithocholic acid, dialkylglycerides, diacylglyceride, a phospholipid, a sphingolipid, naproxen, ibuprofen, vitamin E, biotin, an aptamer that binds a serum protein, etc. Oligonucleotides comprising a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbone may also be used in compositions and/or methods of the invention as ligands.

FY1 dsRNA Agent Compositions

In some embodiments of the invention, a FXI dsRNA agent is in a composition. A composition of the invention may include one or more FXI dsRNA agent and optionally one or more of a pharmaceutically acceptable carrier, a delivery agent, a targeting agent, detectable label, etc. A non-limiting example of a targeting agent that may be useful according to some embodiments of methods of the invention is an agent that directs a FXI dsRNA agent of the invention to and/or into a cell to be treated. A targeting agent of choice will depend upon such elements as: the nature of the FXI-associated disease or condition, and on the cell type being targeted. In a non-limiting example, in some embodiments of the invention it may be desirable to target a FXI dsRNA agent to and/or into a liver cell. It will be understood that in some embodiments of methods of the invention, a therapeutic agent comprises a FXI dsRNA agent with only a delivery agent, such as a delivery agent comprising N-Acetylgalactosamine (GalNAc), without any additional attached elements. For example, in some aspects of the invention a FXI dsRNA agent may be attached to a delivery compound comprising GalNAc and included in a composition comprising a pharmaceutically acceptable carrier and administered to a cell or subject without any detectable labels, or targeting agents, etc. attached to the FXI dsRNA agent.

In cases where a FXI dsRNA agent of the invention is administered with and/or attached to one or more delivery agents, targeting agents, labeling agents, etc. a skilled artisan will be aware of and able to select and use suitable agents for use in methods of the invention. Labeling agents may be used in certain methods of the invention to determine the location of a FXI dsRNA agent in cells and tissues and may be used to determine a cell, tissue, or organ location of a treatment composition comprising a FXI dsRNA agent that has been administered in methods of the invention. Procedures for attaching and utilizing labeling agents such as enzymatic labels, dyes, radiolabels, etc. are well known in the art. It will be understood that in some embodiments of compositions and methods of the invention, a labeling agent is attached to one or both of a sense polynucleotide and an antisense polynucleotide included in a FXI dsRNA agent.

Delivery of FX dsRNA Agents and FXI Antisense Polynucleotide Agents

Certain embodiments of methods of the invention, includes delivery of a FXI dsRNA agent into a cell. As used herein the term, “delivery” means facilitating or effecting uptake or absorption into the cell. Absorption or uptake of a FXI dsRNA agent can occur through unaided diffusive or active cellular processes, or by use of delivery agents, targeting agents, etc. that may be associated with a FXI dsRNA agent of the invention. Delivery means that are suitable for use in methods of the invention include, but are not limited to: in vivo delivery, in which a FXI dsRNA agent is in injected into a tissue site or administered systemically. In some embodiments of the invention, a FXI dsRNA agent is attached to a delivery agent.

Non-limiting examples of methods that can be used to deliver FXI dsRNA agents to cells, tissues and/or subjects include: FXI dsRNA-GalNAc conjugates, SAMiRNA technology, LNP-based delivery methods, and naked RNA delivery. These and other delivery methods have been used successfully in the art to deliver therapeutic RNAi agents for treatment of various diseases and conditions, such as but not limited to: liver diseases, acute intermittent porphyria (AIP), hemophilia, pulmonary fibrosis, etc. Details of various delivery means are found in publications such as: Nikam, R. R. & K. R. Gore (2018) Nucleic Acid Ther, 28 (4), 209-224 August 2018; Springer A. D. & S. F. Dowdy (2018) Nucleic Acid Ther. June 1; 28(3): 109-118; Lee, K. et al., (2018) Arch Pharm Res, 41(9), 867-874; and Nair, J. K. et al., (2014) J. Am. Chem. Soc. 136:16958-16961, the content each of which is incorporated by reference herein.

Some embodiments of the invention comprise use of lipid nanoparticles (LNPs) to deliver a FXI dsRNA agent of the invention to a cell, tissue, and/or subject. LNPs are routinely used for in vivo delivery of FXI dsRNA agents, including therapeutic FXI dsRNA agents. One benefit of using an LNP or other delivery agent is an increased stability of the FXI RNA agent when it is delivered to a subject using the LNP or other delivery agent. In some embodiments of the invention an LNP comprises a cationic LNP that is loaded with one or more FXI RNAi molecules of the invention. The LNP comprising the FXI RNAi molecule(s) is administered to a subject, the LNPs and their attached FXI RNAi molecules are taken up by cells via endocytosis, their presence results in release of RNAi trigger molecules, which mediate RNAi.

Another non-limiting example of a delivery agent that may be used in embodiments of the invention to delivery a FXI dsRNA agent of the invention to a cell, tissue and/or subject is an agent comprising GalNAc that is attached to a FXI dsRNA agent of the invention and delivers the FXI dsRNA agent to a cell, tissue, and/or subject. Examples of certain additional delivery agents comprising GalNAc that can be used in certain embodiments of methods and composition of the invention are disclosed in PCT Application: WO2020191183A1, WO2023/045995A1 (incorporated herein in its entirety). A non-limiting example of a GalNAc targeting ligand that can be used in compositions and methods of the invention to deliver a FXI dsRNA agent to a cell is a targeting ligand cluster. Examples of targeting ligand clusters that are presented herein are referred to as: GalNAc Ligand with phosphodiester link (GLO) and GalNAc Ligand with phosphorothioate link (GLS*). The term “GLX-n” may be used herein to indicate the attached GalNAc-containing compound is any one of compounds GLS-1*, GLS-2*, GLS-3*, GLS-4*, GLS-5*, GLS-6*, GLS-7*, GLS-8*, GLS-9*, GLS-10*, GLS-11*, GLS-12*, GLS-13*, GLS-14*, GLS-15*, GLS-16*, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16, the structure of each of which is shown below, with the below with location of attachment of the GalNAc-targeting ligand to an RNAi agent of the invention at far right of each (shown b [symbol] with “ [symbol] ”). It will be understood that any RNAi and dsRNA molecule of the invention can be attached to the GLS-1*, GLS-2*, GLS-3*, GLS-4*, GLS-5*, GLS-6*, GLS-7*, GLS-8*, GLS-9*, GLS-10*, GLS-11*, GLS-12*, GLS-13*, GLS-14*, GLS-15*, GLS-16*, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16, GLO-1 through GLO-16 and GLS-1*through GLS-16* structures are shown below.

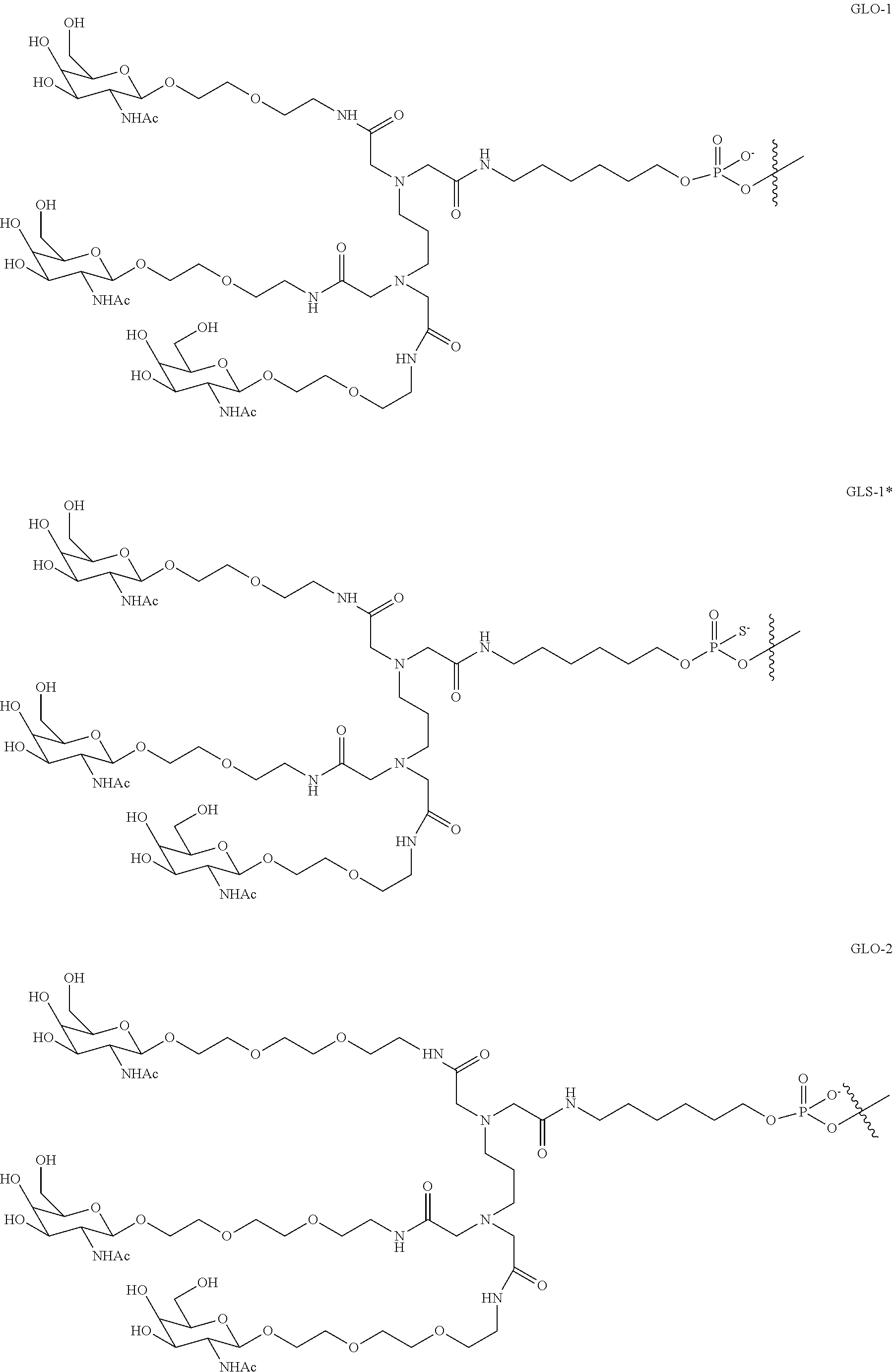 [SMILES: *OP(=O)([O-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCNC(=O)CN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

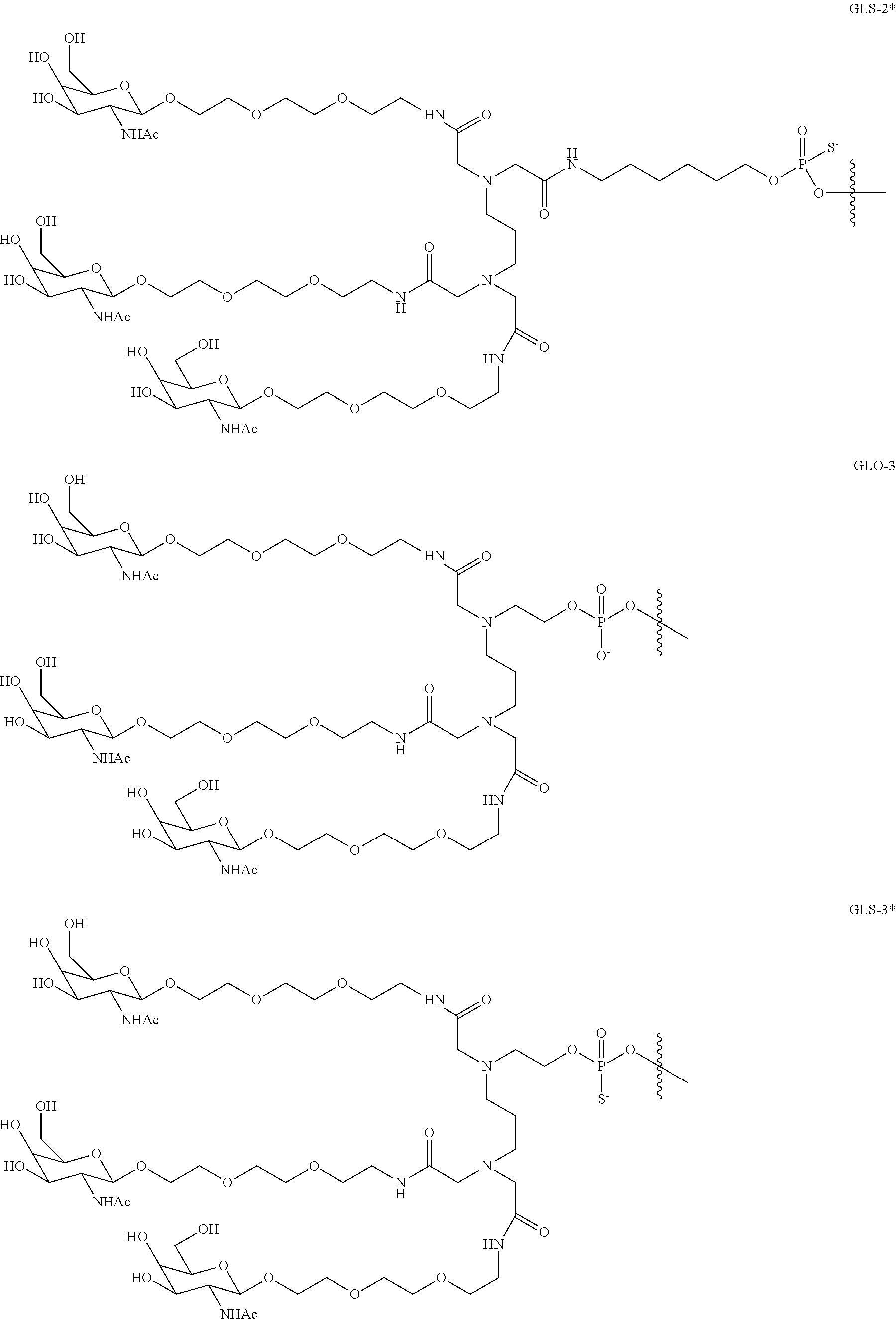 [SMILES: *OP(=O)([S-])OCCCCCCNC(=O)CN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

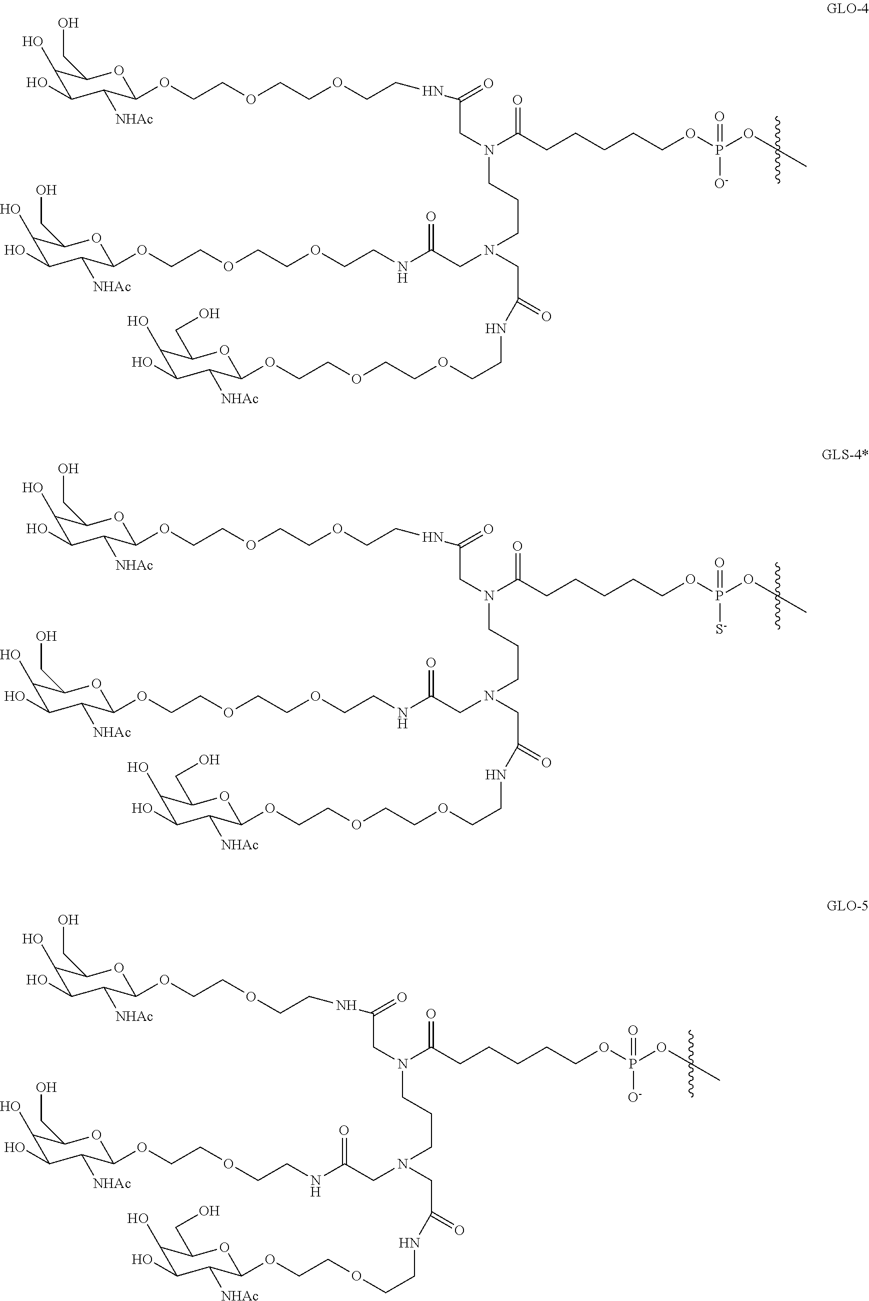 [SMILES: *OP(=O)([O-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

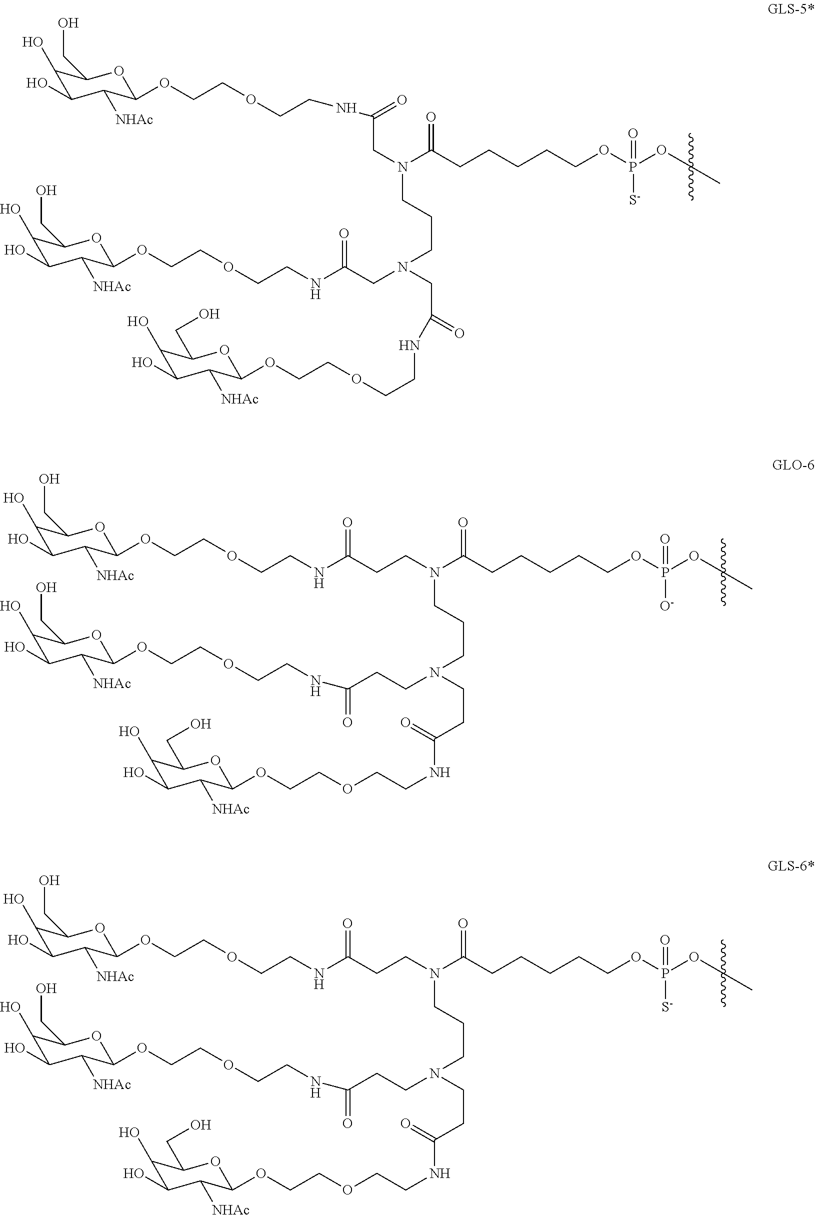 [SMILES: *OP(=O)([S-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O]

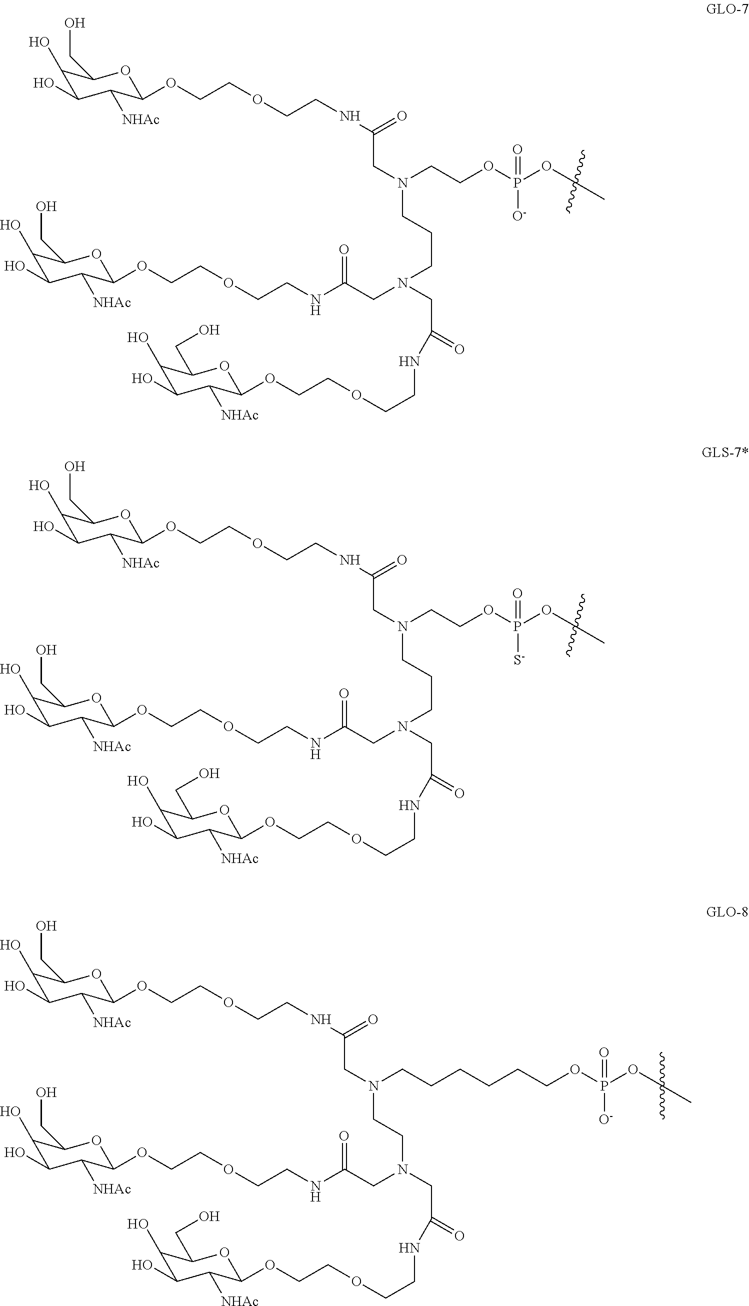 [SMILES: *OP(=O)([O-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCN(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

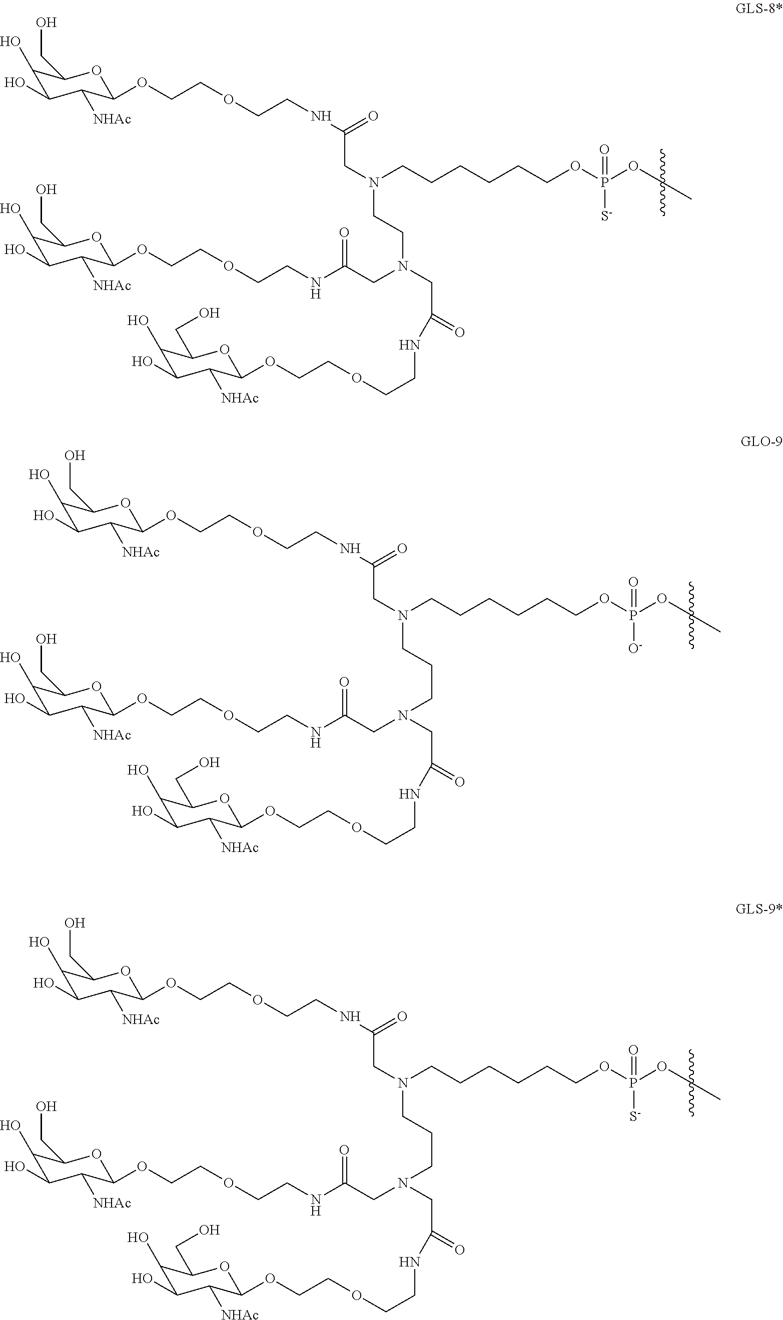 [SMILES: *OP(=O)([S-])OCCCCCCN(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

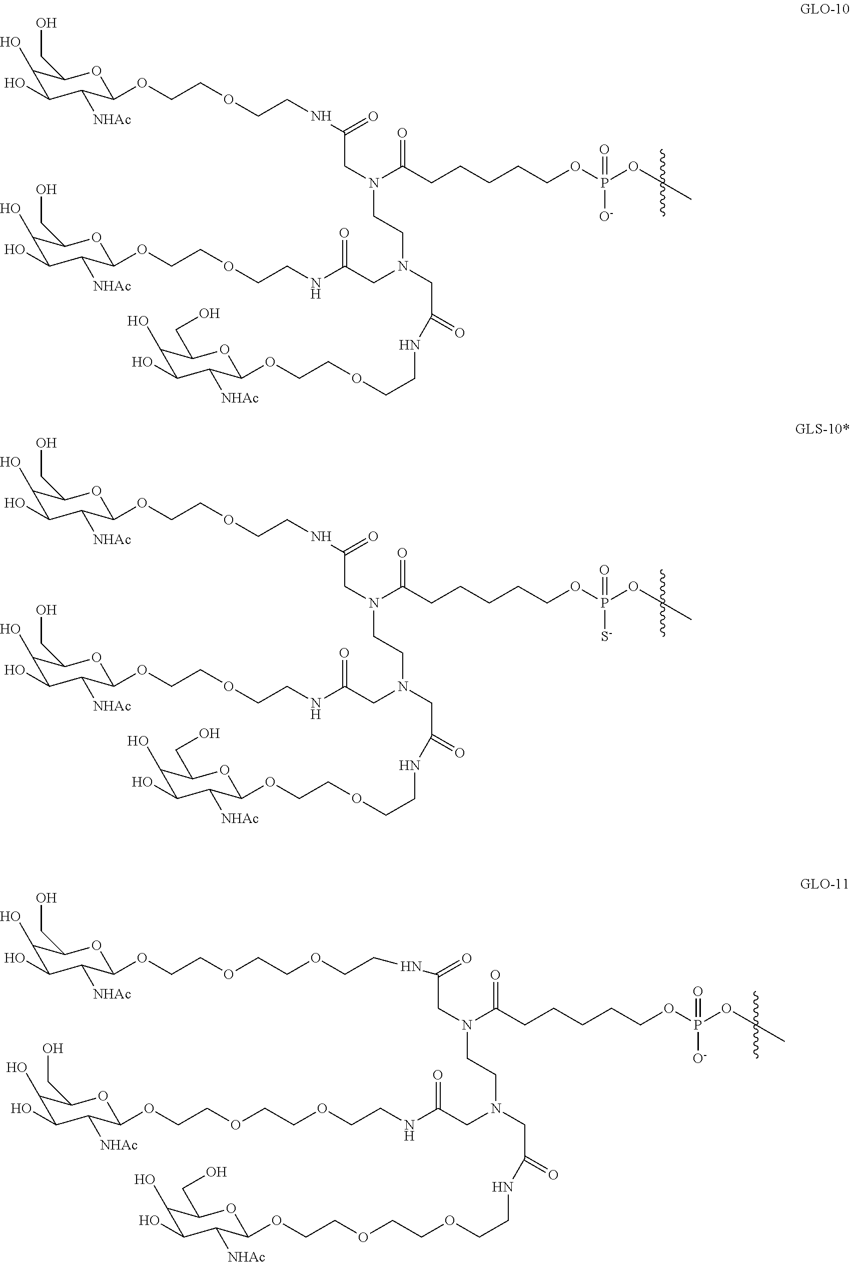 [SMILES: *OP(=O)([O-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

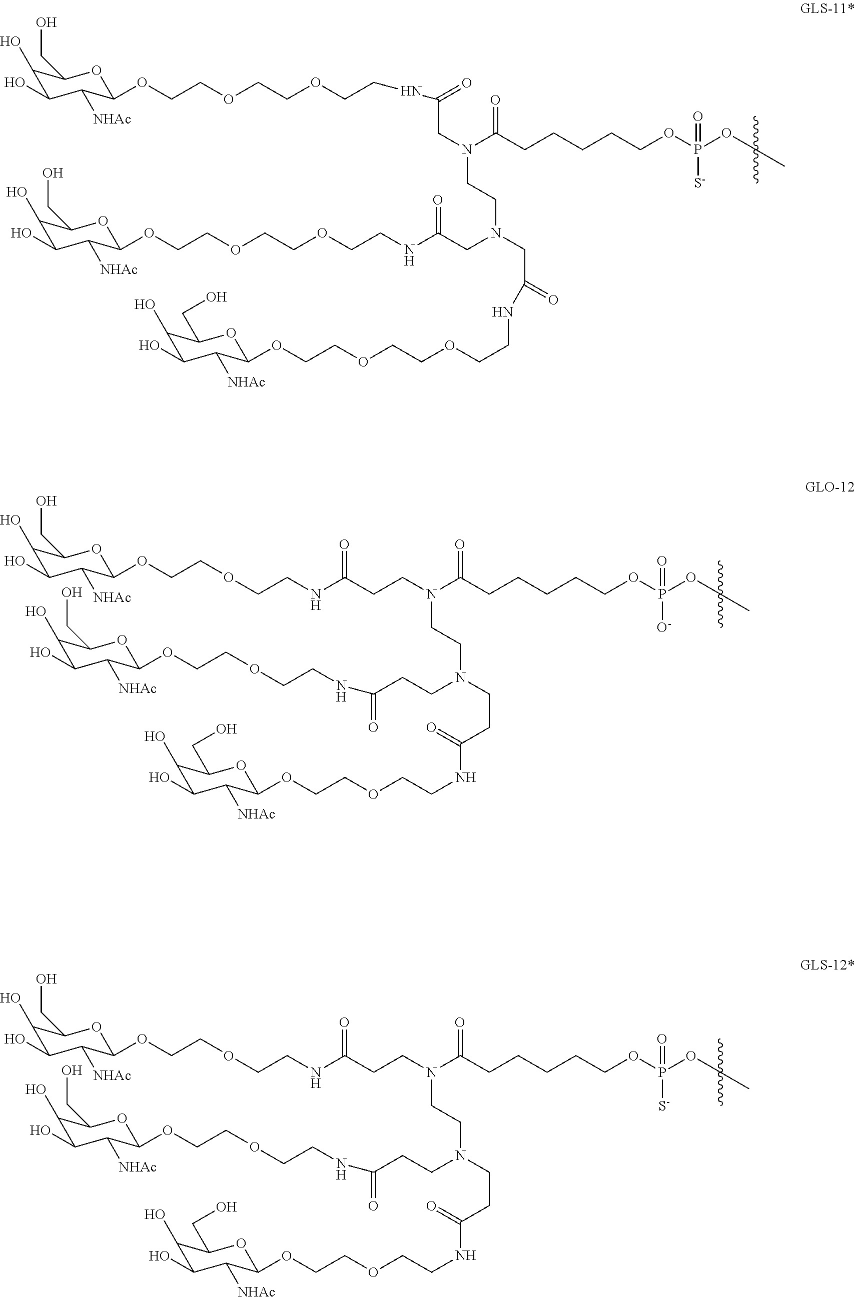 [SMILES: *OP(=O)([S-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O]

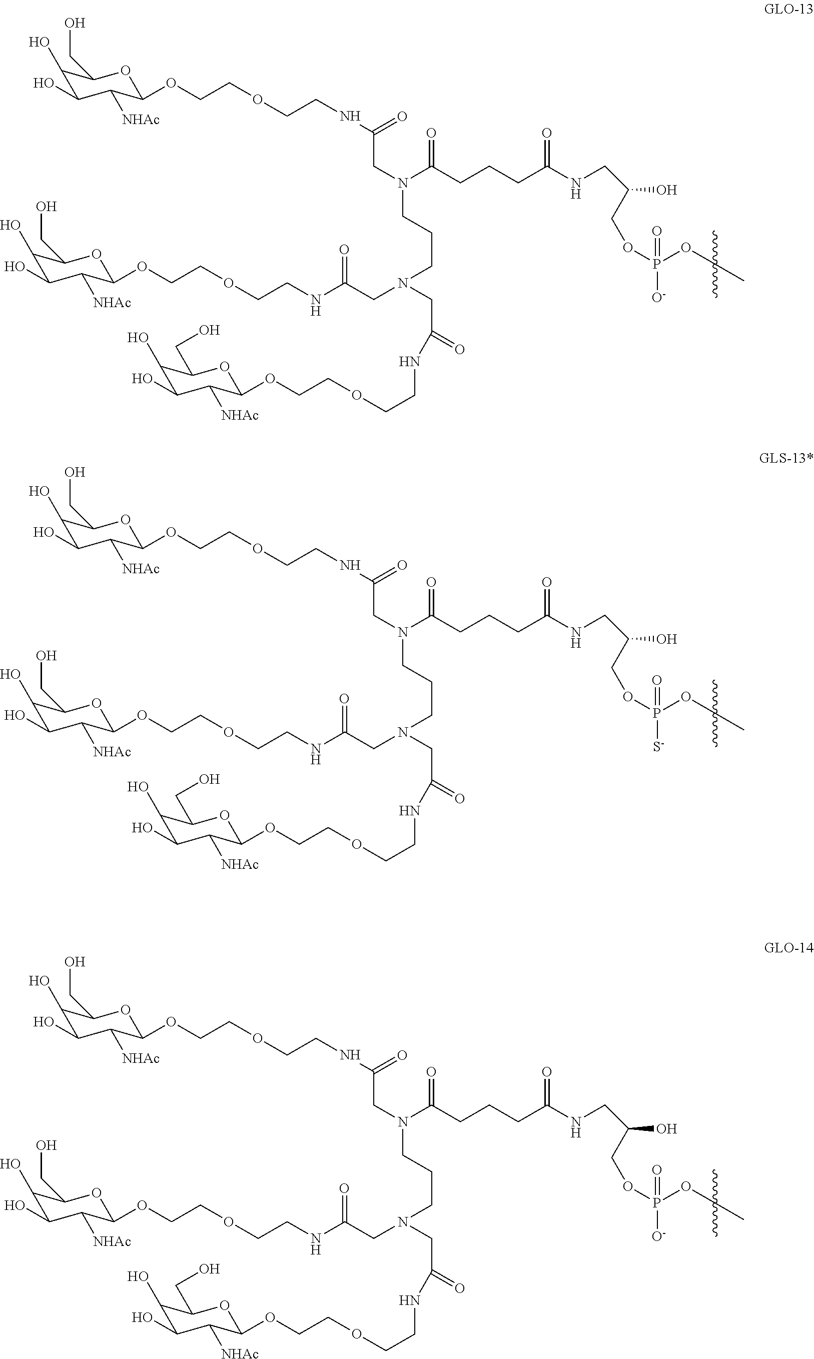 [SMILES: *OP(=O)([O-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

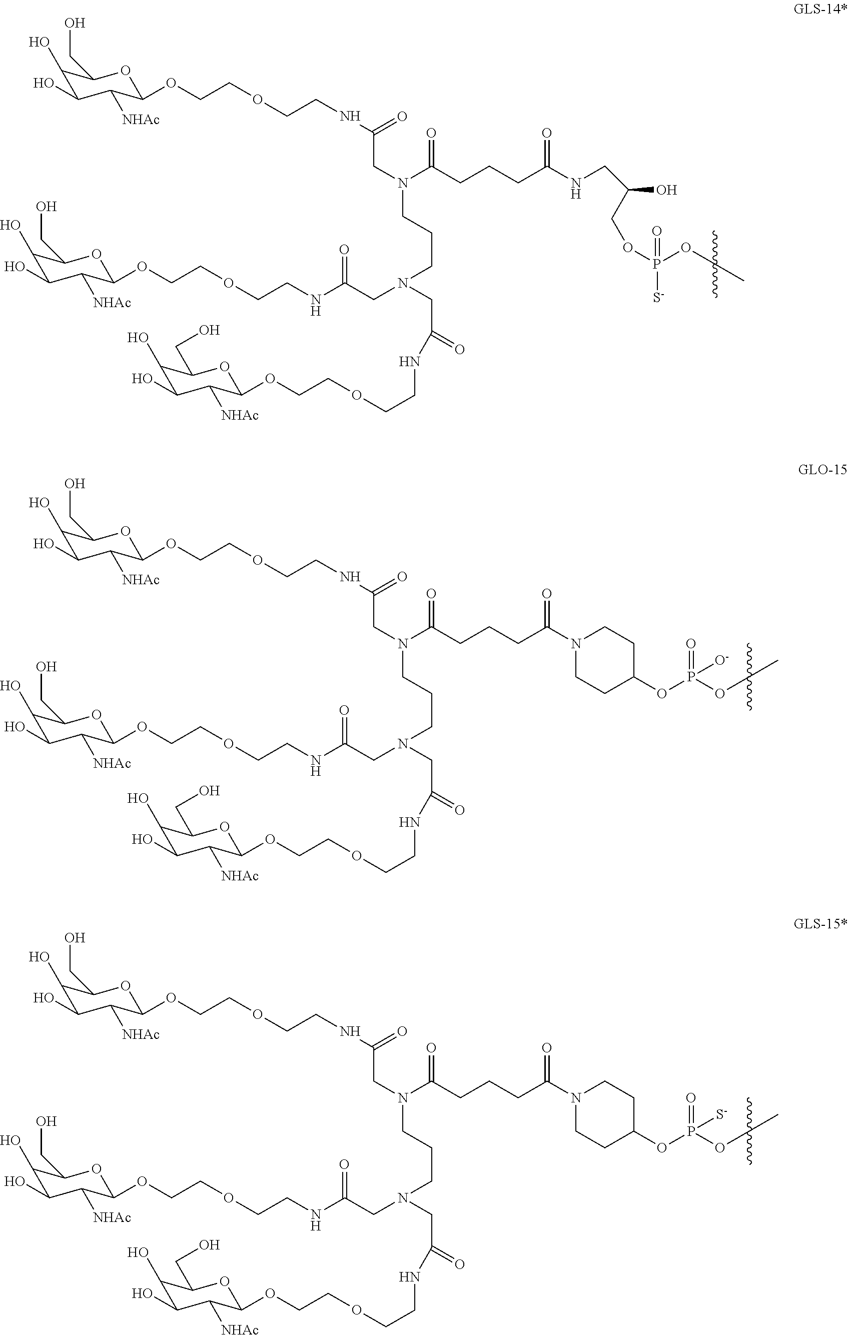 [SMILES: *OP(=O)([S-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1 ; *OP(=O)([S-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1]

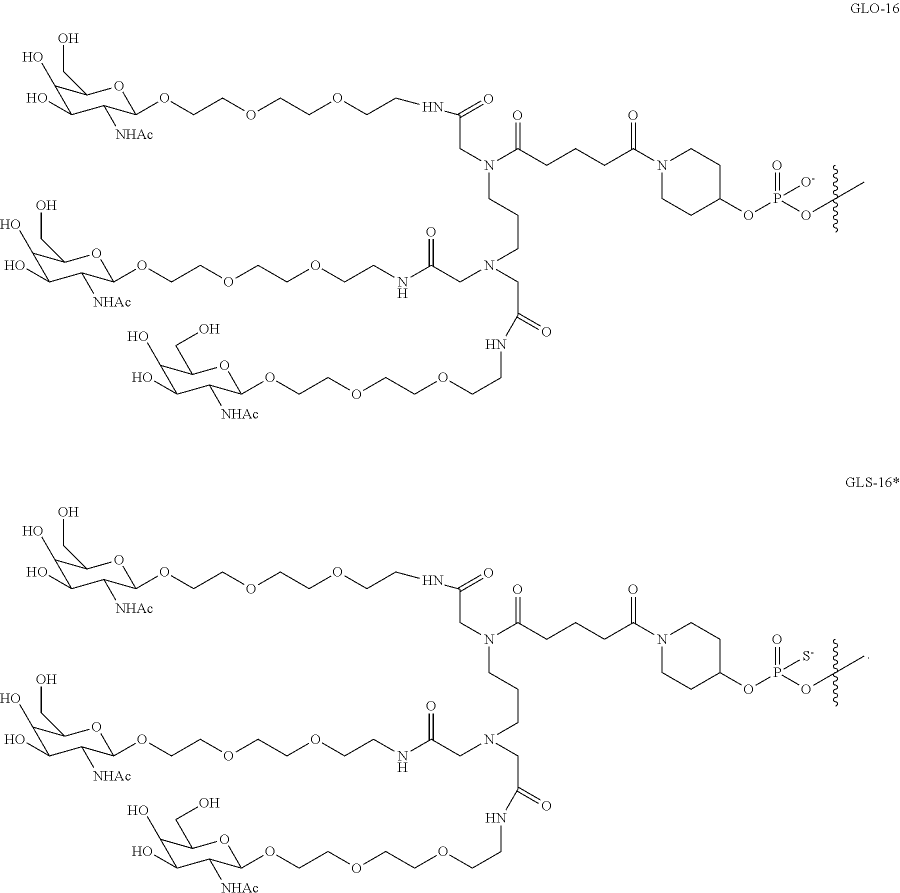 [SMILES: *OP(=O)([O-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1 ; *OP(=O)([S-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1]

In certain embodiments, the aforesaid isomannide nucleotides may further conjugate to one or more GalNAc targeting ligands. Specific examples of isomannide nucleotides conjugated to a GalNAc targeting ligand include, but are not limited to:

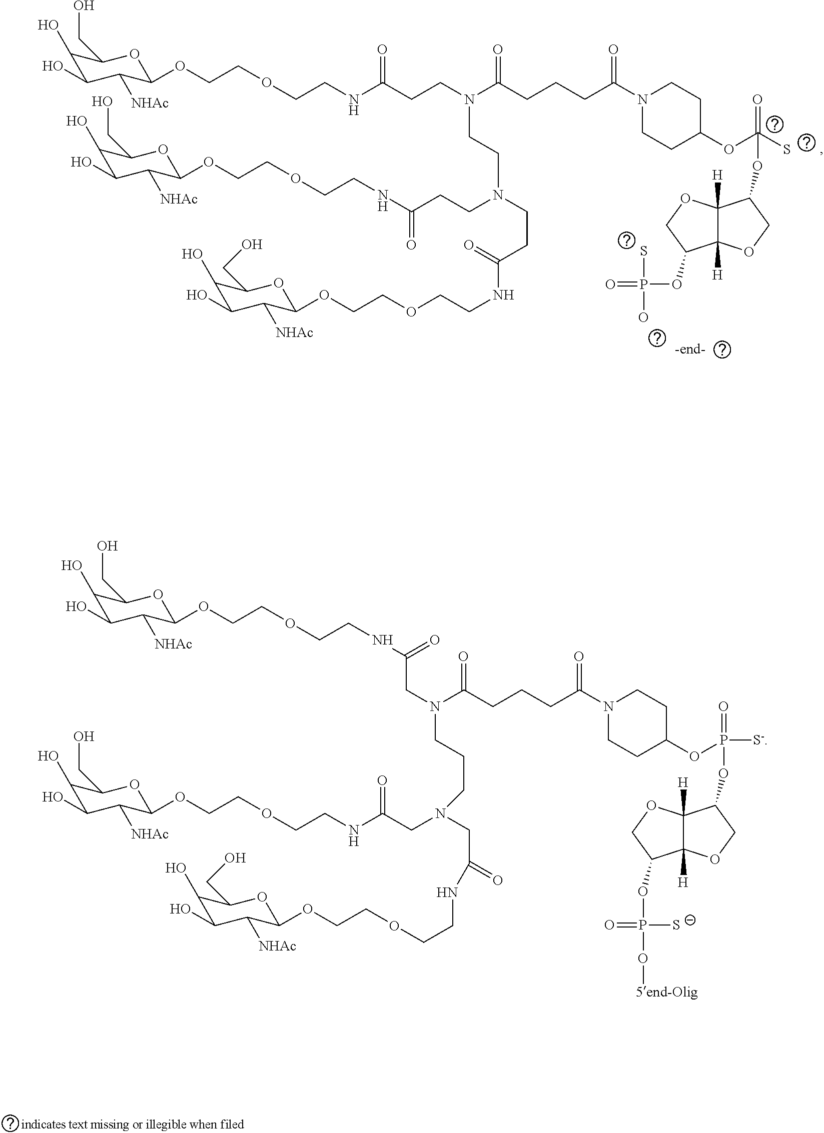

In some embodiments of the invention, in vivo delivery can also be by a beta-glucan delivery system, such as those described in U.S. Pat. Nos. 5,032,401 and 5,607,677, and U.S. Publication No. 2005/0281781, which are hereby incorporated by reference in their entirety. In vitro introduction of a FXI RNAi agent into a cell may also be done using art-known methods such as electroporation and lipofection. In certain embodiments of methods of the invention, a FXI dsRNA is delivered without a targeting agent. These RNAs may be delivered as “naked” RNA molecules. As a non-limiting example, a FXI dsRNA of the invention may be administered to a subject to treat a FXI-associated disease or condition in the subject, such as a liver disease, in a pharmaceutical composition comprising the RNAi agent, but not including a targeting agent such as a GalNAc targeting compound.

In addition to certain delivery means described herein, it will be understood that RNAi delivery means, such as but not limited to those described herein and those used in the art, can be used in conjunction with embodiments of FXI RNAi agents and treatment methods described herein.

FXI dsRNA agents of the invention may be administered to a subject in an amount and manner effective to reduce a level and activity of FXI polypeptide in a cell and/or subject. In some embodiments of methods of the invention one or more FXI dsRNA agents are administered to a cell and/or subject to treat a disease or condition associated with FXI expression and activity. Methods of the invention, in some embodiments, include administering one or more FXI dsRNA agents to a subject in need of such treatment to reduce a disease or condition associated with FXI expression in the subject. FXI dsRNA agents or FXI antisense polynucleotide agents of the invention can be administered to reduce FXI expression and/or activity in one more of in vitro, ex vivo, and in vivo cells.

In some embodiments of the invention, a level, and thus an activity, of FXI polypeptide in a cell is reduced by delivering (e.g. introducing) a FXI dsRNA agent or FXI antisense polynucleotide agent into a cell. Targeting agents and methods may be used to aid in delivery of a FXI dsRNA agent or FXI antisense polynucleotide agent to a specific cell type, cell subtype, organ, spatial region within a subject, and/or to a sub-cellular region within a cell. A FXI dsRNA agent can be administered in certain methods of the invention singly or in combination with one or more additional FXI dsRNA agents. In some embodiments, 2, 3, 4, or more independently selected FXI dsRNA agents are administered to a subject.

In certain embodiments of the invention, a FXI dsRNA agent is administered to a subject to treat a FXI-associated disease or condition in conjunction with one or more additional therapeutic regimens for treating the FXI-associate disease or condition. Non-limiting examples of additional therapeutic regimens are: administering one or more FXI antisense polynucleotides of the invention, administering a non-FXI dsRNA therapeutic agent, and a behavioral modification. An additional therapeutic regimen may be administered at a time that is one or more of: prior to, simultaneous with, and following administration of a FXI dsRNA agent of the invention. It will be understood that simultaneous with as used herein, within five minutes of time zero, within 10 minutes of time zero, within 30 minutes of time zero, within 45 minutes of time zero, and within 60 minutes of time zero, with “time zero” the time of administration of the FXI dsRNA agent of the invention to the subject. Non-limiting examples of non-FXI dsRNA therapeutic agents are: NSAID/Cyclooxygenase inhibitors, such as, aspirin; adenosine diphosphate (ADP) receptor inhibitors, such as, clopidogrel (PLAVIX) and ticlopidine (TICLID); phosphodiesterase inhibitors, such as, cilostazol (PLETAL); glycoprotein IIB/IIIA inhibitors, such as, abciximab (REOPRO), eptifibatide (INTEGRILIN), tirofiban (AGGRASTAT), and defibrotide; adenosine reuptake inhibitors, such as, dipyridamole (PERSANTINE); warfarin (and related coumarins), heparin; direct thrombin inhibitors. such as lepirudin, bivalirudin, apixaban, LOVENOX (enoxaparin); and small molecular compounds that interfere directly with the enzymatic action of particular coagulation factors (e.g. rivaroxaban, which interferes with Factor Xa). These and other therapeutic agents and behavior modifications are known in the art and used to treat a FXI disease or condition in a subject and may be administered to a subject in combination with the administration of one or more FXI dsRNA agents of the invention to treat the FXI disease or condition. A FXI dsRNA agent of the invention administered to a cell or subject to treat a FXI-associated disease or condition may act in a synergistic manner with one or more other therapeutic agents or activities and increase the effectiveness of the one or more therapeutic agents or activities and/or to increase the effectiveness of the FXI dsRNA agent at treating the FXI-associated disease or condition.

Treatment methods of the invention that include administration of a FXI dsRNA agent can be used prior to the onset of a FXI-associated disease or condition and/or when a FXI-associated disease or condition is present, including at an early stage, mid-stage, and late stage of the disease or condition and all times before and after any of these stages. Methods of the invention may also be to treat subjects who have previously been treated for a FXI-associated disease or condition with one or more other therapeutic agents and/or therapeutic activities that were not successful, were minimally successful, and/or are no longer successful at treating the FXI-associated disease or condition in the subject.

Vector Encoded dsRNAs

In certain embodiments of the invention, a FXI dsRNA agent can be delivered into a cell using a vector. FXI dsRNA agent transcription units can be included in a DNA or RNA vector. Prepare and use of such vectors encoding transgenes for delivering sequences into a cell and or subject are well known in the art. Vectors can be used in methods of the invention that result in transient expression of FXI dsRNA, for example for at least 1, 2, 3, 4, 5, 6, 7, 8, 9, 10 or more hours, 1, 2, 3, 4, 5, 6, 7, 8, 9, 10 or more weeks. The length of the transient expression can be determined using routine methods based on elements such as, but not limited to the specific vector construct selected and the target cell and/or tissue. Such transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA (1995) 92:1292).

An individual strand or strands of a FXI dsRNA agent can be transcribed from a promoter on an expression vector. Where two separate strands are to be expressed to generate, for example, a dsRNA, two separate expression vectors can be co-introduced to a cell using means such as transfection or infection. In certain embodiments each individual strand of a FXI dsRNA agent of the invention can be transcribed by promoters that are both included on the same expression vector. In certain embodiments of the invention a FXI dsRNA agent is expressed as inverted repeat polynucleotides joined by a linker polynucleotide sequence such that the FXI dsRNA agent has a stem and loop structure.

Non-limiting examples of RNA expression vectors are DNA plasmids or viral vectors. Expression vectors useful in embodiments of the invention can be compatible with eukaryotic cells. Eukaryotic cell expression vectors are routinely used in the art and are available from a number of commercial sources. Delivery of FXI dsRNA expressing vectors can be systemic, such as by intravenous or intramuscular administration, by administration to target cells ex-planted from a subject followed by reintroduction into the subject, or by any other means that allows for introduction into a desired target cell.

Viral vector systems that may be included in an embodiment of a method of the include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Constructs for the recombinant expression of a FXI dsRNA agent may include regulatory elements, such as promoters, enhancers, etc., which may be selected to provide constitutive or regulated/inducible expression. Viral vector systems, and the use of promoters and enhancers, etc. are routine in the art and can be used in conjunction with methods and compositions described herein.

Certain embodiments of the invention include use of viral vectors for delivery of FXI dsRNA agents into cells. Numerous adenovirus-based delivery systems are routinely used in the art for deliver to, for example, lung, liver, the central nervous system, endothelial cells, and muscle. Non-limiting examples of viral vectors that may be used in methods of the invention are: AAV vectors, a pox virus such as a vaccinia virus, a Modified Virus Ankara (MVA), NYVAC, an avipox such as fowl pox or canary pox.

Certain embodiments of the invention include methods of delivering FXI dsRNA agents into cells using a vector and such vectors may be in a pharmaceutically acceptable carrier that may, but need not, include a slow release matrix in which the gene delivery vehicle is imbedded. In some embodiments, a vector for delivering a FXI dsRNA can be produced from a recombinant cell, and a pharmaceutical composition of the invention may include one or more cells that produced the FXI dsRNA delivery system.

Pharmaceutical Compositions Containing FXI dsRNA or ssRNA Agents

Certain embodiments of the invention include use of pharmaceutical compositions containing a FXI dsRNA agent or FXI antisense polynucleotide agent and a pharmaceutically acceptable carrier. The pharmaceutical composition containing the FXI dsRNA agent or FXI antisense polynucleotide agent can be used in methods of the invention to reduce FXI gene expression and FXI activity in a cell and is useful to treat a FXI-associated disease or condition. Such pharmaceutical compositions can be formulated based on the mode of delivery. Non-limiting examples of formulations for modes of delivery are: a composition formulated for subcutaneous delivery, a composition formulated for systemic administration via parenteral delivery, a composition formulated for intravenous (IV) delivery, a composition formulated for intrathecal delivery, a composition formulated for direct delivery into brain, etc. Administration of a pharmaceutic composition of the invention to deliver a FXI dsRNA agent or FXI antisense polynucleotide agent into a cell may be done using one or more means such as: topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration. A FXI dsRNA agent or FXI antisense polynucleotide agent can also be delivered directly to a target tissue, for example directly into the liver, directly into a kidney, etc. It will be understood that “delivering a FXI dsRNA agent” or “delivering a FXI antisense polynucleotide agent” into a cell encompasses delivering a FXI dsRNA agent or FXI antisense polynucleotide agent, respectively, directly as well as expressing a FXI dsRNA agent in a cell from an encoding vector that is delivered into a cell, or by any suitable means with which the FXI dsRNA or FXI antisense polynucleotide agent becomes present in a cell. Preparation and use of formulations and means for delivering inhibitory RNAs are well known and routinely used in the art.

As used herein, a “pharmaceutical composition” comprises a pharmacologically effective amount of a FXI dsRNA agent or FXI antisense polynucleotide agent of the invention and a pharmaceutically acceptable carrier. The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Agents included in drug formulations are described further herein below.

As used herein terms such as: “pharmacologically effective amount,” “therapeutically effective amount” and “effective amount” refers to that amount of a FXI dsRNA agent or FXI antisense polynucleotide agent of the invention to produce the intended pharmacological, therapeutic or preventive result. For example, if a given clinical treatment is considered effective when there is at least a 10% reduction in a measurable parameter associated with a disease or disorder, a therapeutically effective amount of a drug for the treatment of that disease or disorder is the amount necessary to effect at least a 10% reduction in that parameter. For example, a therapeutically effective amount of a FXI dsRNA agent or FXI antisense polynucleotide agent can reduce FXI polypeptide levels by at least 10%.

### Effective Amounts

Methods of the invention, in some aspects comprise contacting a cell with a FXI dsRNA agent or FXI antisense polynucleotide agent in an effective amount to reduce FXI gene expression in the contacted cell. Certain embodiments of methods of the invention comprise administering a FXI dsRNA agent or a FXI antisense polynucleotide agent to a subject in an amount effective to reduce FXI gene expression and treat a FXI-associated disease or condition in the subject. An “effective amount” used in terms of reducing expression of FXI and/or for treating a FXI-associated disease or condition, is an amount necessary or sufficient to realize a desired biologic effect. For example, an effective amount of a FXI dsRNA agent or FXI antisense polynucleotide agent to treat a FXI-associated disease or condition could be that amount necessary to (i) slow or halt progression of the disease or condition; or (ii) reverse, reduce, or eliminate one or more symptoms of the disease or condition. In some aspects of the invention, an effective amount is that amount of a FXI dsRNA agent or FXI antisense polynucleotide agent that when administered to a subject in need of a treatment of a FXI-associated disease or condition, results in a therapeutic response that prevents and/or treats the disease or condition. According to some aspects of the invention, an effective amount is that amount of a FXI dsRNA agent or FXI antisense polynucleotide agent of the invention that when combined or co-administered with another therapeutic treatment for a FXI-associated disease or condition, results in a therapeutic response that prevents and/or treats the disease or condition. In some embodiments of the invention, a biologic effect of treating a subject with a FXI dsRNA agent or FXI antisense polynucleotide agent of the invention may be the amelioration and or absolute elimination of symptoms resulting from the FXI-associated disease or condition. In some embodiments of the invention, a biologic effect is the complete abrogation of the FXI-associated disease or condition, as evidenced for example, by a diagnostic test that indicates the subject is free of the FXI-associated disease or condition. A non-limiting example of a physiological symptom that may be detected includes a reduction in FXI level in liver of a subject following administration of an agent of the invention. Additional art-known means of assessing the status of a FXI-associated disease or condition can be used to determine an effect of an agent and/or methods of the invention on a FXI-associated disease or condition.

Typically an effective amount of a FXI dsRNA agent or FXI antisense polynucleotide agent to decrease FXI polypeptide activity to a level to treat a FXI-associated disease or condition will be determined in clinical trials, establishing an effective dose for a test population versus a control population in a blind study. In some embodiments, an effective amount will be that results in a desired response, e.g., an amount that diminishes a FXI-associated disease or condition in cells, tissues, and/or subjects with the disease or condition. Thus, an effective amount of a FXI dsRNA agent or FXI antisense polynucleotide agent to treat a FXI-associated disease or condition that can be treated by reducing FXI polypeptide activity may be the amount that when administered decreases the amount of FXI polypeptide activity in the subject to an amount that is less than the amount that would be present in the cell, tissue, and/or subject without the administration of the FXI dsRNA agent or FXI antisense polynucleotide agent. In certain aspects of the invention the level of FXI polypeptide activity, and/or FXI gene expression present in a cell, tissue, and/or subject that has not been contacted with or administered a FXI dsRNA agent or FXI antisense polynucleotide agent of the invention is referred to as a “control” amount. In some embodiments of methods of the invention a control amount for a subject is a pre-treatment amount for the subject, in other words, a level in a subject before administration of a FXI agent can be a control level for that subject and compared to a level of FXI polypeptide activity and/or FXI gene expression in the subject following siRNA administered to the subject. In the case of treating a FXI-associated disease or condition the desired response may be reducing or eliminating one or more symptoms of the disease or condition in the cell, tissue, and/or subject. The reduction or elimination may be temporary or may be permanent. It will be understood that the status of a FXI-associated disease or condition can be monitored using methods of determining FXI polypeptide activity, FXI gene expression, symptom evaluation, clinical testing, etc. In some aspects of the invention, a desired response to treatment of a FXI-associated disease or condition is delaying the onset or even preventing the onset of the disease or condition.

An effective amount of a compound that decreases FXI polypeptide activity may also be determined by assessing physiological effects of administration of a FXI dsRNA agent or FXI antisense polynucleotide agent on a cell or subject, such as a decrease of a FXI-associated disease or condition following administration. Assays and/or symptomatic monitoring of a subject can be used to determine efficacy of a FXI dsRNA agent or FXI antisense polynucleotide agent of the invention, which may be administered in a pharmaceutical compound of the invention, and to determine the presence or absence of a response to the treatment. A non-limiting example, is that one or more art-known tests of activated partial thromboplastin time (aPTT), prothrombin time (PT) test, thrombin time (TCT), bleeding time, or presence of D-dimer in a blood sample of the animal. Another non-limiting example, is that one or more art-known tests of liver function can be used to determine the status of the FXI-associated liver disease or condition in a subject before and after treatment of the subject with a FXI dsRNA agent of the invention.

Some embodiments of the invention include methods of determining an efficacy of an dsRNA agent or FXI antisense polynucleotide agent of the invention administered to a subject, to treat a FXI-associated disease or condition by assessing and/or monitoring one or more “physiological characteristics” of the FXI-associated disease or condition in the subject. Non-limiting examples of physiological characteristics of a FXI-associated disease or condition are the FXI mRNA level, the FXI protein level, or activated partial thromboplastin time (aPTT), prothrombin time (PT) test, thrombin time (TCT), bleeding time, or presence of D-dimer in a plasma, or a tissue sample, and/or accumulation of fat and/or expansion of lipid droplets in the liver, etc. Standard means of determining such physiological characteristic are known in the art and include, but are not limited to, blood tests, imaging studies, physical examination, etc.

It will be understood that the amount of a FXI dsRNA agent or FXI antisense polynucleotide agent administered to a subject can be modified based, at least in part, on such determinations of disease and/or condition status and/or physiological characteristics determined for a subject. The amount of a treatment may be varied for example by increasing or decreasing the amount of a FXI-dsRNA agent or FXI antisense polynucleotide agent, by changing the composition in which the FXI dsRNA agent or FXI antisense polynucleotide agent, respectively, is administered, by changing the route of administration, by changing the dosage timing and so on. The effective amount of a FXI dsRNA agent or FXI antisense polynucleotide agent will vary with the particular condition being treated, the age and physical condition of the subject being treated; the severity of the condition, the duration of the treatment, the nature of the concurrent therapy (if any), the specific route of administration, and additional factors within the knowledge and expertise of the health practitioner. For example, an effective amount may depend upon the desired level of FXI polypeptide activity and or FXI gene expression that is effective to treat the FXI-associated disease or condition. A skilled artisan can empirically determine an effective amount of a particular FXI dsRNA agent or FXI antisense polynucleotide agent of the invention for use in methods of the invention without necessitating undue experimentation. Combined with the teachings provided herein, by selecting from among various FXI dsRNA agents or FXI antisense polynucleotide agents of the invention, and weighing factors such as potency, relative bioavailability, patient body weight, severity of adverse side-effects and preferred mode of administration, an effective prophylactic or therapeutic treatment regimen can be planned that is effective to treat the particular subject. As used in embodiments of the invention, an effective amount of a FXI dsRNA agent or FXI antisense polynucleotide agent of the invention can be that amount that when contacted with a cell results in a desired biological effect in the cell.

It will be recognized that FXI gene silencing may be determined in any cell expressing FXI, either constitutively or by genomic engineering, and by any appropriate assay. In some embodiments of the invention, FXI gene expression is reduced by at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% by administration of a FXI dsRNA agent of the invention. In some embodiments of the invention, FXI gene expression is reduced by at between 5% and 10%, 5% and 25%, 10% and 50%, 10% and 75%, 25% and 75%, 25% and 100%, or 50% and 100% by administration of a FXI dsRNA agent of the invention.

### Dosing

FXI dsRNA agents and FXI antisense polynucleotide agents are delivered in pharmaceutical compositions in dosages sufficient to inhibit expression of FXI genes. In certain embodiments of the invention, a dose of FXI dsRNA agent or FXI antisense polynucleotide agent is in a range of 0.01 to 200.0 milligrams per kilogram body weight of the recipient per day, generally in the range of 1 to 50 mg per kilogram body weight, 5 to 40 mg/kg body weight, 10 to 30 mg/kg body weight, 1 to 20 mg/kg body weight, 1 to 10 mg/kg body weight, 4 to 15 mg/kg body weight per day, inclusive. For example, the FXI dsRNA agent or FXI antisense polynucleotide agent can be administered in an amount that is from about 0.01 mg/kg, 0.05 mg/kg, 0.1 mg/kg, 0.2 mg/kg, 0.3 mg/kg, 0.4 mg/kg, 0.5 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, through 50 mg/kg body per single dose.

Various factors may be considered in the determination of dosage and timing of delivery of a FXI dsRNA agent of the invention. The absolute amount of a FXI dsRNA agent or FXI antisense polynucleotide agent delivered will depend upon a variety of factors including a concurrent treatment, the number of doses and the individual subject parameters including age, physical condition, size and weight. These are factors well known to those of ordinary skill in the art and can be addressed with no more than routine experimentation. In some embodiments, a maximum dose can be used, that is, the highest safe dose according to sound medical judgment.

Methods of the invention may in some embodiments include administering to a subject 1, 2, 3, 4, 5, 6, 7, 8, 9, 10, or more doses of a FXI dsRNA agent or FXI antisense polynucleotide agent. In some instances, a pharmaceutical compound, (e.g., comprising a FXI dsRNA agent or comprising a FXI antisense polynucleotide agent) can be administered to a subject at least daily, every other day, weekly, every other week, monthly, etc. Doses may be administered once per day or more than once per day, for example, 2, 3, 4, 5, or more times in one 24 hour period. A pharmaceutical composition of the invention may be administered once daily, or the FXI dsRNA agent or FXI antisense polynucleotide agent may be administered as two, three, or more sub-doses at appropriate intervals throughout the day or even using continuous infusion or delivery through a controlled release formulation. In some embodiments of methods of the invention, a pharmaceutical composition of the invention is administered to a subject one or more times per day, one or more times per week, one or more times per month, or one or more times per year.

Methods of the invention, in some aspects, include administration of a pharmaceutical compound alone, in combination with one or more other FXI dsRNA agents or FXI antisense polynucleotide agents, and/or in combination with other drug therapies or treatment activities or regimens that are administered to subjects with a FXI-associated disease or condition. Pharmaceutical compounds may be administered in pharmaceutical compositions. Pharmaceutical compositions used in methods of the invention may be sterile and contain an amount of a FXI dsRNA agent or FXI antisense polynucleotide agent that will reduce activity of a FXI polypeptide to a level sufficient to produce the desired response in a unit of weight or volume suitable for administration to a subject. A dose administered to a subject of a pharmaceutical composition that includes a FXI dsRNA agent or FXI antisense polynucleotide agent to reduce FXI protein activity can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

### Treatment

FXI-associated diseases and conditions in which a decrease in a level and/or activity of FXI polypeptide is effective to treat the disease or condition, can be treated using methods and FXI dsRNA agents of the invention to inhibit FXI expression. Examples of diseases and conditions that may be treated with a FXI dsRNA agent or FXI antisense polynucleotide agent of the invention and a treatment method of the invention, include, but are not limited to thromboembolic condition, the thromboembolic condition is deep vein thrombosis, venous or arterial thrombosis, pulmonary embolism, myocardial infarction, stroke, thrombosis associated with chronic kidney disease or end-stage renal disease (ESRD), including thrombosis associated with dialysis, or other procoagulant condition. Also provided are methods useful treating, preventing, or ameliorating a thromboembolic condition without increasing bleeding risk in an individual. In certain embodiments, the individual is at risk for a thromboembolic condition, including, but not limited to infarct, thrombosis, embolism, thromboembolism such as deep vein thrombosis, pulmonary embolism, myocardial infarction, and stroke. Such diseases, disorders, and conditions can have one or more risk factors, causes, or outcomes in common. Certain risk factors and causes for development of a thromboembolic condition include immobility, surgery (particularly orthopedic surgery), dialysis, malignancy, pregnancy, older age, use of oral contraceptives, atrial fibrillation, previous thromboembolic condition, chronic inflammatory disease, inherited or acquired prothrombotic clotting disorders and thrombosis associated with chronic kidney disease or end-stage renal disease (ESRD). Certain outcomes associated with development of a thromboembolic condition include decreased blood flow through an affected vessel, death of tissue, and death. Such diseases and conditions may be referred to herein as “FXI-associated diseases and conditions” and “diseases and conditions caused and/or modulated by FXI.”

In certain aspects of the invention, a subject may be administered a FXI dsRNA agent or FXI antisense polynucleotide agent of the invention at a time that is one or more of before or after diagnosis of a FXI-associated disease or condition. In some aspects of the invention, a subject is at risk of having or developing a FXI-associated disease or condition. A subject at risk of developing a FXI-associated disease or condition is one who has an increased probability of developing the FXI-associated disease or condition, compared to a control risk of developing the FXI-associated disease or condition. In some embodiments of the invention, a level of risk may be statistically significant compared to a control level of risk. A subject at risk may include, for instance, a subject who is, or will be, a subject who has a preexisting disease and/or a genetic abnormality that makes the subject more susceptible to a FXI-associated disease or condition than a control subject without the preexisting disease or genetic abnormality; a subject having a family and/or personal medical history of the FXI-associated disease or condition; and a subject who has previously been treated for a FXI-associated disease or condition. It will be understood that a preexisting disease and/or a genetic abnormality that makes the subject more susceptible to a FXI-associated disease or condition, may be a disease or genetic abnormality that when present has been previously identified as having a correlative relation to a higher likelihood of developing a FXI-associated disease or condition.

It will be understood that a FXI dsRNA agent or FXI antisense polynucleotide agent may be administered to a subject based on a medical status of the individual subject. For example, a health-care provided for a subject may assess a FXI level measured in a sample obtained from a subject and determine it is desirable to reduce the subject's FXI level, by administration of a FXI dsRNA agent or FXI antisense polynucleotide agent of the invention. In this example, the FXI level may be considered to be a physiological characteristic of a FXI-associated condition, even if the subject is not diagnosed as having a FXI-associated disease such as one disclosed herein. A healthcare provider may monitor changes in the subject's FXI level, as a measure of efficacy of the administered FXI dsRNA agent or FXI antisense polynucleotide agent of the invention. In a non-limiting example, a biological sample, such as a blood or serum sample may be obtained from a subject and a FXI level for the subject determined in the sample. A FXI dsRNA agent or FXI antisense polynucleotide agent is administered to the subject and a blood or liver sample is obtained from the subject following the administration and the FXI level determined using the sample and the results compared to the results determined in the subject's pre-administration (prior) sample. A reduction in the subject's FXI level in the later sample compared to the pre-administration level indicates the administered FXI dsRNA agent or FXI antisense polynucleotide agent efficacy in reducing the lipid level in the subject.

Certain embodiments of methods of the invention include adjusting a treatment that includes administering a dsRNA agent or a FXI antisense polynucleotide agent of the invention to a subject based at least in part on assessment of a change in one or more of the subject's physiological characteristics of a FXI-associated disease or condition resulting from the treatment. For example, in some embodiments of the invention, an effect of an administered dsRNA agent or FXI antisense polynucleotide agent of the invention may be determined for a subject and used to assist in adjusting an amount of a dsRNA agent or FXI antisense polynucleotide agent of the invention subsequently administered to the subject. In a non-limiting example, a subject is administered a dsRNA agent or FXI antisense polynucleotide agent of the invention, the subject's FXI level is determined after the administration, and based at least in part on the determined level, a greater amount of the dsRNA agent or FXI antisense polynucleotide agent is determined to be desirable in order to increase the physiological effect of the administered agent, for example to reduce or further reduce the subject's FXI level. In another non-limiting example, a subject is administered a dsRNA agent or FXI antisense polynucleotide agent of the invention, the subject's FXI level is determined after the administration and based at least in part on the determined level, a lower amount of the dsRNA agent or FXI antisense polynucleotide agent is desirable to administer to the subject.

Thus, some embodiments of the invention include assessing a change in one or more physiological characteristics of resulting from a subject's previous treatment to adjust an amount of a dsRNA agent or FXI antisense polynucleotide agent of the invention subsequently administered to the subject. Some embodiments of methods of the invention include 1, 2, 3, 4, 5, 6, or more determinations of a physiological characteristic of a FXI-associated disease or condition to assess and/or monitor the efficacy of an administered FXI dsRNA agent or FXI antisense polynucleotide agent of the invention, and optionally using the determinations to adjust one or more of: a dose, administration regimen, and or administration frequency of a dsRNA agent or FXI antisense polynucleotide agent of the invention to treat a FXI-associated disease or condition in a subject. In some embodiments of methods of the invention, a desired result of administering an effective amount of a dsRNA agent or FXI antisense polynucleotide agent of the invention to a subject is a reduction of the subject's the FXI mRNA level, the FXI protein level in the subject, or activated partial thromboplastin time (aPTT), prothrombin time (PT) test, thrombin time (TCT), bleeding time, or presence of D-dimer in a plasma, or a tissue sample, and/or accumulation of fat and/or expansion of lipid droplets in the liver, etc., as compared to a prior level determined for the subject, or to a control level.

As used herein, the terms “treat”, “treated”, or “treating” when used with respect to a FXI-associated disease or condition may refer to a prophylactic treatment that decreases the likelihood of a subject developing the FXI-associated disease or condition, and also may refer to a treatment after the subject has developed a FXI-associated disease or condition in order to eliminate or reduce the level of the FXI-associated disease or condition, prevent the FXI-associated disease or condition from becoming more advanced (e.g., more severe), and/or slow the progression of the FXI-associated disease or condition in a subject compared to the subject in the absence of the therapy to reduce activity in the subject of FXI polypeptide.

Certain embodiments of agents, compositions, and methods of the invention can be used to inhibit FXI gene expression. As used herein in reference to expression of a FXI gene, the terms “inhibit,” “silence,” “reduce,” “down-regulate,” and “knockdown” mean the expression of the FXI gene, as measured by one or more of: a level of RNA transcribed from the gene, a level of activity of FXI expressed, and a level of FXI polypeptide, protein or protein subunit translated from the mRNA in a cell, group of cells, tissue, organ, or subject in which the FXI gene is transcribed, is reduced when the cell, group of cells, tissue, organ, or subject is contacted with (e.g., treated with) a FXI dsRNA agent or FXI antisense polynucleotide agent of the invention, compared to a control level of RNA transcribed from the FXI gene, a level of activity of expressed FXI, or a level of FXI translated from the MRNA, respectively. In some embodiments, a control level is a level in a cell, tissue, organ or subject that has not been contacted with (e.g. treated with) the FXI dsRNA agent or FXI antisense polynucleotide agent.

### Administration Methods

A variety of administration routes for a FXI dsRNA agent or FXI antisense polynucleotide agent are available for use in methods of the invention. The particular delivery mode selected will depend at least in part, upon the particular condition being treated and the dosage required for therapeutic efficacy. Methods of this invention, generally speaking, may be practiced using any mode of administration that is medically acceptable, meaning any mode that produces effective levels of treatment of a FXI-associated disease or condition without causing clinically unacceptable adverse effects. In some embodiments of the invention, a FXI dsRNA agent or FXI antisense polynucleotide agent may be administered via an oral, enteral, mucosal, subcutaneous, and/or parenteral route. The term “parenteral” includes subcutaneous, intravenous, intrathecal, intramuscular, intraperitoneal, and intrasternal injection, or infusion techniques. Other routes include but are not limited to nasal (e.g., via a gastro-nasal tube), dermal, vaginal, rectal, sublingual, and inhalation. Delivery routes of the invention may include intrathecal, intraventricular, or intracranial. In some embodiments of the invention, a FXI dsRNA agent or FXI antisense polynucleotide agent may be placed within a slow release matrix and administered by placement of the matrix in the subject. In some aspects of the invention, a FXI dsRNA agent or FXI antisense polynucleotide agent may be delivered to a subject cell using nanoparticles coated with a delivery agent that targets a specific cell or organelle. Various delivery means, methods, agents are known in the art. Non-limiting examples of delivery methods and delivery agents are additionally provided elsewhere herein. In some aspects of the invention, the term “delivering” in reference to a FXI dsRNA agent or FXI antisense polynucleotide agent may mean administration to a cell or subject of one or more “naked” FXI dsRNA agent or FXI antisense polynucleotide agent sequences and in certain aspects of the invention “delivering” means administration to a cell or subject via transfection means, delivering a cell comprising a FXI dsRNA agent or FXI antisense polynucleotide agent to a subject, delivering a vector encoding a FXI dsRNA agent or FXI antisense polynucleotide agent into a cell and/or subject, etc. Delivery of a FXI dsRNA agent or FXI antisense polynucleotide agent using a transfection means may include administration of a vector to a cell and/or subject.

In some methods of the invention one or more FXI dsRNA agents or FXI antisense polynucleotide agents may be administered in formulations, which may be administered in pharmaceutically acceptable solutions, which may routinely contain pharmaceutically acceptable concentrations of salt, buffering agents, preservatives, compatible carriers, adjuvants, and optionally other therapeutic ingredients. In some embodiments of the invention a FXI dsRNA agent or FXI antisense polynucleotide agent may be formulated with another therapeutic agent for simultaneous administration. According to methods of the invention, a FXI dsRNA agent or FXI antisense polynucleotide agent may be administered in a pharmaceutical composition. In general, a pharmaceutical composition comprises a FXI dsRNA agent or FXI antisense polynucleotide agent and optionally, a pharmaceutically-acceptable carrier. Pharmaceutically-acceptable carriers are well-known to those of ordinary skill in the art. As used herein, a pharmaceutically-acceptable carrier means a non-toxic material that does not interfere with the effectiveness of the biological activity of the active ingredients, e.g., the ability of the FXI dsRNA agent or FXI antisense polynucleotide agent to inhibit FXI gene expression in a cell or subject. Numerous methods to administer and deliver dsRNA agents or FXI antisense polynucleotide agents for therapeutic use are known in the art and may be utilized in methods of the invention.

Pharmaceutically acceptable carriers include diluents, fillers, salts, buffers, stabilizers, solubilizers and other materials that are well-known in the art. Exemplary pharmaceutically acceptable carriers are described in U.S. Pat. No. 5,211,657 and others are known by those skilled in the art. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Some embodiments of methods of the invention include administering one or more FXI dsRNA agents or FXI antisense polynucleotide agents directly to a tissue. In some embodiments, the tissue to which the compound is administered is a tissue in which the FXI-associated disease or condition is present or is likely to arise, non-limiting examples of which are the liver or kidney. Direct tissue administration may be achieved by direct injection or other means. Many orally delivered compounds naturally travel to and through the liver and kidneys and some embodiments of treatment methods of the invention include oral administration of one or more FXI dsRNA agents to a subject. FXI dsRNA agents or FXI antisense polynucleotide agents, either alone or in conjunction with other therapeutic agents, may be administered once, or alternatively they may be administered in a plurality of administrations. If administered multiple times, the FXI dsRNA agent or FXI antisense polynucleotide agent may be administered via different routes. For example, though not intended to be limiting, a first (or first several) administrations may be made via subcutaneous means and one or more additional administrations may be oral and/or systemic administrations.

For embodiments of the invention in which it is desirable to administer a FXI dsRNA agent or FXI antisense polynucleotide agent systemically, the FXI dsRNA agent or FXI antisense polynucleotide agent may be formulated for parenteral administration by injection, e.g., by bolus injection or continuous infusion. Formulations for injection may be presented in unit dosage form, e.g., in ampoules or in multi-dose containers, with or without an added preservative. FXI dsRNA agent formulations (also referred to as pharmaceutical compositions) may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day may be used as needed to achieve appropriate systemic or local levels of one or more FXI dsRNA agents or FXI antisense polynucleotide agents and to achieve appropriate reduction in FXI protein activity.

In yet other embodiments, methods of the invention include use of a delivery vehicle such as biocompatible microparticle, nanoparticle, or implant suitable for implantation into a recipient, e.g., a subject. Exemplary bioerodible implants that may be useful in accordance with this method are described in PCT Publication No. WO 95/24929 (incorporated by reference herein), which describes a biocompatible, biodegradable polymeric matrix for containing a biological macromolecule.

Both non-biodegradable and biodegradable polymeric matrices can be used in methods of the invention to deliver one or more FXI dsRNA agents or FXI antisense polynucleotide agents to a subject. In some embodiments, a matrix may be biodegradable. Matrix polymers may be natural or synthetic polymers. A polymer can be selected based on the period of time over which release is desired, generally in the order of a few hours to a year or longer. Typically, release over a period ranging from between a few hours and three to twelve months can be used. The polymer optionally is in the form of a hydrogel that can absorb up to about 90% of its weight in water and further, optionally is cross-linked with multivalent ions or other polymers.

In general, FXI dsRNA agents or FXI antisense polynucleotide agents may be delivered in some embodiments of the invention using the bioerodible implant by way of diffusion, or by degradation of the polymeric matrix. Exemplary synthetic polymers for such use are well known in the art. Biodegradable polymers and non-biodegradable polymers can be used for delivery of FXI dsRNA agents or FXI antisense polynucleotide agents using art-known methods. Bioadhesive polymers such as bioerodible hydrogels (see H. S. Sawhney, C. P. Pathak and J. A. Hubell in Macromolecules, 1993, 26, 581-587, the teachings of which are incorporated by reference herein) may also be used to deliver FXI dsRNA agents or FXI antisense polynucleotide agents for treatment of a FXI-associated disease or condition. Additional suitable delivery systems can include time-release, delayed release or sustained release delivery systems. Such systems can avoid repeated administrations of a FXI dsRNA agent or FXI antisense polynucleotide agent, increasing convenience to the subject and the medical care professional. Many types of release delivery systems are available and known to those of ordinary skill in the art. (See for example: U.S. Pat. Nos. 5,075,109; 4,452,775; 4,675,189; 5,736,152; 3,854,480; 5,133,974; and 5,407,686 (the teaching of each of which is incorporated herein by reference). In addition, pump-based hardware delivery systems can be used, some of which are adapted for implantation.

Use of a long-term sustained release implant may be suitable for prophylactic treatment of subjects and for subjects at risk of developing a recurrent FXI-associated disease or condition. Long-term release, as used herein, means that the implant is constructed and arranged to deliver a therapeutic level of a FXI dsRNA agent or FXI antisense polynucleotide agent for at least up to 10 days, 20 days, 30 days, 60 days, 90 days, six months, a year, or longer. Long-term sustained release implants are well-known to those of ordinary skill in the art and include some of the release systems described above.

Therapeutic formulations of FXI dsRNA agents or FXI antisense polynucleotide agents may be prepared for storage by mixing the molecule or compound having the desired degree of purity with optional pharmaceutically acceptable carriers, excipients or stabilizers [Remington's Pharmaceutical Sciences 21^{st} edition, (2006)], in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

### Cells, Subjects, and Controls

Methods of the invention may be used in conjunction with cells, tissues, organs and/or subjects. In some aspects of the invention a subject is a human or vertebrate mammal including but not limited to a dog, cat, horse, cow, goat, mouse, rat, and primate, e.g., monkey. Thus, the invention can be used to treat FXI-associated diseases or conditions in human and non-human subjects. In some aspects of the invention a subject may be a farm animal, a zoo animal, a domesticated animal or non-domesticated animal and methods of the invention can be used in veterinary prevention and treatment regimens. In some embodiments of the invention, the subject is a human and methods of the invention can be used in human prevention and treatment regimens.

Non-limiting examples of subjects to which the present invention can be applied are subjects who are diagnosed with, suspected of having, or at risk of having a disease or condition associated with a higher than desirable FXI expression and/or activity, also referred to as “elevated levels of FXI expression”. Non-limiting examples of diseases and conditions associated with a higher than desirable levels of FXI expression and/or activity are described elsewhere herein. Methods of the invention may be applied to a subject who, at the time of treatment, has been diagnosed as having the disease or condition associated with a higher than desirable FXI expression and/or activity, or a subject who is considered to be at risk for having or developing a disease or condition associated with a higher than desirable FXI expression and/or activity. In some aspects of the invention a disease or condition associated with a higher than desirable FXI level of expression and/or activity is an acute disease or condition, and in certain aspects of the invention a disease or condition associated with a higher than desirable FXI level of expression and/or activity is a chronic disease or condition.

In a non-limiting example, a FXI dsRNA agent of the invention is administered to a subject diagnosed with, suspected of having, or at risk of having, statin resistant hypercholesterolemia, which is a disease in which it is desirable to reduce FXI expression. Methods of the invention may be applied to the subject who, at the time of treatment, has been diagnosed as having the disease or condition, or a subject who is considered to be at risk for having or developing the disease or condition.

In another non-limiting example, a FXI dsRNA agent of the invention is administered to a subject diagnosed with, suspected of having, or at risk of having, hyperlipidemia, which is a disease in which it is desirable to reduce FXI expression. Methods of the invention may be applied to the subject who, at the time of treatment, has been diagnosed as having the disease or condition, or a subject who is considered to be at risk for having or developing the disease or condition.

A cell to which methods of the invention may be applied include cells that are in vitro, in vivo, ex vivo cells. Cells may be in a subject, in culture, and/or in suspension, or in any other suitable state or condition. A cell to which a method of the invention may be applied can be a liver cell, a hepatocyte, a cardiac cell, a pancreatic cell, a cardiovascular cell, kidney cell or other type of vertebrate cell, including human and non-human mammalian cells. In certain aspects of the invention, a cell to which methods of the invention may be applied is a healthy, normal cell that is not known to be a disease cell. In certain embodiments of the invention a cell to which methods and compositions of the invention are applied to a liver cell, a hepatocyte, a cardiac cell, a pancreatic cell, a cardiovascular cell, and/or a kidney cell. In certain aspects of the invention, a control cell is a normal cell, but it will be understood that a cell having a disease or condition may also serve as a control cell in particular circumstances for example to compare results in a treated cell having a disease or condition versus an untreated cell having the disease or condition, etc.

A level of FXI polypeptide activity can be determined and compared to control level of FXI polypeptide activity, according to methods of the invention. A control may be a predetermined value, which can take a variety of forms. It can be a single cut-off value, such as a median or mean. It can be established based upon comparative groups, such as in groups having normal levels of FXI polypeptide and/or FXI polypeptide activity and groups having increased levels of FXI polypeptide and/or FXI polypeptide activity. Another non-limiting example of comparative groups may be groups having one or more symptoms of or a diagnosis of a FXI-associated disease or condition; groups without having one or more symptoms of or a diagnosis of the disease or condition; groups of subjects to whom an siRNA treatment of the invention has been administered; groups of subjects to whom an siRNA treatment of the invention has not been administered. Typically, a control may be based on apparently healthy normal individuals in an appropriate age bracket or apparently healthy cells. It will be understood that controls according to the invention may be, in addition to predetermined values, samples of materials tested in parallel with the experimental materials. Examples include samples from control populations or control samples generated through manufacture to be tested in parallel with the experimental samples. In some embodiments of the invention, a control may include a cell or subject not contacted or treated with a FXI dsRNA agent of the invention and in such instances, a control level of FXI polypeptide and/or FXI polypeptide activity can be compared to a level of FXI polypeptide and/or FXI polypeptide activity in a cell or subject contacted with a FXI dsRNA agent or FXI antisense polynucleotide agent of the invention.

In some embodiments of the invention a level of FXI polypeptide determined for a subject can be a control level against which a level of FXI polypeptide determined for the same subject at a different time is compared. In a non-limiting example, a level of FXI is determined in a biological sample obtained from a subject who has not been administered a FXI treatment of the invention. In some embodiments, the biological sample is a serum sample. In some embodiments, the biological sample is a liver sample. The level of FXI polypeptide determined in the sample obtained from the subject can serve as a baseline or control value for the subject. After one or more administrations of a FXI dsRNA agent to the subject in a treatment method of the invention, one or more additional serum samples can be obtained from the subject and the level of FXI polypeptide in the subsequent sample or samples can be compared to the control/baseline level for the subject. Such comparisons can be used to assess onset, progression, or recession of a FXI associated disease or condition in the subject. For example, a level of FXI polypeptide in the baseline sample obtained from the subject that is higher than a level obtained from the same subject after the subject has been administered a FXI dsRNA agent or FXI antisense polynucleotide agent of the invention indicates regression of the FXI-associated disease or condition and indicates efficacy of the administered FXI dsRNA agent of the invention for treatment of the FXI-associated disease or condition.

In some aspects of the invention, values of one or more of a level of FXI polypeptide and/or FXI polypeptide activity determined for a subject may serve as control values for later comparison of levels of FXI polypeptide and/or FXI activity, in that same subject, thus permitting assessment of changes from a “baseline” FXI polypeptide activity in a subject. Thus, an initial FXI polypeptide level and/or initial FXI polypeptide activity level may be present and/or determined in a subject and methods and compounds of the invention may be used to decrease the level of FXI polypeptide and/or FXI polypeptide activity in the subject, with the initial level serving as a control level for that subject.

Using methods of the invention, FXI dsRNA agents and/or FXI antisense polynucleotide agents of the invention may be administered to a subject. Efficacy of the administration and treatment of the invention can be assessed when a level of FXI polypeptide in a serum sample obtained from a subject is decreased by at least 0.5%, 1%, 5%, 10%, 20%, 30%, 40%, 50%, 60%, 70%, 80%, 90%, 95%, or more compared to a pre-administration level of FXI polypeptide in a serum sample obtained from the subject at a prior time point, or compared to a non-contacted control level, for example a level of FXI polypeptide in a control serum sample. It will be understood that a level of FXI polypeptide and a level of FXI polypeptide activity both correlate with a level of FXI gene expression. Certain embodiments of methods of the invention comprise administering a FXI dsRNA and/or FXI antisense agent of the invention to a subject in an amount effective to inhibit FXI gene expression and thereby reduce a level of FXI polypeptide and reduce a level of FXI polypeptide activity in the subject.

Some embodiments of the invention, include determining presence, absence, and/or an amount (also referred to herein as a level) of FXI polypeptide in one or more biological samples obtained from one or more subjects. The determination can be used to assess efficacy of a treatment method of the invention. For example, methods and compositions of the invention can be used to determine a level of FXI polypeptide in a biological sample obtained from a subject previously treated with administration of a FXI dsRNA agent and/or a FXI antisense agent of the invention. A level of FXI polypeptide determined in a serum sample obtained from the treated subject that is lower by at least 0.5%, 1%, 5%, 10%, 20%, 30%, 40%, 50%, 60%, 70%, 80%, 90%, 95%, or more compared to a pretreatment level of FXI polypeptide determined for the subject, or compared to a non-contacted control biological sample level, indicates a level of efficacy of the treatment administered to the subject.

In some embodiments of the invention a physiological characteristic of a FXI-associated disease or condition determined for a subject can be a control determination against which a determination of the physiological characteristic in the same subject at a different time is compared. In a non-limiting example, a physiological characteristic such as the FXI mRNA level, the FXI protein level in the subject, or activated partial thromboplastin time (aPTT), prothrombin time (PT) test, thrombin time (TCT), bleeding time, or presence of D-dimer in a blood sample of the animal. The FXI mRNA level (and/or other physiological characteristic of a FXI disease or condition) determined in the sample obtained from the subject can serve as a baseline or control value for the subject. After one or more administrations of a FXI dsRNA agent to the subject in a treatment method of the invention, one or more additional liver or serum samples can be obtained from the subject and FXI mRNA level and/or FXI protein level in the subsequent sample or samples are compared to the control/baseline level and/or ratio, respectively, for the subject. Such comparisons can be used to assess onset, progression, or recession of a FXI associated disease or condition in the subject. For example, FXI mRNA level in the baseline sample obtained from the subject that is higher than FXI mRNA level determined in a sample obtained from the same subject after the subject has been administered a FXI dsRNA agent or FXI antisense polynucleotide agent of the invention indicates regression of the FXI-associated disease or condition and indicates efficacy of the administered FXI dsRNA agent of the invention for treatment of the FXI-associated disease or condition.

In some aspects of the invention, values of one or more of a physiological characteristic of a FXI-associated disease or condition determined for a subject may serve as control values for later comparison of the physiological characteristics in that same subject, thus permitting assessment of changes from a “baseline” physiological characteristic in a subject. Thus, an initial physiological characteristic may be present and/or determined in a subject and methods and compounds of the invention may be used to decrease the level of FXI polypeptide and/or FXI polypeptide activity in the subject, with the initial physiological characteristic determination serving as a control for that subject.

Using methods of the invention, FXI dsRNA agents and/or FXI antisense polynucleotide agents of the invention may be administered to a subject in an effective amount to treat a FXI disease or condition. Efficacy of the administration and treatment of the invention can be assessed by determining a change in one or more physiological characteristics of the FXI disease or condition. In a non-limiting example, a FXI mRNA level in a serum sample obtained from a subject is decreased by at least 0.5%, 1%, 5%, 10%, 20%, 30%, 40%, 50%, 60%, 70%, 80%, 90%, 95%, or more compared to a pre-administration lipid in a serum sample obtained from the subject at a prior time point, or compared to a non-contacted control level, for example FXI mRNA level in a control serum sample. It will be understood that the FXI mRNA level, the FXI protein level in the subject, or activated partial thromboplastin time (aPTT), prothrombin time (PT) test, thrombin time (TCT), bleeding time, or presence of D-dimer in a blood each correlates with a level of FXI gene expression. Certain embodiments of methods of the invention comprise administering a FXI dsRNA and/or FXI antisense agent of the invention to a subject in an amount effective to inhibit FXI gene expression and thereby reduce the FXI mRNA level, the FXI protein level in the subject, or otherwise positively impact a physiological characteristic of a FXI-associated disease or condition in the subject.

Some embodiments of the invention, include determining presence, absence, and/or a change in a physiological characteristic of a FXI-associated disease or condition using methods such as but not limited to: (1) assessing one or more biological samples obtained from one or more subjects for the physiological characteristic; (2) imaging a subject (for example but not limited to obtaining a liver image); and (3) or physical examination of the subject. The determination can be used to assess efficacy of a treatment method of the invention.

### Kits

Also within the scope of the invention are kits that comprise one or more FXI dsRNA agents and/or FXI antisense polynucleotide agents and instructions for its use in methods of the invention. Kits of the invention may include one or more of a FXI dsRNA agent, FXI sense polynucleotide, and FXI antisense polynucleotide agent that may be used to treat a FXI-associated disease or condition. Kits containing one or more FXI dsRNA agents, FXI sense polynucleotides, and FXI antisense polynucleotide agents can be prepared for use in treatment methods of the invention. Components of kits of the invention may be packaged either in aqueous medium or in lyophilized form. A kit of the invention may comprise a carrier being compartmentalized to receive in close confinement therein one or more container means or series of container means such as test tubes, vials, flasks, bottles, syringes, or the like. A first container means or series of container means may contain one or more compounds such as a FXI dsRNA agent and/or FXI sense or antisense polynucleotide agent. A second container means or series of container means may contain a targeting agent, a labelling agent, a delivery agent, etc. that may be included as a portion of a FXI dsRNA agent and/or FXI antisense polynucleotide to be administered in an embodiment of a treatment method of the invention.

A kit of the invention may also include instructions. Instructions typically will be in written form and will provide guidance for carrying-out a treatment embodied by the kit and for making a determination based upon that treatment.

The following examples are provided to illustrate specific instances of the practice of the present invention and are not intended to limit the scope of the invention. As will be apparent to one of ordinary skill in the art, the present invention will find application in a variety of compositions and methods.

### Examples

### Example 1

### Phosphoramidite Compound 2

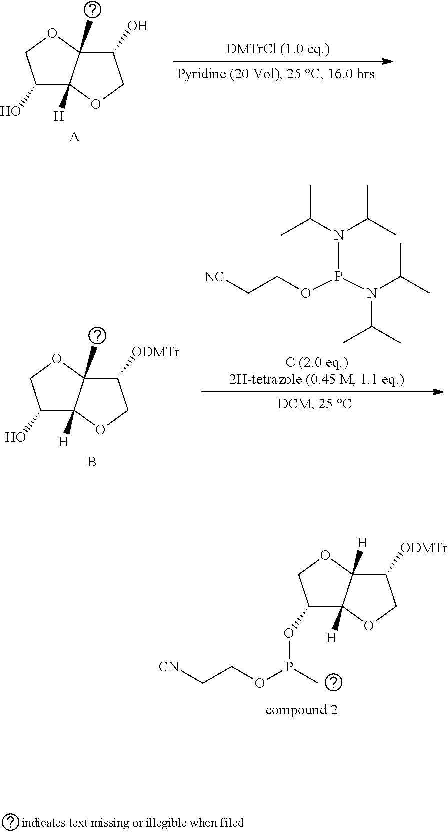

DMTrCl (232 g, 684 mmol, 1.0 eq) in pyridine (400 mL) was added to the solution of isomannide compound A (100 g, 684 mmol, 1.0 eq) in pyridine (600 mL), and the mixture was stirred at 25° C. for 12 hrs. LC-MS showed compound A was consumed completely and one main peak with desired mass was detected. The resulting reaction mixture was diluted with water (500 mL), extracted with DCM (500 mL*2), and the combined organic phases were washed with brine (500 mL), dried over Na₂SO₄ and concentrated in vacuum to get the residue. The residue was purified by column chromatography (DCM/MeOH=100/1 to 50/1, 0.1% Et₃N) to give compound B (150 g, 48.9% yield) a yellow solid.

¹H NMR: EC4783-404-P1B1_C (400 MHz, DMSO-d₆)

δ ppm 7.46 (br d, J=7.63 Hz, 2H) 7.28-7.37 (m, 6H) 7.19-7.25 (m, 1H) 6.90 (br d, J=7.88 Hz, 4H) 4.70 (d, J=6.50 Hz, 1H) 3.99-4.09 (m, 6H) 3.88-3.96 (m, 2H) 3.83 (br dd, J=7.82, 6.94 Hz, 1H) 3.74 (s, 6H) 3.41 (br t, J=8.13 Hz, 1H) 3.05 (t, J=8.44 Hz, 1H) 2.85 (br t, J=7.50 Hz, 1H).

To a solution of compound B (80.0 g, 178 mmol, 1.0 eq) in DCM (800 mL) at 25° C. under N₂ atmosphere was added dropwise 2H-tetrazole (0.45 M, 436 mL, 1.1 eq), then compound C (80.6 g, 267 mmol, 85.0 mL, 1.5 eq) in DCM (200 mL) was added dropwise to the mixture. The reaction mixture was stirred at 25° C. under for 1.0 hr. LC-MS showed compound B was consumed completely and one main peak with desired mass was detected. The resulting reaction mixture was cooled to −20° C. and poured into ice cold sat·NaHCO₃ (500 mL), extracted with DCM (500 mL*3), the combined organic layers were washed with sat·NaHCO₃/brine=1:1 (3 00 mL/300 mL), dried over Na₂SO₄ and concentrated in vacuum (35° C.) to get the residue (100 mL). The residue was purified by column chromatography (Al₂O₃, DCM/MeOH=100/1 to 50/1, 0.1% Et₃N) to give compound 2 (77 g, 119 mmol, 66.5% yield) as a white solid.

¹H NMR: EC4783-423-P1B1_C (400 MHz, DMSO-d₆)

δ ppm 7.22 (br d, J=7.50 Hz, 2H) 7.05-7.14 (m, 6H) 6.96-7.02 (m, 1H) 6.67 (br dd, J=8.82, 1.81 Hz, 4H) 3.95-4.07 (m, 2H) 3.73-3.83 (m, 1H) 3.62-3.72 (m, 2H) 3.48-3.53 (m, 6H) 3.27-3.37 (m, 3H) 3.11 (s, 6H) 2.82 (td, J=8.54, 2.31 Hz, 1H) 2.47-2.63 (m, 3H) 2.28 (br d, J=1.63 Hz, 3H) 0.82-1.00 (m, 13H).

### Phosphoramidite Compound 1

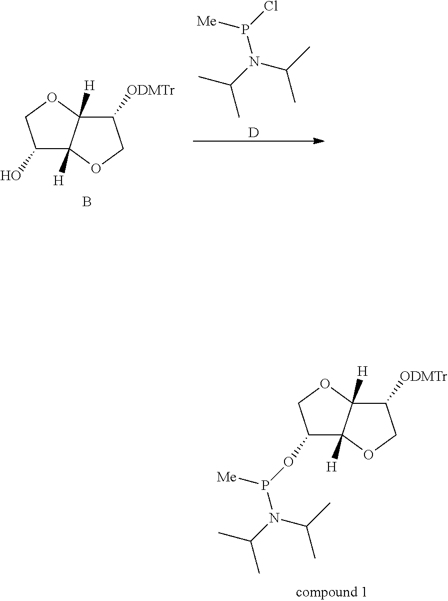

To a solution of compound B (500 mg, 1.11 mmol, 1.0 eq) in DCM (5.0 mL) was added compound D (607 mg, 3.34 mmol, 3.0 eq) and DIEA (432 mg, 3.34 mmol, 582 μL, 3.0 eq) under N₂ atmosphere at 0-5° C., the mixture was stirred at 25° C. for 1.0 hrs. LC-MS showed Compound B was consumed completely, several new peaks were shown on LC-MS and ~70.9% of desired compound was detected. The resulting reaction mixture was cooled to −20° C. and poured into cold (0-5° C.) sat·NaHCO₃ (5.0 mL) solution, extracted with DCM (5.0 mL*2), the combined organic layers were washed with cold (0-5° C.) sat·NaHCO₃/brine=1:1 (5.0 mL/5.0 mL), dried over Na₂SO₄ and concentrated in vacuum to get the residue (~5 mL). The residue was purified by column chromatography (alkaline Al₂O₃, Petroleum ether/Ethyl acetate=10/1 to 5/1, 0.1% Et₃N) to give compound 1 (280 mg, 471 mol, 42.3% yield) was obtained as a white solid.

¹H NMR: EC10615-49-P1N (400 MHz, DMSO-d₆)

δ ppm 7.44 (br d, J=7.63 Hz, 2H), 7.31 (br t, J=7.94 Hz, 6H), 7.18-7.26 (m, 1H), 6.89 (brd, J=8.00 Hz, 4H), 4.08-4.13 (m, 1H), 3.95-4.03 (m, 1H), 3.84-3.93 (m, 1H), 3.77-3.83 (m, 1H), 3.74 (s, 6H), 3.43-3.53 (m, 3H), 3.38 (br d, J=6.75 Hz, 1H), 2.94-3.04 (m, 1H), 2.70-2.85 (m, 1H), 1.09-1.15 (m, 12H), 1.07 (br s, 3H).

Other phosphoramidites may be prepared according to procedures described herein and/or prior arts such as, but are not limited to, US426,220 and WO02/36743.

### Example 2. Preparation of a Solid Support Comprising Phosphoramidites Monomers of the Present Invention

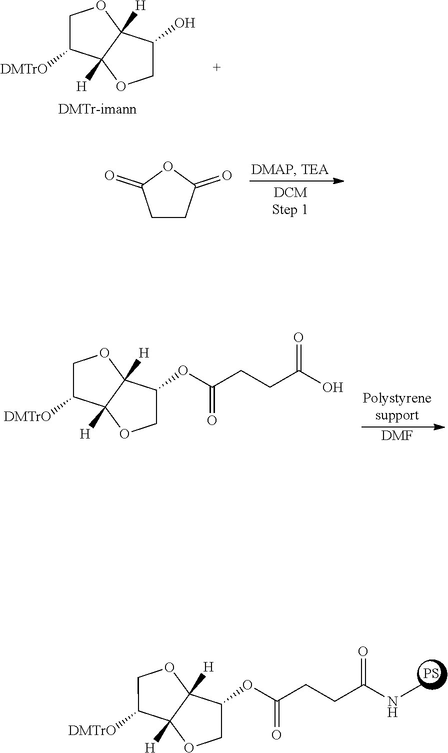

[symbol] represents amine methyl polyethylene macroporous resin carrier part

Dichloromethane (19.50 kg) was added to the 50 L glass kettle under the protection of nitrogen and started stirring. The temperature was controlled at 20~30° C., and DMTr imann (1.47 kg), triethylamine (1.50 kg), 4-dimethylaminopyridine (0.164 kg) and succinic anhydride (1.34 kg) was added to the glass kettle. The system was kept at 20~30° C. for 18 h, samples were taken and the reaction was ended. Saturated sodium bicarbonate solution (22.50 kg) was added into the reaction system, stirred for 10-20 min, and allowed to separate into layers. The organic phase was separated, and the aqueous phase was extracted twice with dichloromethane, and the organic phase was combined and dried over anhydrous sodium sulfate, filtered, and concentrated in vacuum to get the residue forming a gray to off-white solid of 1.83 kg.

N, N-dimethylformamide (23.50 kg) was added into a 100 L glass kettle and stirred. The temperature was controlled at 20~30° C. Under the protection of nitrogen, the products of the previous step, O-benzotriazole tetramethylurea hexafluorophosphate (0.33 kg) and N, N-diisopropylethylamine (0.13 kg) were added into the aforesaid 100 L glass kettle through the solid feeding funnel and stirred for 10~30 minutes and were discharged into a 50 L zinc barrel for use. Macroporous amine methyl resin (3.25 kg) (purchased from Tianjin Nankai Hecheng Science and Technology Co., Ltd., batch number HA2X1209, load capacity 0.48 mmol/g) were added into the aforesaid 100 L solid phase synthesis reactor through the solid feeding funnel, the temperature was controlled at 20~30° C., N, N-dimethylformamide (21.00 kg+21.00 kg) and the reaction solution in the zinc barrel of the previous step were add into the solid phase synthesis reactor. The system was subject to thermal insulation reaction, and the solid load was tracked to ≥250 umol/g, and the load detection method was UV. The system was filtered under the pressure of nitrogen, the filter cake was washed with N, N-dimethylformamide for three times (26.00 kg+26.10 kg+26.00 kg), and the filter cake was left in the kettle. CAP.A (50% acetonitrile and 50% acetic anhydride, 4.40 kg+4.42 kg+4.30 kg) and CAP.B (20% pyridine and 30% N-methylimidazole and 50% acetonitrile, 4.40 kg+4.40 kg+4.47 kg) were added into the 80 L glass kettle, and stirred for 3~8 min before use. This operation was repeated for three times to cap, and acetonitrile (18.00 kg+18.00 kg+18.00 kg+17.50 kg+17.50 kg) was added into the solid phase synthesis kettle. Filter-pressed after nitrogen bubbling for 10~30 min. This operation was repeated for four times, the filter cake was purged in the solid phase synthesis kettle with nitrogen for 2-4 h, and then was transferred to a 50 L filter press tank. The temperature was controlled at 15~30° C., continued drying, and obtained yellow to white solid product after drying, weight: 3.516 kg.

### Example 3. Synthesis of FXI RNAi Agents

FXI RNAi agent duplexes shown in Table 2-3, above, were synthesized in accordance with the following general procedures:
Sense and antisense strand sequences of siRNA were synthesized on oligonucleotide synthesizers using a well-established solid phase synthesis method based on phosphoramidite chemistry. Oligonucleotide chain propagation is achieved through 4-step cycles: a deprotection, a condensation, a capping and an oxidation or a sulfurization step for addition of each nucleotide. Syntheses were performed on a solid support made of controlled pore glass (CPG, 1000 Å). Monomer phosphoramidites may be purchased from commercial sources or may be the phosporamidite compounds in example 3. The phosporamidite compounds herein may be attached to the 3′-end as a monomeric phosphoramidite, and further be attached to the CPG solid support. In the case of attachment at the 5′-end, the phosphoramidite compounds may be used for the final coupling reaction, and can be further conjugated to target ligands if necessary. For siRNAs used for in vitro screening (Table 2), syntheses were carried out at 2 gmol scale, and for siRNAs used for in vivo testing (Table 3), syntheses were carried out at scale of 5 gmol or larger. In the case where the GalNAc ligand (GLO-0 as a non-limiting example) is attached at 3′-end of sense strand, GalNAc ligand attached CPG solid support was used. In the case where the GalNAc ligand (GLS-5* or GLS-15* as non-limiting example, GLS-5* or GLS-15* Phosphoramidites with GalNAc ligand cluster were public in WO2023/045995A1 (incorporated herein in its entirety)) is attached at 5′-end of sense strand, a GalNAc phosphoramidite was used for the last coupling reaction. Trichloroacetic acid (TCA) 3% in dichloromethane was used for deprotection of 4,4′-dimethoxytrityl protecting group (DMT). 5-Ethylthio-1H-tetrazole was used as an activator. 12 in THF/Py/H₂O and phenylacetyl disulfide (PADS) in pyridine/MeCN was used for oxidation and sulfurization reactions, respectively. After the final solid phase synthesis step, solid support bound oligomer was cleaved and protecting groups were removed by treating with a 1:1 volume solution of 40 wt. % methylamine in water and 28% ammonium hydroxide solution. For the synthesis of siRNAs used for in vitro screening, crude mixture was concentrated. The remaining solid was dissolved in 1.0 M NaOAc, and ice cold EtOH was added to precipitate out the single strand product as the sodium salt, which was used for annealing without further purification. For the synthesis of siRNAs used for in vivo testing, crude single strand product was further purified by ion pairing reversed phase HPLC (IP-RP-HPLC). Purified single strand oligonucleotide product from IP-RP-HPLC was converted to sodium salt by dissolving in 1.0 M NaOAc and precipitation by addition of ice cold EtOH. Annealing of equimolar complementary sense stand and antisense strand oligonucleotide in water was performed to form the double strand siRNA product, which was lyophilized to afford a fluffy white solid.

TABLE 4
Abbreviations of nucleotide monomers used in nucleic acid sequence representation. It will be
understood that these monomers, when present in an oligonucleotide, are mutually linked by 5′-3′-
phosphodiester bonds, and it is understood that when the nucleotide contains a 2′-fluoro modification, then
the fluoro replaces the hydroxy at that position of the parent nucleotide (i.e., it is a 2′-deoxy-2′-
fluoronucleotide). It will also be understood that the abbreviations correspond to nucleotides which omit
the 3′-phosphate when found at the 3′-terminal position (i.e., they are 3′-OH). A skilled artisan will
understand that these abbreviations are just identification symbols and are able to determine the linking
modes of the nucleotide sequences containing these abbreviations.

| Abbreviations | Nucleotide(s) | |
|---|---|---|
| A | adenosine-3′-phosphate | |
| C | cytidine-3′-phosphate | |
| G | guanosine-3′-phosphate | |
| T | 5-methyluridine-3′-phosphate | |
| U | uridine-3′-phosphate | |
| a | 2′-O-methyladenosine-3′-phosphate | |
| c | 2′-O-methylcytidine-3′-phosphate | |
| g | 2′-O-methylguanosine-3′-phosphate | |
| t | 2′-O-methyl-5-methyluridine-3′-phosphate | |
| u | 2′-O-methyluridine-3′-phosphate | |
| Af | 2′-fluoroadenosine-3′-phosphate | |
| Cf | 2′-fluorocytidine-3′-phosphate | |
| Gf | 2′-fluoroguanosine-3′-phosphate | |
| Tf | 2′-fluoro-5-methyluridine-3′-phosphate | |
| Uf | 2′-fluorouridine-3′-phosphate | |
| Af* | 2′-fluoroadenosine-3′-phosphorothioate | |
| Cf* | 2′-fluorocytidine-3′-phosphorothioate | |
| Gf* | 2′-fluoroguanosine-3′-phosphorothioate | |
| Tf* | 2′-fluoro-5-methyluridine-3′-phosphorothioate | |
| Uf* | 2′-fluorouridine-3′-phosphorothioate | |
| a* | 2′-O-methyladenosine-3′-phosphorothioate | |
| c* | 2′-O-methylcytidine-3′-phosphorothioate | |
| g* | 2′-O-methylguanosine-3′-phosphorothioate | |
| t* | 2′-O-methyl-5-methyluridine-3′-phosphorothioate | |
| u* | 2′-O-methyluridine-3′-phosphorothioate | |
| aUNA | 2′,3′-seco-adenosine-3′-phosphate | |
| cUNA | 2′,3′-seco-cytidine-3′-phosphate | |
| gUNA | 2′,3′-seco-guanosine-3′-phosphate | |
| tUNA | 2′,3′-seco-5-methyluridine-3′-phosphate | |
| uUNA | 2′,3′-seco-uridine-3′-phosphate | |
| Vp | Vinyl-phosphate | |
| phos | 5′-Phosphate | |
| invab | inverted abasic-5′-phosphate. linked to adjacent nucleotide via a substituent at its 3′ position | |
| | (a 3′-3′ linkage) when on the 3′ end of a strand; linked to adjacent nucleotide via a substituent | |
| | at its 5′ position (a 5′-5′ internucleotide linkage) when on the 5′ end of a strand, It will also be | |
| | understood that the abbreviations correspond to invab which omit the 5′-phosphate when | |
| | found at the 3′-terminal position (i.e., they are 5′-OH) | |
| invab* | inverted abasic-5′-phosphorothioate. linked to adjacent nucleotide via a substituent at its 3′ | |
| | position (a 3′-3′ linkage) when on the 3′ end of a strandlinked to adjacent nucleotide via a | |
| | substituent at its 5′ position (a 5′-5′ internucleotide linkage) when on the 5′ end of a strand, It | |
| | will also be understood that the abbreviations correspond to invab* which omit the 5′- | |
| | phosphorothioate when found at the 3′-terminal position (i.e., they are 5′-OH); | |
| imann | imann when at the end of each strand | imann when in the middle of each strand |
| | 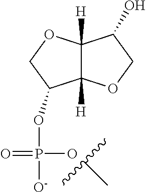 | 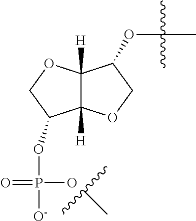 |
| imann* | isomannide residue-phosphorothioate | |
| | imann* when at the end of each strand | imann* when in the middle of each strand |
| | 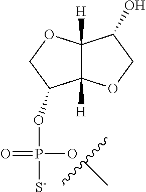 | 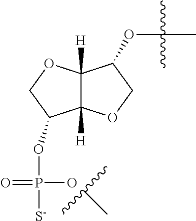 |

### Example 4. In Vitro Screening of FXI siRNA Duplexes

Huh7 cells were trypsinized and adjusted to appropriate density, mixed with the complexes of psiCHECK(TM)-2 Vector plasmid and Lipofectamine 2000 (Invitrogen-11668-019) and seeded into 96-well plates. Cells were transfected with test siRNAs or a control siRNA using Lipofectamine RNAiMax (Invitrogen-13778-150) at the same time of seeding following the protocol according to manufacturer's recommendation. The siRNAs were tested at two concentrations (1 nM and 0.1 nM) in triplicate.

Day 1, psiCHECK(TM)-2 Vector transfection (one plate)
(1) Transfer 2.5 μg psiCHECK(TM)-2 Vector plasmid into an RNASE free Eppendorf tube (solution mix #1)
(2) Add trypsin to disassociate Huh7 cells in one flask, and count cells using Vi-Cell counting machine, adjust the cell density to 1*10\hat{ }5/ml
(3) Transfer 7.5 μL Lipofectamine 2000 (Invitrogen-11668-019) into solution mix #1 tube, mix.
(4) Add solution in Step 3 into cell suspension, mix, and dispense suspension into the 96 well plate (100 μl/well)

Day 2, siRNAs transfection
(1) Dilute Lipofectamine®RNAiMAX Reagent with Opti-MEM® Medium.
(2) Dilute the siRNA with RNA-free water to make 12× stock.
(3) Mix equal volume of diluted RNAiMax and siRNA. Incubate the mixture at RT for 15 min to allow complex formation.
(4) Add 45 μl/well compound Lipofectamine® RNAiMAX(Opti-MEM) mix into 225 μl/well DMEM fresh medium, and discard the supernatants in assay plate, add 120 μl/well compound mix into 96 well plates.
(5) No compound control well was defined as cells transfected with psiCHECK(TM)-2 Vector and without siRNA treatment; blank control was cell only wells.

Day 3, Dual-Glo® Luciferase Assay
(1) Add Reagent to assay plate, wait 10 minutes to allow for cell lysis to occur.
(2) Transfer 100 μl cell lysates into a plate, then measure the firefly luminescence.
(3) Add 50 μl of Dual-Gbio Stop & G Reagent to the assay plates and mix, wait 10 minutes, then measure Renilla luminescence.
(4) Calculate the relative expression

### Data Analysis

Ratio of sample well=(sample Renilla luminescence-background blank)/(sample Fireflyluminescence-background blank)

Ratio of no compound control well=(control Renilla luminescence-background blank)/(control sample Fireflyluminescence-background blank)

% inhibition=100−(Ratio of sample well/the average Ratio of no compound control)×100%.

Table 5 provides experimental results of in vitro studies using various FXI RNAi agents to inhibit FXI expression. The duplex sequences used correspond to those shown in Table 2.

| | |
|---|---|
| Duplex | Average Inhibition % (Mean ± SD) |

| AV# | 1 nM | 0.1 nM |
|---|---|---|
| AV01117 | 0.26 ± 2.95 | −11.90 ± 18.49 |
| AV01118 | 1.08 ± 11.05 | 22.01 ± 11.15 |
| AV01119 | 4.59 ± 7.36 | −7.30 ± 7.40 |
| AV01120 | −2.77 ± 11.32 | 1.56 ± 6.52 |
| AV01121 | 24.47 ± 5.03 | −3.01 ± 6.29 |
| AV01122 | 81.75 ± 6.08 | 39.09 ± 11.84 |
| AV01123 | 81.77 ± 2.84 | 43.00 ± 9.33 |
| AV01124 | 56.74 ± 4.37 | 19.87 ± 19.34 |
| AV01126 | 52.24 ± 8.28 | 11.22 ± 16.81 |
| AV01127 | 44.23 ± 9.36 | 3.10 ± 21.98 |
| AV01128 | 30.80 ± 16.34 | 1.74 ± 11.10 |
| AV01129 | 25.21 ± 8.40 | −12.48 ± 1.16 |
| AV01130 | 28.50 ± 15.20 | 7.35 ± 10.77 |
| AV01131 | 15.59 ± 15.26 | 21.76 ± 23.89 |
| AV01132 | 21.75 ± 8.82 | 14.96 ± 8.32 |
| AV01133 | 22.80 ± 13.62 | 13.92 ± 9.18 |
| AV01134 | 5.47 ± 10.44 | 16.80 ± 12.49 |
| AV01135 | 27.84 ± 7.52 | 14.05 ± 15.30 |
| AV01136 | 18.96 ± 28.28 | 4.22 ± 8.31 |
| AV01137 | 33.34 ± 14.65 | 26.26 ± 10.32 |
| AV01138 | 48.10 ± 5.22 | 21.30 ± 11.12 |
| AV01139 | 40.60 ± 28.81 | 3.65 ± 7.32 |
| AV01141 | 52.94 ± 15.68 | 19.49 ± 13.32 |
| AV01142 | 68.60 ± 7.56 | 26.69 ± 9.29 |
| AV01143 | 55.74 ± 8.85 | 3.10 ± 6.00 |
| AV01145 | 26.81 ± 10.58 | −6.48 ± 27.31 |
| AV01146 | 52.62 ± 2.67 | 24.54 ± 6.62 |
| AV01147 | 12.00 ± 18.31 | 0.45 ± 15.35 |
| AV01148 | 6.03 ± 19.71 | 2.61 ± 31.65 |
| AV01149 | −1.83 ± 5.11 | 7.56 ± 34.29 |
| AV01150 | 28.99 ± 14.10 | 0.51 ± 23.46 |
| AV01151 | 26.05 ± 8.99 | −5.17 ± 21.34 |
| AV01152 | 37.58 ± 14.06 | 11.18 ± 12.60 |
| AV01154 | 54.59 ± 5.67 | 8.66 ± 22.94 |
| AV01155 | 25.69 ± 2.05 | 5.46 ± 13.79 |
| AV01156 | 26.78 ± 12.43 | 13.48 ± 12.05 |
| AV01157 | 34.36 ± 16.61 | 12.24 ± 16.79 |
| AV01158 | 36.92 ± 4.19 | 17.75 ± 5.61 |
| AV01159 | 23.11 ± 21.81 | 22.31 ± 7.81 |
| AV01160 | 31.63 ± 11.96 | 1.69 ± 34.12 |
| AV01161 | 54.85 ± 4.87 | 6.96 ± 13.14 |
| AV01162 | 29.77 ± 11.39 | 16.24 ± 13.39 |
| AV01163 | 30.94 ± 8.92 | 16.35 ± 10.82 |
| AV01164 | 18.08 ± 15.04 | 6.41 ± 4.09 |
| AV01165 | 13.00 ± 4.04 | 1.55 ± 19.80 |
| AV01166 | 27.13 ± 3.91 | 24.17 ± 17.81 |
| AV01167 | 24.66 ± 12.25 | 25.74 ± 10.42 |
| AV01168 | 49.35 ± 10.88 | 5.23 ± 17.85 |
| AV01169 | 13.28 ± 16.64 | −15.92 ± 16.77 |
| AV01170 | 5.02 ± 13.96 | −11.70 ± 8.90 |
| AV01171 | 37.00 ± 11.60 | 3.39 ± 6.49 |
| AV01172 | 1.30 ± 5.01 | −22.90 ± 7.17 |
| AV01173 | 22.82 ± 5.54 | −1.87 ± 5.86 |
| AV01174 | 8.08 ± 11.70 | −2.71 ± 8.17 |
| AV01175 | 43.69 ± 9.15 | 9.57 ± 15.70 |
| AV01176 | 12.74 ± 16.21 | −6.71 ± 2.45 |
| AV01177 | 13.81 ± 3.68 | −13.37 ± 8.36 |
| AV01178 | −11.01 ± 8.68 | 2.61 ± 8.10 |
| AV01179 | 7.36 ± 18.41 | −18.22 ± 5.66 |
| AV01181 | 44.57 ± 11.06 | 2.91 ± 8.10 |
| AV01182 | 24.01 ± 1.76 | 4.74 ± 14.48 |
| AV01183 | 22.76 ± 10.95 | 11.46 ± 1.69 |
| AV01184 | 8.07 ± 6.11 | −3.87 ± 15.94 |
| AV01185 | 14.19 ± 12.67 | −11.91 ± 13.99 |
| AV01186 | 25.59 ± 13.43 | 1.91 ± 5.41 |
| AV01187 | −3.14 ± 2.19 | −9.44 ± 9.09 |
| AV01188 | −1.45 ± 4.31 | −8.54 ± 8.93 |
| AV01189 | 33.69 ± 31.77 | −4.88 ± 20.57 |
| AV01190 | −2.06 ± 9.93 | 1.43 ± 12.31 |
| AV01191 | 20.30 ± 17.54 | 15.39 ± 20.78 |
| AV01192 | 23.78 ± 4.83 | 4.27 ± 6.34 |
| AV01193 | 19.68 ± 12.52 | −2.98 ± 18.96 |
| AV01194 | 8.96 ± 13.33 | −4.94 ± 7.13 |
| AV01195 | 27.54 ± 3.63 | 2.43 ± 19.09 |
| AV01196 | 18.36 ± 34.34 | −0.24 ± 1.74 |
| AV01197 | 14.00 ± 23.45 | 3.70 ± 7.81 |
| AV01198 | 26.48 ± 0.79 | −3.38 ± 12.06 |
| AV01199 | 23.16 ± 7.33 | 10.76 ± 4.88 |
| AV01200 | 59.22 ± 2.97 | 27.28 ± 5.00 |
| AV01201 | 38.67 ± 6.89 | 14.47 ± 4.39 |
| AV01203 | 45.91 ± 8.65 | 17.58 ± 10.08 |
| AV01204 | 64.97 ± 3.26 | 45.64 ± 6.65 |
| AV01205 | 66.01 ± 0.60 | 33.58 ± 2.13 |
| AV01206 | 13.09 ± 14.51 | 5.19 ± 28.38 |
| AV01207 | 13.23 ± 2.65 | 11.98 ± 6.41 |
| AV01208 | 31.62 ± 8.88 | 9.33 ± 3.28 |
| AV01209 | 34.67 ± 21.61 | 8.92 ± 3.25 |
| AV01210 | 9.20 ± 11.38 | 12.36 ± 2.26 |
| AV01211 | −1.01 ± 6.57 | 4.72 ± 14.97 |
| AV01212 | 8.02 ± 12.30 | 8.51 ± 10.80 |
| AV01213 | 1.81 ± 9.11 | 1.12 ± 0.69 |
| / | / | / |

### Example 5 In Vivo Testing of FXI siRNA Duplexes

At day 1, female C7BL/6J mice (4-6 weeks, 4 in each group) were infected by intravenous administration of a solution of adeno-associated virus (AAV-GFP) vector encoding Human Factor XI and luciferase gene. At day 8, mice were subcutaneously administered a single 3 mg/kg of FXI siRNA agents or PBS. Blood samples were collected at day 8, before dosing of siRNA, at day 15, and day 22. Plasma samples were isolated and serum samples were collected for quantification protein level through ELISA protocol. The results are shown in Tables 6-8.

Table 6 provides experimental results of in vivo studies using various FXI RNAi agents to inhibit FXI expression. The duplex sequences used correspond to those shown in Table 3.

| Duplex | D 15 remaining percent | D 22 remaining percent |
|---|---|---|
| AD# | relative to D 8 (Mean ± SD) | relative to D 8 (Mean ± SD) |
| AD00764 | 1.00 ± 0.10 | 0.82 ± 0.11 |
| AD00766 | 0.79 ± 0.12 | 0.62 ± 0.05 |
| AD00767 | 0.91 ± 0.13 | 0.73 ± 0.19 |
| AD00769 | 0.66 ± 0.10 | 0.61 ± 0.10 |
| AD00770 | 0.59 ± 0.14 | 0.61 ± 0.03 |
| AD00771 | 0.78 ± 0.07 | 1.02 ± 0.11 |
| AD00772 | 0.74 ± 0.06 | 0.60 ± 0.08 |
| AD00774 | 0.93 ± 0.14 | 0.72 ± 0.12 |
| AD00775 | 0.80 ± 0.09 | 0.56 ± 0.02 |

Table 7 provides experimental results of in vivo studies using various FXI RNAi agents to inhibit FXI expression. The duplex sequences used correspond to those shown in Table 3.

| Duplex | D 15 remaining percent | D 22 remaining percent |
|---|---|---|
| AD# | relative to D 8 (Mean ± SD) | relative to D 8 (Mean ± SD) |
| AD00776 | 0.24 ± 0.05 | 0.28 ± 0.04 |
| AD00988 | 0.4 ± 0.03 | 0.46 ± 0.06 |
| AD00989 | 0.47 ± 0.06 | 0.41 ± 0.08 |
| AD00990 | 0.18 ± 0.05 | 0.12 ± 0.06 |
| AD00991 | 0.18 ± 0.03 | 0.21 ± 0.02 |
| AD00992 | 0.78 ± 0.05 | 0.6 ± 0.08 |
| AD00993 | 0.46 ± 0.03 | 0.43 ± 0.1 |
| AD00994 | 0.97 ± 0.19 | 0.8 ± 0.11 |
| AD00995 | 0.51 ± 0.21 | 0.49 ± 0.17 |
| AD00996 | 0.25 ± 0.04 | 0.25 ± 0.04 |
| AD00997 | 0.22 ± 0.04 | 0.14 ± 0.03 |
| AD00998 | 0.15 ± 0.03 | 0.14 ± 0.03 |
| AD00999 | 0.23 ± 0.07 | 0.21 ± 0.03 |
| AD01000 | 0.25 ± 0.06 | 0.24 ± 0.09 |
| AD01001 | 0.71 ± 0.04 | 0.66 ± 0.07 |
| AD01002 | 0.15 ± 0.01 | 0.13 ± 0.02 |
| AD01003 | 0.34 ± 0.04 | 0.41 ± 0.1 |
| AD01004 | 0.57 ± 0.16 | 0.75 ± 0.19 |
| AD01005 | 0.46 ± 0.05 | 0.57 ± 0.05 |
| AD01006 | 0.55 ± 0.09 | 0.92 ± 0.14 |
| AD01007 | 0.49 ± 0.06 | 0.63 ± 0.02 |
| AD01008 | 0.52 ± 0.06 | 0.71 ± 0.09 |
| AD01009 | 0.32 ± 0.04 | 0.28 ± 0.06 |
| AD01010 | 0.22 ± 0.06 | 0.29 ± 0.04 |
| AD01011 | 0.46 ± 0.1 | 0.66 ± 0.14 |
| AD01012 | 0.34 ± 0.05 | 0.49 ± 0.05 |
| AD01013 | 0.7 ± 0.23 | 0.95 ± 0.17 |
| AD01014 | 0.23 ± 0.07 | 0.3 ± 0.12 |
| AD01015 | 0.22 ± 0.05 | 0.33 ± 0.04 |
| AD01016 | 0.11 ± 0.03 | 0.27 ± 0.06 |
| AD01018 | 0.2 ± 0.03 | 0.32 ± 0.01 |
| AD01019 | 0.2 ± 0.1 | 0.32 ± 0.07 |
| AD01020 | 0.29 ± 0.06 | 0.36 ± 0.03 |

Table 8 provides experimental results of in vivo studies using various FXI RNAi agents to inhibit FXI expression. The duplex sequences used correspond to those shown in Table 3.

| Duplex | D 15 remaining percent | D 22 remaining percent |
|---|---|---|
| AD# | relative to D 8 (Mean ± SD) | relative to D 8 (Mean ± SD) |
| AD01484 | 0.35 ± 0.05 | 0.32 ± 0.04 |
| AD01485 | 0.46 ± 0.06 | 0.44 ± 0.05 |
| AD01486 | 0.41 ± 0.02 | 0.28 ± 0.04 |

### Example 6. In Vitro Screening of FXI siRNA Duplexes

Huh7 cells were trypsinized and adjusted to appropriate density, mixed with the complexes of psiCHECK(TM)-2 Vector plasmid and Lipofectamine 2000 and seeded into 96-well plates. Cells were transfected with test siRNAs or a control siRNA using Lipofectamine RNAiMax at the same time of seeding following the protocol according to manufacturer's recommendation. The siRNAs were tested at five concentrations (10 nM, 3 nM, 1 nM 0.1 nM and 0.03 nM) in triplicate. Dual-Glo® Luciferase analysis. The results are shown in Tables 9.

Table 9 provides experimental results of in vitro studies using various FXI RNAi agents to inhibit FXI expression. The duplex sequences used correspond to those shown in Table 2.

| | Inhibition % | | | | |
|---|---|---|---|---|---|
| Duplex | 10 nM | 3 nM | 1 nM | 0.1 nM | 0.05 nM |
| AV# | MEAN ± SD | | | | |
| AV01214 | 82.79 ± 2.35 | 71.95 ± 3.97 | 55.13 ± 2.49 | 17.63 ± 7.45 | 4.90 ± 9.04 |
| AV01215 | 86.18 ± 0.55 | 86.34 ± 1.35 | 82.71 ± 1.09 | 44.66 ± 3.34 | 28.52 ± 7.19 |
| AV01216 | 85.72 ± 0.65 | 83.45 ± 0.83 | 77.35 ± 3.58 | 38.95 ± 5.65 | 19.59 ± 2.29 |
| AV01217 | 83.19 ± 2.46 | 80.36 ± 1.21 | 72.82 ± 3.67 | 39.09 ± 3.08 | 23.22 ± 4.10 |
| AV01218 | 87.00 ± 4.36 | 88.18 ± 2.63 | 78.66 ± 2.89 | 18.96 ± 9.62 | 9.57 ± 5.89 |
| AV01219 | 51.57 ± 9.36 | 49.46 ± 4.88 | 43.98 ± 5.84 | 12.31 ± 4.44 | 2.96 ± 5.12 |

### Example 7. In Vitro Screening of FXI siRNA Duplexes

Huh7 cells were trypsinized and adjusted to appropriate density, mixed with the complexes of psiCHECK(TM)-2 Vector plasmid and Lipofectamine 2000 and seeded into 96-well plates. Cells were transfected with test siRNAs or a control siRNA using Lipofectamine RNAiMax (Invitrogen-13778-150) at the same time of seeding following the protocol according to manufacturer's recommendation. The siRNAs were tested at two concentrations (1 nM and 0.1 nM) in triplicate.

Day 1, psiCHECK(TM)-2 Vector transfection (one plate)
(1) Transfer 2.5 μg psiCHECK(TM)-2 Vector plasmid into an RNASE free Eppendorf tube (solution mix #1) (2) Add trypsin to disassociate Huh7 cells in one flask, and count cells using Vi-Cell counting machine, adjust the cell density to 1*10\hat{ }5/ml
(3) Transfer 7.5 μL Lipofectamine 2000 into solution mix #1 tube, mix.
(4) Add solution in Step 3 into cell suspension, mix, and dispense suspension into the 96 well plate (100 1/well)

Day 2, siRNAs transfection
(1) Dilute Lipofectamine®RNAiMAX Reagent with Opti-MEM® Medium.
(2) Dilute the siRNA with RNA-free water to make 12× stock.
(3) Mix equal volume of diluted RNAiMax and siRNA. Incubate the mixture at RT for 15 min to allow complex formation.
(4) Add 45 μl/well compound Lipofectamine® RNAiMAX(Opti-MEM) mix into 225 μl/well DMEM fresh medium, and discard the supernatants in assay plate, add 120 μl/well compound mix into 96 well plates.
(5) No compound control well was defined as cells transfected with psiCHECK(TM)-2 Vector and without siRNA treatment; blank control was cell only wells.

Day 3, Dual-Glo® Luciferase Assay
(1) Add Reagent to assay plate, wait 10 minutes to allow for cell lysis to occur.
(2) Transfer 100 W cell lysates into a plate, then measure the firefly luminescence.
(3) Add 50 μl of Dual-Glo® Stop & Glo® Reagent to the assay plates and mix, wait 10 minutes, then measure Renilla luminescence.
(4) Calculate the relative expression

Data analysis, The results are shown in Tables 10, and table 11.

Table 10 provides experimental results of in vitro studies using various FXI RNAi agents to inhibit FXI expression. The duplex sequences used correspond to those shown in Table 2.

Inhibition %

| | | |
|---|---|---|
| Duplex | 1 nM | 0.1 nM |

| | |
|---|---|
| AV# | MEAN ± SD |

| | | |
|---|---|---|
| AV05205 | 79.08 ± 5.23 | 41.26 ± 2.29 |
| AV05206 | 91.29 ± 0.58 | 66.85 ± 0.86 |
| AV05207 | 79.35 ± 1.75 | 28.65 ± 4.11 |
| AV05208 | 56.84 ± 4.79 | 8.90 ± 8.74 |
| AV05209 | 87.16 ± 1.70 | 56.61 ± 2.06 |
| AV05210 | 82.01 ± 1.31 | 45.25 ± 2.38 |
| AV05211 | 30.19 ± 2.05 | −3.98 ± 5.12 |
| AV05213 | 39.61 ± 3.98 | 28.17 ± 18.62 |
| AV05214 | 5.45 ± 5.88 | −4.48 ± 3.22 |
| AV05215 | 43.76 ± 4.51 | 13.99 ± 1.77 |
| AV05216 | 8.87 ± 4.42 | −2.27 ± 3.69 |
| AV05217 | 60.01 ± 3.12 | 19.48 ± 5.84 |
| AV05218 | 43.72 ± 2.01 | 0.03 ± 7.72 |
| AV05219 | 3.17 ± 0.73 | 3.96 ± 4.96 |
| AV05221 | 42.92 ± 10.11 | 8.74 ± 7.94 |
| AV05222 | 69.28 ± 1.06 | 19.52 ± 2.39 |
| AV05223 | 61.55 ± 3.49 | 11.99 ± 3.62 |
| AV05224 | 74.87 ± 1.29 | 27.33 ± 2.75 |
| AV05225 | −0.64 ± 3.22 | −16.23 ± 1.90 |
| AV05226 | 61.86 ± 2.87 | 11.34 ± 6.13 |
| AV05227 | −0.52 ± 9.53 | −8.22 ± 5.89 |
| AV05229 | −6.10 ± 5.40 | −0.76 ± 12.82 |
| AV05230 | 50.11 ± 2.12 | 0.40 ± 5.72 |
| AV05231 | 70.19 ± 3.31 | 18.91 ± 4.33 |
| AV05232 | 55.20 ± 0.89 | 3.69 ± 0.51 |
| AV05233 | 76.46 ± 1.97 | 24.38 ± 13.78 |
| AV05234 | 35.09 ± 5.64 | −8.01 ± 9.45 |
| AV05235 | 73.71 ± 1.43 | 35.06 ± 4.04 |
| AV05237 | 57.59 ± 10.70 | 6.28 ± 3.00 |
| AV05238 | 60.49 ± 5.68 | 6.94 ± 6.83 |
| AV05239 | −5.94 ± 4.40 | −12.10 ± 2.20 |
| AV05240 | 54.76 ± 0.65 | 3.25 ± 7.96 |
| AV05241 | 46.85 ± 1.56 | −4.89 ± 1.53 |
| AV05242 | 20.87 ± 1.56 | −1.35 ± 4.25 |
| AV05243 | 33.47 ± 5.54 | −10.40 ± 6.93 |
| AV05245 | 57.35 ± 3.05 | 25.34 ± 12.07 |
| AV05246 | 8.12 ± 5.07 | −9.35 ± 5.97 |
| AV05247 | 28.82 ± 7.40 | −1.96 ± 3.85 |
| AV05248 | 7.00 ± 9.93 | −8.32 ± 5.48 |
| AV05249 | 17.18 ± 4.84 | −15.84 ± 2.11 |
| AV05250 | 0.92 ± 6.78 | 0.96 ± 1.51 |
| AV05251 | 58.67 ± 3.92 | 10.05 ± 4.33 |
| AV05253 | 80.69 ± 3.39 | 32.48 ± 3.25 |
| AV05254 | −5.05 ± 14.10 | −10.79 ± 4.76 |
| AV05255 | −9.88 ± 7.86 | −17.31 ± 8.70 |
| AV05256 | 27.08 ± 3.23 | −9.97 ± 6.02 |
| AV05257 | 53.62 ± 5.53 | 11.62 ± 6.03 |
| AV05258 | 13.11 ± 2.42 | −6.85 ± 10.64 |
| AV01215 | 77.87 ± 2.50 | 47.38 ± 4.29 |
| AV05212 | 67.36 ± 3.51 | 44.24 ± 12.76 |
| AV05220 | 32.06 ± 1.13 | 3.84 ± 5.78 |
| AV05228 | 55.09 ± 0.72 | 9.90 ± 3.40 |
| AV05236 | 35.24 ± 1.57 | −10.97 ± 6.51 |
| AV05244 | 67.60 ± 2.38 | 14.12 ± 4.20 |
| AV05252 | 12.91 ± 6.68 | −22.66 ± 6.06 |
| AV01219 | 26.76 ± 6.38 | −1.25 ± 9.41 |

Table 11 provides experimental results of in vitro studies using various FXI RNAi agents to inhibit FXI expression. The duplex sequences used correspond to those shown in Table 2.

Inhibition %

| | | |
|---|---|---|
| Duplex | 1 nM | 0.1 nM |

| | |
|---|---|
| AV# | MEAN ± SD |

| | | |
|---|---|---|
| AV05499 | 16.81 ± 13.10 | −46.45 ± 9.63 |
| AV05500 | 19.76 ± 17.17 | 52.58 ± 10.45 |
| AV05501 | −17.54 ± 1.65 | −33.96 ± 9.99 |
| AV05502 | 98.14 ± 0.23 | 86.47 ± 1.69 |
| AV05503 | 74.46 ± 2.28 | 0.43 ± 4.04 |
| AV05504 | 44.32 ± 4.94 | −2.68 ± 9.07 |
| AV05505 | 50.27 ± 0.91 | 6.65 ± 1.70 |
| AV05507 | 61.92 ± 1.10 | −7.92 ± 9.63 |
| AV05508 | 71.09 ± 0.47 | −6.98 ± 3.77 |
| AV05509 | −46.01 ± 6.65 | −44.48 ± 4.58 |
| AV05510 | 57.15 ± 1.94 | −22.07 ± 6.00 |
| AV05511 | 69.78 ± 0.53 | 6.30 ± 5.93 |
| AV05512 | 76.56 ± 0.42 | 32.35 ± 1.55 |
| AV05513 | 86.53 ± 0.50 | 54.15 ± 2.76 |
| AV05515 | 68.01 ± 0.62 | 25.37 ± 2.93 |
| AV05516 | 72.66 ± 1.26 | 9.42 ± 5.55 |
| AV05517 | 61.89 ± 0.97 | 8.12 ± 5.15 |
| AV05518 | 1.23 ± 8.21 | −14.40 ± 4.47 |
| AV05519 | 79.60 ± 1.12 | 56.83 ± 0.47 |
| AV05520 | 16.90 ± 3.04 | −7.90 ± 6.13 |
| AV05521 | 1.19 ± 9.66 | −14.68 ± 6.46 |
| AV05523 | 17.20 ± 4.36 | −7.00 ± 5.07 |
| AV05524 | −20.54 ± 3.99 | −24.28 ± 7.87 |
| AV05525 | 32.83 ± 4.22 | −12.59 ± 4.56 |
| AV05526 | 81.66 ± 2.64 | 35.24 ± 3.93 |
| AV05527 | 0.51 ± 5.39 | −14.88 ± 12.98 |
| AV05528 | 23.56 ± 0.67 | −8.78 ± 5.28 |
| AV05529 | 38.26 ± 1.62 | 11.94 ± 3.56 |
| AV05531 | 25.29 ± 3.76 | −21.26 ± 7.13 |
| AV05532 | 79.96 ± 0.14 | 26.30 ± 1.18 |
| AV05533 | 41.86 ± 0.60 | −15.67 ± 4.86 |
| AV05534 | 27.49 ± 0.37 | −9.27 ± 6.25 |
| AV05535 | 66.68 ± 0.94 | 41.37 ± 6.31 |
| AV05536 | 44.23 ± 2.20 | 0.37 ± 5.69 |
| AV05537 | 44.60 ± 3.79 | 1.71 ± 2.37 |
| AV05539 | 67.27 ± 0.47 | 16.50 ± 9.81 |
| AV05540 | 39.71 ± 3.01 | −20.69 ± 0.25 |
| AV05541 | 12.49 ± 2.50 | −21.61 ± 2.69 |
| AV05542 | 77.36 ± 0.43 | 32.15 ± 3.86 |
| AV05544 | 30.74 ± 5.26 | −8.35 ± 10.91 |
| AV05545 | 29.91 ± 5.61 | 1.75 ± 5.23 |
| AV05547 | 21.33 ± 4.35 | −31.09 ± 2.62 |
| AV05548 | 33.89 ± 10.18 | −8.06 ± 3.11 |
| AV05549 | 29.00 ± 8.42 | −19.67 ± 6.14 |
| AV05550 | 25.19 ± 0.13 | −24.85 ± 3.66 |
| AV05551 | 17.15 ± 1.90 | −10.25 ± 0.63 |
| AV05552 | 43.57 ± 2.38 | 1.44 ± 2.66 |
| AV05553 | 61.17 ± 3.03 | 13.62 ± 3.15 |
| AV05555 | 75.59 ± 0.99 | 11.43 ± 8.58 |
| AV05556 | 55.61 ± 1.35 | −10.31 ± 5.81 |
| AV05557 | 51.26 ± 1.92 | −16.26 ± 10.43 |
| AV05558 | 13.00 ± 1.19 | −32.22 ± 2.91 |
| AV05559 | 50.47 ± 2.93 | −14.17 ± 9.83 |
| AV05560 | −14.87 ± 1.03 | −20.64 ± 7.62 |
| AV05561 | 66.63 ± 1.91 | 14.95 ± 3.95 |
| AV05563 | 57.57 ± 1.70 | 8.09 ± 2.37 |
| AV05564 | 40.29 ± 4.22 | −31.86 ± 7.78 |
| AV05565 | 12.18 ± 2.62 | −27.45 ± 8.77 |
| AV05566 | 65.80 ± 3.05 | 15.15 ± 5.49 |
| AV05567 | 71.54 ± 1.47 | 14.41 ± 3.00 |
| AV05568 | 83.41 ± 0.37 | 22.03 ± 3.94 |
| AV05569 | 84.68 ± 0.07 | 46.50 ± 4.47 |
| AV05506 | 65.48 ± 1.53 | −10.03 ± 5.11 |
| AV05514 | 42.67 ± 2.09 | −18.15 ± 1.89 |
| AV05522 | 10.68 ± 3.31 | −30.88 ± 3.21 |
| AV05530 | −1.47 ± 7.63 | −31.54 ± 5.14 |
| AV05538 | 81.21 ± 0.93 | 34.38 ± 2.87 |
| AV05546 | 5.58 ± 5.00 | −23.08 ± 6.45 |
| AV05554 | 83.96 ± 0.64 | 50.15 ± 1.74 |
| AV05562 | 63.32 ± 2.13 | −1.08 ± 4.57 |
| AV05570 | 75.09 ± 1.61 | 32.17 ± 5.48 |
| AV01215 | 81.20 ± 0.46 | 44.87 ± 0.80 |
| AV01219 | 33.96 ± 1.76 | −13.89 ± 6.12 |

### Example 8 In Vivo Testing of FXI siRNA Duplexes

At day 1, female C57BL/6J mice (4-6 weeks, 4 in each group) were infected by intravenous administration of a solution of adeno-associated virus (AAV-GFP) vector encoding Human Factor XI and luciferase gene. At day 8, mice were subcutaneously administered a single 2 or 3 mg/kg of FXI siRNA agents or PBS. Blood samples were collected at day 8, before dosing of siRNA, at day 15, and day 22. Plasma samples were isolated and serum samples were collected for quantification protein level through ELISA protocol. The results are shown in Tables 12-14.

Table 12 provides experimental results of in vivo studies using various FXI RNAi agents to inhibit FXI expression. The duplex sequences used correspond to those shown in Table 3.

| | remaining percent relative to D 8 | | |
|---|---|---|---|
| Duplex | (Mean ± SD) | | Dosage |
| AD# | D 15 | D 22 | mg/kg |
| AD01002 | 0.11 ± 0.03 | / | 3 |
| AD00997-1 | 0.22 ± 0.07 | / | 3 |
| AD01606 | 0.33 ± 0.08 | / | 3 |

Table 13 provides experimental results of in vivo studies using various FXI RNAi agents to inhibit FXI expression. The duplex sequences used correspond to those shown in Table 3.

| | FXI remaining compared to | | |
|---|---|---|---|
| Duplex | day 8 (Mean ± SEM) | | |
| AD# | Day 15 | Day 22 | Dose(mg/kg) |
| AD00990 | 0.14 ± 0.02 | 0.12 ± 0.02 | 3 |
| AD00991 | 0.18 ± 0.07 | 0.18 ± 0.02 | 3 |
| AD00997 | 0.17 ± 0.02 | 0.19 ± 0.07 | 3 |
| AD001002 | 0.08 ± 0.03 | 0.07 ± 0.03 | 3 |
| AD02349 | 1.18 ± 0.13 | 1.02 ± 0.13 | 3 |
| AD02350 | 0.78 ± 0.07 | 0.86 ± 0.02 | 3 |
| AD02351 | 0.4 ± 0.01 | 0.33 ± 0.01 | 3 |
| AD02352 | 0.12 ± 0.01 | 0.12 ± 0.03 | 3 |
| AD02353 | 0.2 ± 0.03 | 0.23 ± 0.03 | 3 |
| AD02354 | 0.27 ± 0.01 | 0.3 ± 0.04 | 3 |
| AD02355 | 0.52 ± 0.07 | 0.39 ± 0.06 | 3 |
| AD02356 | 0.27 ± 0.04 | 0.35 ± 0.06 | 3 |
| AD02357 | 0.3 ± 0.02 | 0.23 ± 0.02 | 3 |
| AD02358 | 0.31 ± 0.05 | 0.25 ± 0.06 | 3 |
| AD02359 | 0.18 ± 0.06 | 0.22 ± 0.04 | 3 |
| AD02360 | 0.19 ± 0.02 | 0.28 ± 0.02 | 3 |
| AD02361 | 0.26 ± 0.05 | 0.28 ± 0.06 | 3 |
| AD02362 | 0.17 ± 0.02 | 0.15 ± 0.01 | 3 |
| AD02363 | 0.36 ± 0.05 | 0.38 ± 0.03 | 3 |
| AD02364 | 0.43 ± 0.05 | 0.41 ± 0.08 | 3 |
| AD02365 | 0.3 ± 0.03 | 0.23 ± 0.03 | 3 |
| AD02366 | 0.23 ± 0.03 | 0.16 ± 0.02 | 3 |

Table 14 provides experimental results of in vivo studies using various FXI RNAi agents to inhibit FXI expression. The duplex sequences used correspond to those shown in Table 3.

| | FXI remaining compared to day 8 | | |
|---|---|---|---|
| Duplex | (Mean ± SEM) | | |
| AD# | Day 15 | Day 22 | Dose(mg/kg) |
| AD00991 | 0.11 ± 0.03 | 0.16 ± 0.01 | 3 |
| AD00997 | 0.11 ± 0.01 | 0.11 ± 0.03 | 3 |
| AD02369 | 1.17 ± 0.24 | 1.12 ± 0.26 | 3 |
| AD02370 | 1.01 ± 0.18 | 1.11 ± 0.2 | 3 |
| AD02371 | 0.41 ± 0.02 | 0.46 ± 0.07 | 3 |
| AD02372 | 0.13 ± 0.03 | 0.15 ± 0.02 | 3 |
| AD02373 | 0.65 ± 0.11 | 0.73 ± 0.07 | 3 |
| AD02374 | 0.55 ± 0.06 | 0.54 ± 0.1 | 3 |
| AD02375 | 0.05 ± 0 | 0.07 ± 0.02 | 3 |
| AD02376 | 0.26 ± 0 | 0.25 ± 0.03 | 3 |
| AD02377 | 0.14 ± 0.02 | 0.12 ± 0.01 | 3 |
| AD02378 | 0.75 ± 0.11 | 0.53 ± 0.09 | 3 |
| AD02379 | 0.28 ± 0.03 | 0.29 ± 0.05 | 3 |
| AD02380 | 0.18 ± 0.03 | 0.2 ± 0.06 | 3 |
| AD02381 | 1.16 ± 0.13 | 1.13 ± 0.23 | 3 |
| AD02382 | 0.35 ± 0.05 | 0.28 ± 0.06 | 3 |
| AD02383 | 0.38 ± 0.06 | 0.28 ± 0.04 | 3 |
| AD02384 | 0.44 ± 0.05 | 0.4 ± 0.03 | 3 |
| AD02385 | 0.22 ± 0.04 | 0.16 ± 0.04 | 3 |
| AD02386 | 0.47 ± 0.05 | 0.37 ± 0.03 | 3 |
| AD02387 | 0.39 ± 0.05 | 0.22 ± 0.02 | 3 |
| AD02388 | 0.72 ± 0.2 | 0.57 ± 0.26 | 3 |
| AD02389 | 0.54 ± 0.07 | 0.45 ± 0.05 | 3 |
| AD02390 | 0.91 ± 0.01 | 0.91 ± 0.05 | 3 |
| AD02391 | 0.6 ± 0.03 | 0.53 ± 0.06 | 3 |
| AD02392 | 0.25 ± 0.07 | 0.21 ± 0.06 | 3 |
| AD02393 | 0.8 ± 0.12 | 0.78 ± 0.11 | 3 |
| AD02394 | 0.28 ± 0.01 | 0.2 ± 0.02 | 3 |
| AD02395 | 0.52 ± 0.03 | 0.58 ± 0.08 | 3 |
| AD02396 | 0.46 ± 0.03 | 0.45 ± 0 | 3 |
| AD02397 | 0.3 ± 0.04 | 0.26 ± 0.03 | 3 |
| AD02398 | 0.4 ± 0.04 | 0.28 ± 0.07 | 3 |
| AD02399 | 0.08 ± 0 | 0.07 ± 0 | 3 |
| AD02400 | 0.53 ± 0.01 | 0.39 ± 0.03 | 3 |
| AD02401 | 0.76 ± 0.07 | 0.6 ± 0.11 | 3 |
| AD02402 | 0.26 ± 0.06 | 0.16 ± 0.04 | 3 |
| AD02403 | 0.54 ± 0.06 | 0.43 ± 0.12 | 3 |
| AD02404 | 0.19 ± 0.01 | 0.14 ± 0.01 | 3 |

At day 1, female C57BL/6J mice (4-6 weeks, 4 in each group) were infected by intravenous administration of a solution of adeno-associated virus (AAV-GFP) vector encoding Human Factor XI and luciferase gene. At day 8, mice were subcutaneously administered a single 2 mg/kg of FXI siRNA agents or PBS. Blood samples were collected at day 8, before dosing of siRNA, at day 21, and day 28. Plasma samples were isolated and serum samples were collected for quantification protein level through ELISA protocol. The results are shown in Tables 15.

Table 15 provides experimental results of in vivo studies using various FXI RNAi agents to inhibit FXI expression. The duplex sequences used correspond to those shown in Table 3.

| | FXI remaining compared | | |
|---|---|---|---|
| Duplex | to day 8 (Mean ± SEM) | | |
| AD# | Day 21 | Day 28 | Dose(mg/kg) |
| AD00990 | 0.15 ± 0.04 | 0.17 ± 0.05 | 2 |
| AD00991 | 0.14 ± 0.01 | 0.22 ± 0.02 | 2 |
| AD00997 | 0.16 ± 0.04 | 0.15 ± 0.05 | 2 |
| AD02432 | 0.25 ± 0.01 | 0.4 ± 0.03 | 2 |
| AD02433 | 0.4 ± 0.1 | 0.58 ± 0.05 | 2 |
| AD02434 | 0.52 ± 0.08 | 0.63 ± 0.12 | 2 |
| AD02435 | 1.16 ± 0.12 | 1.68 ± 0.16 | 2 |
| AD02436 | 0.66 ± 0.11 | 0.67 ± 0.07 | 2 |
| AD02437 | 0.42 ± 0.04 | 0.66 ± 0.06 | 2 |
| AD02438 | 0.55 ± 0.09 | 0.65 ± 0.07 | 2 |
| AD02439 | 0.24 ± 0.02 | 0.35 ± 0.06 | 2 |
| AD02440 | 0.19 ± 0.02 | 0.21 ± 0.05 | 2 |
| AD02441 | 0.24 ± 0.04 | 0.4 ± 0.14 | 2 |
| AD02442 | 0.82 ± 0.12 | 0.98 ± 0.03 | 2 |
| AD02443 | 0.15 ± 0.02 | 0.17 ± 0.03 | 2 |
| AD02444 | 0.09 ± 0.02 | 0.05 ± 0.01 | 2 |
| AD02445 | 0.15 ± 0.01 | 0.1 ± 0.03 | 2 |
| AD02446 | 0.1 ± 0.02 | 0.09 ± 0.02 | 2 |
| AD02447 | 0.05 ± 0.01 | 0.04 ± 0 | 2 |

### Example 9. In Vivo Testing of FXI siRNA Duplexes in NHP PD Model

Cynomolgus monkeys (3-5 years old, 3-6 kg of weights, 3 monkeys in each group) were enrolled in the study. Each monkey was subcutaneously administered a single 4 mg/kg of FXI siRNA agents or PBS at day 1 (pre-dosing of siRNA). After overnight fast, Plasma were drawn at day −14 (pre-dose), day −7 (pre-dose), 1 (pre-dose), 8, 15, 22, 29, 43, 50 and 57; liver biopsy were drawn at day −7 (pre-dose), 29 and 57. FXI protein concentration in serum were measured by ELISA method (FXI ELISA kit, Abcam, 1002579-4), the result is shown in table 16. FXI mRNA level in liver were measured by QPCR method, the result is shown in table 17. FXI activity were also measured by automatic blood clotting analyzer (siemens) the result are shown in table 18.

TABLE 16
The results of FXI protein concentration in serum were measured by ELISA method

| Duplex | FXI remaining compared to pre-dosing (%) (Mean ± SEM) | | | | | | | | |
|---|---|---|---|---|---|---|---|---|---|
| AD# | Day 8 | Day 15 | Day 22 | Day 29 | Day 36 | Day 43 | Day 50 | Day 57 | Dose(mg/kg) |
| AD00990 | 53.41 ± 3.31 | 36.31 ± 5.61 | 22.88 ± 3.47 | 21.06 ± 2.6 | 18.28 ± 3.11 | 19.6 ± 3.23 | 21.96 ± 3.58 | 23.54 ± 3.28 | 4 |
| AD00991 | 51.14 ± 3.45 | 30.19 ± 2.1 | 24.18 ± 5.99 | 16.8 ± 2.38 | 21.7 ± 0.59 | 18.92 ± 0.71 | 22.87 ± 1.93 | 23.13 ± 3.04 | 4 |
| AD00997 | 51.17 ± 0.64 | 27.64 ± 2.22 | 20.48 ± 1.76 | 12.76 ± 1.83 | 15.2 ± 1.74 | 16.04 ± 2.01 | 24.49 ± 2.97 | 21.21 ± 1.49 | 4 |
| AD00999 | 55.43 ± 1.55 | 50.06 ± 6.84 | 39.88 ± 5.89 | 32.11 ± 5.57 | 32.94 ± 8.68 | 27.98 ± 7.69 | 38.45 ± 13.76 | 37.63 ± 9.47 | 4 |
| AD00998 | 69.26 ± 1.14 | 51.84 ± 2.42 | 40.36 ± 2.34 | 26.97 ± 1.05 | 29.18 ± 2.61 | 32.98 ± 1.26 | 41.79 ± 2.96 | 41.49 ± 2.81 | 4 |
| AD01002 | 51.89 ± 4.55 | 33.71 ± 5.67 | 26.52 ± 5.36 | 23.95 ± 4.66 | 24.36 ± 4.32 | 25.17 ± 7.02 | 29.96 ± 8.5 | 36.04 ± 5.18 | 4 |
| AD01016 | 52.82 ± 3.14 | 36.16 ± 0.99 | 29.16 ± 0.91 | 27.77 ± 2.92 | 28.06 ± 1.77 | 26.81 ± 3.23 | 36.35 ± 3.96 | 35.78 ± 4.25 | 4 |
| AD01606 | 59.18 ± 1.71 | 44.86 ± 2.79 | 36.93 ± 5.81 | 44.21 ± 8.24 | 42.59 ± 6.26 | 46.44 ± 5.53 | 53.87 ± 6.85 | 57.25 ± 6.59 | 4 |

TABLE 17
The results of FXI mRNA level in liver
were measured by QPCR method.

| | F11 remaining | F11 remaining | |
|---|---|---|---|
| | relative to pre- | relative to pre- | |
| | dosing baseline | dosing baseline | |
| Duplex | (Mean ± SEM) | (Mean ± SEM) | Dosage |
| AD# | D 29 | D 57 | mg/kg |
| AD00990 | 0.25 ± 0.05 | 0.22 ± 0.04 | 4 |
| AD00991 | 0.17 ± 0.04 | 0.22 ± 0 | 4 |
| AD00997 | 0.2 ± 0.03 | 0.22 ± 0.02 | 4 |
| AD00999 | 0.24 ± 0.04 | 0.32 ± 0.09 | 4 |
| AD00998 | 0.21 ± 0.03 | 0.3 ± 0.09 | 4 |
| AD01002 | 0.2 ± 0.01 | 0.23 ± 0.06 | 4 |
| AD01016 | 0.2 ± 0.02 | 0.27 ± 0.06 | 4 |
| AD01606 | 0.38 ± 0.15 | 0.57 ± 0.15 | 4 |

TABLE 18
The results of FXI activity

| Duplex | FXI activity remaining compared to pre-dosing (%)(Mean ± SEM) | | | | | | | |
|---|---|---|---|---|---|---|---|---|
| AD# | Day 8 | Day 15 | Day 22 | Day 29 | Day 36 | Day 43 | Day 50 | Day 57 |
| AD00990 | 40.12 ± 5.28 | 23.3 ± 5.19 | 14.72 ± 3.71 | 15.23 ± 3.92 | 21.6 ± 6.31 | 17.46 ± 4.59 | 24.87 ± 6.51 | 24.09 ± 6.51 |
| AD00991 | 30.55 ± 1.56 | 15.47 ± 1.11 | 13.17 ± 0.67 | 11.91 ± 0.96 | 18.07 ± 1.51 | 13.25 ± 1.27 | 19.47 ± 2.08 | 17.57 ± 0.95 |
| AD00997 | 34.18 ± 1.62 | 16.64 ± 0.26 | 12.1 ± 1.55 | 13.25 ± 1.28 | 16.67 ± 1.24 | 16.28 ± 3.1 | 23.01 ± 4.12 | 21.06 ± 3.99 |
| AD00999 | 48.51 ± 5.04 | 25.61 ± 1.98 | 24.54 ± 1.56 | 24.76 ± 3.56 | 32.85 ± 7.92 | 37.58 ± 11.96 | 38.98 ± 12.72 | 36.38 ± 9.58 |
| AD00998 | 50.7 ± 5.02 | 27.61 ± 6.33 | 26.17 ± 8.28 | 25.87 ± 7.87 | 28.65 ± 8.97 | 28.36 ± 9.19 | 38.29 ± 11.19 | 41.64 ± 12.94 |
| AD01002 | 37.76 ± 3.41 | 20.59 ± 0.5 | 16.64 ± 1.22 | 17.39 ± 2.23 | 21.51 ± 0.51 | 21.05 ± 1.61 | 29.83 ± 3.48 | 31.94 ± 4.5 |
| AD01016 | 40.68 ± 7.97 | 24.69 ± 6.09 | 21.24 ± 7.01 | 21.03 ± 5.78 | 30.17 ± 10.1 | 28.1 ± 8.71 | 39.88 ± 13.17 | 37.48 ± 12.76 |
| AD01606 | 41.65 ± 2.36 | 23.39 ± 2.59 | 21.55 ± 3.47 | 27.11 ± 5.31 | 30.22 ± 6.3 | 33.89 ± 10.61 | 47.97 ± 12.23 | 60.26 ± 20.67 |

### Example 10. In Vivo Testing of FXI siRNA Duplexes in NHP PD Model

Cynomolgus monkeys (3-5 years old, 3-6 kg of weights, 3 monkeys in each group) were enrolled in the study. Each monkey was subcutaneously administered a single 6 mg/kg of FXI siRNA agents or PBS at day 1 (pre-dosing of siRNA). After overnight fast, Plasma was drawn at day −14 (pre-dose), day −7 (pre-dose), 1 (pre-dose), 8, 15, 22, and 29; liver biopsy were drawn at day −7 (pre-dose), 29 and 57. FXI activity were also measured by automatic blood clotting analyzer (siemens). The result are shown in table 19.

TABLE 19
The results of FXI activity

| | FXI activity remaining compared to pre-dosing (%)(Mean ± SEM) | | | | |
|---|---|---|---|---|---|
| Duplex AD# | Day 8 | Day 15 | Day 22 | Day 29 | dosage |
| AD00990-2 | 45.66 ± 5.86 | 23 ± 5.13 | 12.54 ± 2.8 | 9.69 ± 2.6 | 6 mpk |
| AD00991 | 49.84 ± 7.32 | 23.09 ± 5.4 | 17 ± 6.19 | 15.85 ± 6.39 | 6 mpk |
| AD00997-2 | 34.38 ± 7 | 16.49 ± 4.34 | 8.03 ± 1.82 | 5.32 ± 1.05 | 6 mpk |
| AD02352 | 32.15 ± 3.25 | 17.58 ± 6.17 | 9.27 ± 2.26 | 7.96 ± 2.14 | 6 mpk |
| AD02362 | 40.95 ± 1.84 | 20.81 ± 3.48 | 11.19 ± 0.26 | 11.35 ± 0.59 | 6 mpk |
| AD02375-1 | 34.58 ± 3.39 | 13.16 ± 1.35 | 6.68 ± 1.32 | 6.04 ± 1.52 | 6 mpk |
| AD02399 | 38.11 ± 3.81 | 16.69 ± 4.67 | 11.01 ± 3.68 | 9.6 ± 3.73 | 6 mpk |
| AD02444 | 53.17 ± 1.32 | 30.61 ± 4.2 | 18.27 ± 3.46 | 14.76 ± 3.1 | 6 mpk |
| AD02445 | 57.14 ± 13.11 | 27.83 ± 8.31 | 19.93 ± 6.53 | 15.29 ± 5.09 | 6 mpk |
| AD02446 | 58.26 ± 6.77 | 24.03 ± 2.58 | 14.51 ± 1.39 | 12.91 ± 1.19 | 6 mpk |
| AD02447 | 43.24 ± 3.1 | 21.41 ± 4.34 | 14.99 ± 2.73 | 12.8 ± 2.18 | 6 mpk |

### Example 11. In Vitro Screening of FXI siRNA Duplexes

Huh7 cells were trypsinized and adjusted to appropriate density, mixed with the complexes of psiCHECK(TM)-2 Vector plasmid and Lipofectamine 2000 and seeded into 96-well plates. Cells were transfected with test siRNAs or a control siRNA using Lipofectamine RNAiMax (Invitrogen-13778-150) at the same time of seeding following the protocol according to manufacturer's recommendation. The siRNAs were tested at two concentrations (1 nM and 0.1 nM) in triplicate by Dual-Glo® Luciferase Assay. The results are shown in Tables 20.

Table 20 provides experimental results of in vitro studies using various FXI RNAi agents to inhibit FXI expression. The duplex sequences used correspond to those shown in Table 2.

Inhibition %

| | | |
|---|---|---|
| Duplex | 1 nM | 0.1 nM |

| | |
|---|---|
| AV# | MEAN ± SD |

| | | |
|---|---|---|
| AV06227 | 75.49 ± 1.35 | 30.45 ± 5.55 |
| AV06228 | 77.41 ± 0.49 | 35.10 ± 2.74 |
| AV06230 | 76.94 ± 1.64 | 36.96 ± 0.66 |
| AV06231 | 65.75 ± 0.06 | 18.32 ± 2.69 |
| AV06232 | 64.70 ± 1.25 | 17.66 ± 1.35 |
| AV06233 | 84.42 ± 0.71 | 38.39 ± 3.03 |
| AV06235 | 76.96 ± 0.69 | 28.80 ± 3.09 |
| AV06236 | 71.58 ± 1.47 | 20.93 ± 6.90 |
| AV06237 | 78.34 ± 0.95 | 38.08 ± 2.96 |
| AV06238 | 67.44 ± 1.13 | 20.26 ± 0.84 |
| AV06239 | 74.15 ± 1.42 | 32.56 ± 2.32 |
| AV06240 | 43.19 ± 2.15 | 11.90 ± 5.32 |
| AV06241 | 28.94 ± 3.22 | 5.57 ± 2.16 |
| AV06234 | 72.02 ± 0.51 | 20.75 ± 2.98 |
| AV01215 | 82.28 ± 0.69 | 43.50 ± 1.75 |
| AV01219 | 32.13 ± 0.28 | −5.74 ± 1.24 |

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

1. A double-stranded ribonucleic acid (dsRNA) agent for inhibiting expression of coagulation factor XI (FXI) is provided, wherein the dsRNA agent includes a sense strand and an antisense strand, nucleotide positions 2 to 18 in the antisense strand comprising a region of complementarity to a FXI RNA transcript, wherein the region of complementarity includes at least 15, 16, 17, 18, 19, 20 or 21 contiguous nucleotides that differ by 0, 1, 2, or 3 nucleotides from one of the antisense sequences listed in one of Tables 1-3, and optionally comprising a targeting ligand.

2. A double-stranded ribonucleic acid (dsRNA) agent for inhibiting expression of coagulation factor XI (FXI) is provided, wherein the dsRNA agent includes a sense strand and an antisense strand, wherein the sense strand comprises at least 15, 16, 17, 18, 19, 20 or 21 contiguous nucleotides that differ by 0, 1, 2, or 3 nucleotides from any one of the nucleotide sequences of nucleotides 143-173, 150-180, 380-410, 384-414,389-419, 402-432, 419-449, 536-566, 1141-1171, 1488-1518, 1561-1591, 2111-2141, 2112-2142, 2113-2143, 2115-2145, 2111-2145 of SEQ ID NO: 1, and the antisense strand comprises at least 15, 16, 17, 18, 19, 20 or 21 contiguous nucleotides differing by 0, 1, 2, or 3 nucleotides from the corresponding nucleotide sequence of SEQ ID NO:2, wherein the sense strand and the antisense strand can be partially, substantially, or fully complementary to each other.

3. The dsRNA agent of claim 2, wherein the sense strand comprises at least 15, 16, 17, 18, 19, 20 or 21 contiguous nucleotides that differ by 0, 1, 2, or 3 nucleotides from any one of the nucleotide sequences of nucleotides 148-168, 155-175, 385-405, 389-409, 394-414, 407-427, 424-444, 541-561, 1146-1166,1493-1513, 1566-1586,2116-2136, 2117-2137,2118-2138, 2120-2140, 2116-2140 of SEQ ID NO: 1, and the antisense strand comprises at least 15, 16, 17, 18, 19, 20 or 21 contiguous nucleotides differing by 0, 1, 2, or 3 nucleotides from the corresponding nucleotide sequence of SEQ ID NO:2.

4. The dsRNA agent of claim 1, wherein the antisense strand of dsRNA is substantially or fully complementary to any one of a target region of SEQ ID NO: 1, and preferably the dsRNA agent comprises an antisense strand sequence set forth in any one of Tables 1-3; or
wherein the sense strand sequence is at least substantially complementary to or fully complementary to the antisense strand sequence in the dsRNA agent, preferably, wherein the dsRNA agent comprises a sense strand sequence set forth in any one of Tables 1-3.

5. (canceled)

6. The dsRNA agent of claim 1, wherein the dsRNA agent comprises the sequences set forth as a duplex sequence in any of Tables 1-3.

7. The dsRNA of claim 1, wherein the dsRNA agent comprises at least one modified nucleotide; optionally, wherein all or substantially all of the nucleotides of the sense strand and/or the antisense strand are modified nucleotides.

8. (canceled)

9. The dsRNA agent of claim 1, wherein the dsRNA agent is represented by II, III or IV,
the dsRNA agent II including a sense strand and an antisense strand, wherein the sense strand is complementary to the antisense strand, wherein the antisense strand comprises a region complementary to a FXI RNA transcript, wherein each strand is about 15 to about 30 nucleotides in length, wherein the sense strand sequence may be represented by formula (II):
5′-(N′_{L})ₙ′N′_{L}N′_{L}N′_{L}N′_{L}N′_{F}N′L N′_{F}N′_{L}N′_{N1}N′_{N2}N′_{L}N′_{L}N′_{L}N′_{L}N′_{L}(N′_{L})_{m′}-3′ formula (II)
wherein:
each N′_{F} represents a 2′-fluoro-modified nucleotide; each N′_{N1} and N′_{N2} independently represents a modified or unmodified nucleotide, N′_{N1} and N′_{N2} include only one 2′-fluorine modified nucleotides; each N′L independently represents a modified or unmodified nucleotide but not a 2′-fluoro-modified nucleotide, and m‘ and n’ are each independently an integer of 0 to 7;
the dsRNA agent III including a sense strand and an antisense strand, wherein the sense strand is complementary to the antisense strand, wherein the antisense strand comprises a region complementary to a FXI RNA transcript, wherein each strand is about 18 to about 30 nucleotides in length, wherein the antisense strand sequence may be represented by formula (III):
3′-(N_{L})ₙ N_{M1}N_{L} N_{M2}N_{L} N_{F} N_{L} N_{M3}N_{L} N_{L} N_{L} N_{L} N_{M4}N_{L} N_{M5}N_{L} N_{L} N_{F} N_{L}-5′ formula (III)
wherein:
each N_{F} represents a 2′-fluoro-modified nucleotide; each N_{M1}, N_{M2}, N_{M3}, N_{M4}, and N_{M5} independently represents a modified or unmodified nucleotide; N_{M1}, N_{M2}, N_{M3}, N_{M4}, and N_{M5} have only three 2′-fluoro-modified nucleotides; each N_{L} independently represents a modified or unmodified nucleotide but not a 2′-fluoro-modified nucleotide, and n is an integer of 0 to 7;
the dsRNA agent IV including a sense strand and an antisense strand, wherein the sense strand and the antisense strand form a dsRNA duplex, wherein said sense strand is complementary to the antisense strand, wherein said antisense strand comprises a region of complementarity to an mRNA encoding FXI, wherein the region of complementarity comprises at least 15 contiguous nucleotides, wherein the dsRNA duplex represented by formula (IV):
sense:5′-(N′_{L})_{n′}N′_{L} N′_{L} N′_{L} N′_{L} N′_{F}N′_{L} N′_{F}N′_{L} N′_{N1}N′_{N2}N′_{L} N′_{L} N′_{L} N′_{L} N′_{L}(N′_{L})_{m′}-3
antisense:3′-(N_{L})ₙ N_{M1}N_{L} N_{M2}N_{L} N_{F}N_{L} N_{M3}N_{L} N_{L} N_{L} N_{L} N_{M4}N_{L} N_{M5}N_{L} N_{L} N_{F}N_{L}-5′ formula (IV)
wherein:
each strand is about 18 to about 30 nucleotides in length;
each N_{F} and N′_{F} independently represents a 2′-fluoro-modified nucleotide; N_{M1}, N_{M2}, N_{M3}, N_{M4}, N_{M5}, N′_{N1}, and N′_{N2} each independently represents a modified or unmodified nucleotide; N_{M1}, N_{M2}, N_{M3}, N_{M4}, and N_{M5} have only three 2′-fluoro-modified nucleotides; N′_{N1} and N′_{N2} include only one 2′-fluorine modified nucleotides; each N_{L} and N′_{L} independently represents a modified or unmodified nucleotide but not a 2′-fluoro-modified nucleotide, and m′, n′ and n are each independently an integer of 0 to 7.

10-11. (canceled)

12. The dsRNA agent of claim 9, wherein the one or more modified nucleotides are independently selected from the group consisting of: a 2′-O-methyl nucleotide, a 2′-Fluoro nucleotide, a 2′-deoxy nucleotide, a 2′3′-seco nucleotide mimic, a locked nucleotide, an unlocked nucleic acid nucleotide (UNA), a glycol nucleic acid nucleotide (GNA), a 2′-F-Arabino nucleotide, a 2′-methoyxyethyl nucleotide, an abasic nucleotide, an ribitol, inverted nucleotide, an inverted abasic nucleotide, an isomannide nucleotide, an inverted 2′-Ome nucleotide, an inverted 2′-deoxy nucleotide, a 2′-amino-modified nucleotide, a 2′-alkyl-modified nucleotide, a mopholino nucleotide, a 3′-OMe nucleotide, a nucleotide comprising a 5′-phosphorothioate group, a terminal nucleotide linked to a cholesteryl derivative or dodecanoic acid bisdecylamide group, a 2′-amino-modified nucleotide, a phosphoramidate, or a non-natural base comprising nucleotide;
preferably, the dsRNA agent comprises an E-vinylphosphonate nucleotide at the 5′ end of the guide strand;
optionally, wherein the dsRNA agent comprises at least one phosphorothioate internucleoside linkage,
and optionally, wherein the sense strand comprises at least one phosphorothioate internucleoside linkage, preferably, the sense strand comprises 1, 2, 3, 4, 5, or 6 phosphorothioate internucleoside linkages: or wherein the antisense strand comprises at least one phosphorothioate internucleoside linkage, preferably, the antisense strand comprises 1, 2, 3, 4, 5, or 6 phosphorothioate internucleoside linkages: or
wherein the modified sense strand is a modified sense strand sequence set forth in one of Tables 2-3, and optionally the modified antisense strand is a modified antisense strand sequence set forth in one of Tables 2-3.

13-20. (canceled)

21. The dsRNA agent of claim 1, wherein the sense strand is complementary or substantially complementary to the antisense strand for a region of 16-23 or 19-21 nucleotides in length.

22. (canceled)

23. The dsRNA agent of claim 1, wherein each strand is no more than 30, 25 or 3 nucleotides in length.

24-25. (canceled)

26. The dsRNA agent of claim 1, wherein the dsRNA agent comprises at least one modified nucleotide and further comprises one or more targeting groups or linking groups, optionally, wherein the one or more targeting groups or linking groups are conjugated to the sense strand, preferably, wherein the targeting group or linking group comprises N-acetyl-galactosamine (GalNAc),
wherein the targeting group has a structure:

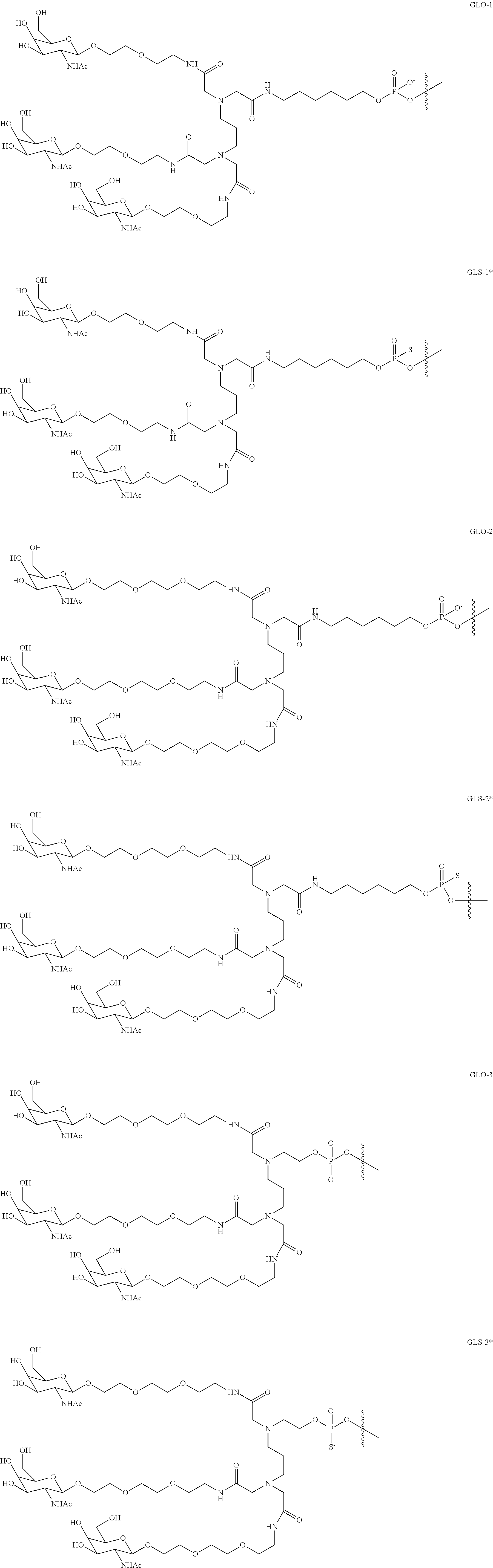

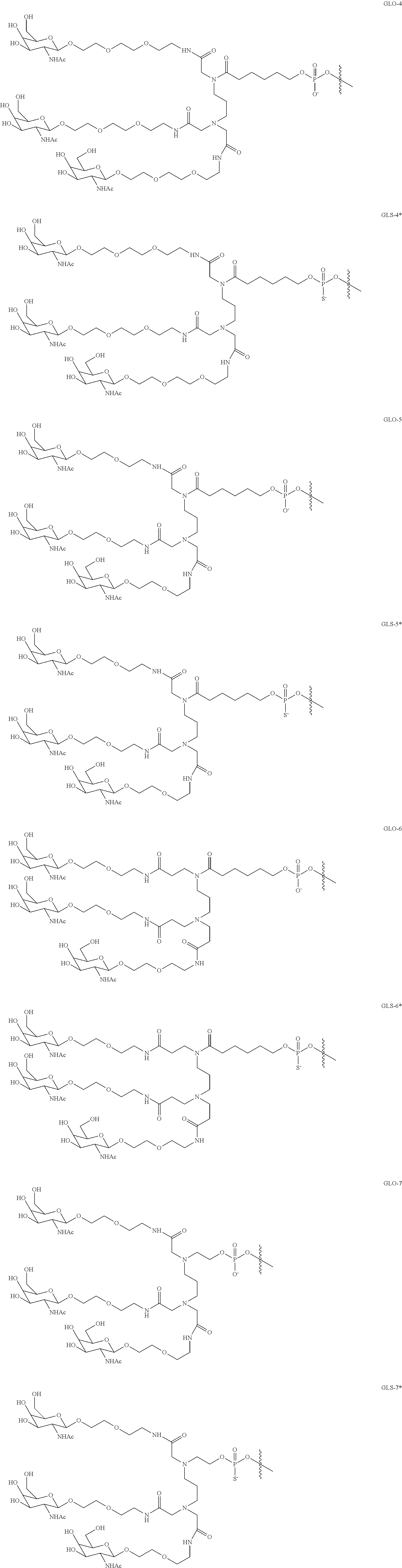

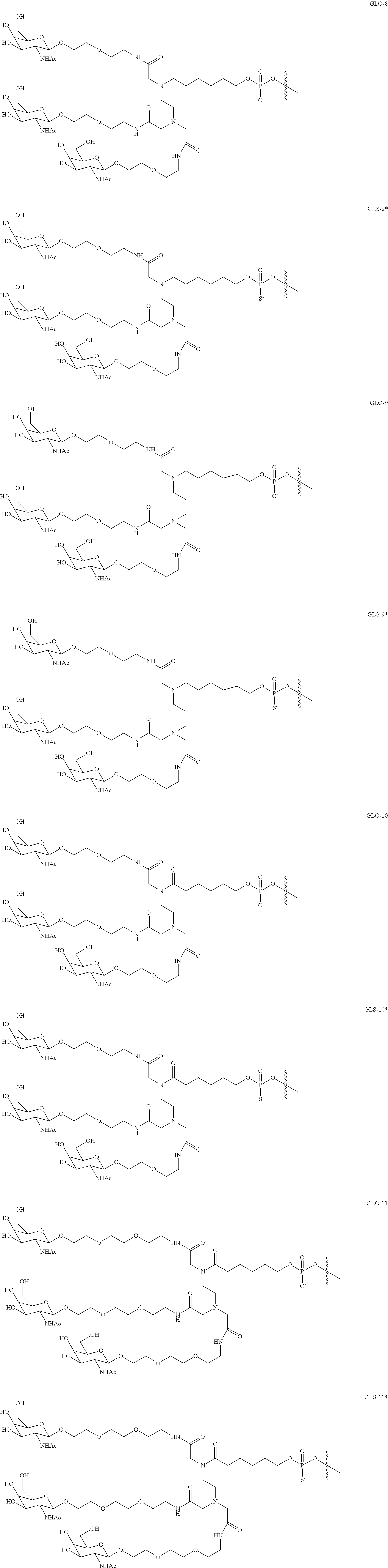

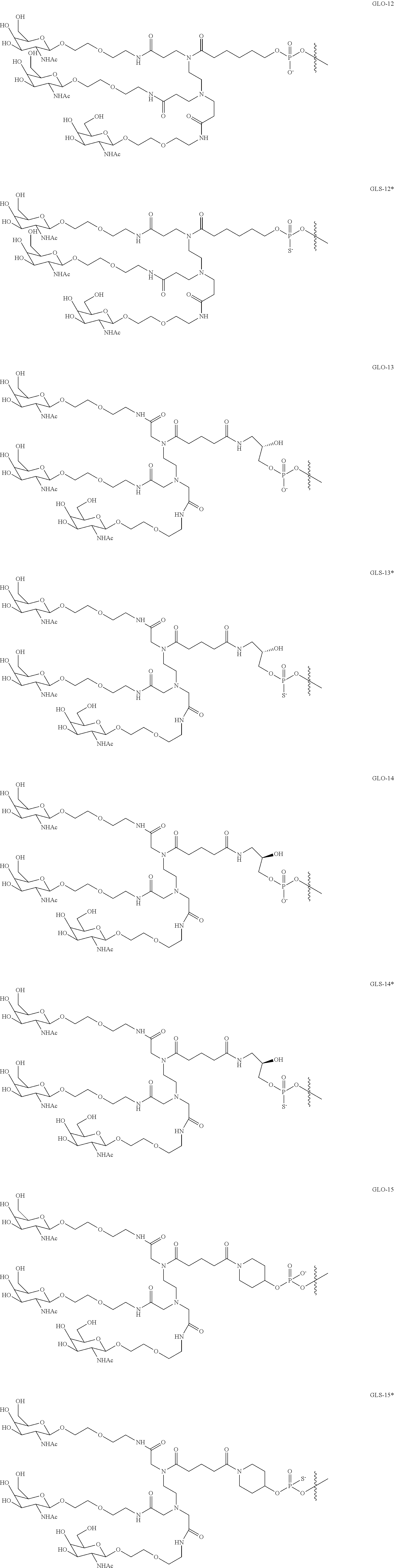

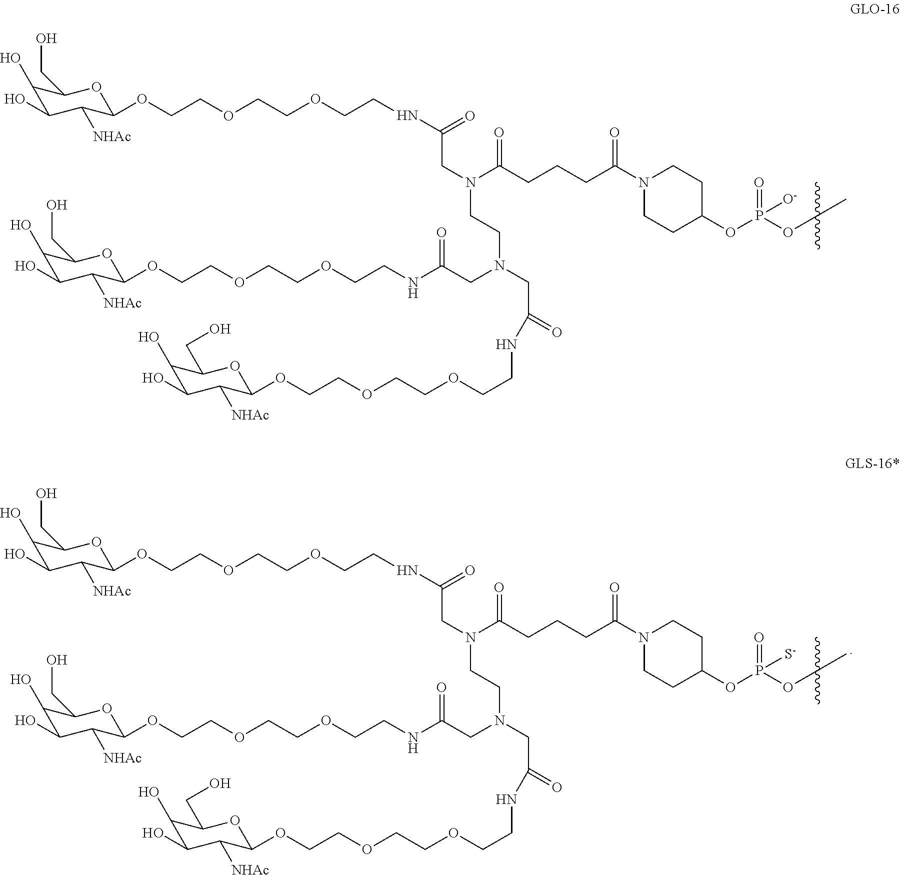 [SMILES: *OP(=O)([O-])OC1CCN(C(=O)CCCC(=O)N(CCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1 ; *OP(=O)([S-])OC1CCN(C(=O)CCCC(=O)N(CCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1]

27-29. (canceled)

30. The dsRNA agent of claim 1, wherein the dsRNA agent comprises a targeting group that is conjugated to the 5′-terminal end of the sense strand, or wherein the dsRNA agent comprises a targeting group that is conjugated to the 3-terminal end of the sense strand.

31. (canceled)

32. The dsRNA agent of claim 1, wherein the antisense strand comprises one inverted abasic residue at 3′-terminal end, or wherein the sense strand comprises one or two inverted abasic residues or imann residues at 3′ or/and 5′ terminal end;
preferably, wherein the dsRNA agent has two blunt ends, or
at least one strand comprises a 3′ overhang of at least 1 or 2 nucleotides.

33-37. (canceled)

38. A composition comprising one, two, three, or more dsRNA agents of claim 1, preferably, the composition further comprises a pharmaceutically acceptable carrier;
preferably, the composition further comprises one or more additional therapeutic agents;
preferably, the composition is packaged in a kit, container, pack, dispenser, pre-filled syringe, or vial;
preferably, the composition is formulated for subcutaneous administration or is formulated for intravenous (IV) administration.

39-42. (canceled)

43. A cell comprising a dsRNA agent of claim 1, preferably, the cell is a mammalian cell, optionally a human cell.

44. (canceled)

45. A method of inhibiting the expression of a FXI gene in a cell, the method comprising:
(i) preparing a cell or plurality of cells comprising an effective amount of one or more double-stranded ribonucleic acid (dsRNA) agents of claim 1,
and optionally comprising (ii) maintaining the cell or plurality of cells prepared in (i) for a time sufficient to obtain degradation of the mRNA transcript of a FXI gene, thereby inhibiting expression of the FXI gene in the cell or plurality of cells and reducing a level of the FXI polypeptide in the cell or plurality of cells,
and preferably, the cell or plurality of cells is in a subject and the dsRNA agent is administered to the subject subcutaneously or by IV administration, or the cell or plurality of cells is outside a subject and is contacted with the dsRNA agent,
preferably, wherein 2, 3, 4, or more dsRNA agents are administered to the subject or contacted with the cell or plurality of cells,
preferably, the method further comprises assessing inhibition of the FXI gene in the subject, following the administration of the dsRNA agent(s) to the subject, wherein a means for the assessing comprises:
(a) determining one or more physiological characteristics of a FXI-associated disease or condition in the subject and
(b) comparing the determined physiological characteristic(s) to a baseline pre-administration physiological characteristic of the FXI-associated disease or condition and/or to a control physiological characteristic of the FXI-associated disease or condition, wherein the comparison indicates one or more of a presence or absence of inhibition of expression of the FXI gene in the subject,
preferably, the determined physiological characteristic in the subject is one or more of: a level of plasma FXI mRNA level, the FXI protein level, or active form (FXIa) level, partial thromboplastin time (PTT) test, activated partial thromboplastin time test (aPTT or APTT), or the determined physiological characteristic is determined in a biological sample obtained from the subject, preferably, the biological sample comprises one or more of: a blood sample, a serum sample, a tissue sample, a cell sample, and a liver sample.

46-56. (canceled)

57. A method of inhibiting expression of a FXI gene in a subject, the method comprising administering to the subject an effective amount of a double-stranded ribonucleic acid (dsRNA) agent of claim 1, and optionally, the dsRNA agent is administered to the subject subcutaneously or by IV administration, and optionally the method further comprises assessing inhibition of the FXI gene, following the administration of the dsRNA agent, wherein a means for the assessing comprises:
(i) determining one or more physiological characteristics of a FXI-associated disease or condition in the subject and
(ii) comparing the determined physiological characteristic(s) to a baseline pre-treatment physiological characteristic of the FXI-associated disease or condition and/or to a control physiological characteristic of the FXI-associated disease or condition,
wherein the comparison indicates one or more of a presence or absence of inhibition of expression of the FXI gene in the subject,
and preferably, the determined physiological characteristic is one or more of: the FXI mRNA level, the FXI protein level in the subject, or active form (FXIa) level, partial thromboplastin time (PTT) test, activated partial thromboplastin time test (aPTT or APTT).

58-60. (canceled)

61. A method of treating a disease or condition associated with the presence of FXI protein, the method comprising administering to a subject an effective amount of a double-stranded ribonucleic acid (dsRNA) agent of claim 1, to inhibit FXI gene expression, preferably, the disease or condition is one or more of: thromboembolic condition, such as deep vein thrombosis, venous or arterial thrombosis, pulmonary embolism, myocardial infarction, thrombosis associated with chronic kidney disease or end-stage renal disease (ESRD), and stroke,
and optionally the method further comprises administering an additional therapeutic regimen to the subject, and preferably, the additional therapeutic regimen comprises: administering to the subject one or more FXI antisense polynucleotides of the invention, administering to the subject a non-FXI dsRNA therapeutic agent, and a behavioral modification in the subject, preferably, the non-FXI dsRNA therapeutic agent is one of more of: NSAID/Cyclooxygenase inhibitors, such as, aspirin; adenosine diphosphate (ADP) receptor inhibitors, such as, clopidogrel (PLAVIX) and ticlopidine (TICLID); phosphodiesterase inhibitors, such as, cilostazol (PLETAL); glycoprotein IIB/IIIA inhibitors, such as, abciximab (REOPRO), eptifibatide (INTEGRILIN), tirofiban (AGGRASTAT), and defibrotide; adenosine reuptake inhibitors, such as, dipyridamole (PERSANTINE); warfarin (and related coumarins); heparin; direct thrombin inhibitors, such as, lepirudin, bivalirudin; apixaban, LOVENOX (enoxaparin); small molecular compounds that interfere directly with the enzymatic action of particular coagulation factors (e.g. rivaroxaban, which interferes with Factor Xa)
preferably, wherein the dsRNA agent is administered to the subject subcutaneously or IV administration;
and optionally the method further comprises determining an efficacy of the administered double-stranded ribonucleic acid (dsRNA) agent in the subject.

62-67. (canceled)

68. A method of decreasing a level of FXI protein in a subject compared to a baseline pre-treatment level of FXI protein in the subject, the method comprising administering to the subject an effective amount of a double-stranded ribonucleic acid (dsRNA) agent of claim 1, to decrease the level of FXI gene expression, preferably, the dsRNA agent is administered to the subject subcutaneously or is administered to the subject by IV administration.

69. (canceled)

70. A method of altering a physiological characteristic of a FXI-associated disease or condition in a subject compared to a baseline pre-treatment physiological characteristic of the FXI-associated disease or condition in the subject, the method comprising administering to the subject an effective amount of a double-stranded ribonucleic acid (dsRNA) agent of claim 1, to alter the physiological characteristic of the FXI-associated disease or condition in the subject, preferably, the dsRNA agent is administered to the subject subcutaneously or is administered to the subject by IV administration, preferably, the physiological characteristic is one or more of: the FXI mRNA level, the FXI protein level in the subject, or active form (FXIa) level, partial thromboplastin time (PTT) test, activated partial thromboplastin time test (aPTT or APTT).

71-72. (canceled)


SEQUENCE LISTING (1734 sequences)

SEQ ID NO: 1 - RNA, 3053 nt, Homo sapiens
  source 1..3053: mol_type = mRNA
gaccatttcctacacaagctcattcagaggaggatgaagaccattttggaggaagaaaagcacccttattaagaattgcagcaagtaagccaacaaggtcttttcaggatgattttcttatatcaagtggtacatttcattttatttacttcagtttctggtgaatgtgtgactcagttgttgaaggacacctgctttgaaggaggggacattactacggtcttcacaccaagcgccaagtactgccaggtagtctgcacttaccacccaagatgtttactcttcactttcacggcggaatcaccatctgaggatcccacccgatggtttacttgtgtcctgaaagacagtgttacagaaacactgccaagagtgaataggacagcagcgatttctgggtattctttcaagcaatgctcacaccaaataagcgcttgcaacaaagacatttatgtggacctagacatgaagggcataaactataacagctcagttgccaagagtgctcaagaatgccaagaaagatgcacggatgacgtccactgccactttttcacgtacgccacaaggcagtttcccagcctggagcatcgtaacatttgtctactgaagcacacccaaacagggacaccaaccagaataacgaagctcgataaagtggtgtctggattttcactgaaatcctgtgcactttctaatctggcttgtattagggacattttccctaatacggtgtttgcagacagcaacatcgacagtgtcatggctcccgatgcttttgtctgtggccgaatctgcactcatcatcccggttgcttgttttttaccttcttttcccaggaatggcccaaagaatctcaaagaaatctttgtctccttaaaacatctgagagtggattgcccagtacacgcattaaaaagagcaaagctctttctggtttcagtctacaaagctgcaggcacagcatcccagtgttctgccattcttcattttaccatgacactgatttcttgggagaagaactggatattgttgctgcaaaaagtcacgaggcctgccagaaactgtgcaccaatgccgtccgctgccagttttttacctataccccagcccaagcatcctgcaacgaagggaagggcaagtgttacttaaagctttcttcaaacggatctccaactaaaatacttcacgggagaggaggcatctctggatacacattaaggttgtgtaaaatggataatgagtgtaccaccaaaatcaagcccaggatcgttggaggaactgcgtctgttcgtggtgagtggccgtggcaggtgaccctgcacacaacctcacccactcagagacacctgtgtggaggctccatcattggaaaccagtggatattaacagccgctcactgtttctatggggtagagtcacctaagattttgcgtgtctacagtggcattttaaatcaatctgaaataaaagaggacacatctttctttggggttcaagaaataataatccatgatcagtataaaatggcagaaagcgggtatgatattgccttgttgaaactggaaaccacagtgaattacacagattctcaacgacccatatgcctgccttccaaaggagatagaaatgtaatatacactgattgctgggtgactggatgggggtacagaaaactaagagacaaaatacaaaatactctccagaaagccaagatacccttagtgaccaacgaagagtgccagaagagatacagaggacataaaataacccataagatgatctgtgccggctacagggaaggagggaaggacgcttgcaagggagattcgggaggccctctgtcctgcaaacacaatgaggtctggcatctggtaggcatcacgagctggggcgaaggctgtgctcaaagggagcggccaggtgtttacaccaacgtggtcgagtacgtggactggattctggagaaaactcaagcagtgtgaatgggttcccaggggccattggagtccctgaaggacccaggatttgctgggagagggtgttgagttcactgtgccagcatgcttcctccacagtaacacgctgaaggggcttggtgtttgtaagaaaatgctagaagaaaacaaactgtcacaagttgttatgtccaaaactcccgttctatgatcgttgtagtttgtttgagcattcagtctctttgtttttgatcacgcttctatggagtccaagaattaccataaggcaatatttctgaagattactatataggcagatatagcagaaaataaccaagtagtggcagtggggatcaggcagaagaactggtaaaagaagccaccataaatagatttgttcgatgaaagatgaaaactggaagaaaggagaacaaagacagtcttcaccattttgcaggaatctacactctgcctatgtgaacacatttcttttgtaaagaaagaaattgattgcatttaatggcagattttcagaatagtcaggaattcttgtcatttccattttaaaatatatattaaaaaaaatcagttcgagtagacacgagctaagagtgaatgtgaagataacagaatttctgtgtggaagaggattacaagcagcaatttacctggaagtgataccttaggggcaatcttgaagatacactttcctgaaaaatgatttgtgatggattgtatatttatttaaaatatcttgggaggggaggctgatggagatagggagcatgctcaaacctccctaagacaagctgctgctgtgactatgggctcccaaagagctagatcgtatatttatttgacaaaaatcaccatagactgcatccatactacagagaaaaaacaattagggcgcaaatggatagttacagtaaagtcttcagcaagcagctgcctgtattctaagcactgggattttctgtttcgtgcaaatatttatctcattattgttgtgatctagttcaataacctagaatttgaattgtcaccacatagctttcaatctgtgccaacaactatacaattcatcaagtgtg

SEQ ID NO: 2 - RNA, 3053 nt, Homo sapiens
  source 1..3053: mol_type = mRNA
cacacttgatgaattgtatagttgttggcacagattgaaagctatgtggtgacaattcaaattctaggttattgaactagatcacaacaataatgagataaatatttgcacgaaacagaaaatcccagtgcttagaatacaggcagctgcttgctgaagactttactgtaactatccatttgcgccctaattgttttttctctgtagtatggatgcagtctatggtgatttttgtcaaataaatatacgatctagctctttgggagcccatagtcacagcagcagcttgtcttagggaggtttgagcatgctccctatctccatcagcctcccctcccaagatattttaaataaatatacaatccatcacaaatcatttttcaggaaagtgtatcttcaagattgcccctaaggtatcacttccaggtaaattgctgcttgtaatcctcttccacacagaaattctgttatcttcacattcactcttagctcgtgtctactcgaactgattttttttaatatatattttaaaatggaaatgacaagaattcctgactattctgaaaatctgccattaaatgcaatcaatttctttctttacaaaagaaatgtgttcacataggcagagtgtagattcctgcaaaatggtgaagactgtctttgttctcctttcttccagttttcatctttcatcgaacaaatctatttatggtggcttcttttaccagttcttctgcctgatccccactgccactacttggttattttctgctatatctgcctatatagtaatcttcagaaatattgccttatggtaattcttggactccatagaagcgtgatcaaaaacaaagagactgaatgctcaaacaaactacaacgatcatagaacgggagttttggacataacaacttgtgacagtttgttttcttctagcattttcttacaaacaccaagccccttcagcgtgttactgtggaggaagcatgctggcacagtgaactcaacaccctctcccagcaaatcctgggtccttcagggactccaatggcccctgggaacccattcacactgcttgagttttctccagaatccagtccacgtactcgaccacgttggtgtaaacacctggccgctccctttgagcacagccttcgccccagctcgtgatgcctaccagatgccagacctcattgtgtttgcaggacagagggcctcccgaatctcccttgcaagcgtccttccctccttccctgtagccggcacagatcatcttatgggttattttatgtcctctgtatctcttctggcactcttcgttggtcactaagggtatcttggctttctggagagtattttgtattttgtctcttagttttctgtacccccatccagtcacccagcaatcagtgtatattacatttctatctcctttggaaggcaggcatatgggtcgttgagaatctgtgtaattcactgtggtttccagtttcaacaaggcaatatcatacccgctttctgccattttatactgatcatggattattatttcttgaaccccaaagaaagatgtgtcctcttttatttcagattgatttaaaatgccactgtagacacgcaaaatcttaggtgactctaccccatagaaacagtgagcggctgttaatatccactggtttccaatgatggagcctccacacaggtgtctctgagtgggtgaggttgtgtgcagggtcacctgccacggccactcaccacgaacagacgcagttcctccaacgatcctgggcttgattttggtggtacactcattatccattttacacaaccttaatgtgtatccagagatgcctcctctcccgtgaagtattttagttggagatccgtttgaagaaagctttaagtaacacttgcccttcccttcgttgcaggatgcttgggctggggtataggtaaaaaactggcagcggacggcattggtgcacagtttctggcaggcctcgtgactttttgcagcaacaatatccagttcttctcccaagaaatcagtgtcatggtaaaatgaagaatggcagaacactgggatgctgtgcctgcagctttgtagactgaaaccagaaagagctttgctctttttaatgcgtgtactgggcaatccactctcagatgttttaaggagacaaagatttctttgagattctttgggccattcctgggaaaagaaggtaaaaaacaagcaaccgggatgatgagtgcagattcggccacagacaaaagcatcgggagccatgacactgtcgatgttgctgtctgcaaacaccgtattagggaaaatgtccctaatacaagccagattagaaagtgcacaggatttcagtgaaaatccagacaccactttatcgagcttcgttattctggttggtgtccctgtttgggtgtgcttcagtagacaaatgttacgatgctccaggctgggaaactgccttgtggcgtacgtgaaaaagtggcagtggacgtcatccgtgcatctttcttggcattcttgagcactcttggcaactgagctgttatagtttatgcccttcatgtctaggtccacataaatgtctttgttgcaagcgcttatttggtgtgagcattgcttgaaagaatacccagaaatcgctgctgtcctattcactcttggcagtgtttctgtaacactgtctttcaggacacaagtaaaccatcgggtgggatcctcagatggtgattccgccgtgaaagtgaagagtaaacatcttgggtggtaagtgcagactacctggcagtacttggcgcttggtgtgaagaccgtagtaatgtcccctccttcaaagcaggtgtccttcaacaactgagtcacacattcaccagaaactgaagtaaataaaatgaaatgtaccacttgatataagaaaatcatcctgaaaagaccttgttggcttacttgctgcaattcttaataagggtgcttttcttcctccaaaatggtcttcatcctcctctgaatgagcttgtgtaggaaatggtc

SEQ ID NO: 3 - RNA, 2654 nt, Mus musculus
  source 1..2654: mol_type = mRNA
aaacactccgccgaggactacagcaaagacagaaagtatctgcagaatgcgttactaaggtcttcaaagacatcagctttcaaggaggtgacctgagtactgttttcacaccgagcgccacatactgccgcttggtctgcactcaccacccacggtgcttgctcttcacgttcatggctgagtcatcttcggatgatcctaccaaatggtttgcctgcatcctgaaggacagcgtcacagaaatattgccaatggtaaacatgacaggcgcgatctctggatattccttcaagcaatgccctcagcaattaagtacttgcagcaaagatgtgtacgtgaacctagacatgaagggcatgaactataacagctctgtcgtgaagaatgctcgagaatgccaggagagatgcacagacgatgcccactgccagtttttcacatacgcaacagggtattttcccagtgtggaccatcgtaaaatgtgtcttttgaagtacacccgaacggggacgccaaccacaataacgaagctcaatggcgtggtatctggattttcactgaagtcctgtggactttcaaacttggcttgtatcagggacattttccctaacacggtgctggcagaccttaacattgacagcgtggtggccccagatgcttttgtctgtcgtcgcatctgcacgcatcaccccacttgtttgttcttcacattcttttcccaagcatggccgaaagaatctcagagacatctttgtctccttaaaacctctgaaagtggattaccaagcacacgcattacaaagagccacgccctttcgggcttcagtctccagcactgcaggcacagtgtcccagtattctgccatccgtccttttacaacgacactgatttcttgggagaagagctggacatcgtcgatgtgaaaggccaagaaacctgtcagaaaacgtgtaccaataacgcccgctgccagttctttacctactatccatcgcacagactgtgcaatgagaggaaccgcaggggcagatgttacctaaagctttcctccaatggatctccaacgagaatacttcatgggaggggaggcatctctggatactcactgaggctgtgcaaaatggataatgtgtgcacaactaaaatcaaccccagagtggtaggaggagctgcgtctgttcacggtgagtggccatggcaggtgactctgcacatcagccagggacacctgtgtggaggctccatcattggaaaccaatggatactgacagcagctcattgtttctctgggatagagacacctaaaaagctgcgtgtctacggtggcattgtaaatcaatcagaaataaatgaagggactgctttcttcagggttcaagaaatgataattcatgatcagtatacgacagcagaaagtgggtatgatattgccctgttaaaactggaatcagccatgaattacacagattttcagcggccaatatgcctgccttccaaaggagatagaaacgcagtgcacacagaatgctgggtgactggatgggggtacacagcactaagaggtgaagtacaaagtactcttcagaaagccaaggttccattggtgtcaaatgaagaatgtcagacaagatacagaagacacaaaataaccaataagatgatctgtgcaggctacaaagaaggagggaaggatacgtgcaagggagattctggagggcccctgtcctgcaaatacaatggggtctggcacttggtgggcatcacaagctggggtgaaggctgtggtcagaaggagagaccgggggtctacacgaacgtggccaagtacgtggactggattctggagaaaactcaaacagtctgaaagagttcaactggtatcactttgtggccctggaagattattccatagaaatgagcttgacgtctctgatgaagacactgggatactgactcttccactgtaaccaattgaatggccttgatgtacgtaagaacacccagaaagaaaactattattttcagaattcctgatctgggagaaccactggttgttttctgcatccagctactactcaaggaaacaaatacagcaaggagattttaaaaataaaaacacatcagatatataaggaaaatatcaagtaagggtgctgtctgcctttttagtctctgtgacaaatacctaaagtagttcacaaaaggaaaaatttcttttgcacacctttcctcaggtttcagcctacgatctggttggctggccccattgctttagcctgaggtgaggcagaaccatatatccataggaggctgtggagaaggagtctgctcagttcatggtaggcaggaagcaaatggaaacaggaatgtattggggacacgaatggtccttcaagaatatactgtcaatcatttacttcttccagagacatcctgctccctaacctccctttccttcccagataacacctctgtcctagctggccccaagagatcaggtagaaaggcagaggaaaccatataaagagttgttaagtgcaaaatcaaaaccagaaggaatgcagacaggagctcaaaatgtccatttataagaatctttttttttctctgcctatatgaatccccctcctgtataaaggactgactcaattca

SEQ ID NO: 4 - RNA, 2654 nt, Mus musculus
  source 1..2654: mol_type = mRNA
tgaattgagtcagtcctttatacaggagggggattcatataggcagagaaaaaaaaagattcttataaatggacattttgagctcctgtctgcattccttctggttttgattttgcacttaacaactctttatatggtttcctctgcctttctacctgatctcttggggccagctaggacagaggtgttatctgggaaggaaagggaggttagggagcaggatgtctctggaagaagtaaatgattgacagtatattcttgaaggaccattcgtgtccccaatacattcctgtttccatttgcttcctgcctaccatgaactgagcagactccttctccacagcctcctatggatatatggttctgcctcacctcaggctaaagcaatggggccagccaaccagatcgtaggctgaaacctgaggaaaggtgtgcaaaagaaatttttccttttgtgaactactttaggtatttgtcacagagactaaaaaggcagacagcacccttacttgatattttccttatatatctgatgtgtttttatttttaaaatctccttgctgtatttgtttccttgagtagtagctggatgcagaaaacaaccagtggttctcccagatcaggaattctgaaaataatagttttctttctgggtgttcttacgtacatcaaggccattcaattggttacagtggaagagtcagtatcccagtgtcttcatcagagacgtcaagctcatttctatggaataatcttccagggccacaaagtgataccagttgaactctttcagactgtttgagttttctccagaatccagtccacgtacttggccacgttcgtgtagacccccggtctctccttctgaccacagccttcaccccagcttgtgatgcccaccaagtgccagaccccattgtatttgcaggacaggggccctccagaatctcccttgcacgtatccttccctccttctttgtagcctgcacagatcatcttattggttattttgtgtcttctgtatcttgtctgacattcttcatttgacaccaatggaaccttggctttctgaagagtactttgtacttcacctcttagtgctgtgtacccccatccagtcacccagcattctgtgtgcactgcgtttctatctcctttggaaggcaggcatattggccgctgaaaatctgtgtaattcatggctgattccagttttaacagggcaatatcatacccactttctgctgtcgtatactgatcatgaattatcatttcttgaaccctgaagaaagcagtcccttcatttatttctgattgatttacaatgccaccgtagacacgcagctttttaggtgtctctatcccagagaaacaatgagctgctgtcagtatccattggtttccaatgatggagcctccacacaggtgtccctggctgatgtgcagagtcacctgccatggccactcaccgtgaacagacgcagctcctcctaccactctggggttgattttagttgtgcacacattatccattttgcacagcctcagtgagtatccagagatgcctcccctcccatgaagtattctcgttggagatccattggaggaaagctttaggtaacatctgcccctgcggttcctctcattgcacagtctgtgcgatggatagtaggtaaagaactggcagcgggcgttattggtacacgttttctgacaggtttcttggcctttcacatcgacgatgtccagctcttctcccaagaaatcagtgtcgttgtaaaaggacggatggcagaatactgggacactgtgcctgcagtgctggagactgaagcccgaaagggcgtggctctttgtaatgcgtgtgcttggtaatccactttcagaggttttaaggagacaaagatgtctctgagattctttcggccatgcttgggaaaagaatgtgaagaacaaacaagtggggtgatgcgtgcagatgcgacgacagacaaaagcatctggggccaccacgctgtcaatgttaaggtctgccagcaccgtgttagggaaaatgtccctgatacaagccaagtttgaaagtccacaggacttcagtgaaaatccagataccacgccattgagcttcgttattgtggttggcgtccccgttcgggtgtacttcaaaagacacattttacgatggtccacactgggaaaataccctgttgcgtatgtgaaaaactggcagtgggcatcgtctgtgcatctctcctggcattctcgagcattcttcacgacagagctgttatagttcatgcccttcatgtctaggttcacgtacacatctttgctgcaagtacttaattgctgagggcattgcttgaaggaatatccagagatcgcgcctgtcatgtttaccattggcaatatttctgtgacgctgtccttcaggatgcaggcaaaccatttggtaggatcatccgaagatgactcagccatgaacgtgaagagcaagcaccgtgggtggtgagtgcagaccaagcggcagtatgtggcgctcggtgtgaaaacagtactcaggtcacctccttgaaagctgatgtctttgaagaccttagtaacgcattctgcagatactttctgtctttgctgtagtcctcggcggagtgttt

SEQ ID NO: 5 - RNA, 21 nt, synthetic construct
  source 1..21: mol_type = other RNA
gaacaaggtcttttcaggata

SEQ ID NO: 6 - RNA, 21 nt, synthetic construct
  source 1..21: mol_type = other RNA
gcaaggtcttttcaggatgaa

SEQ ID NO: 7 - RNA, 21 nt, synthetic construct
  source 1..21: mol_type = other RNA
gaggtcttttcaggatgatta

SEQ ID NO: 8 - RNA, 21 nt, synthetic construct
  source 1..21: mol_type = other RNA
ctcttttcaggatgattttca

SEQ ID NO: 9 - RNA, 21 nt, synthetic construct
  source 1..21: mol_type = other RNA
gtttcaggatgattttcttaa

SEQ ID NO: 10 - RNA, 21 nt, synthetic construct
  source 1..21: mol_type = other RNA
gaggatgattttcttatatca

SEQ ID NO: 11 - RNA, 21 nt, synthetic construct
  source 1..21: mol_type = other RNA
cgatgattttcttatatcaaa

SEQ ID NO: 12 - RNA, 21 nt, synthetic construct
  source 1..21: mol_type = other RNA
gcttcagtttctggtgaatga

SEQ ID NO: 13 - RNA, 21 nt, synthetic construct
  source 1..21: mol_type = other RNA
gtcagtttctggtgaatgtga

SEQ ID NO: 14 - RNA, 21 nt, synthetic construct
  source 1..21: mol_type = other RNA
gttctggtgaatgtgtgacta

SEQ ID NO: 15 - RNA, 21 nt, synthetic construct
  source 1..21: mol_type = other RNA
gtggtttacttgtgtcctgaa

SEQ ID NO: 16 - RNA, 21 nt, synthetic construct
  source 1..21: mol_type = other RNA
gggtttacttgtgtcctgaaa

SEQ ID NO: 17 - RNA, 21 nt, synthetic construct
  source 1..21: mol_type = other RNA
caaagacagtgttacagaaaa

SEQ ID NO: 18 - RNA, 21 nt, synthetic construct
  source 1..21: mol_type = other RNA
gaagacagtgttacagaaaca

SEQ ID NO: 19 - RNA, 21 nt, synthetic construct
  source 1..21: mol_type = other RNA
ggacagtgttacagaaacaca

SEQ ID NO: 20 - RNA, 21 nt, synthetic construct
  source 1..21: mol_type = other RNA
gtacagaaacactgccaagaa

SEQ ID NO: 21 - RNA, 21 nt, synthetic construct
  source 1..21: mol_type = other RNA
gacagaaacactgccaagaga

SEQ ID NO: 22 - RNA, 21 nt, synthetic construct
  source 1..21: mol_type = other RNA
caaacactgccaagagtgaaa

SEQ ID NO: 23 - RNA, 21 nt, synthetic construct
  source 1..21: mol_type = other RNA
gaacactgccaagagtgaata

SEQ ID NO: 24 - RNA, 21 nt, synthetic construct
  source 1..21: mol_type = other RNA
gactgccaagagtgaatagga

SEQ ID NO: 25 - RNA, 21 nt, synthetic construct
  source 1..21: mol_type = other RNA
gagcagcgatttctgggtata

SEQ ID NO: 26 - RNA, 21 nt, synthetic construct
  source 1..21: mol_type = other RNA
ccagcgatttctgggtattca

SEQ ID NO: 27 - RNA, 21 nt, synthetic construct
  source 1..21: mol_type = other RNA
ggatttctgggtattctttca

SEQ ID NO: 28 - RNA, 21 nt, synthetic construct
  source 1..21: mol_type = other RNA
gtctgggtattctttcaagca

SEQ ID NO: 29 - RNA, 21 nt, synthetic construct
  source 1..21: mol_type = other RNA
gctgggtattctttcaagcaa

SEQ ID NO: 30 - RNA, 21 nt, synthetic construct
  source 1..21: mol_type = other RNA
gtgggtattctttcaagcaaa

SEQ ID NO: 31 - RNA, 21 nt, synthetic construct
  source 1..21: mol_type = other RNA
ggggtattctttcaagcaata

SEQ ID NO: 32 - RNA, 21 nt, synthetic construct
  source 1..21: mol_type = other RNA
cgtattctttcaagcaatgca

SEQ ID NO: 33 - RNA, 21 nt, synthetic construct
  source 1..21: mol_type = other RNA
gttctttcaagcaatgctcaa

SEQ ID NO: 34 - RNA, 21 nt, synthetic construct
  source 1..21: mol_type = other RNA
gtctttcaagcaatgctcaca

SEQ ID NO: 35 - RNA, 21 nt, synthetic construct
  source 1..21: mol_type = other RNA
ccaatgctcacaccaaataaa

SEQ ID NO: 36 - RNA, 21 nt, synthetic construct
  source 1..21: mol_type = other RNA
gaatgctcacaccaaataaga

SEQ ID NO: 37 - RNA, 21 nt, synthetic construct
  source 1..21: mol_type = other RNA
gatgctcacaccaaataagca

SEQ ID NO: 38 - RNA, 21 nt, synthetic construct
  source 1..21: mol_type = other RNA
gcaccaaataagcgcttgcaa

SEQ ID NO: 39 - RNA, 21 nt, synthetic construct
  source 1..21: mol_type = other RNA
gaccaaataagcgcttgcaaa

SEQ ID NO: 40 - RNA, 21 nt, synthetic construct
  source 1..21: mol_type = other RNA
gaaataagcgcttgcaacaaa

SEQ ID NO: 41 - RNA, 21 nt, synthetic construct
  source 1..21: mol_type = other RNA
gaataagcgcttgcaacaaaa

SEQ ID NO: 42 - RNA, 21 nt, synthetic construct
  source 1..21: mol_type = other RNA
ccttgcaacaaagacatttaa

SEQ ID NO: 43 - RNA, 21 nt, synthetic construct
  source 1..21: mol_type = other RNA
ggcaacaaagacatttatgta

SEQ ID NO: 44 - RNA, 21 nt, synthetic construct
  source 1..21: mol_type = other RNA
ccaacaaagacatttatgtga

SEQ ID NO: 45 - RNA, 21 nt, synthetic construct
  source 1..21: mol_type = other RNA
gaacaaagacatttatgtgga

SEQ ID NO: 46 - RNA, 21 nt, synthetic construct
  source 1..21: mol_type = other RNA
gacaaagacatttatgtggaa

SEQ ID NO: 47 - RNA, 21 nt, synthetic construct
  source 1..21: mol_type = other RNA
gaagacatttatgtggaccta

SEQ ID NO: 48 - RNA, 21 nt, synthetic construct
  source 1..21: mol_type = other RNA
gagacatttatgtggacctaa

SEQ ID NO: 49 - RNA, 21 nt, synthetic construct
  source 1..21: mol_type = other RNA
ggacatttatgtggacctaga

SEQ ID NO: 50 - RNA, 21 nt, synthetic construct
  source 1..21: mol_type = other RNA
cacatttatgtggacctagaa

SEQ ID NO: 51 - RNA, 21 nt, synthetic construct
  source 1..21: mol_type = other RNA
gatgtggacctagacatgaaa

SEQ ID NO: 52 - RNA, 21 nt, synthetic construct
  source 1..21: mol_type = other RNA
gtagacatgaagggcataaaa

SEQ ID NO: 53 - RNA, 21 nt, synthetic construct
  source 1..21: mol_type = other RNA
gagacatgaagggcataaaca

SEQ ID NO: 54 - RNA, 21 nt, synthetic construct
  source 1..21: mol_type = other RNA
ggacatgaagggcataaacta

SEQ ID NO: 55 - RNA, 21 nt, synthetic construct
  source 1..21: mol_type = other RNA
gcatgaagggcataaactata

SEQ ID NO: 56 - RNA, 21 nt, synthetic construct
  source 1..21: mol_type = other RNA
gatgaagggcataaactataa

SEQ ID NO: 57 - RNA, 21 nt, synthetic construct
  source 1..21: mol_type = other RNA
gtgaagggcataaactataaa

SEQ ID NO: 58 - RNA, 21 nt, synthetic construct
  source 1..21: mol_type = other RNA
ggaagggcataaactataaca

SEQ ID NO: 59 - RNA, 21 nt, synthetic construct
  source 1..21: mol_type = other RNA
caagggcataaactataacaa

SEQ ID NO: 60 - RNA, 21 nt, synthetic construct
  source 1..21: mol_type = other RNA
gagggcataaactataacaga

SEQ ID NO: 61 - RNA, 21 nt, synthetic construct
  source 1..21: mol_type = other RNA
cggcataaactataacagcta

SEQ ID NO: 62 - RNA, 21 nt, synthetic construct
  source 1..21: mol_type = other RNA
ccataaactataacagctcaa

SEQ ID NO: 63 - RNA, 21 nt, synthetic construct
  source 1..21: mol_type = other RNA
cccaagagtgctcaagaatga

SEQ ID NO: 64 - RNA, 21 nt, synthetic construct
  source 1..21: mol_type = other RNA
gcaagaatgccaagaaagata

SEQ ID NO: 65 - RNA, 21 nt, synthetic construct
  source 1..21: mol_type = other RNA
gaagaatgccaagaaagatga

SEQ ID NO: 66 - RNA, 21 nt, synthetic construct
  source 1..21: mol_type = other RNA
gccactgccactttttcacga

SEQ ID NO: 67 - RNA, 21 nt, synthetic construct
  source 1..21: mol_type = other RNA
gcactgccactttttcacgta

SEQ ID NO: 68 - RNA, 21 nt, synthetic construct
  source 1..21: mol_type = other RNA
gactgccactttttcacgtaa

SEQ ID NO: 69 - RNA, 21 nt, synthetic construct
  source 1..21: mol_type = other RNA
gagaaatctttgtctccttaa

SEQ ID NO: 70 - RNA, 21 nt, synthetic construct
  source 1..21: mol_type = other RNA
gatctttgtctccttaaaaca

SEQ ID NO: 71 - RNA, 21 nt, synthetic construct
  source 1..21: mol_type = other RNA
gtttgtctccttaaaacatca

SEQ ID NO: 72 - RNA, 21 nt, synthetic construct
  source 1..21: mol_type = other RNA
gctccttaaaacatctgagaa

SEQ ID NO: 73 - RNA, 21 nt, synthetic construct
  source 1..21: mol_type = other RNA
gccttaaaacatctgagagta

SEQ ID NO: 74 - RNA, 21 nt, synthetic construct
  source 1..21: mol_type = other RNA
gaaacatctgagagtggatta

SEQ ID NO: 75 - RNA, 21 nt, synthetic construct
  source 1..21: mol_type = other RNA
gcacgcattaaaaagagcaaa

SEQ ID NO: 76 - RNA, 21 nt, synthetic construct
  source 1..21: mol_type = other RNA
gcgcattaaaaagagcaaaga

SEQ ID NO: 77 - RNA, 21 nt, synthetic construct
  source 1..21: mol_type = other RNA
gtaaaaagagcaaagctctta

SEQ ID NO: 78 - RNA, 21 nt, synthetic construct
  source 1..21: mol_type = other RNA
gaagagcaaagctctttctga

SEQ ID NO: 79 - RNA, 21 nt, synthetic construct
  source 1..21: mol_type = other RNA
ggagcaaagctctttctggta

SEQ ID NO: 80 - RNA, 21 nt, synthetic construct
  source 1..21: mol_type = other RNA
cagcaaagctctttctggtta

SEQ ID NO: 81 - RNA, 21 nt, synthetic construct
  source 1..21: mol_type = other RNA
ccaaagctctttctggtttca

SEQ ID NO: 82 - RNA, 21 nt, synthetic construct
  source 1..21: mol_type = other RNA
gaaagctctttctggtttcaa

SEQ ID NO: 83 - RNA, 21 nt, synthetic construct
  source 1..21: mol_type = other RNA
caagaactggatattgttgca

SEQ ID NO: 84 - RNA, 21 nt, synthetic construct
  source 1..21: mol_type = other RNA
gagaactggatattgttgcta

SEQ ID NO: 85 - RNA, 21 nt, synthetic construct
  source 1..21: mol_type = other RNA
gtgccagaaactgtgcaccaa

SEQ ID NO: 86 - RNA, 21 nt, synthetic construct
  source 1..21: mol_type = other RNA
cccagaaactgtgcaccaata

SEQ ID NO: 87 - RNA, 21 nt, synthetic construct
  source 1..21: mol_type = other RNA
gcgctgccagttttttaccta

SEQ ID NO: 88 - RNA, 21 nt, synthetic construct
  source 1..21: mol_type = other RNA
cctgccagttttttacctata

SEQ ID NO: 89 - RNA, 21 nt, synthetic construct
  source 1..21: mol_type = other RNA
gagtgttacttaaagctttca

SEQ ID NO: 90 - RNA, 21 nt, synthetic construct
  source 1..21: mol_type = other RNA
ggtgttacttaaagctttcta

SEQ ID NO: 91 - RNA, 21 nt, synthetic construct
  source 1..21: mol_type = other RNA
ctgttacttaaagctttctta

SEQ ID NO: 92 - RNA, 21 nt, synthetic construct
  source 1..21: mol_type = other RNA
gtacttaaagctttcttcaaa

SEQ ID NO: 93 - RNA, 21 nt, synthetic construct
  source 1..21: mol_type = other RNA
gacttaaagctttcttcaaaa

SEQ ID NO: 94 - RNA, 21 nt, synthetic construct
  source 1..21: mol_type = other RNA
gggatctccaactaaaataca

SEQ ID NO: 95 - RNA, 21 nt, synthetic construct
  source 1..21: mol_type = other RNA
cgatctccaactaaaatacta

SEQ ID NO: 96 - RNA, 21 nt, synthetic construct
  source 1..21: mol_type = other RNA
catctccaactaaaatactta

SEQ ID NO: 97 - RNA, 21 nt, synthetic construct
  source 1..21: mol_type = other RNA
ccatctctggatacacattaa

SEQ ID NO: 98 - RNA, 21 nt, synthetic construct
  source 1..21: mol_type = other RNA
gatctctggatacacattaaa

SEQ ID NO: 99 - RNA, 21 nt, synthetic construct
  source 1..21: mol_type = other RNA
gtctctggatacacattaaga

SEQ ID NO: 100 - RNA, 21 nt, synthetic construct
  source 1..21: mol_type = other RNA
gctctggatacacattaagga

SEQ ID NO: 101 - RNA, 21 nt, synthetic construct
  source 1..21: mol_type = other RNA
gtgtgtaaaatggataatgaa

SEQ ID NO: 102 - RNA, 19 nt, synthetic construct
  source 1..19: mol_type = other RNA
acaaggtcttttcaggata

SEQ ID NO: 103 - RNA, 19 nt, synthetic construct
  source 1..19: mol_type = other RNA
aaggtcttttcaggatgaa

SEQ ID NO: 104 - RNA, 19 nt, synthetic construct
  source 1..19: mol_type = other RNA
ggtcttttcaggatgatta

SEQ ID NO: 105 - RNA, 19 nt, synthetic construct
  source 1..19: mol_type = other RNA
cttttcaggatgattttca

SEQ ID NO: 106 - RNA, 19 nt, synthetic construct
  source 1..19: mol_type = other RNA
ttcaggatgattttcttaa

SEQ ID NO: 107 - RNA, 19 nt, synthetic construct
  source 1..19: mol_type = other RNA
ggatgattttcttatatca

SEQ ID NO: 108 - RNA, 19 nt, synthetic construct
  source 1..19: mol_type = other RNA
atgattttcttatatcaaa

SEQ ID NO: 109 - RNA, 19 nt, synthetic construct
  source 1..19: mol_type = other RNA
ttcagtttctggtgaatga

SEQ ID NO: 110 - RNA, 19 nt, synthetic construct
  source 1..19: mol_type = other RNA
cagtttctggtgaatgtga

SEQ ID NO: 111 - RNA, 19 nt, synthetic construct
  source 1..19: mol_type = other RNA
tctggtgaatgtgtgacta

SEQ ID NO: 112 - RNA, 19 nt, synthetic construct
  source 1..19: mol_type = other RNA
ggtttacttgtgtcctgaa

SEQ ID NO: 113 - RNA, 19 nt, synthetic construct
  source 1..19: mol_type = other RNA
gtttacttgtgtcctgaaa

SEQ ID NO: 114 - RNA, 19 nt, synthetic construct
  source 1..19: mol_type = other RNA
aagacagtgttacagaaaa

SEQ ID NO: 115 - RNA, 19 nt, synthetic construct
  source 1..19: mol_type = other RNA
agacagtgttacagaaaca

SEQ ID NO: 116 - RNA, 19 nt, synthetic construct
  source 1..19: mol_type = other RNA
acagtgttacagaaacaca

SEQ ID NO: 117 - RNA, 19 nt, synthetic construct
  source 1..19: mol_type = other RNA
acagaaacactgccaagaa

SEQ ID NO: 118 - RNA, 19 nt, synthetic construct
  source 1..19: mol_type = other RNA
cagaaacactgccaagaga

SEQ ID NO: 119 - RNA, 19 nt, synthetic construct
  source 1..19: mol_type = other RNA
aacactgccaagagtgaaa

SEQ ID NO: 120 - RNA, 19 nt, synthetic construct
  source 1..19: mol_type = other RNA
acactgccaagagtgaata

SEQ ID NO: 121 - RNA, 19 nt, synthetic construct
  source 1..19: mol_type = other RNA
ctgccaagagtgaatagga

SEQ ID NO: 122 - RNA, 19 nt, synthetic construct
  source 1..19: mol_type = other RNA
gcagcgatttctgggtata

SEQ ID NO: 123 - RNA, 19 nt, synthetic construct
  source 1..19: mol_type = other RNA
agcgatttctgggtattca

SEQ ID NO: 124 - RNA, 19 nt, synthetic construct
  source 1..19: mol_type = other RNA
atttctgggtattctttca

SEQ ID NO: 125 - RNA, 19 nt, synthetic construct
  source 1..19: mol_type = other RNA
ctgggtattctttcaagca

SEQ ID NO: 126 - RNA, 19 nt, synthetic construct
  source 1..19: mol_type = other RNA
tgggtattctttcaagcaa

SEQ ID NO: 127 - RNA, 19 nt, synthetic construct
  source 1..19: mol_type = other RNA
gggtattctttcaagcaaa

SEQ ID NO: 128 - RNA, 19 nt, synthetic construct
  source 1..19: mol_type = other RNA
ggtattctttcaagcaata

SEQ ID NO: 129 - RNA, 19 nt, synthetic construct
  source 1..19: mol_type = other RNA
tattctttcaagcaatgca

SEQ ID NO: 130 - RNA, 19 nt, synthetic construct
  source 1..19: mol_type = other RNA
tctttcaagcaatgctcaa

SEQ ID NO: 131 - RNA, 19 nt, synthetic construct
  source 1..19: mol_type = other RNA
ctttcaagcaatgctcaca

SEQ ID NO: 132 - RNA, 19 nt, synthetic construct
  source 1..19: mol_type = other RNA
aatgctcacaccaaataaa

SEQ ID NO: 133 - RNA, 19 nt, synthetic construct
  source 1..19: mol_type = other RNA
atgctcacaccaaataaga

SEQ ID NO: 134 - RNA, 19 nt, synthetic construct
  source 1..19: mol_type = other RNA
tgctcacaccaaataagca

SEQ ID NO: 135 - RNA, 19 nt, synthetic construct
  source 1..19: mol_type = other RNA
accaaataagcgcttgcaa

SEQ ID NO: 136 - RNA, 19 nt, synthetic construct
  source 1..19: mol_type = other RNA
ccaaataagcgcttgcaaa

SEQ ID NO: 137 - RNA, 19 nt, synthetic construct
  source 1..19: mol_type = other RNA
aataagcgcttgcaacaaa

SEQ ID NO: 138 - RNA, 19 nt, synthetic construct
  source 1..19: mol_type = other RNA
ataagcgcttgcaacaaaa

SEQ ID NO: 139 - RNA, 19 nt, synthetic construct
  source 1..19: mol_type = other RNA
ttgcaacaaagacatttaa

SEQ ID NO: 140 - RNA, 19 nt, synthetic construct
  source 1..19: mol_type = other RNA
caacaaagacatttatgta

SEQ ID NO: 141 - RNA, 19 nt, synthetic construct
  source 1..19: mol_type = other RNA
aacaaagacatttatgtga

SEQ ID NO: 142 - RNA, 19 nt, synthetic construct
  source 1..19: mol_type = other RNA
acaaagacatttatgtgga

SEQ ID NO: 143 - RNA, 19 nt, synthetic construct
  source 1..19: mol_type = other RNA
caaagacatttatgtggaa

SEQ ID NO: 144 - RNA, 19 nt, synthetic construct
  source 1..19: mol_type = other RNA
agacatttatgtggaccta

SEQ ID NO: 145 - RNA, 19 nt, synthetic construct
  source 1..19: mol_type = other RNA
gacatttatgtggacctaa

SEQ ID NO: 146 - RNA, 19 nt, synthetic construct
  source 1..19: mol_type = other RNA
acatttatgtggacctaga

SEQ ID NO: 147 - RNA, 19 nt, synthetic construct
  source 1..19: mol_type = other RNA
catttatgtggacctagaa

SEQ ID NO: 148 - RNA, 19 nt, synthetic construct
  source 1..19: mol_type = other RNA
tgtggacctagacatgaaa

SEQ ID NO: 149 - RNA, 19 nt, synthetic construct
  source 1..19: mol_type = other RNA
agacatgaagggcataaaa

SEQ ID NO: 150 - RNA, 19 nt, synthetic construct
  source 1..19: mol_type = other RNA
gacatgaagggcataaaca

SEQ ID NO: 151 - RNA, 19 nt, synthetic construct
  source 1..19: mol_type = other RNA
acatgaagggcataaacta

SEQ ID NO: 152 - RNA, 19 nt, synthetic construct
  source 1..19: mol_type = other RNA
atgaagggcataaactata

SEQ ID NO: 153 - RNA, 19 nt, synthetic construct
  source 1..19: mol_type = other RNA
tgaagggcataaactataa

SEQ ID NO: 154 - RNA, 19 nt, synthetic construct
  source 1..19: mol_type = other RNA
gaagggcataaactataaa

SEQ ID NO: 155 - RNA, 19 nt, synthetic construct
  source 1..19: mol_type = other RNA
aagggcataaactataaca

SEQ ID NO: 156 - RNA, 19 nt, synthetic construct
  source 1..19: mol_type = other RNA
agggcataaactataacaa

SEQ ID NO: 157 - RNA, 19 nt, synthetic construct
  source 1..19: mol_type = other RNA
gggcataaactataacaga

SEQ ID NO: 158 - RNA, 19 nt, synthetic construct
  source 1..19: mol_type = other RNA
gcataaactataacagcta

SEQ ID NO: 159 - RNA, 19 nt, synthetic construct
  source 1..19: mol_type = other RNA
ataaactataacagctcaa

SEQ ID NO: 160 - RNA, 19 nt, synthetic construct
  source 1..19: mol_type = other RNA
caagagtgctcaagaatga

SEQ ID NO: 161 - RNA, 19 nt, synthetic construct
  source 1..19: mol_type = other RNA
aagaatgccaagaaagata

SEQ ID NO: 162 - RNA, 19 nt, synthetic construct
  source 1..19: mol_type = other RNA
agaatgccaagaaagatga

SEQ ID NO: 163 - RNA, 19 nt, synthetic construct
  source 1..19: mol_type = other RNA
cactgccactttttcacga

SEQ ID NO: 164 - RNA, 19 nt, synthetic construct
  source 1..19: mol_type = other RNA
actgccactttttcacgta

SEQ ID NO: 165 - RNA, 19 nt, synthetic construct
  source 1..19: mol_type = other RNA
ctgccactttttcacgtaa

SEQ ID NO: 166 - RNA, 19 nt, synthetic construct
  source 1..19: mol_type = other RNA
gaaatctttgtctccttaa

SEQ ID NO: 167 - RNA, 19 nt, synthetic construct
  source 1..19: mol_type = other RNA
tctttgtctccttaaaaca

SEQ ID NO: 168 - RNA, 19 nt, synthetic construct
  source 1..19: mol_type = other RNA
ttgtctccttaaaacatca

SEQ ID NO: 169 - RNA, 19 nt, synthetic construct
  source 1..19: mol_type = other RNA
tccttaaaacatctgagaa

SEQ ID NO: 170 - RNA, 19 nt, synthetic construct
  source 1..19: mol_type = other RNA
cttaaaacatctgagagta

SEQ ID NO: 171 - RNA, 19 nt, synthetic construct
  source 1..19: mol_type = other RNA
aacatctgagagtggatta

SEQ ID NO: 172 - RNA, 19 nt, synthetic construct
  source 1..19: mol_type = other RNA
acgcattaaaaagagcaaa

SEQ ID NO: 173 - RNA, 19 nt, synthetic construct
  source 1..19: mol_type = other RNA
gcattaaaaagagcaaaga

SEQ ID NO: 174 - RNA, 19 nt, synthetic construct
  source 1..19: mol_type = other RNA
aaaaagagcaaagctctta

SEQ ID NO: 175 - RNA, 19 nt, synthetic construct
  source 1..19: mol_type = other RNA
agagcaaagctctttctga

SEQ ID NO: 176 - RNA, 19 nt, synthetic construct
  source 1..19: mol_type = other RNA
agcaaagctctttctggta

SEQ ID NO: 177 - RNA, 19 nt, synthetic construct
  source 1..19: mol_type = other RNA
gcaaagctctttctggtta

SEQ ID NO: 178 - RNA, 19 nt, synthetic construct
  source 1..19: mol_type = other RNA
aaagctctttctggtttca

SEQ ID NO: 179 - RNA, 19 nt, synthetic construct
  source 1..19: mol_type = other RNA
aagctctttctggtttcaa

SEQ ID NO: 180 - RNA, 19 nt, synthetic construct
  source 1..19: mol_type = other RNA
agaactggatattgttgca

SEQ ID NO: 181 - RNA, 19 nt, synthetic construct
  source 1..19: mol_type = other RNA
gaactggatattgttgcta

SEQ ID NO: 182 - RNA, 19 nt, synthetic construct
  source 1..19: mol_type = other RNA
gccagaaactgtgcaccaa

SEQ ID NO: 183 - RNA, 19 nt, synthetic construct
  source 1..19: mol_type = other RNA
cagaaactgtgcaccaata

SEQ ID NO: 184 - RNA, 19 nt, synthetic construct
  source 1..19: mol_type = other RNA
gctgccagttttttaccta

SEQ ID NO: 185 - RNA, 19 nt, synthetic construct
  source 1..19: mol_type = other RNA
tgccagttttttacctata

SEQ ID NO: 186 - RNA, 19 nt, synthetic construct
  source 1..19: mol_type = other RNA
gtgttacttaaagctttca

SEQ ID NO: 187 - RNA, 19 nt, synthetic construct
  source 1..19: mol_type = other RNA
tgttacttaaagctttcta

SEQ ID NO: 188 - RNA, 19 nt, synthetic construct
  source 1..19: mol_type = other RNA
gttacttaaagctttctta

SEQ ID NO: 189 - RNA, 19 nt, synthetic construct
  source 1..19: mol_type = other RNA
acttaaagctttcttcaaa

SEQ ID NO: 190 - RNA, 19 nt, synthetic construct
  source 1..19: mol_type = other RNA
cttaaagctttcttcaaaa

SEQ ID NO: 191 - RNA, 19 nt, synthetic construct
  source 1..19: mol_type = other RNA
gatctccaactaaaataca

SEQ ID NO: 192 - RNA, 19 nt, synthetic construct
  source 1..19: mol_type = other RNA
atctccaactaaaatacta

SEQ ID NO: 193 - RNA, 19 nt, synthetic construct
  source 1..19: mol_type = other RNA
tctccaactaaaatactta

SEQ ID NO: 194 - RNA, 19 nt, synthetic construct
  source 1..19: mol_type = other RNA
atctctggatacacattaa

SEQ ID NO: 195 - RNA, 19 nt, synthetic construct
  source 1..19: mol_type = other RNA
tctctggatacacattaaa

SEQ ID NO: 196 - RNA, 19 nt, synthetic construct
  source 1..19: mol_type = other RNA
ctctggatacacattaaga

SEQ ID NO: 197 - RNA, 19 nt, synthetic construct
  source 1..19: mol_type = other RNA
tctggatacacattaagga

SEQ ID NO: 198 - RNA, 19 nt, synthetic construct
  source 1..19: mol_type = other RNA
gtgtaaaatggataatgaa

SEQ ID NO: 199 - RNA, 21 nt, synthetic construct
  source 1..21: mol_type = other RNA
tatcctgaaaagaccttgttc

SEQ ID NO: 200 - RNA, 21 nt, synthetic construct
  source 1..21: mol_type = other RNA
ttcatcctgaaaagaccttgc

SEQ ID NO: 201 - RNA, 21 nt, synthetic construct
  source 1..21: mol_type = other RNA
taatcatcctgaaaagacctc

SEQ ID NO: 202 - RNA, 21 nt, synthetic construct
  source 1..21: mol_type = other RNA
tgaaaatcatcctgaaaagag

SEQ ID NO: 203 - RNA, 21 nt, synthetic construct
  source 1..21: mol_type = other RNA
ttaagaaaatcatcctgaaac

SEQ ID NO: 204 - RNA, 21 nt, synthetic construct
  source 1..21: mol_type = other RNA
tgatataagaaaatcatcctc

SEQ ID NO: 205 - RNA, 21 nt, synthetic construct
  source 1..21: mol_type = other RNA
tttgatataagaaaatcatcg

SEQ ID NO: 206 - RNA, 21 nt, synthetic construct
  source 1..21: mol_type = other RNA
tcattcaccagaaactgaagc

SEQ ID NO: 207 - RNA, 21 nt, synthetic construct
  source 1..21: mol_type = other RNA
tcacattcaccagaaactgac

SEQ ID NO: 208 - RNA, 21 nt, synthetic construct
  source 1..21: mol_type = other RNA
tagtcacacattcaccagaac

SEQ ID NO: 209 - RNA, 21 nt, synthetic construct
  source 1..21: mol_type = other RNA
ttcaggacacaagtaaaccac

SEQ ID NO: 210 - RNA, 21 nt, synthetic construct
  source 1..21: mol_type = other RNA
tttcaggacacaagtaaaccc

SEQ ID NO: 211 - RNA, 21 nt, synthetic construct
  source 1..21: mol_type = other RNA
ttttctgtaacactgtctttg

SEQ ID NO: 212 - RNA, 21 nt, synthetic construct
  source 1..21: mol_type = other RNA
tgtttctgtaacactgtcttc

SEQ ID NO: 213 - RNA, 21 nt, synthetic construct
  source 1..21: mol_type = other RNA
tgtgtttctgtaacactgtcc

SEQ ID NO: 214 - RNA, 21 nt, synthetic construct
  source 1..21: mol_type = other RNA
ttcttggcagtgtttctgtac

SEQ ID NO: 215 - RNA, 21 nt, synthetic construct
  source 1..21: mol_type = other RNA
tctcttggcagtgtttctgtc

SEQ ID NO: 216 - RNA, 21 nt, synthetic construct
  source 1..21: mol_type = other RNA
tttcactcttggcagtgtttg

SEQ ID NO: 217 - RNA, 21 nt, synthetic construct
  source 1..21: mol_type = other RNA
tattcactcttggcagtgttc

SEQ ID NO: 218 - RNA, 21 nt, synthetic construct
  source 1..21: mol_type = other RNA
tcctattcactcttggcagtc

SEQ ID NO: 219 - RNA, 21 nt, synthetic construct
  source 1..21: mol_type = other RNA
tatacccagaaatcgctgctc

SEQ ID NO: 220 - RNA, 21 nt, synthetic construct
  source 1..21: mol_type = other RNA
tgaatacccagaaatcgctgg

SEQ ID NO: 221 - RNA, 21 nt, synthetic construct
  source 1..21: mol_type = other RNA
tgaaagaatacccagaaatcc

SEQ ID NO: 222 - RNA, 21 nt, synthetic construct
  source 1..21: mol_type = other RNA
tgcttgaaagaatacccagac

SEQ ID NO: 223 - RNA, 21 nt, synthetic construct
  source 1..21: mol_type = other RNA
ttgcttgaaagaatacccagc

SEQ ID NO: 224 - RNA, 21 nt, synthetic construct
  source 1..21: mol_type = other RNA
tttgcttgaaagaatacccac

SEQ ID NO: 225 - RNA, 21 nt, synthetic construct
  source 1..21: mol_type = other RNA
tattgcttgaaagaatacccc

SEQ ID NO: 226 - RNA, 21 nt, synthetic construct
  source 1..21: mol_type = other RNA
tgcattgcttgaaagaatacg

SEQ ID NO: 227 - RNA, 21 nt, synthetic construct
  source 1..21: mol_type = other RNA
ttgagcattgcttgaaagaac

SEQ ID NO: 228 - RNA, 21 nt, synthetic construct
  source 1..21: mol_type = other RNA
tgtgagcattgcttgaaagac

SEQ ID NO: 229 - RNA, 21 nt, synthetic construct
  source 1..21: mol_type = other RNA
tttatttggtgtgagcattgg

SEQ ID NO: 230 - RNA, 21 nt, synthetic construct
  source 1..21: mol_type = other RNA
tcttatttggtgtgagcattc

SEQ ID NO: 231 - RNA, 21 nt, synthetic construct
  source 1..21: mol_type = other RNA
tgcttatttggtgtgagcatc

SEQ ID NO: 232 - RNA, 21 nt, synthetic construct
  source 1..21: mol_type = other RNA
ttgcaagcgcttatttggtgc

SEQ ID NO: 233 - RNA, 21 nt, synthetic construct
  source 1..21: mol_type = other RNA
tttgcaagcgcttatttggtc

SEQ ID NO: 234 - RNA, 21 nt, synthetic construct
  source 1..21: mol_type = other RNA
tttgttgcaagcgcttatttc

SEQ ID NO: 235 - RNA, 21 nt, synthetic construct
  source 1..21: mol_type = other RNA
ttttgttgcaagcgcttattc

SEQ ID NO: 236 - RNA, 21 nt, synthetic construct
  source 1..21: mol_type = other RNA
ttaaatgtctttgttgcaagg

SEQ ID NO: 237 - RNA, 21 nt, synthetic construct
  source 1..21: mol_type = other RNA
tacataaatgtctttgttgcc

SEQ ID NO: 238 - RNA, 21 nt, synthetic construct
  source 1..21: mol_type = other RNA
tcacataaatgtctttgttgg

SEQ ID NO: 239 - RNA, 21 nt, synthetic construct
  source 1..21: mol_type = other RNA
tccacataaatgtctttgttc

SEQ ID NO: 240 - RNA, 21 nt, synthetic construct
  source 1..21: mol_type = other RNA
ttccacataaatgtctttgtc

SEQ ID NO: 241 - RNA, 21 nt, synthetic construct
  source 1..21: mol_type = other RNA
taggtccacataaatgtcttc

SEQ ID NO: 242 - RNA, 21 nt, synthetic construct
  source 1..21: mol_type = other RNA
ttaggtccacataaatgtctc

SEQ ID NO: 243 - RNA, 21 nt, synthetic construct
  source 1..21: mol_type = other RNA
tctaggtccacataaatgtcc

SEQ ID NO: 244 - RNA, 21 nt, synthetic construct
  source 1..21: mol_type = other RNA
ttctaggtccacataaatgtg

SEQ ID NO: 245 - RNA, 21 nt, synthetic construct
  source 1..21: mol_type = other RNA
tttcatgtctaggtccacatc

SEQ ID NO: 246 - RNA, 21 nt, synthetic construct
  source 1..21: mol_type = other RNA
ttttatgcccttcatgtctac

SEQ ID NO: 247 - RNA, 21 nt, synthetic construct
  source 1..21: mol_type = other RNA
tgtttatgcccttcatgtctc

SEQ ID NO: 248 - RNA, 21 nt, synthetic construct
  source 1..21: mol_type = other RNA
tagtttatgcccttcatgtcc

SEQ ID NO: 249 - RNA, 21 nt, synthetic construct
  source 1..21: mol_type = other RNA
tatagtttatgcccttcatgc

SEQ ID NO: 250 - RNA, 21 nt, synthetic construct
  source 1..21: mol_type = other RNA
ttatagtttatgcccttcatc

SEQ ID NO: 251 - RNA, 21 nt, synthetic construct
  source 1..21: mol_type = other RNA
tttatagtttatgcccttcac

SEQ ID NO: 252 - RNA, 21 nt, synthetic construct
  source 1..21: mol_type = other RNA
tgttatagtttatgcccttcc

SEQ ID NO: 253 - RNA, 21 nt, synthetic construct
  source 1..21: mol_type = other RNA
ttgttatagtttatgcccttg

SEQ ID NO: 254 - RNA, 21 nt, synthetic construct
  source 1..21: mol_type = other RNA
tctgttatagtttatgccctc

SEQ ID NO: 255 - RNA, 21 nt, synthetic construct
  source 1..21: mol_type = other RNA
tagctgttatagtttatgccg

SEQ ID NO: 256 - RNA, 21 nt, synthetic construct
  source 1..21: mol_type = other RNA
ttgagctgttatagtttatgg

SEQ ID NO: 257 - RNA, 21 nt, synthetic construct
  source 1..21: mol_type = other RNA
tcattcttgagcactcttggg

SEQ ID NO: 258 - RNA, 21 nt, synthetic construct
  source 1..21: mol_type = other RNA
tatctttcttggcattcttgc

SEQ ID NO: 259 - RNA, 21 nt, synthetic construct
  source 1..21: mol_type = other RNA
tcatctttcttggcattcttc

SEQ ID NO: 260 - RNA, 21 nt, synthetic construct
  source 1..21: mol_type = other RNA
tcgtgaaaaagtggcagtggc

SEQ ID NO: 261 - RNA, 21 nt, synthetic construct
  source 1..21: mol_type = other RNA
tacgtgaaaaagtggcagtgc

SEQ ID NO: 262 - RNA, 21 nt, synthetic construct
  source 1..21: mol_type = other RNA
ttacgtgaaaaagtggcagtc

SEQ ID NO: 263 - RNA, 21 nt, synthetic construct
  source 1..21: mol_type = other RNA
ttaaggagacaaagatttctc

SEQ ID NO: 264 - RNA, 21 nt, synthetic construct
  source 1..21: mol_type = other RNA
tgttttaaggagacaaagatc

SEQ ID NO: 265 - RNA, 21 nt, synthetic construct
  source 1..21: mol_type = other RNA
tgatgttttaaggagacaaac

SEQ ID NO: 266 - RNA, 21 nt, synthetic construct
  source 1..21: mol_type = other RNA
ttctcagatgttttaaggagc

SEQ ID NO: 267 - RNA, 21 nt, synthetic construct
  source 1..21: mol_type = other RNA
tactctcagatgttttaaggc

SEQ ID NO: 268 - RNA, 21 nt, synthetic construct
  source 1..21: mol_type = other RNA
taatccactctcagatgtttc

SEQ ID NO: 269 - RNA, 21 nt, synthetic construct
  source 1..21: mol_type = other RNA
tttgctctttttaatgcgtgc

SEQ ID NO: 270 - RNA, 21 nt, synthetic construct
  source 1..21: mol_type = other RNA
tctttgctctttttaatgcgc

SEQ ID NO: 271 - RNA, 21 nt, synthetic construct
  source 1..21: mol_type = other RNA
taagagctttgctctttttac

SEQ ID NO: 272 - RNA, 21 nt, synthetic construct
  source 1..21: mol_type = other RNA
tcagaaagagctttgctcttc

SEQ ID NO: 273 - RNA, 21 nt, synthetic construct
  source 1..21: mol_type = other RNA
taccagaaagagctttgctcc

SEQ ID NO: 274 - RNA, 21 nt, synthetic construct
  source 1..21: mol_type = other RNA
taaccagaaagagctttgctg

SEQ ID NO: 275 - RNA, 21 nt, synthetic construct
  source 1..21: mol_type = other RNA
tgaaaccagaaagagctttgg

SEQ ID NO: 276 - RNA, 21 nt, synthetic construct
  source 1..21: mol_type = other RNA
ttgaaaccagaaagagctttc

SEQ ID NO: 277 - RNA, 21 nt, synthetic construct
  source 1..21: mol_type = other RNA
tgcaacaatatccagttcttg

SEQ ID NO: 278 - RNA, 21 nt, synthetic construct
  source 1..21: mol_type = other RNA
tagcaacaatatccagttctc

SEQ ID NO: 279 - RNA, 21 nt, synthetic construct
  source 1..21: mol_type = other RNA
ttggtgcacagtttctggcac

SEQ ID NO: 280 - RNA, 21 nt, synthetic construct
  source 1..21: mol_type = other RNA
tattggtgcacagtttctggg

SEQ ID NO: 281 - RNA, 21 nt, synthetic construct
  source 1..21: mol_type = other RNA
taggtaaaaaactggcagcgc

SEQ ID NO: 282 - RNA, 21 nt, synthetic construct
  source 1..21: mol_type = other RNA
tataggtaaaaaactggcagg

SEQ ID NO: 283 - RNA, 21 nt, synthetic construct
  source 1..21: mol_type = other RNA
tgaaagctttaagtaacactc

SEQ ID NO: 284 - RNA, 21 nt, synthetic construct
  source 1..21: mol_type = other RNA
tagaaagctttaagtaacacc

SEQ ID NO: 285 - RNA, 21 nt, synthetic construct
  source 1..21: mol_type = other RNA
taagaaagctttaagtaacag

SEQ ID NO: 286 - RNA, 21 nt, synthetic construct
  source 1..21: mol_type = other RNA
tttgaagaaagctttaagtac

SEQ ID NO: 287 - RNA, 21 nt, synthetic construct
  source 1..21: mol_type = other RNA
ttttgaagaaagctttaagtc

SEQ ID NO: 288 - RNA, 21 nt, synthetic construct
  source 1..21: mol_type = other RNA
tgtattttagttggagatccc

SEQ ID NO: 289 - RNA, 21 nt, synthetic construct
  source 1..21: mol_type = other RNA
tagtattttagttggagatcg

SEQ ID NO: 290 - RNA, 21 nt, synthetic construct
  source 1..21: mol_type = other RNA
taagtattttagttggagatg

SEQ ID NO: 291 - RNA, 21 nt, synthetic construct
  source 1..21: mol_type = other RNA
ttaatgtgtatccagagatgg

SEQ ID NO: 292 - RNA, 21 nt, synthetic construct
  source 1..21: mol_type = other RNA
tttaatgtgtatccagagatc

SEQ ID NO: 293 - RNA, 21 nt, synthetic construct
  source 1..21: mol_type = other RNA
tcttaatgtgtatccagagac

SEQ ID NO: 294 - RNA, 21 nt, synthetic construct
  source 1..21: mol_type = other RNA
tccttaatgtgtatccagagc

SEQ ID NO: 295 - RNA, 21 nt, synthetic construct
  source 1..21: mol_type = other RNA
ttcattatccattttacacac

SEQ ID NO: 296 - RNA, 19 nt, synthetic construct
  source 1..19: mol_type = other RNA
tatcctgaaaagaccttgt

SEQ ID NO: 297 - RNA, 19 nt, synthetic construct
  source 1..19: mol_type = other RNA
ttcatcctgaaaagacctt

SEQ ID NO: 298 - RNA, 19 nt, synthetic construct
  source 1..19: mol_type = other RNA
taatcatcctgaaaagacc

SEQ ID NO: 299 - RNA, 19 nt, synthetic construct
  source 1..19: mol_type = other RNA
tgaaaatcatcctgaaaag

SEQ ID NO: 300 - RNA, 19 nt, synthetic construct
  source 1..19: mol_type = other RNA
ttaagaaaatcatcctgaa

SEQ ID NO: 301 - RNA, 19 nt, synthetic construct
  source 1..19: mol_type = other RNA
tgatataagaaaatcatcc

SEQ ID NO: 302 - RNA, 19 nt, synthetic construct
  source 1..19: mol_type = other RNA
tttgatataagaaaatcat

SEQ ID NO: 303 - RNA, 19 nt, synthetic construct
  source 1..19: mol_type = other RNA
tcattcaccagaaactgaa

SEQ ID NO: 304 - RNA, 19 nt, synthetic construct
  source 1..19: mol_type = other RNA
tcacattcaccagaaactg

SEQ ID NO: 305 - RNA, 19 nt, synthetic construct
  source 1..19: mol_type = other RNA
tagtcacacattcaccaga

SEQ ID NO: 306 - RNA, 19 nt, synthetic construct
  source 1..19: mol_type = other RNA
ttcaggacacaagtaaacc

SEQ ID NO: 307 - RNA, 19 nt, synthetic construct
  source 1..19: mol_type = other RNA
tttcaggacacaagtaaac

SEQ ID NO: 308 - RNA, 19 nt, synthetic construct
  source 1..19: mol_type = other RNA
ttttctgtaacactgtctt

SEQ ID NO: 309 - RNA, 19 nt, synthetic construct
  source 1..19: mol_type = other RNA
tgtttctgtaacactgtct

SEQ ID NO: 310 - RNA, 19 nt, synthetic construct
  source 1..19: mol_type = other RNA
tgtgtttctgtaacactgt

SEQ ID NO: 311 - RNA, 19 nt, synthetic construct
  source 1..19: mol_type = other RNA
ttcttggcagtgtttctgt

SEQ ID NO: 312 - RNA, 19 nt, synthetic construct
  source 1..19: mol_type = other RNA
tctcttggcagtgtttctg

SEQ ID NO: 313 - RNA, 19 nt, synthetic construct
  source 1..19: mol_type = other RNA
tttcactcttggcagtgtt

SEQ ID NO: 314 - RNA, 19 nt, synthetic construct
  source 1..19: mol_type = other RNA
tattcactcttggcagtgt

SEQ ID NO: 315 - RNA, 19 nt, synthetic construct
  source 1..19: mol_type = other RNA
tcctattcactcttggcag

SEQ ID NO: 316 - RNA, 19 nt, synthetic construct
  source 1..19: mol_type = other RNA
tatacccagaaatcgctgc

SEQ ID NO: 317 - RNA, 19 nt, synthetic construct
  source 1..19: mol_type = other RNA
tgaatacccagaaatcgct

SEQ ID NO: 318 - RNA, 19 nt, synthetic construct
  source 1..19: mol_type = other RNA
tgaaagaatacccagaaat

SEQ ID NO: 319 - RNA, 19 nt, synthetic construct
  source 1..19: mol_type = other RNA
tgcttgaaagaatacccag

SEQ ID NO: 320 - RNA, 19 nt, synthetic construct
  source 1..19: mol_type = other RNA
ttgcttgaaagaataccca

SEQ ID NO: 321 - RNA, 19 nt, synthetic construct
  source 1..19: mol_type = other RNA
tttgcttgaaagaataccc

SEQ ID NO: 322 - RNA, 19 nt, synthetic construct
  source 1..19: mol_type = other RNA
tattgcttgaaagaatacc

SEQ ID NO: 323 - RNA, 19 nt, synthetic construct
  source 1..19: mol_type = other RNA
tgcattgcttgaaagaata

SEQ ID NO: 324 - RNA, 19 nt, synthetic construct
  source 1..19: mol_type = other RNA
ttgagcattgcttgaaaga

SEQ ID NO: 325 - RNA, 19 nt, synthetic construct
  source 1..19: mol_type = other RNA
tgtgagcattgcttgaaag

SEQ ID NO: 326 - RNA, 19 nt, synthetic construct
  source 1..19: mol_type = other RNA
tttatttggtgtgagcatt

SEQ ID NO: 327 - RNA, 19 nt, synthetic construct
  source 1..19: mol_type = other RNA
tcttatttggtgtgagcat

SEQ ID NO: 328 - RNA, 19 nt, synthetic construct
  source 1..19: mol_type = other RNA
tgcttatttggtgtgagca

SEQ ID NO: 329 - RNA, 19 nt, synthetic construct
  source 1..19: mol_type = other RNA
ttgcaagcgcttatttggt

SEQ ID NO: 330 - RNA, 19 nt, synthetic construct
  source 1..19: mol_type = other RNA
tttgcaagcgcttatttgg

SEQ ID NO: 331 - RNA, 19 nt, synthetic construct
  source 1..19: mol_type = other RNA
tttgttgcaagcgcttatt

SEQ ID NO: 332 - RNA, 19 nt, synthetic construct
  source 1..19: mol_type = other RNA
ttttgttgcaagcgcttat

SEQ ID NO: 333 - RNA, 19 nt, synthetic construct
  source 1..19: mol_type = other RNA
ttaaatgtctttgttgcaa

SEQ ID NO: 334 - RNA, 19 nt, synthetic construct
  source 1..19: mol_type = other RNA
tacataaatgtctttgttg

SEQ ID NO: 335 - RNA, 19 nt, synthetic construct
  source 1..19: mol_type = other RNA
tcacataaatgtctttgtt

SEQ ID NO: 336 - RNA, 19 nt, synthetic construct
  source 1..19: mol_type = other RNA
tccacataaatgtctttgt

SEQ ID NO: 337 - RNA, 19 nt, synthetic construct
  source 1..19: mol_type = other RNA
ttccacataaatgtctttg

SEQ ID NO: 338 - RNA, 19 nt, synthetic construct
  source 1..19: mol_type = other RNA
taggtccacataaatgtct

SEQ ID NO: 339 - RNA, 19 nt, synthetic construct
  source 1..19: mol_type = other RNA
ttaggtccacataaatgtc

SEQ ID NO: 340 - RNA, 19 nt, synthetic construct
  source 1..19: mol_type = other RNA
tctaggtccacataaatgt

SEQ ID NO: 341 - RNA, 19 nt, synthetic construct
  source 1..19: mol_type = other RNA
ttctaggtccacataaatg

SEQ ID NO: 342 - RNA, 19 nt, synthetic construct
  source 1..19: mol_type = other RNA
tttcatgtctaggtccaca

SEQ ID NO: 343 - RNA, 19 nt, synthetic construct
  source 1..19: mol_type = other RNA
ttttatgcccttcatgtct

SEQ ID NO: 344 - RNA, 19 nt, synthetic construct
  source 1..19: mol_type = other RNA
tgtttatgcccttcatgtc

SEQ ID NO: 345 - RNA, 19 nt, synthetic construct
  source 1..19: mol_type = other RNA
tagtttatgcccttcatgt

SEQ ID NO: 346 - RNA, 19 nt, synthetic construct
  source 1..19: mol_type = other RNA
tatagtttatgcccttcat

SEQ ID NO: 347 - RNA, 19 nt, synthetic construct
  source 1..19: mol_type = other RNA
ttatagtttatgcccttca

SEQ ID NO: 348 - RNA, 19 nt, synthetic construct
  source 1..19: mol_type = other RNA
tttatagtttatgcccttc

SEQ ID NO: 349 - RNA, 19 nt, synthetic construct
  source 1..19: mol_type = other RNA
tgttatagtttatgccctt

SEQ ID NO: 350 - RNA, 19 nt, synthetic construct
  source 1..19: mol_type = other RNA
ttgttatagtttatgccct

SEQ ID NO: 351 - RNA, 19 nt, synthetic construct
  source 1..19: mol_type = other RNA
tctgttatagtttatgccc

SEQ ID NO: 352 - RNA, 19 nt, synthetic construct
  source 1..19: mol_type = other RNA
tagctgttatagtttatgc

SEQ ID NO: 353 - RNA, 19 nt, synthetic construct
  source 1..19: mol_type = other RNA
ttgagctgttatagtttat

SEQ ID NO: 354 - RNA, 19 nt, synthetic construct
  source 1..19: mol_type = other RNA
tcattcttgagcactcttg

SEQ ID NO: 355 - RNA, 19 nt, synthetic construct
  source 1..19: mol_type = other RNA
tatctttcttggcattctt

SEQ ID NO: 356 - RNA, 19 nt, synthetic construct
  source 1..19: mol_type = other RNA
tcatctttcttggcattct

SEQ ID NO: 357 - RNA, 19 nt, synthetic construct
  source 1..19: mol_type = other RNA
tcgtgaaaaagtggcagtg

SEQ ID NO: 358 - RNA, 19 nt, synthetic construct
  source 1..19: mol_type = other RNA
tacgtgaaaaagtggcagt

SEQ ID NO: 359 - RNA, 19 nt, synthetic construct
  source 1..19: mol_type = other RNA
ttacgtgaaaaagtggcag

SEQ ID NO: 360 - RNA, 19 nt, synthetic construct
  source 1..19: mol_type = other RNA
ttaaggagacaaagatttc

SEQ ID NO: 361 - RNA, 19 nt, synthetic construct
  source 1..19: mol_type = other RNA
tgttttaaggagacaaaga

SEQ ID NO: 362 - RNA, 19 nt, synthetic construct
  source 1..19: mol_type = other RNA
tgatgttttaaggagacaa

SEQ ID NO: 363 - RNA, 19 nt, synthetic construct
  source 1..19: mol_type = other RNA
ttctcagatgttttaagga

SEQ ID NO: 364 - RNA, 19 nt, synthetic construct
  source 1..19: mol_type = other RNA
tactctcagatgttttaag

SEQ ID NO: 365 - RNA, 19 nt, synthetic construct
  source 1..19: mol_type = other RNA
taatccactctcagatgtt

SEQ ID NO: 366 - RNA, 19 nt, synthetic construct
  source 1..19: mol_type = other RNA
tttgctctttttaatgcgt

SEQ ID NO: 367 - RNA, 19 nt, synthetic construct
  source 1..19: mol_type = other RNA
tctttgctctttttaatgc

SEQ ID NO: 368 - RNA, 19 nt, synthetic construct
  source 1..19: mol_type = other RNA
taagagctttgctcttttt

SEQ ID NO: 369 - RNA, 19 nt, synthetic construct
  source 1..19: mol_type = other RNA
tcagaaagagctttgctct

SEQ ID NO: 370 - RNA, 19 nt, synthetic construct
  source 1..19: mol_type = other RNA
taccagaaagagctttgct

SEQ ID NO: 371 - RNA, 19 nt, synthetic construct
  source 1..19: mol_type = other RNA
taaccagaaagagctttgc

SEQ ID NO: 372 - RNA, 19 nt, synthetic construct
  source 1..19: mol_type = other RNA
tgaaaccagaaagagcttt

SEQ ID NO: 373 - RNA, 19 nt, synthetic construct
  source 1..19: mol_type = other RNA
ttgaaaccagaaagagctt

SEQ ID NO: 374 - RNA, 19 nt, synthetic construct
  source 1..19: mol_type = other RNA
tgcaacaatatccagttct

SEQ ID NO: 375 - RNA, 19 nt, synthetic construct
  source 1..19: mol_type = other RNA
tagcaacaatatccagttc

SEQ ID NO: 376 - RNA, 19 nt, synthetic construct
  source 1..19: mol_type = other RNA
ttggtgcacagtttctggc

SEQ ID NO: 377 - RNA, 19 nt, synthetic construct
  source 1..19: mol_type = other RNA
tattggtgcacagtttctg

SEQ ID NO: 378 - RNA, 19 nt, synthetic construct
  source 1..19: mol_type = other RNA
taggtaaaaaactggcagc

SEQ ID NO: 379 - RNA, 19 nt, synthetic construct
  source 1..19: mol_type = other RNA
tataggtaaaaaactggca

SEQ ID NO: 380 - RNA, 19 nt, synthetic construct
  source 1..19: mol_type = other RNA
tgaaagctttaagtaacac

SEQ ID NO: 381 - RNA, 19 nt, synthetic construct
  source 1..19: mol_type = other RNA
tagaaagctttaagtaaca

SEQ ID NO: 382 - RNA, 19 nt, synthetic construct
  source 1..19: mol_type = other RNA
taagaaagctttaagtaac

SEQ ID NO: 383 - RNA, 19 nt, synthetic construct
  source 1..19: mol_type = other RNA
tttgaagaaagctttaagt

SEQ ID NO: 384 - RNA, 19 nt, synthetic construct
  source 1..19: mol_type = other RNA
ttttgaagaaagctttaag

SEQ ID NO: 385 - RNA, 19 nt, synthetic construct
  source 1..19: mol_type = other RNA
tgtattttagttggagatc

SEQ ID NO: 386 - RNA, 19 nt, synthetic construct
  source 1..19: mol_type = other RNA
tagtattttagttggagat

SEQ ID NO: 387 - RNA, 19 nt, synthetic construct
  source 1..19: mol_type = other RNA
taagtattttagttggaga

SEQ ID NO: 388 - RNA, 19 nt, synthetic construct
  source 1..19: mol_type = other RNA
ttaatgtgtatccagagat

SEQ ID NO: 389 - RNA, 19 nt, synthetic construct
  source 1..19: mol_type = other RNA
tttaatgtgtatccagaga

SEQ ID NO: 390 - RNA, 19 nt, synthetic construct
  source 1..19: mol_type = other RNA
tcttaatgtgtatccagag

SEQ ID NO: 391 - RNA, 19 nt, synthetic construct
  source 1..19: mol_type = other RNA
tccttaatgtgtatccaga

SEQ ID NO: 392 - RNA, 19 nt, synthetic construct
  source 1..19: mol_type = other RNA
ttcattatccattttacac

SEQ ID NO: 393 - RNA, 21 nt, synthetic construct
  source 1..21: mol_type = other RNA
gaacaaggtcttttcaggata

SEQ ID NO: 394 - RNA, 21 nt, synthetic construct
  source 1..21: mol_type = other RNA
gcaaggtcttttcaggatgaa

SEQ ID NO: 395 - RNA, 21 nt, synthetic construct
  source 1..21: mol_type = other RNA
gaggtcttttcaggatgatta

SEQ ID NO: 396 - RNA, 21 nt, synthetic construct
  source 1..21: mol_type = other RNA
ctcttttcaggatgattttca

SEQ ID NO: 397 - RNA, 21 nt, synthetic construct
  source 1..21: mol_type = other RNA
gtttcaggatgattttcttaa

SEQ ID NO: 398 - RNA, 21 nt, synthetic construct
  source 1..21: mol_type = other RNA
gaggatgattttcttatatca

SEQ ID NO: 399 - RNA, 21 nt, synthetic construct
  source 1..21: mol_type = other RNA
cgatgattttcttatatcaaa

SEQ ID NO: 400 - RNA, 21 nt, synthetic construct
  source 1..21: mol_type = other RNA
gcttcagtttctggtgaatga

SEQ ID NO: 401 - RNA, 21 nt, synthetic construct
  source 1..21: mol_type = other RNA
gtcagtttctggtgaatgtga

SEQ ID NO: 402 - RNA, 21 nt, synthetic construct
  source 1..21: mol_type = other RNA
gttctggtgaatgtgtgacta

SEQ ID NO: 403 - RNA, 21 nt, synthetic construct
  source 1..21: mol_type = other RNA
gtggtttacttgtgtcctgaa

SEQ ID NO: 404 - RNA, 21 nt, synthetic construct
  source 1..21: mol_type = other RNA
gggtttacttgtgtcctgaaa

SEQ ID NO: 405 - RNA, 21 nt, synthetic construct
  source 1..21: mol_type = other RNA
caaagacagtgttacagaaaa

SEQ ID NO: 406 - RNA, 21 nt, synthetic construct
  source 1..21: mol_type = other RNA
gaagacagtgttacagaaaca

SEQ ID NO: 407 - RNA, 21 nt, synthetic construct
  source 1..21: mol_type = other RNA
ggacagtgttacagaaacaca

SEQ ID NO: 408 - RNA, 21 nt, synthetic construct
  source 1..21: mol_type = other RNA
gtacagaaacactgccaagaa

SEQ ID NO: 409 - RNA, 21 nt, synthetic construct
  source 1..21: mol_type = other RNA
gacagaaacactgccaagaga

SEQ ID NO: 410 - RNA, 21 nt, synthetic construct
  source 1..21: mol_type = other RNA
caaacactgccaagagtgaaa

SEQ ID NO: 411 - RNA, 21 nt, synthetic construct
  source 1..21: mol_type = other RNA
gaacactgccaagagtgaata

SEQ ID NO: 412 - RNA, 21 nt, synthetic construct
  source 1..21: mol_type = other RNA
gactgccaagagtgaatagga

SEQ ID NO: 413 - RNA, 21 nt, synthetic construct
  source 1..21: mol_type = other RNA
gagcagcgatttctgggtata

SEQ ID NO: 414 - RNA, 21 nt, synthetic construct
  source 1..21: mol_type = other RNA
ccagcgatttctgggtattca

SEQ ID NO: 415 - RNA, 21 nt, synthetic construct
  source 1..21: mol_type = other RNA
ggatttctgggtattctttca

SEQ ID NO: 416 - RNA, 21 nt, synthetic construct
  source 1..21: mol_type = other RNA
gtctgggtattctttcaagca

SEQ ID NO: 417 - RNA, 21 nt, synthetic construct
  source 1..21: mol_type = other RNA
gctgggtattctttcaagcaa

SEQ ID NO: 418 - RNA, 21 nt, synthetic construct
  source 1..21: mol_type = other RNA
gtgggtattctttcaagcaaa

SEQ ID NO: 419 - RNA, 21 nt, synthetic construct
  source 1..21: mol_type = other RNA
ggggtattctttcaagcaata

SEQ ID NO: 420 - RNA, 21 nt, synthetic construct
  source 1..21: mol_type = other RNA
cgtattctttcaagcaatgca

SEQ ID NO: 421 - RNA, 21 nt, synthetic construct
  source 1..21: mol_type = other RNA
gttctttcaagcaatgctcaa

SEQ ID NO: 422 - RNA, 21 nt, synthetic construct
  source 1..21: mol_type = other RNA
gtctttcaagcaatgctcaca

SEQ ID NO: 423 - RNA, 21 nt, synthetic construct
  source 1..21: mol_type = other RNA
ccaatgctcacaccaaataaa

SEQ ID NO: 424 - RNA, 21 nt, synthetic construct
  source 1..21: mol_type = other RNA
gaatgctcacaccaaataaga

SEQ ID NO: 425 - RNA, 21 nt, synthetic construct
  source 1..21: mol_type = other RNA
gatgctcacaccaaataagca

SEQ ID NO: 426 - RNA, 21 nt, synthetic construct
  source 1..21: mol_type = other RNA
gcaccaaataagcgcttgcaa

SEQ ID NO: 427 - RNA, 21 nt, synthetic construct
  source 1..21: mol_type = other RNA
gaccaaataagcgcttgcaaa

SEQ ID NO: 428 - RNA, 21 nt, synthetic construct
  source 1..21: mol_type = other RNA
gaaataagcgcttgcaacaaa

SEQ ID NO: 429 - RNA, 21 nt, synthetic construct
  source 1..21: mol_type = other RNA
gaataagcgcttgcaacaaaa

SEQ ID NO: 430 - RNA, 21 nt, synthetic construct
  source 1..21: mol_type = other RNA
ccttgcaacaaagacatttaa

SEQ ID NO: 431 - RNA, 21 nt, synthetic construct
  source 1..21: mol_type = other RNA
ggcaacaaagacatttatgta

SEQ ID NO: 432 - RNA, 21 nt, synthetic construct
  source 1..21: mol_type = other RNA
ccaacaaagacatttatgtga

SEQ ID NO: 433 - RNA, 21 nt, synthetic construct
  source 1..21: mol_type = other RNA
gaacaaagacatttatgtgga

SEQ ID NO: 434 - RNA, 21 nt, synthetic construct
  source 1..21: mol_type = other RNA
gacaaagacatttatgtggaa

SEQ ID NO: 435 - RNA, 21 nt, synthetic construct
  source 1..21: mol_type = other RNA
gaagacatttatgtggaccta

SEQ ID NO: 436 - RNA, 21 nt, synthetic construct
  source 1..21: mol_type = other RNA
gagacatttatgtggacctaa

SEQ ID NO: 437 - RNA, 21 nt, synthetic construct
  source 1..21: mol_type = other RNA
ggacatttatgtggacctaga

SEQ ID NO: 438 - RNA, 21 nt, synthetic construct
  source 1..21: mol_type = other RNA
cacatttatgtggacctagaa

SEQ ID NO: 439 - RNA, 21 nt, synthetic construct
  source 1..21: mol_type = other RNA
gatgtggacctagacatgaaa

SEQ ID NO: 440 - RNA, 21 nt, synthetic construct
  source 1..21: mol_type = other RNA
gtagacatgaagggcataaaa

SEQ ID NO: 441 - RNA, 21 nt, synthetic construct
  source 1..21: mol_type = other RNA
gagacatgaagggcataaaca

SEQ ID NO: 442 - RNA, 21 nt, synthetic construct
  source 1..21: mol_type = other RNA
ggacatgaagggcataaacta

SEQ ID NO: 443 - RNA, 21 nt, synthetic construct
  source 1..21: mol_type = other RNA
gcatgaagggcataaactata

SEQ ID NO: 444 - RNA, 21 nt, synthetic construct
  source 1..21: mol_type = other RNA
gatgaagggcataaactataa

SEQ ID NO: 445 - RNA, 21 nt, synthetic construct
  source 1..21: mol_type = other RNA
gtgaagggcataaactataaa

SEQ ID NO: 446 - RNA, 21 nt, synthetic construct
  source 1..21: mol_type = other RNA
ggaagggcataaactataaca

SEQ ID NO: 447 - RNA, 21 nt, synthetic construct
  source 1..21: mol_type = other RNA
caagggcataaactataacaa

SEQ ID NO: 448 - RNA, 21 nt, synthetic construct
  source 1..21: mol_type = other RNA
gagggcataaactataacaga

SEQ ID NO: 449 - RNA, 21 nt, synthetic construct
  source 1..21: mol_type = other RNA
cggcataaactataacagcta

SEQ ID NO: 450 - RNA, 21 nt, synthetic construct
  source 1..21: mol_type = other RNA
ccataaactataacagctcaa

SEQ ID NO: 451 - RNA, 21 nt, synthetic construct
  source 1..21: mol_type = other RNA
cccaagagtgctcaagaatga

SEQ ID NO: 452 - RNA, 21 nt, synthetic construct
  source 1..21: mol_type = other RNA
gcaagaatgccaagaaagata

SEQ ID NO: 453 - RNA, 21 nt, synthetic construct
  source 1..21: mol_type = other RNA
gaagaatgccaagaaagatga

SEQ ID NO: 454 - RNA, 21 nt, synthetic construct
  source 1..21: mol_type = other RNA
gccactgccactttttcacga

SEQ ID NO: 455 - RNA, 21 nt, synthetic construct
  source 1..21: mol_type = other RNA
gcactgccactttttcacgta

SEQ ID NO: 456 - RNA, 21 nt, synthetic construct
  source 1..21: mol_type = other RNA
gactgccactttttcacgtaa

SEQ ID NO: 457 - RNA, 21 nt, synthetic construct
  source 1..21: mol_type = other RNA
gagaaatctttgtctccttaa

SEQ ID NO: 458 - RNA, 21 nt, synthetic construct
  source 1..21: mol_type = other RNA
gatctttgtctccttaaaaca

SEQ ID NO: 459 - RNA, 21 nt, synthetic construct
  source 1..21: mol_type = other RNA
gtttgtctccttaaaacatca

SEQ ID NO: 460 - RNA, 21 nt, synthetic construct
  source 1..21: mol_type = other RNA
gctccttaaaacatctgagaa

SEQ ID NO: 461 - RNA, 21 nt, synthetic construct
  source 1..21: mol_type = other RNA
gccttaaaacatctgagagta

SEQ ID NO: 462 - RNA, 21 nt, synthetic construct
  source 1..21: mol_type = other RNA
gaaacatctgagagtggatta

SEQ ID NO: 463 - RNA, 21 nt, synthetic construct
  source 1..21: mol_type = other RNA
gcacgcattaaaaagagcaaa

SEQ ID NO: 464 - RNA, 21 nt, synthetic construct
  source 1..21: mol_type = other RNA
gcgcattaaaaagagcaaaga

SEQ ID NO: 465 - RNA, 21 nt, synthetic construct
  source 1..21: mol_type = other RNA
gtaaaaagagcaaagctctta

SEQ ID NO: 466 - RNA, 21 nt, synthetic construct
  source 1..21: mol_type = other RNA
gaagagcaaagctctttctga

SEQ ID NO: 467 - RNA, 21 nt, synthetic construct
  source 1..21: mol_type = other RNA
ggagcaaagctctttctggta

SEQ ID NO: 468 - RNA, 21 nt, synthetic construct
  source 1..21: mol_type = other RNA
cagcaaagctctttctggtta

SEQ ID NO: 469 - RNA, 21 nt, synthetic construct
  source 1..21: mol_type = other RNA
ccaaagctctttctggtttca

SEQ ID NO: 470 - RNA, 21 nt, synthetic construct
  source 1..21: mol_type = other RNA
gaaagctctttctggtttcaa

SEQ ID NO: 471 - RNA, 21 nt, synthetic construct
  source 1..21: mol_type = other RNA
caagaactggatattgttgca

SEQ ID NO: 472 - RNA, 21 nt, synthetic construct
  source 1..21: mol_type = other RNA
gagaactggatattgttgcta

SEQ ID NO: 473 - RNA, 21 nt, synthetic construct
  source 1..21: mol_type = other RNA
gtgccagaaactgtgcaccaa

SEQ ID NO: 474 - RNA, 21 nt, synthetic construct
  source 1..21: mol_type = other RNA
cccagaaactgtgcaccaata

SEQ ID NO: 475 - RNA, 21 nt, synthetic construct
  source 1..21: mol_type = other RNA
gcgctgccagttttttaccta

SEQ ID NO: 476 - RNA, 21 nt, synthetic construct
  source 1..21: mol_type = other RNA
cctgccagttttttacctata

SEQ ID NO: 477 - RNA, 21 nt, synthetic construct
  source 1..21: mol_type = other RNA
gagtgttacttaaagctttca

SEQ ID NO: 478 - RNA, 21 nt, synthetic construct
  source 1..21: mol_type = other RNA
ggtgttacttaaagctttcta

SEQ ID NO: 479 - RNA, 21 nt, synthetic construct
  source 1..21: mol_type = other RNA
ctgttacttaaagctttctta

SEQ ID NO: 480 - RNA, 21 nt, synthetic construct
  source 1..21: mol_type = other RNA
gtacttaaagctttcttcaaa

SEQ ID NO: 481 - RNA, 21 nt, synthetic construct
  source 1..21: mol_type = other RNA
gacttaaagctttcttcaaaa

SEQ ID NO: 482 - RNA, 21 nt, synthetic construct
  source 1..21: mol_type = other RNA
gggatctccaactaaaataca

SEQ ID NO: 483 - RNA, 21 nt, synthetic construct
  source 1..21: mol_type = other RNA
cgatctccaactaaaatacta

SEQ ID NO: 484 - RNA, 21 nt, synthetic construct
  source 1..21: mol_type = other RNA
catctccaactaaaatactta

SEQ ID NO: 485 - RNA, 21 nt, synthetic construct
  source 1..21: mol_type = other RNA
ccatctctggatacacattaa

SEQ ID NO: 486 - RNA, 21 nt, synthetic construct
  source 1..21: mol_type = other RNA
gatctctggatacacattaaa

SEQ ID NO: 487 - RNA, 21 nt, synthetic construct
  source 1..21: mol_type = other RNA
gtctctggatacacattaaga

SEQ ID NO: 488 - RNA, 21 nt, synthetic construct
  source 1..21: mol_type = other RNA
gctctggatacacattaagga

SEQ ID NO: 489 - RNA, 21 nt, synthetic construct
  source 1..21: mol_type = other RNA
gtgtgtaaaatggataatgaa

SEQ ID NO: 490 - RNA, 21 nt, synthetic construct
  source 1..21: mol_type = other RNA
tatcctgaaaagaccttgttc

SEQ ID NO: 491 - RNA, 21 nt, synthetic construct
  source 1..21: mol_type = other RNA
ttcatcctgaaaagaccttgc

SEQ ID NO: 492 - RNA, 21 nt, synthetic construct
  source 1..21: mol_type = other RNA
taatcatcctgaaaagacctc

SEQ ID NO: 493 - RNA, 21 nt, synthetic construct
  source 1..21: mol_type = other RNA
tgaaaatcatcctgaaaagag

SEQ ID NO: 494 - RNA, 21 nt, synthetic construct
  source 1..21: mol_type = other RNA
ttaagaaaatcatcctgaaac

SEQ ID NO: 495 - RNA, 21 nt, synthetic construct
  source 1..21: mol_type = other RNA
tgatataagaaaatcatcctc

SEQ ID NO: 496 - RNA, 21 nt, synthetic construct
  source 1..21: mol_type = other RNA
tttgatataagaaaatcatcg

SEQ ID NO: 497 - RNA, 21 nt, synthetic construct
  source 1..21: mol_type = other RNA
tcattcaccagaaactgaagc

SEQ ID NO: 498 - RNA, 21 nt, synthetic construct
  source 1..21: mol_type = other RNA
tcacattcaccagaaactgac

SEQ ID NO: 499 - RNA, 21 nt, synthetic construct
  source 1..21: mol_type = other RNA
tagtcacacattcaccagaac

SEQ ID NO: 500 - RNA, 21 nt, synthetic construct
  source 1..21: mol_type = other RNA
ttcaggacacaagtaaaccac

SEQ ID NO: 501 - RNA, 21 nt, synthetic construct
  source 1..21: mol_type = other RNA
tttcaggacacaagtaaaccc

SEQ ID NO: 502 - RNA, 21 nt, synthetic construct
  source 1..21: mol_type = other RNA
ttttctgtaacactgtctttg

SEQ ID NO: 503 - RNA, 21 nt, synthetic construct
  source 1..21: mol_type = other RNA
tgtttctgtaacactgtcttc

SEQ ID NO: 504 - RNA, 21 nt, synthetic construct
  source 1..21: mol_type = other RNA
tgtgtttctgtaacactgtcc

SEQ ID NO: 505 - RNA, 21 nt, synthetic construct
  source 1..21: mol_type = other RNA
ttcttggcagtgtttctgtac

SEQ ID NO: 506 - RNA, 21 nt, synthetic construct
  source 1..21: mol_type = other RNA
tctcttggcagtgtttctgtc

SEQ ID NO: 507 - RNA, 21 nt, synthetic construct
  source 1..21: mol_type = other RNA
tttcactcttggcagtgtttg

SEQ ID NO: 508 - RNA, 21 nt, synthetic construct
  source 1..21: mol_type = other RNA
tattcactcttggcagtgttc

SEQ ID NO: 509 - RNA, 21 nt, synthetic construct
  source 1..21: mol_type = other RNA
tcctattcactcttggcagtc

SEQ ID NO: 510 - RNA, 21 nt, synthetic construct
  source 1..21: mol_type = other RNA
tatacccagaaatcgctgctc

SEQ ID NO: 511 - RNA, 21 nt, synthetic construct
  source 1..21: mol_type = other RNA
tgaatacccagaaatcgctgg

SEQ ID NO: 512 - RNA, 21 nt, synthetic construct
  source 1..21: mol_type = other RNA
tgaaagaatacccagaaatcc

SEQ ID NO: 513 - RNA, 21 nt, synthetic construct
  source 1..21: mol_type = other RNA
tgcttgaaagaatacccagac

SEQ ID NO: 514 - RNA, 21 nt, synthetic construct
  source 1..21: mol_type = other RNA
ttgcttgaaagaatacccagc

SEQ ID NO: 515 - RNA, 21 nt, synthetic construct
  source 1..21: mol_type = other RNA
tttgcttgaaagaatacccac

SEQ ID NO: 516 - RNA, 21 nt, synthetic construct
  source 1..21: mol_type = other RNA
tattgcttgaaagaatacccc

SEQ ID NO: 517 - RNA, 21 nt, synthetic construct
  source 1..21: mol_type = other RNA
tgcattgcttgaaagaatacg

SEQ ID NO: 518 - RNA, 21 nt, synthetic construct
  source 1..21: mol_type = other RNA
ttgagcattgcttgaaagaac

SEQ ID NO: 519 - RNA, 21 nt, synthetic construct
  source 1..21: mol_type = other RNA
tgtgagcattgcttgaaagac

SEQ ID NO: 520 - RNA, 21 nt, synthetic construct
  source 1..21: mol_type = other RNA
tttatttggtgtgagcattgg

SEQ ID NO: 521 - RNA, 21 nt, synthetic construct
  source 1..21: mol_type = other RNA
tcttatttggtgtgagcattc

SEQ ID NO: 522 - RNA, 21 nt, synthetic construct
  source 1..21: mol_type = other RNA
tgcttatttggtgtgagcatc

SEQ ID NO: 523 - RNA, 21 nt, synthetic construct
  source 1..21: mol_type = other RNA
ttgcaagcgcttatttggtgc

SEQ ID NO: 524 - RNA, 21 nt, synthetic construct
  source 1..21: mol_type = other RNA
tttgcaagcgcttatttggtc

SEQ ID NO: 525 - RNA, 21 nt, synthetic construct
  source 1..21: mol_type = other RNA
tttgttgcaagcgcttatttc

SEQ ID NO: 526 - RNA, 21 nt, synthetic construct
  source 1..21: mol_type = other RNA
ttttgttgcaagcgcttattc

SEQ ID NO: 527 - RNA, 21 nt, synthetic construct
  source 1..21: mol_type = other RNA
ttaaatgtctttgttgcaagg

SEQ ID NO: 528 - RNA, 21 nt, synthetic construct
  source 1..21: mol_type = other RNA
tacataaatgtctttgttgcc

SEQ ID NO: 529 - RNA, 21 nt, synthetic construct
  source 1..21: mol_type = other RNA
tcacataaatgtctttgttgg

SEQ ID NO: 530 - RNA, 21 nt, synthetic construct
  source 1..21: mol_type = other RNA
tccacataaatgtctttgttc

SEQ ID NO: 531 - RNA, 21 nt, synthetic construct
  source 1..21: mol_type = other RNA
ttccacataaatgtctttgtc

SEQ ID NO: 532 - RNA, 21 nt, synthetic construct
  source 1..21: mol_type = other RNA
taggtccacataaatgtcttc

SEQ ID NO: 533 - RNA, 21 nt, synthetic construct
  source 1..21: mol_type = other RNA
ttaggtccacataaatgtctc

SEQ ID NO: 534 - RNA, 21 nt, synthetic construct
  source 1..21: mol_type = other RNA
tctaggtccacataaatgtcc

SEQ ID NO: 535 - RNA, 21 nt, synthetic construct
  source 1..21: mol_type = other RNA
ttctaggtccacataaatgtg

SEQ ID NO: 536 - RNA, 21 nt, synthetic construct
  source 1..21: mol_type = other RNA
tttcatgtctaggtccacatc

SEQ ID NO: 537 - RNA, 21 nt, synthetic construct
  source 1..21: mol_type = other RNA
ttttatgcccttcatgtctac

SEQ ID NO: 538 - RNA, 21 nt, synthetic construct
  source 1..21: mol_type = other RNA
tgtttatgcccttcatgtctc

SEQ ID NO: 539 - RNA, 21 nt, synthetic construct
  source 1..21: mol_type = other RNA
tagtttatgcccttcatgtcc

SEQ ID NO: 540 - RNA, 21 nt, synthetic construct
  source 1..21: mol_type = other RNA
tatagtttatgcccttcatgc

SEQ ID NO: 541 - RNA, 21 nt, synthetic construct
  source 1..21: mol_type = other RNA
ttatagtttatgcccttcatc

SEQ ID NO: 542 - RNA, 21 nt, synthetic construct
  source 1..21: mol_type = other RNA
tttatagtttatgcccttcac

SEQ ID NO: 543 - RNA, 21 nt, synthetic construct
  source 1..21: mol_type = other RNA
tgttatagtttatgcccttcc

SEQ ID NO: 544 - RNA, 21 nt, synthetic construct
  source 1..21: mol_type = other RNA
ttgttatagtttatgcccttg

SEQ ID NO: 545 - RNA, 21 nt, synthetic construct
  source 1..21: mol_type = other RNA
tctgttatagtttatgccctc

SEQ ID NO: 546 - RNA, 21 nt, synthetic construct
  source 1..21: mol_type = other RNA
tagctgttatagtttatgccg

SEQ ID NO: 547 - RNA, 21 nt, synthetic construct
  source 1..21: mol_type = other RNA
ttgagctgttatagtttatgg

SEQ ID NO: 548 - RNA, 21 nt, synthetic construct
  source 1..21: mol_type = other RNA
tcattcttgagcactcttggg

SEQ ID NO: 549 - RNA, 21 nt, synthetic construct
  source 1..21: mol_type = other RNA
tatctttcttggcattcttgc

SEQ ID NO: 550 - RNA, 21 nt, synthetic construct
  source 1..21: mol_type = other RNA
tcatctttcttggcattcttc

SEQ ID NO: 551 - RNA, 21 nt, synthetic construct
  source 1..21: mol_type = other RNA
tcgtgaaaaagtggcagtggc

SEQ ID NO: 552 - RNA, 21 nt, synthetic construct
  source 1..21: mol_type = other RNA
tacgtgaaaaagtggcagtgc

SEQ ID NO: 553 - RNA, 21 nt, synthetic construct
  source 1..21: mol_type = other RNA
ttacgtgaaaaagtggcagtc

SEQ ID NO: 554 - RNA, 21 nt, synthetic construct
  source 1..21: mol_type = other RNA
ttaaggagacaaagatttctc

SEQ ID NO: 555 - RNA, 21 nt, synthetic construct
  source 1..21: mol_type = other RNA
tgttttaaggagacaaagatc

SEQ ID NO: 556 - RNA, 21 nt, synthetic construct
  source 1..21: mol_type = other RNA
tgatgttttaaggagacaaac

SEQ ID NO: 557 - RNA, 21 nt, synthetic construct
  source 1..21: mol_type = other RNA
ttctcagatgttttaaggagc

SEQ ID NO: 558 - RNA, 21 nt, synthetic construct
  source 1..21: mol_type = other RNA
tactctcagatgttttaaggc

SEQ ID NO: 559 - RNA, 21 nt, synthetic construct
  source 1..21: mol_type = other RNA
taatccactctcagatgtttc

SEQ ID NO: 560 - RNA, 21 nt, synthetic construct
  source 1..21: mol_type = other RNA
tttgctctttttaatgcgtgc

SEQ ID NO: 561 - RNA, 21 nt, synthetic construct
  source 1..21: mol_type = other RNA
tctttgctctttttaatgcgc

SEQ ID NO: 562 - RNA, 21 nt, synthetic construct
  source 1..21: mol_type = other RNA
taagagctttgctctttttac

SEQ ID NO: 563 - RNA, 21 nt, synthetic construct
  source 1..21: mol_type = other RNA
tcagaaagagctttgctcttc

SEQ ID NO: 564 - RNA, 21 nt, synthetic construct
  source 1..21: mol_type = other RNA
taccagaaagagctttgctcc

SEQ ID NO: 565 - RNA, 21 nt, synthetic construct
  source 1..21: mol_type = other RNA
taaccagaaagagctttgctg

SEQ ID NO: 566 - RNA, 21 nt, synthetic construct
  source 1..21: mol_type = other RNA
tgaaaccagaaagagctttgg

SEQ ID NO: 567 - RNA, 21 nt, synthetic construct
  source 1..21: mol_type = other RNA
ttgaaaccagaaagagctttc

SEQ ID NO: 568 - RNA, 21 nt, synthetic construct
  source 1..21: mol_type = other RNA
tgcaacaatatccagttcttg

SEQ ID NO: 569 - RNA, 21 nt, synthetic construct
  source 1..21: mol_type = other RNA
tagcaacaatatccagttctc

SEQ ID NO: 570 - RNA, 21 nt, synthetic construct
  source 1..21: mol_type = other RNA
ttggtgcacagtttctggcac

SEQ ID NO: 571 - RNA, 21 nt, synthetic construct
  source 1..21: mol_type = other RNA
tattggtgcacagtttctggg

SEQ ID NO: 572 - RNA, 21 nt, synthetic construct
  source 1..21: mol_type = other RNA
taggtaaaaaactggcagcgc

SEQ ID NO: 573 - RNA, 21 nt, synthetic construct
  source 1..21: mol_type = other RNA
tataggtaaaaaactggcagg

SEQ ID NO: 574 - RNA, 21 nt, synthetic construct
  source 1..21: mol_type = other RNA
tgaaagctttaagtaacactc

SEQ ID NO: 575 - RNA, 21 nt, synthetic construct
  source 1..21: mol_type = other RNA
tagaaagctttaagtaacacc

SEQ ID NO: 576 - RNA, 21 nt, synthetic construct
  source 1..21: mol_type = other RNA
taagaaagctttaagtaacag

SEQ ID NO: 577 - RNA, 21 nt, synthetic construct
  source 1..21: mol_type = other RNA
tttgaagaaagctttaagtac

SEQ ID NO: 578 - RNA, 21 nt, synthetic construct
  source 1..21: mol_type = other RNA
ttttgaagaaagctttaagtc

SEQ ID NO: 579 - RNA, 21 nt, synthetic construct
  source 1..21: mol_type = other RNA
tgtattttagttggagatccc

SEQ ID NO: 580 - RNA, 21 nt, synthetic construct
  source 1..21: mol_type = other RNA
tagtattttagttggagatcg

SEQ ID NO: 581 - RNA, 21 nt, synthetic construct
  source 1..21: mol_type = other RNA
taagtattttagttggagatg

SEQ ID NO: 582 - RNA, 21 nt, synthetic construct
  source 1..21: mol_type = other RNA
ttaatgtgtatccagagatgg

SEQ ID NO: 583 - RNA, 21 nt, synthetic construct
  source 1..21: mol_type = other RNA
tttaatgtgtatccagagatc

SEQ ID NO: 584 - RNA, 21 nt, synthetic construct
  source 1..21: mol_type = other RNA
tcttaatgtgtatccagagac

SEQ ID NO: 585 - RNA, 21 nt, synthetic construct
  source 1..21: mol_type = other RNA
tccttaatgtgtatccagagc

SEQ ID NO: 586 - RNA, 21 nt, synthetic construct
  source 1..21: mol_type = other RNA
ttcattatccattttacacac

SEQ ID NO: 587 - RNA, 21 nt, synthetic construct
  source 1..21: mol_type = other RNA
gcaaggtcttttcaggatgaa

SEQ ID NO: 588 - RNA, 21 nt, synthetic construct
  source 1..21: mol_type = other RNA
gaggatgattttcttatatca

SEQ ID NO: 589 - RNA, 21 nt, synthetic construct
  source 1..21: mol_type = other RNA
gggtttacttgtgtcctgaaa

SEQ ID NO: 590 - RNA, 21 nt, synthetic construct
  source 1..21: mol_type = other RNA
caaagacagtgttacagaaaa

SEQ ID NO: 591 - RNA, 21 nt, synthetic construct
  source 1..21: mol_type = other RNA
gctgggtattctttcaagcaa

SEQ ID NO: 592 - RNA, 21 nt, synthetic construct
  source 1..21: mol_type = other RNA
gaataagcgcttgcaacaaaa

SEQ ID NO: 593 - RNA, 21 nt, synthetic construct
  source 1..21: mol_type = other RNA
gacaaagacatttatgtggaa

SEQ ID NO: 594 - RNA, 21 nt, synthetic construct
  source 1..21: mol_type = other RNA
gaagacatttatgtggaccta

SEQ ID NO: 595 - RNA, 21 nt, synthetic construct
  source 1..21: mol_type = other RNA
gatgaagggcataaactataa

SEQ ID NO: 596 - RNA, 21 nt, synthetic construct
  source 1..21: mol_type = other RNA
gactgccactttttcacgtaa

SEQ ID NO: 597 - RNA, 21 nt, synthetic construct
  source 1..21: mol_type = other RNA
gccttaaaacatctgagagta

SEQ ID NO: 598 - RNA, 21 nt, synthetic construct
  source 1..21: mol_type = other RNA
ccaaagctctttctggtttca

SEQ ID NO: 599 - RNA, 21 nt, synthetic construct
  source 1..21: mol_type = other RNA
gtacttaaagctttcttcaaa

SEQ ID NO: 600 - RNA, 21 nt, synthetic construct
  source 1..21: mol_type = other RNA
gaggatgattttcttatatca

SEQ ID NO: 601 - RNA, 21 nt, synthetic construct
  source 1..21: mol_type = other RNA
cgatgattttcttatatcaaa

SEQ ID NO: 602 - RNA, 21 nt, synthetic construct
  source 1..21: mol_type = other RNA
gcttcagtttctggtgaatga

SEQ ID NO: 603 - RNA, 21 nt, synthetic construct
  source 1..21: mol_type = other RNA
gttctggtgaatgtgtgacta

SEQ ID NO: 604 - RNA, 21 nt, synthetic construct
  source 1..21: mol_type = other RNA
gtggtttacttgtgtcctgaa

SEQ ID NO: 605 - RNA, 21 nt, synthetic construct
  source 1..21: mol_type = other RNA
gaagacagtgttacagaaaca

SEQ ID NO: 606 - RNA, 21 nt, synthetic construct
  source 1..21: mol_type = other RNA
ggacagtgttacagaaacaca

SEQ ID NO: 607 - RNA, 21 nt, synthetic construct
  source 1..21: mol_type = other RNA
caaacactgccaagagtgaaa

SEQ ID NO: 608 - RNA, 21 nt, synthetic construct
  source 1..21: mol_type = other RNA
gagcagcgatttctgggtata

SEQ ID NO: 609 - RNA, 21 nt, synthetic construct
  source 1..21: mol_type = other RNA
ccagcgatttctgggtattca

SEQ ID NO: 610 - RNA, 21 nt, synthetic construct
  source 1..21: mol_type = other RNA
gactgccactttttcacgtaa

SEQ ID NO: 611 - RNA, 21 nt, synthetic construct
  source 1..21: mol_type = other RNA
gctgggtattctttcaagcaa

SEQ ID NO: 612 - RNA, 21 nt, synthetic construct
  source 1..21: mol_type = other RNA
ggggtattctttcaagcaata

SEQ ID NO: 613 - RNA, 21 nt, synthetic construct
  source 1..21: mol_type = other RNA
gtctttcaagcaatgctcaca

SEQ ID NO: 614 - RNA, 21 nt, synthetic construct
  source 1..21: mol_type = other RNA
gaaataagcgcttgcaacaaa

SEQ ID NO: 615 - RNA, 21 nt, synthetic construct
  source 1..21: mol_type = other RNA
ccttgcaacaaagacatttaa

SEQ ID NO: 616 - RNA, 21 nt, synthetic construct
  source 1..21: mol_type = other RNA
gaacaaagacatttatgtgga

SEQ ID NO: 617 - RNA, 21 nt, synthetic construct
  source 1..21: mol_type = other RNA
ggacatttatgtggacctaga

SEQ ID NO: 618 - RNA, 21 nt, synthetic construct
  source 1..21: mol_type = other RNA
gatgtggacctagacatgaaa

SEQ ID NO: 619 - RNA, 21 nt, synthetic construct
  source 1..21: mol_type = other RNA
ggacatgaagggcataaacta

SEQ ID NO: 620 - RNA, 21 nt, synthetic construct
  source 1..21: mol_type = other RNA
gcatgaagggcataaactata

SEQ ID NO: 621 - RNA, 21 nt, synthetic construct
  source 1..21: mol_type = other RNA
caagggcataaactataacaa

SEQ ID NO: 622 - RNA, 21 nt, synthetic construct
  source 1..21: mol_type = other RNA
cccaagagtgctcaagaatga

SEQ ID NO: 623 - RNA, 21 nt, synthetic construct
  source 1..21: mol_type = other RNA
gagaaatctttgtctccttaa

SEQ ID NO: 624 - RNA, 21 nt, synthetic construct
  source 1..21: mol_type = other RNA
gtttgtctccttaaaacatca

SEQ ID NO: 625 - RNA, 21 nt, synthetic construct
  source 1..21: mol_type = other RNA
gtaaaaagagcaaagctctta

SEQ ID NO: 626 - RNA, 21 nt, synthetic construct
  source 1..21: mol_type = other RNA
caagaactggatattgttgca

SEQ ID NO: 627 - RNA, 21 nt, synthetic construct
  source 1..21: mol_type = other RNA
cctgccagttttttacctata

SEQ ID NO: 628 - RNA, 21 nt, synthetic construct
  source 1..21: mol_type = other RNA
gagtgttacttaaagctttca

SEQ ID NO: 629 - RNA, 21 nt, synthetic construct
  source 1..21: mol_type = other RNA
ctgttacttaaagctttctta

SEQ ID NO: 630 - RNA, 21 nt, synthetic construct
  source 1..21: mol_type = other RNA
gtacttaaagctttcttcaaa

SEQ ID NO: 631 - RNA, 21 nt, synthetic construct
  source 1..21: mol_type = other RNA
gacttaaagctttcttcaaaa

SEQ ID NO: 632 - RNA, 21 nt, synthetic construct
  source 1..21: mol_type = other RNA
ccatctctggatacacattaa

SEQ ID NO: 633 - RNA, 21 nt, synthetic construct
  source 1..21: mol_type = other RNA
gtcagtttctggtgaatgtga

SEQ ID NO: 634 - RNA, 21 nt, synthetic construct
  source 1..21: mol_type = other RNA
gtctgggtattctttcaagca

SEQ ID NO: 635 - RNA, 21 nt, synthetic construct
  source 1..21: mol_type = other RNA
gttctttcaagcaatgctcaa

SEQ ID NO: 636 - RNA, 21 nt, synthetic construct
  source 1..21: mol_type = other RNA
ccaatgctcacaccaaataaa

SEQ ID NO: 637 - RNA, 21 nt, synthetic construct
  source 1..21: mol_type = other RNA
gaatgctcacaccaaataaga

SEQ ID NO: 638 - RNA, 21 nt, synthetic construct
  source 1..21: mol_type = other RNA
gatgctcacaccaaataagca

SEQ ID NO: 639 - RNA, 21 nt, synthetic construct
  source 1..21: mol_type = other RNA
gcaccaaataagcgcttgcaa

SEQ ID NO: 640 - RNA, 21 nt, synthetic construct
  source 1..21: mol_type = other RNA
gaccaaataagcgcttgcaaa

SEQ ID NO: 641 - RNA, 21 nt, synthetic construct
  source 1..21: mol_type = other RNA
gccactgccactttttcacga

SEQ ID NO: 642 - RNA, 21 nt, synthetic construct
  source 1..21: mol_type = other RNA
gcactgccactttttcacgta

SEQ ID NO: 643 - RNA, 21 nt, synthetic construct
  source 1..21: mol_type = other RNA
ggtgttacttaaagctttcta

SEQ ID NO: 644 - RNA, 21 nt, synthetic construct
  source 1..21: mol_type = other RNA
gcttcagtttctggtgaatga

SEQ ID NO: 645 - RNA, 21 nt, synthetic construct
  source 1..21: mol_type = other RNA
gttctggtgaatgtgtgacta

SEQ ID NO: 646 - RNA, 21 nt, synthetic construct
  source 1..21: mol_type = other RNA
ccagcgatttctgggtattca

SEQ ID NO: 647 - RNA, 21 nt, synthetic construct
  source 1..21: mol_type = other RNA
gactgccactttttcacgtaa

SEQ ID NO: 648 - RNA, 21 nt, synthetic construct
  source 1..21: mol_type = other RNA
gctgggtattctttcaagcaa

SEQ ID NO: 649 - RNA, 21 nt, synthetic construct
  source 1..21: mol_type = other RNA
gaaataagcgcttgcaacaaa

SEQ ID NO: 650 - RNA, 21 nt, synthetic construct
  source 1..21: mol_type = other RNA
gagtgttacttaaagctttca

SEQ ID NO: 651 - RNA, 21 nt, synthetic construct
  source 1..21: mol_type = other RNA
ttcatcctgaaaagaccttgc

SEQ ID NO: 652 - RNA, 21 nt, synthetic construct
  source 1..21: mol_type = other RNA
tgatataagaaaatcatcctc

SEQ ID NO: 653 - RNA, 21 nt, synthetic construct
  source 1..21: mol_type = other RNA
tttcaggacacaagtaaaccc

SEQ ID NO: 654 - RNA, 21 nt, synthetic construct
  source 1..21: mol_type = other RNA
ttttctgtaacactgtctttg

SEQ ID NO: 655 - RNA, 21 nt, synthetic construct
  source 1..21: mol_type = other RNA
ttgcttgaaagaatacccagc

SEQ ID NO: 656 - RNA, 21 nt, synthetic construct
  source 1..21: mol_type = other RNA
ttttgttgcaagcgcttattc

SEQ ID NO: 657 - RNA, 21 nt, synthetic construct
  source 1..21: mol_type = other RNA
ttccacataaatgtctttgtc

SEQ ID NO: 658 - RNA, 21 nt, synthetic construct
  source 1..21: mol_type = other RNA
taggtccacataaatgtcttc

SEQ ID NO: 659 - RNA, 21 nt, synthetic construct
  source 1..21: mol_type = other RNA
ttatagtttatgcccttcatc

SEQ ID NO: 660 - RNA, 21 nt, synthetic construct
  source 1..21: mol_type = other RNA
ttacgtgaaaaagtggcagtc

SEQ ID NO: 661 - RNA, 21 nt, synthetic construct
  source 1..21: mol_type = other RNA
tactctcagatgttttaaggc

SEQ ID NO: 662 - RNA, 21 nt, synthetic construct
  source 1..21: mol_type = other RNA
tgaaaccagaaagagctttgg

SEQ ID NO: 663 - RNA, 21 nt, synthetic construct
  source 1..21: mol_type = other RNA
tttgaagaaagctttaagtac

SEQ ID NO: 664 - RNA, 21 nt, synthetic construct
  source 1..21: mol_type = other RNA
tgatataagaaaatcatcctc

SEQ ID NO: 665 - RNA, 21 nt, synthetic construct
  source 1..21: mol_type = other RNA
tttgatataagaaaatcatcg

SEQ ID NO: 666 - RNA, 21 nt, synthetic construct
  source 1..21: mol_type = other RNA
tcattcaccagaaactgaagc

SEQ ID NO: 667 - RNA, 21 nt, synthetic construct
  source 1..21: mol_type = other RNA
tagtcacacattcaccagaac

SEQ ID NO: 668 - RNA, 21 nt, synthetic construct
  source 1..21: mol_type = other RNA
ttcaggacacaagtaaaccac

SEQ ID NO: 669 - RNA, 21 nt, synthetic construct
  source 1..21: mol_type = other RNA
tgtttctgtaacactgtcttc

SEQ ID NO: 670 - RNA, 21 nt, synthetic construct
  source 1..21: mol_type = other RNA
tgtgtttctgtaacactgtcc

SEQ ID NO: 671 - RNA, 21 nt, synthetic construct
  source 1..21: mol_type = other RNA
tttcactcttggcagtgtttg

SEQ ID NO: 672 - RNA, 21 nt, synthetic construct
  source 1..21: mol_type = other RNA
tatacccagaaatcgctgctc

SEQ ID NO: 673 - RNA, 21 nt, synthetic construct
  source 1..21: mol_type = other RNA
tgaatacccagaaatcgctgg

SEQ ID NO: 674 - RNA, 21 nt, synthetic construct
  source 1..21: mol_type = other RNA
ttacgtgaaaaagtggcagtc

SEQ ID NO: 675 - RNA, 21 nt, synthetic construct
  source 1..21: mol_type = other RNA
ttgcttgaaagaatacccagc

SEQ ID NO: 676 - RNA, 21 nt, synthetic construct
  source 1..21: mol_type = other RNA
tattgcttgaaagaatacccc

SEQ ID NO: 677 - RNA, 21 nt, synthetic construct
  source 1..21: mol_type = other RNA
tgtgagcattgcttgaaagac

SEQ ID NO: 678 - RNA, 21 nt, synthetic construct
  source 1..21: mol_type = other RNA
tttgttgcaagcgcttatttc

SEQ ID NO: 679 - RNA, 21 nt, synthetic construct
  source 1..21: mol_type = other RNA
ttaaatgtctttgttgcaagg

SEQ ID NO: 680 - RNA, 21 nt, synthetic construct
  source 1..21: mol_type = other RNA
tccacataaatgtctttgttc

SEQ ID NO: 681 - RNA, 21 nt, synthetic construct
  source 1..21: mol_type = other RNA
tctaggtccacataaatgtcc

SEQ ID NO: 682 - RNA, 21 nt, synthetic construct
  source 1..21: mol_type = other RNA
tttcatgtctaggtccacatc

SEQ ID NO: 683 - RNA, 21 nt, synthetic construct
  source 1..21: mol_type = other RNA
tagtttatgcccttcatgtcc

SEQ ID NO: 684 - RNA, 21 nt, synthetic construct
  source 1..21: mol_type = other RNA
tatagtttatgcccttcatgc

SEQ ID NO: 685 - RNA, 21 nt, synthetic construct
  source 1..21: mol_type = other RNA
ttgttatagtttatgcccttg

SEQ ID NO: 686 - RNA, 21 nt, synthetic construct
  source 1..21: mol_type = other RNA
tcattcttgagcactcttggg

SEQ ID NO: 687 - RNA, 21 nt, synthetic construct
  source 1..21: mol_type = other RNA
ttaaggagacaaagatttctc

SEQ ID NO: 688 - RNA, 21 nt, synthetic construct
  source 1..21: mol_type = other RNA
tgatgttttaaggagacaaac

SEQ ID NO: 689 - RNA, 21 nt, synthetic construct
  source 1..21: mol_type = other RNA
taagagctttgctctttttac

SEQ ID NO: 690 - RNA, 21 nt, synthetic construct
  source 1..21: mol_type = other RNA
tgcaacaatatccagttcttg

SEQ ID NO: 691 - RNA, 21 nt, synthetic construct
  source 1..21: mol_type = other RNA
tataggtaaaaaactggcagg

SEQ ID NO: 692 - RNA, 21 nt, synthetic construct
  source 1..21: mol_type = other RNA
tgaaagctttaagtaacactc

SEQ ID NO: 693 - RNA, 21 nt, synthetic construct
  source 1..21: mol_type = other RNA
taagaaagctttaagtaacag

SEQ ID NO: 694 - RNA, 21 nt, synthetic construct
  source 1..21: mol_type = other RNA
tttgaagaaagctttaagtac

SEQ ID NO: 695 - RNA, 21 nt, synthetic construct
  source 1..21: mol_type = other RNA
ttttgaagaaagctttaagtc

SEQ ID NO: 696 - RNA, 21 nt, synthetic construct
  source 1..21: mol_type = other RNA
ttaatgtgtatccagagatgg

SEQ ID NO: 697 - RNA, 21 nt, synthetic construct
  source 1..21: mol_type = other RNA
tcacattcaccagaaactgac

SEQ ID NO: 698 - RNA, 21 nt, synthetic construct
  source 1..21: mol_type = other RNA
tgcttgaaagaatacccagac

SEQ ID NO: 699 - RNA, 21 nt, synthetic construct
  source 1..21: mol_type = other RNA
ttgagcattgcttgaaagaac

SEQ ID NO: 700 - RNA, 21 nt, synthetic construct
  source 1..21: mol_type = other RNA
tttatttggtgtgagcattgg

SEQ ID NO: 701 - RNA, 21 nt, synthetic construct
  source 1..21: mol_type = other RNA
tcttatttggtgtgagcattc

SEQ ID NO: 702 - RNA, 21 nt, synthetic construct
  source 1..21: mol_type = other RNA
tgcttatttggtgtgagcatc

SEQ ID NO: 703 - RNA, 21 nt, synthetic construct
  source 1..21: mol_type = other RNA
ttgcaagcgcttatttggtgc

SEQ ID NO: 704 - RNA, 21 nt, synthetic construct
  source 1..21: mol_type = other RNA
tttgcaagcgcttatttggtc

SEQ ID NO: 705 - RNA, 21 nt, synthetic construct
  source 1..21: mol_type = other RNA
tcgtgaaaaagtggcagtggc

SEQ ID NO: 706 - RNA, 21 nt, synthetic construct
  source 1..21: mol_type = other RNA
tacgtgaaaaagtggcagtgc

SEQ ID NO: 707 - RNA, 21 nt, synthetic construct
  source 1..21: mol_type = other RNA
tagaaagctttaagtaacacc

SEQ ID NO: 708 - RNA, 21 nt, synthetic construct
  source 1..21: mol_type = other RNA
tcattcaccagaaactgaagc

SEQ ID NO: 709 - RNA, 21 nt, synthetic construct
  source 1..21: mol_type = other RNA
tagtcacacattcaccagaac

SEQ ID NO: 710 - RNA, 21 nt, synthetic construct
  source 1..21: mol_type = other RNA
tgaatacccagaaatcgctgg

SEQ ID NO: 711 - RNA, 21 nt, synthetic construct
  source 1..21: mol_type = other RNA
ttacgtgaaaaagtggcagtc

SEQ ID NO: 712 - RNA, 21 nt, synthetic construct
  source 1..21: mol_type = other RNA
ttgcttgaaagaatacccagc

SEQ ID NO: 713 - RNA, 21 nt, synthetic construct
  source 1..21: mol_type = other RNA
tttgttgcaagcgcttatttc

SEQ ID NO: 714 - RNA, 21 nt, synthetic construct
  source 1..21: mol_type = other RNA
tgaaagctttaagtaacactc

SEQ ID NO: 715 - RNA, 21 nt, synthetic construct
  source 1..21: mol_type = other RNA
gttcaggatgattttcttata

SEQ ID NO: 716 - RNA, 21 nt, synthetic construct
  source 1..21: mol_type = other RNA
gtcaggatgattttcttataa

SEQ ID NO: 717 - RNA, 21 nt, synthetic construct
  source 1..21: mol_type = other RNA
gagtttctggtgaatgtgtga

SEQ ID NO: 718 - RNA, 21 nt, synthetic construct
  source 1..21: mol_type = other RNA
cggtattctttcaagcaatga

SEQ ID NO: 719 - RNA, 21 nt, synthetic construct
  source 1..21: mol_type = other RNA
gcaagcaatgctcacaccaaa

SEQ ID NO: 720 - RNA, 21 nt, synthetic construct
  source 1..21: mol_type = other RNA
gaagcaatgctcacaccaaaa

SEQ ID NO: 721 - RNA, 21 nt, synthetic construct
  source 1..21: mol_type = other RNA
gtgcaacaaagacatttatga

SEQ ID NO: 722 - RNA, 21 nt, synthetic construct
  source 1..21: mol_type = other RNA
cacatgaagggcataaactaa

SEQ ID NO: 723 - RNA, 21 nt, synthetic construct
  source 1..21: mol_type = other RNA
ggggcataaactataacagca

SEQ ID NO: 724 - RNA, 21 nt, synthetic construct
  source 1..21: mol_type = other RNA
gtgagtgtaccaccaaaatca

SEQ ID NO: 725 - RNA, 21 nt, synthetic construct
  source 1..21: mol_type = other RNA
ggagtgtaccaccaaaatcaa

SEQ ID NO: 726 - RNA, 21 nt, synthetic construct
  source 1..21: mol_type = other RNA
cagtgtaccaccaaaatcaaa

SEQ ID NO: 727 - RNA, 21 nt, synthetic construct
  source 1..21: mol_type = other RNA
cggtagagtcacctaagatta

SEQ ID NO: 728 - RNA, 21 nt, synthetic construct
  source 1..21: mol_type = other RNA
ctcacctaagattttgcgtga

SEQ ID NO: 729 - RNA, 21 nt, synthetic construct
  source 1..21: mol_type = other RNA
gcacctaagattttgcgtgta

SEQ ID NO: 730 - RNA, 21 nt, synthetic construct
  source 1..21: mol_type = other RNA
gcctaagattttgcgtgtcta

SEQ ID NO: 731 - RNA, 21 nt, synthetic construct
  source 1..21: mol_type = other RNA
gctaagattttgcgtgtctaa

SEQ ID NO: 732 - RNA, 21 nt, synthetic construct
  source 1..21: mol_type = other RNA
gaagattttgcgtgtctacaa

SEQ ID NO: 733 - RNA, 21 nt, synthetic construct
  source 1..21: mol_type = other RNA
gcagtggcattttaaatcaaa

SEQ ID NO: 734 - RNA, 21 nt, synthetic construct
  source 1..21: mol_type = other RNA
gagtggcattttaaatcaata

SEQ ID NO: 735 - RNA, 21 nt, synthetic construct
  source 1..21: mol_type = other RNA
gtttaaatcaatctgaaataa

SEQ ID NO: 736 - RNA, 21 nt, synthetic construct
  source 1..21: mol_type = other RNA
gtaaatcaatctgaaataaaa

SEQ ID NO: 737 - RNA, 21 nt, synthetic construct
  source 1..21: mol_type = other RNA
gcacatctttctttggggtta

SEQ ID NO: 738 - RNA, 21 nt, synthetic construct
  source 1..21: mol_type = other RNA
gcatctttctttggggttcaa

SEQ ID NO: 739 - RNA, 21 nt, synthetic construct
  source 1..21: mol_type = other RNA
gatctttctttggggttcaaa

SEQ ID NO: 740 - RNA, 21 nt, synthetic construct
  source 1..21: mol_type = other RNA
gttctttggggttcaagaaaa

SEQ ID NO: 741 - RNA, 21 nt, synthetic construct
  source 1..21: mol_type = other RNA
gtctttggggttcaagaaata

SEQ ID NO: 742 - RNA, 21 nt, synthetic construct
  source 1..21: mol_type = other RNA
gctttggggttcaagaaataa

SEQ ID NO: 743 - RNA, 21 nt, synthetic construct
  source 1..21: mol_type = other RNA
cggttcaagaaataataatca

SEQ ID NO: 744 - RNA, 21 nt, synthetic construct
  source 1..21: mol_type = other RNA
ggaaataataatccatgatca

SEQ ID NO: 745 - RNA, 21 nt, synthetic construct
  source 1..21: mol_type = other RNA
gcagtataaaatggcagaaaa

SEQ ID NO: 746 - RNA, 21 nt, synthetic construct
  source 1..21: mol_type = other RNA
gagtataaaatggcagaaaga

SEQ ID NO: 747 - RNA, 21 nt, synthetic construct
  source 1..21: mol_type = other RNA
cggtatgatattgccttgtta

SEQ ID NO: 748 - RNA, 21 nt, synthetic construct
  source 1..21: mol_type = other RNA
ggatattgccttgttgaaaca

SEQ ID NO: 749 - RNA, 21 nt, synthetic construct
  source 1..21: mol_type = other RNA
catattgccttgttgaaacta

SEQ ID NO: 750 - RNA, 21 nt, synthetic construct
  source 1..21: mol_type = other RNA
gattgccttgttgaaactgga

SEQ ID NO: 751 - RNA, 21 nt, synthetic construct
  source 1..21: mol_type = other RNA
cccttgttgaaactggaaaca

SEQ ID NO: 752 - RNA, 21 nt, synthetic construct
  source 1..21: mol_type = other RNA
ggttgaaactggaaaccacaa

SEQ ID NO: 753 - RNA, 21 nt, synthetic construct
  source 1..21: mol_type = other RNA
gggaaaccacagtgaattaca

SEQ ID NO: 754 - RNA, 21 nt, synthetic construct
  source 1..21: mol_type = other RNA
gaaccacagtgaattacacaa

SEQ ID NO: 755 - RNA, 21 nt, synthetic construct
  source 1..21: mol_type = other RNA
gccacagtgaattacacagaa

SEQ ID NO: 756 - RNA, 21 nt, synthetic construct
  source 1..21: mol_type = other RNA
gcaaaatactctccagaaaga

SEQ ID NO: 757 - RNA, 21 nt, synthetic construct
  source 1..21: mol_type = other RNA
gctctccagaaagccaagata

SEQ ID NO: 758 - RNA, 21 nt, synthetic construct
  source 1..21: mol_type = other RNA
gaagccaagatacccttagta

SEQ ID NO: 759 - RNA, 21 nt, synthetic construct
  source 1..21: mol_type = other RNA
caagagatacagaggacataa

SEQ ID NO: 760 - RNA, 21 nt, synthetic construct
  source 1..21: mol_type = other RNA
cagatacagaggacataaaaa

SEQ ID NO: 761 - RNA, 21 nt, synthetic construct
  source 1..21: mol_type = other RNA
gacagaggacataaaataaca

SEQ ID NO: 762 - RNA, 21 nt, synthetic construct
  source 1..21: mol_type = other RNA
gcagaggacataaaataacca

SEQ ID NO: 763 - RNA, 21 nt, synthetic construct
  source 1..21: mol_type = other RNA
caggacataaaataacccata

SEQ ID NO: 764 - RNA, 21 nt, synthetic construct
  source 1..21: mol_type = other RNA
cacataaaataacccataaga

SEQ ID NO: 765 - RNA, 21 nt, synthetic construct
  source 1..21: mol_type = other RNA
gcataaaataacccataagaa

SEQ ID NO: 766 - RNA, 21 nt, synthetic construct
  source 1..21: mol_type = other RNA
gacccataagatgatctgtga

SEQ ID NO: 767 - RNA, 21 nt, synthetic construct
  source 1..21: mol_type = other RNA
cgactggattctggagaaaaa

SEQ ID NO: 768 - RNA, 21 nt, synthetic construct
  source 1..21: mol_type = other RNA
cactggattctggagaaaaca

SEQ ID NO: 769 - RNA, 21 nt, synthetic construct
  source 1..21: mol_type = other RNA
gtcattcagaggaggatgaaa

SEQ ID NO: 770 - RNA, 21 nt, synthetic construct
  source 1..21: mol_type = other RNA
gaaggtcttttcaggatgata

SEQ ID NO: 771 - RNA, 21 nt, synthetic construct
  source 1..21: mol_type = other RNA
gcttttcaggatgattttcta

SEQ ID NO: 772 - RNA, 21 nt, synthetic construct
  source 1..21: mol_type = other RNA
gggatgattttcttatatcaa

SEQ ID NO: 773 - RNA, 21 nt, synthetic construct
  source 1..21: mol_type = other RNA
ggatggtttacttgtgtccta

SEQ ID NO: 774 - RNA, 21 nt, synthetic construct
  source 1..21: mol_type = other RNA
ggccaagagtgaataggacaa

SEQ ID NO: 775 - RNA, 21 nt, synthetic construct
  source 1..21: mol_type = other RNA
gtttctgggtattctttcaaa

SEQ ID NO: 776 - RNA, 21 nt, synthetic construct
  source 1..21: mol_type = other RNA
gttctgggtattctttcaaga

SEQ ID NO: 777 - RNA, 21 nt, synthetic construct
  source 1..21: mol_type = other RNA
gcaaataagcgcttgcaacaa

SEQ ID NO: 778 - RNA, 21 nt, synthetic construct
  source 1..21: mol_type = other RNA
ggcttgcaacaaagacattta

SEQ ID NO: 779 - RNA, 21 nt, synthetic construct
  source 1..21: mol_type = other RNA
gcaaagacatttatgtggaca

SEQ ID NO: 780 - RNA, 21 nt, synthetic construct
  source 1..21: mol_type = other RNA
gtatgtggacctagacatgaa

SEQ ID NO: 781 - RNA, 21 nt, synthetic construct
  source 1..21: mol_type = other RNA
cttgccaagagtgctcaagaa

SEQ ID NO: 782 - RNA, 21 nt, synthetic construct
  source 1..21: mol_type = other RNA
cccaagaaagatgcacggata

SEQ ID NO: 783 - RNA, 21 nt, synthetic construct
  source 1..21: mol_type = other RNA
ggaaatctttgtctccttaaa

SEQ ID NO: 784 - RNA, 21 nt, synthetic construct
  source 1..21: mol_type = other RNA
caaatctttgtctccttaaaa

SEQ ID NO: 785 - RNA, 21 nt, synthetic construct
  source 1..21: mol_type = other RNA
gaatctttgtctccttaaaaa

SEQ ID NO: 786 - RNA, 21 nt, synthetic construct
  source 1..21: mol_type = other RNA
gctttgtctccttaaaacata

SEQ ID NO: 787 - RNA, 21 nt, synthetic construct
  source 1..21: mol_type = other RNA
gttgtctccttaaaacatcta

SEQ ID NO: 788 - RNA, 21 nt, synthetic construct
  source 1..21: mol_type = other RNA
gtgtctccttaaaacatctga

SEQ ID NO: 789 - RNA, 21 nt, synthetic construct
  source 1..21: mol_type = other RNA
ggtctccttaaaacatctgaa

SEQ ID NO: 790 - RNA, 21 nt, synthetic construct
  source 1..21: mol_type = other RNA
gtccttaaaacatctgagaga

SEQ ID NO: 791 - RNA, 21 nt, synthetic construct
  source 1..21: mol_type = other RNA
gttaaaacatctgagagtgga

SEQ ID NO: 792 - RNA, 21 nt, synthetic construct
  source 1..21: mol_type = other RNA
gagctctttctggtttcagta

SEQ ID NO: 793 - RNA, 21 nt, synthetic construct
  source 1..21: mol_type = other RNA
ggaactggatattgttgctga

SEQ ID NO: 794 - RNA, 21 nt, synthetic construct
  source 1..21: mol_type = other RNA
caggaggcatctctggataca

SEQ ID NO: 795 - RNA, 21 nt, synthetic construct
  source 1..21: mol_type = other RNA
cgcatctctggatacacatta

SEQ ID NO: 796 - RNA, 21 nt, synthetic construct
  source 1..21: mol_type = other RNA
cttgtgtaaaatggataatga

SEQ ID NO: 797 - RNA, 21 nt, synthetic construct
  source 1..21: mol_type = other RNA
ggtgtaaaatggataatgaga

SEQ ID NO: 798 - RNA, 21 nt, synthetic construct
  source 1..21: mol_type = other RNA
ctgtaccaccaaaatcaagca

SEQ ID NO: 799 - RNA, 21 nt, synthetic construct
  source 1..21: mol_type = other RNA
gccaggatcgttggaggaaca

SEQ ID NO: 800 - RNA, 21 nt, synthetic construct
  source 1..21: mol_type = other RNA
ctagagtcacctaagatttta

SEQ ID NO: 801 - RNA, 21 nt, synthetic construct
  source 1..21: mol_type = other RNA
gggcattttaaatcaatctga

SEQ ID NO: 802 - RNA, 21 nt, synthetic construct
  source 1..21: mol_type = other RNA
cgcattttaaatcaatctgaa

SEQ ID NO: 803 - RNA, 21 nt, synthetic construct
  source 1..21: mol_type = other RNA
ccattttaaatcaatctgaaa

SEQ ID NO: 804 - RNA, 21 nt, synthetic construct
  source 1..21: mol_type = other RNA
cgacacatctttctttgggga

SEQ ID NO: 805 - RNA, 21 nt, synthetic construct
  source 1..21: mol_type = other RNA
gctttctttggggttcaagaa

SEQ ID NO: 806 - RNA, 21 nt, synthetic construct
  source 1..21: mol_type = other RNA
gtttggggttcaagaaataaa

SEQ ID NO: 807 - RNA, 21 nt, synthetic construct
  source 1..21: mol_type = other RNA
cttcaagaaataataatccaa

SEQ ID NO: 808 - RNA, 21 nt, synthetic construct
  source 1..21: mol_type = other RNA
gtcaagaaataataatccata

SEQ ID NO: 809 - RNA, 21 nt, synthetic construct
  source 1..21: mol_type = other RNA
ggggtatgatattgccttgta

SEQ ID NO: 810 - RNA, 21 nt, synthetic construct
  source 1..21: mol_type = other RNA
gtattgccttgttgaaactga

SEQ ID NO: 811 - RNA, 21 nt, synthetic construct
  source 1..21: mol_type = other RNA
gcttgttgaaactggaaacca

SEQ ID NO: 812 - RNA, 21 nt, synthetic construct
  source 1..21: mol_type = other RNA
cgaaaccacagtgaattacaa

SEQ ID NO: 813 - RNA, 21 nt, synthetic construct
  source 1..21: mol_type = other RNA
gtctccagaaagccaagataa

SEQ ID NO: 814 - RNA, 21 nt, synthetic construct
  source 1..21: mol_type = other RNA
gtccagaaagccaagatacca

SEQ ID NO: 815 - RNA, 21 nt, synthetic construct
  source 1..21: mol_type = other RNA
caaagccaagatacccttaga

SEQ ID NO: 816 - RNA, 21 nt, synthetic construct
  source 1..21: mol_type = other RNA
ggccaagatacccttagtgaa

SEQ ID NO: 817 - RNA, 21 nt, synthetic construct
  source 1..21: mol_type = other RNA
gcaagatacccttagtgacca

SEQ ID NO: 818 - RNA, 21 nt, synthetic construct
  source 1..21: mol_type = other RNA
gaagatacccttagtgaccaa

SEQ ID NO: 819 - RNA, 21 nt, synthetic construct
  source 1..21: mol_type = other RNA
ggaagagtgccagaagagata

SEQ ID NO: 820 - RNA, 21 nt, synthetic construct
  source 1..21: mol_type = other RNA
ctgccagaagagatacagaga

SEQ ID NO: 821 - RNA, 21 nt, synthetic construct
  source 1..21: mol_type = other RNA
ggaagagatacagaggacata

SEQ ID NO: 822 - RNA, 21 nt, synthetic construct
  source 1..21: mol_type = other RNA
gagagatacagaggacataaa

SEQ ID NO: 823 - RNA, 21 nt, synthetic construct
  source 1..21: mol_type = other RNA
ggatacagaggacataaaata

SEQ ID NO: 824 - RNA, 21 nt, synthetic construct
  source 1..21: mol_type = other RNA
catacagaggacataaaataa

SEQ ID NO: 825 - RNA, 21 nt, synthetic construct
  source 1..21: mol_type = other RNA
gggacataaaataacccataa

SEQ ID NO: 826 - RNA, 21 nt, synthetic construct
  source 1..21: mol_type = other RNA
gaaaataacccataagatgaa

SEQ ID NO: 827 - RNA, 21 nt, synthetic construct
  source 1..21: mol_type = other RNA
gaaataacccataagatgata

SEQ ID NO: 828 - RNA, 21 nt, synthetic construct
  source 1..21: mol_type = other RNA
gtaacccataagatgatctga

SEQ ID NO: 829 - RNA, 21 nt, synthetic construct
  source 1..21: mol_type = other RNA
gaacccataagatgatctgta

SEQ ID NO: 830 - RNA, 21 nt, synthetic construct
  source 1..21: mol_type = other RNA
ggtacgtggactggattctga

SEQ ID NO: 831 - RNA, 21 nt, synthetic construct
  source 1..21: mol_type = other RNA
ctggactggattctggagaaa

SEQ ID NO: 832 - RNA, 21 nt, synthetic construct
  source 1..21: mol_type = other RNA
gggattctggagaaaactcaa

SEQ ID NO: 833 - RNA, 21 nt, synthetic construct
  source 1..21: mol_type = other RNA
cgattctggagaaaactcaaa

SEQ ID NO: 834 - RNA, 21 nt, synthetic construct
  source 1..21: mol_type = other RNA
cattctggagaaaactcaaga

SEQ ID NO: 835 - RNA, 21 nt, synthetic construct
  source 1..21: mol_type = other RNA
ggcatgcttcctccacagtaa

SEQ ID NO: 836 - RNA, 21 nt, synthetic construct
  source 1..21: mol_type = other RNA
ggctagaagaaaacaaactga

SEQ ID NO: 837 - RNA, 21 nt, synthetic construct
  source 1..21: mol_type = other RNA
cctagaagaaaacaaactgta

SEQ ID NO: 838 - RNA, 21 nt, synthetic construct
  source 1..21: mol_type = other RNA
gtagaagaaaacaaactgtca

SEQ ID NO: 839 - RNA, 21 nt, synthetic construct
  source 1..21: mol_type = other RNA
gagaagaaaacaaactgtcaa

SEQ ID NO: 840 - RNA, 21 nt, synthetic construct
  source 1..21: mol_type = other RNA
caagaaaacaaactgtcacaa

SEQ ID NO: 841 - RNA, 21 nt, synthetic construct
  source 1..21: mol_type = other RNA
aaaatgctagaagaaaacaaa

SEQ ID NO: 842 - RNA, 21 nt, synthetic construct
  source 1..21: mol_type = other RNA
aaatgctagaagaaaacaaaa

SEQ ID NO: 843 - RNA, 21 nt, synthetic construct
  source 1..21: mol_type = other RNA
aatgctagaagaaaacaaaca

SEQ ID NO: 844 - RNA, 21 nt, synthetic construct
  source 1..21: mol_type = other RNA
atgctagaagaaaacaaacta

SEQ ID NO: 845 - RNA, 21 nt, synthetic construct
  source 1..21: mol_type = other RNA
tgctagaagaaaacaaactga

SEQ ID NO: 846 - RNA, 21 nt, synthetic construct
  source 1..21: mol_type = other RNA
gctagaagaaaacaaactgta

SEQ ID NO: 847 - RNA, 21 nt, synthetic construct
  source 1..21: mol_type = other RNA
ctagaagaaaacaaactgtca

SEQ ID NO: 848 - RNA, 21 nt, synthetic construct
  source 1..21: mol_type = other RNA
tagaagaaaacaaactgtcaa

SEQ ID NO: 849 - RNA, 21 nt, synthetic construct
  source 1..21: mol_type = other RNA
agaagaaaacaaactgtcaca

SEQ ID NO: 850 - RNA, 21 nt, synthetic construct
  source 1..21: mol_type = other RNA
gaagaaaacaaactgtcacaa

SEQ ID NO: 851 - RNA, 21 nt, synthetic construct
  source 1..21: mol_type = other RNA
aagaaaacaaactgtcacaaa

SEQ ID NO: 852 - RNA, 21 nt, synthetic construct
  source 1..21: mol_type = other RNA
agaaaacaaactgtcacaaga

SEQ ID NO: 853 - RNA, 21 nt, synthetic construct
  source 1..21: mol_type = other RNA
gaaaacaaactgtcacaagta

SEQ ID NO: 854 - RNA, 21 nt, synthetic construct
  source 1..21: mol_type = other RNA
aaaacaaactgtcacaagtta

SEQ ID NO: 855 - RNA, 21 nt, synthetic construct
  source 1..21: mol_type = other RNA
aaacaaactgtcacaagttga

SEQ ID NO: 856 - RNA, 21 nt, synthetic construct
  source 1..21: mol_type = other RNA
gcgtcagtttctggtgaatga

SEQ ID NO: 857 - RNA, 21 nt, synthetic construct
  source 1..21: mol_type = other RNA
cgagcgatttctgggtattca

SEQ ID NO: 858 - RNA, 19 nt, synthetic construct
  source 1..19: mol_type = other RNA
tcaggatgattttcttata

SEQ ID NO: 859 - RNA, 19 nt, synthetic construct
  source 1..19: mol_type = other RNA
caggatgattttcttataa

SEQ ID NO: 860 - RNA, 19 nt, synthetic construct
  source 1..19: mol_type = other RNA
gtttctggtgaatgtgtga

SEQ ID NO: 861 - RNA, 19 nt, synthetic construct
  source 1..19: mol_type = other RNA
gtattctttcaagcaatga

SEQ ID NO: 862 - RNA, 19 nt, synthetic construct
  source 1..19: mol_type = other RNA
aagcaatgctcacaccaaa

SEQ ID NO: 863 - RNA, 19 nt, synthetic construct
  source 1..19: mol_type = other RNA
agcaatgctcacaccaaaa

SEQ ID NO: 864 - RNA, 19 nt, synthetic construct
  source 1..19: mol_type = other RNA
gcaacaaagacatttatga

SEQ ID NO: 865 - RNA, 19 nt, synthetic construct
  source 1..19: mol_type = other RNA
catgaagggcataaactaa

SEQ ID NO: 866 - RNA, 19 nt, synthetic construct
  source 1..19: mol_type = other RNA
ggcataaactataacagca

SEQ ID NO: 867 - RNA, 19 nt, synthetic construct
  source 1..19: mol_type = other RNA
gagtgtaccaccaaaatca

SEQ ID NO: 868 - RNA, 19 nt, synthetic construct
  source 1..19: mol_type = other RNA
agtgtaccaccaaaatcaa

SEQ ID NO: 869 - RNA, 19 nt, synthetic construct
  source 1..19: mol_type = other RNA
gtgtaccaccaaaatcaaa

SEQ ID NO: 870 - RNA, 19 nt, synthetic construct
  source 1..19: mol_type = other RNA
gtagagtcacctaagatta

SEQ ID NO: 871 - RNA, 19 nt, synthetic construct
  source 1..19: mol_type = other RNA
cacctaagattttgcgtga

SEQ ID NO: 872 - RNA, 19 nt, synthetic construct
  source 1..19: mol_type = other RNA
acctaagattttgcgtgta

SEQ ID NO: 873 - RNA, 19 nt, synthetic construct
  source 1..19: mol_type = other RNA
ctaagattttgcgtgtcta

SEQ ID NO: 874 - RNA, 19 nt, synthetic construct
  source 1..19: mol_type = other RNA
taagattttgcgtgtctaa

SEQ ID NO: 875 - RNA, 19 nt, synthetic construct
  source 1..19: mol_type = other RNA
agattttgcgtgtctacaa

SEQ ID NO: 876 - RNA, 19 nt, synthetic construct
  source 1..19: mol_type = other RNA
agtggcattttaaatcaaa

SEQ ID NO: 877 - RNA, 19 nt, synthetic construct
  source 1..19: mol_type = other RNA
gtggcattttaaatcaata

SEQ ID NO: 878 - RNA, 19 nt, synthetic construct
  source 1..19: mol_type = other RNA
ttaaatcaatctgaaataa

SEQ ID NO: 879 - RNA, 19 nt, synthetic construct
  source 1..19: mol_type = other RNA
aaatcaatctgaaataaaa

SEQ ID NO: 880 - RNA, 19 nt, synthetic construct
  source 1..19: mol_type = other RNA
acatctttctttggggtta

SEQ ID NO: 881 - RNA, 19 nt, synthetic construct
  source 1..19: mol_type = other RNA
atctttctttggggttcaa

SEQ ID NO: 882 - RNA, 19 nt, synthetic construct
  source 1..19: mol_type = other RNA
tctttctttggggttcaaa

SEQ ID NO: 883 - RNA, 19 nt, synthetic construct
  source 1..19: mol_type = other RNA
tctttggggttcaagaaaa

SEQ ID NO: 884 - RNA, 19 nt, synthetic construct
  source 1..19: mol_type = other RNA
ctttggggttcaagaaata

SEQ ID NO: 885 - RNA, 19 nt, synthetic construct
  source 1..19: mol_type = other RNA
tttggggttcaagaaataa

SEQ ID NO: 886 - RNA, 19 nt, synthetic construct
  source 1..19: mol_type = other RNA
gttcaagaaataataatca

SEQ ID NO: 887 - RNA, 19 nt, synthetic construct
  source 1..19: mol_type = other RNA
aaataataatccatgatca

SEQ ID NO: 888 - RNA, 19 nt, synthetic construct
  source 1..19: mol_type = other RNA
agtataaaatggcagaaaa

SEQ ID NO: 889 - RNA, 19 nt, synthetic construct
  source 1..19: mol_type = other RNA
gtataaaatggcagaaaga

SEQ ID NO: 890 - RNA, 19 nt, synthetic construct
  source 1..19: mol_type = other RNA
gtatgatattgccttgtta

SEQ ID NO: 891 - RNA, 19 nt, synthetic construct
  source 1..19: mol_type = other RNA
atattgccttgttgaaaca

SEQ ID NO: 892 - RNA, 19 nt, synthetic construct
  source 1..19: mol_type = other RNA
tattgccttgttgaaacta

SEQ ID NO: 893 - RNA, 19 nt, synthetic construct
  source 1..19: mol_type = other RNA
ttgccttgttgaaactgga

SEQ ID NO: 894 - RNA, 19 nt, synthetic construct
  source 1..19: mol_type = other RNA
cttgttgaaactggaaaca

SEQ ID NO: 895 - RNA, 19 nt, synthetic construct
  source 1..19: mol_type = other RNA
ttgaaactggaaaccacaa

SEQ ID NO: 896 - RNA, 19 nt, synthetic construct
  source 1..19: mol_type = other RNA
gaaaccacagtgaattaca

SEQ ID NO: 897 - RNA, 19 nt, synthetic construct
  source 1..19: mol_type = other RNA
accacagtgaattacacaa

SEQ ID NO: 898 - RNA, 19 nt, synthetic construct
  source 1..19: mol_type = other RNA
cacagtgaattacacagaa

SEQ ID NO: 899 - RNA, 19 nt, synthetic construct
  source 1..19: mol_type = other RNA
aaaatactctccagaaaga

SEQ ID NO: 900 - RNA, 19 nt, synthetic construct
  source 1..19: mol_type = other RNA
tctccagaaagccaagata

SEQ ID NO: 901 - RNA, 19 nt, synthetic construct
  source 1..19: mol_type = other RNA
agccaagatacccttagta

SEQ ID NO: 902 - RNA, 19 nt, synthetic construct
  source 1..19: mol_type = other RNA
agagatacagaggacataa

SEQ ID NO: 903 - RNA, 19 nt, synthetic construct
  source 1..19: mol_type = other RNA
gatacagaggacataaaaa

SEQ ID NO: 904 - RNA, 19 nt, synthetic construct
  source 1..19: mol_type = other RNA
cagaggacataaaataaca

SEQ ID NO: 905 - RNA, 19 nt, synthetic construct
  source 1..19: mol_type = other RNA
agaggacataaaataacca

SEQ ID NO: 906 - RNA, 19 nt, synthetic construct
  source 1..19: mol_type = other RNA
ggacataaaataacccata

SEQ ID NO: 907 - RNA, 19 nt, synthetic construct
  source 1..19: mol_type = other RNA
cataaaataacccataaga

SEQ ID NO: 908 - RNA, 19 nt, synthetic construct
  source 1..19: mol_type = other RNA
ataaaataacccataagaa

SEQ ID NO: 909 - RNA, 19 nt, synthetic construct
  source 1..19: mol_type = other RNA
cccataagatgatctgtga

SEQ ID NO: 910 - RNA, 19 nt, synthetic construct
  source 1..19: mol_type = other RNA
actggattctggagaaaaa

SEQ ID NO: 911 - RNA, 19 nt, synthetic construct
  source 1..19: mol_type = other RNA
ctggattctggagaaaaca

SEQ ID NO: 912 - RNA, 19 nt, synthetic construct
  source 1..19: mol_type = other RNA
cattcagaggaggatgaaa

SEQ ID NO: 913 - RNA, 19 nt, synthetic construct
  source 1..19: mol_type = other RNA
aggtcttttcaggatgata

SEQ ID NO: 914 - RNA, 19 nt, synthetic construct
  source 1..19: mol_type = other RNA
ttttcaggatgattttcta

SEQ ID NO: 915 - RNA, 19 nt, synthetic construct
  source 1..19: mol_type = other RNA
gatgattttcttatatcaa

SEQ ID NO: 916 - RNA, 19 nt, synthetic construct
  source 1..19: mol_type = other RNA
atggtttacttgtgtccta

SEQ ID NO: 917 - RNA, 19 nt, synthetic construct
  source 1..19: mol_type = other RNA
ccaagagtgaataggacaa

SEQ ID NO: 918 - RNA, 19 nt, synthetic construct
  source 1..19: mol_type = other RNA
ttctgggtattctttcaaa

SEQ ID NO: 919 - RNA, 19 nt, synthetic construct
  source 1..19: mol_type = other RNA
tctgggtattctttcaaga

SEQ ID NO: 920 - RNA, 19 nt, synthetic construct
  source 1..19: mol_type = other RNA
aaataagcgcttgcaacaa

SEQ ID NO: 921 - RNA, 19 nt, synthetic construct
  source 1..19: mol_type = other RNA
cttgcaacaaagacattta

SEQ ID NO: 922 - RNA, 19 nt, synthetic construct
  source 1..19: mol_type = other RNA
aaagacatttatgtggaca

SEQ ID NO: 923 - RNA, 19 nt, synthetic construct
  source 1..19: mol_type = other RNA
atgtggacctagacatgaa

SEQ ID NO: 924 - RNA, 19 nt, synthetic construct
  source 1..19: mol_type = other RNA
tgccaagagtgctcaagaa

SEQ ID NO: 925 - RNA, 19 nt, synthetic construct
  source 1..19: mol_type = other RNA
caagaaagatgcacggata

SEQ ID NO: 926 - RNA, 19 nt, synthetic construct
  source 1..19: mol_type = other RNA
aaatctttgtctccttaaa

SEQ ID NO: 927 - RNA, 19 nt, synthetic construct
  source 1..19: mol_type = other RNA
aatctttgtctccttaaaa

SEQ ID NO: 928 - RNA, 19 nt, synthetic construct
  source 1..19: mol_type = other RNA
atctttgtctccttaaaaa

SEQ ID NO: 929 - RNA, 19 nt, synthetic construct
  source 1..19: mol_type = other RNA
tttgtctccttaaaacata

SEQ ID NO: 930 - RNA, 19 nt, synthetic construct
  source 1..19: mol_type = other RNA
tgtctccttaaaacatcta

SEQ ID NO: 931 - RNA, 19 nt, synthetic construct
  source 1..19: mol_type = other RNA
gtctccttaaaacatctga

SEQ ID NO: 932 - RNA, 19 nt, synthetic construct
  source 1..19: mol_type = other RNA
tctccttaaaacatctgaa

SEQ ID NO: 933 - RNA, 19 nt, synthetic construct
  source 1..19: mol_type = other RNA
ccttaaaacatctgagaga

SEQ ID NO: 934 - RNA, 19 nt, synthetic construct
  source 1..19: mol_type = other RNA
taaaacatctgagagtgga

SEQ ID NO: 935 - RNA, 19 nt, synthetic construct
  source 1..19: mol_type = other RNA
gctctttctggtttcagta

SEQ ID NO: 936 - RNA, 19 nt, synthetic construct
  source 1..19: mol_type = other RNA
aactggatattgttgctga

SEQ ID NO: 937 - RNA, 19 nt, synthetic construct
  source 1..19: mol_type = other RNA
ggaggcatctctggataca

SEQ ID NO: 938 - RNA, 19 nt, synthetic construct
  source 1..19: mol_type = other RNA
catctctggatacacatta

SEQ ID NO: 939 - RNA, 19 nt, synthetic construct
  source 1..19: mol_type = other RNA
tgtgtaaaatggataatga

SEQ ID NO: 940 - RNA, 19 nt, synthetic construct
  source 1..19: mol_type = other RNA
tgtaaaatggataatgaga

SEQ ID NO: 941 - RNA, 19 nt, synthetic construct
  source 1..19: mol_type = other RNA
gtaccaccaaaatcaagca

SEQ ID NO: 942 - RNA, 19 nt, synthetic construct
  source 1..19: mol_type = other RNA
caggatcgttggaggaaca

SEQ ID NO: 943 - RNA, 19 nt, synthetic construct
  source 1..19: mol_type = other RNA
agagtcacctaagatttta

SEQ ID NO: 944 - RNA, 19 nt, synthetic construct
  source 1..19: mol_type = other RNA
gcattttaaatcaatctga

SEQ ID NO: 945 - RNA, 19 nt, synthetic construct
  source 1..19: mol_type = other RNA
cattttaaatcaatctgaa

SEQ ID NO: 946 - RNA, 19 nt, synthetic construct
  source 1..19: mol_type = other RNA
attttaaatcaatctgaaa

SEQ ID NO: 947 - RNA, 19 nt, synthetic construct
  source 1..19: mol_type = other RNA
acacatctttctttgggga

SEQ ID NO: 948 - RNA, 19 nt, synthetic construct
  source 1..19: mol_type = other RNA
tttctttggggttcaagaa

SEQ ID NO: 949 - RNA, 19 nt, synthetic construct
  source 1..19: mol_type = other RNA
ttggggttcaagaaataaa

SEQ ID NO: 950 - RNA, 19 nt, synthetic construct
  source 1..19: mol_type = other RNA
tcaagaaataataatccaa

SEQ ID NO: 951 - RNA, 19 nt, synthetic construct
  source 1..19: mol_type = other RNA
caagaaataataatccata

SEQ ID NO: 952 - RNA, 19 nt, synthetic construct
  source 1..19: mol_type = other RNA
ggtatgatattgccttgta

SEQ ID NO: 953 - RNA, 19 nt, synthetic construct
  source 1..19: mol_type = other RNA
attgccttgttgaaactga

SEQ ID NO: 954 - RNA, 19 nt, synthetic construct
  source 1..19: mol_type = other RNA
ttgttgaaactggaaacca

SEQ ID NO: 955 - RNA, 19 nt, synthetic construct
  source 1..19: mol_type = other RNA
aaaccacagtgaattacaa

SEQ ID NO: 956 - RNA, 19 nt, synthetic construct
  source 1..19: mol_type = other RNA
ctccagaaagccaagataa

SEQ ID NO: 957 - RNA, 19 nt, synthetic construct
  source 1..19: mol_type = other RNA
ccagaaagccaagatacca

SEQ ID NO: 958 - RNA, 19 nt, synthetic construct
  source 1..19: mol_type = other RNA
aagccaagatacccttaga

SEQ ID NO: 959 - RNA, 19 nt, synthetic construct
  source 1..19: mol_type = other RNA
ccaagatacccttagtgaa

SEQ ID NO: 960 - RNA, 19 nt, synthetic construct
  source 1..19: mol_type = other RNA
aagatacccttagtgacca

SEQ ID NO: 961 - RNA, 19 nt, synthetic construct
  source 1..19: mol_type = other RNA
agatacccttagtgaccaa

SEQ ID NO: 962 - RNA, 19 nt, synthetic construct
  source 1..19: mol_type = other RNA
aagagtgccagaagagata

SEQ ID NO: 963 - RNA, 19 nt, synthetic construct
  source 1..19: mol_type = other RNA
gccagaagagatacagaga

SEQ ID NO: 964 - RNA, 19 nt, synthetic construct
  source 1..19: mol_type = other RNA
aagagatacagaggacata

SEQ ID NO: 965 - RNA, 19 nt, synthetic construct
  source 1..19: mol_type = other RNA
gagatacagaggacataaa

SEQ ID NO: 966 - RNA, 19 nt, synthetic construct
  source 1..19: mol_type = other RNA
atacagaggacataaaata

SEQ ID NO: 967 - RNA, 19 nt, synthetic construct
  source 1..19: mol_type = other RNA
tacagaggacataaaataa

SEQ ID NO: 968 - RNA, 19 nt, synthetic construct
  source 1..19: mol_type = other RNA
gacataaaataacccataa

SEQ ID NO: 969 - RNA, 19 nt, synthetic construct
  source 1..19: mol_type = other RNA
aaataacccataagatgaa

SEQ ID NO: 970 - RNA, 19 nt, synthetic construct
  source 1..19: mol_type = other RNA
aataacccataagatgata

SEQ ID NO: 971 - RNA, 19 nt, synthetic construct
  source 1..19: mol_type = other RNA
aacccataagatgatctga

SEQ ID NO: 972 - RNA, 19 nt, synthetic construct
  source 1..19: mol_type = other RNA
acccataagatgatctgta

SEQ ID NO: 973 - RNA, 19 nt, synthetic construct
  source 1..19: mol_type = other RNA
tacgtggactggattctga

SEQ ID NO: 974 - RNA, 19 nt, synthetic construct
  source 1..19: mol_type = other RNA
ggactggattctggagaaa

SEQ ID NO: 975 - RNA, 19 nt, synthetic construct
  source 1..19: mol_type = other RNA
gattctggagaaaactcaa

SEQ ID NO: 976 - RNA, 19 nt, synthetic construct
  source 1..19: mol_type = other RNA
attctggagaaaactcaaa

SEQ ID NO: 977 - RNA, 19 nt, synthetic construct
  source 1..19: mol_type = other RNA
ttctggagaaaactcaaga

SEQ ID NO: 978 - RNA, 19 nt, synthetic construct
  source 1..19: mol_type = other RNA
catgcttcctccacagtaa

SEQ ID NO: 979 - RNA, 19 nt, synthetic construct
  source 1..19: mol_type = other RNA
ctagaagaaaacaaactga

SEQ ID NO: 980 - RNA, 19 nt, synthetic construct
  source 1..19: mol_type = other RNA
tagaagaaaacaaactgta

SEQ ID NO: 981 - RNA, 19 nt, synthetic construct
  source 1..19: mol_type = other RNA
agaagaaaacaaactgtca

SEQ ID NO: 982 - RNA, 19 nt, synthetic construct
  source 1..19: mol_type = other RNA
gaagaaaacaaactgtcaa

SEQ ID NO: 983 - RNA, 19 nt, synthetic construct
  source 1..19: mol_type = other RNA
agaaaacaaactgtcacaa

SEQ ID NO: 984 - RNA, 19 nt, synthetic construct
  source 1..19: mol_type = other RNA
aatgctagaagaaaacaaa

SEQ ID NO: 985 - RNA, 19 nt, synthetic construct
  source 1..19: mol_type = other RNA
atgctagaagaaaacaaaa

SEQ ID NO: 986 - RNA, 19 nt, synthetic construct
  source 1..19: mol_type = other RNA
tgctagaagaaaacaaaca

SEQ ID NO: 987 - RNA, 19 nt, synthetic construct
  source 1..19: mol_type = other RNA
gctagaagaaaacaaacta

SEQ ID NO: 988 - RNA, 19 nt, synthetic construct
  source 1..19: mol_type = other RNA
ctagaagaaaacaaactga

SEQ ID NO: 989 - RNA, 19 nt, synthetic construct
  source 1..19: mol_type = other RNA
tagaagaaaacaaactgta

SEQ ID NO: 990 - RNA, 19 nt, synthetic construct
  source 1..19: mol_type = other RNA
agaagaaaacaaactgtca

SEQ ID NO: 991 - RNA, 19 nt, synthetic construct
  source 1..19: mol_type = other RNA
gaagaaaacaaactgtcaa

SEQ ID NO: 992 - RNA, 19 nt, synthetic construct
  source 1..19: mol_type = other RNA
aagaaaacaaactgtcaca

SEQ ID NO: 993 - RNA, 19 nt, synthetic construct
  source 1..19: mol_type = other RNA
agaaaacaaactgtcacaa

SEQ ID NO: 994 - RNA, 19 nt, synthetic construct
  source 1..19: mol_type = other RNA
gaaaacaaactgtcacaaa

SEQ ID NO: 995 - RNA, 19 nt, synthetic construct
  source 1..19: mol_type = other RNA
aaaacaaactgtcacaaga

SEQ ID NO: 996 - RNA, 19 nt, synthetic construct
  source 1..19: mol_type = other RNA
aaacaaactgtcacaagta

SEQ ID NO: 997 - RNA, 19 nt, synthetic construct
  source 1..19: mol_type = other RNA
aacaaactgtcacaagtta

SEQ ID NO: 998 - RNA, 19 nt, synthetic construct
  source 1..19: mol_type = other RNA
acaaactgtcacaagttga

SEQ ID NO: 999 - RNA, 19 nt, synthetic construct
  source 1..19: mol_type = other RNA
gtcagtttctggtgaatga

SEQ ID NO: 1000 - RNA, 19 nt, synthetic construct
  source 1..19: mol_type = other RNA
agcgatttctgggtattca

SEQ ID NO: 1001 - RNA, 21 nt, synthetic construct
  source 1..21: mol_type = other RNA
tataagaaaatcatcctgaac

SEQ ID NO: 1002 - RNA, 21 nt, synthetic construct
  source 1..21: mol_type = other RNA
ttataagaaaatcatcctgac

SEQ ID NO: 1003 - RNA, 21 nt, synthetic construct
  source 1..21: mol_type = other RNA
tcacacattcaccagaaactc

SEQ ID NO: 1004 - RNA, 21 nt, synthetic construct
  source 1..21: mol_type = other RNA
tcattgcttgaaagaataccg

SEQ ID NO: 1005 - RNA, 21 nt, synthetic construct
  source 1..21: mol_type = other RNA
tttggtgtgagcattgcttgc

SEQ ID NO: 1006 - RNA, 21 nt, synthetic construct
  source 1..21: mol_type = other RNA
ttttggtgtgagcattgcttc

SEQ ID NO: 1007 - RNA, 21 nt, synthetic construct
  source 1..21: mol_type = other RNA
tcataaatgtctttgttgcac

SEQ ID NO: 1008 - RNA, 21 nt, synthetic construct
  source 1..21: mol_type = other RNA
ttagtttatgcccttcatgtg

SEQ ID NO: 1009 - RNA, 21 nt, synthetic construct
  source 1..21: mol_type = other RNA
tgctgttatagtttatgcccc

SEQ ID NO: 1010 - RNA, 21 nt, synthetic construct
  source 1..21: mol_type = other RNA
tgattttggtggtacactcac

SEQ ID NO: 1011 - RNA, 21 nt, synthetic construct
  source 1..21: mol_type = other RNA
ttgattttggtggtacactcc

SEQ ID NO: 1012 - RNA, 21 nt, synthetic construct
  source 1..21: mol_type = other RNA
tttgattttggtggtacactg

SEQ ID NO: 1013 - RNA, 21 nt, synthetic construct
  source 1..21: mol_type = other RNA
taatcttaggtgactctaccg

SEQ ID NO: 1014 - RNA, 21 nt, synthetic construct
  source 1..21: mol_type = other RNA
tcacgcaaaatcttaggtgag

SEQ ID NO: 1015 - RNA, 21 nt, synthetic construct
  source 1..21: mol_type = other RNA
tacacgcaaaatcttaggtgc

SEQ ID NO: 1016 - RNA, 21 nt, synthetic construct
  source 1..21: mol_type = other RNA
tagacacgcaaaatcttaggc

SEQ ID NO: 1017 - RNA, 21 nt, synthetic construct
  source 1..21: mol_type = other RNA
ttagacacgcaaaatcttagc

SEQ ID NO: 1018 - RNA, 21 nt, synthetic construct
  source 1..21: mol_type = other RNA
ttgtagacacgcaaaatcttc

SEQ ID NO: 1019 - RNA, 21 nt, synthetic construct
  source 1..21: mol_type = other RNA
tttgatttaaaatgccactgc

SEQ ID NO: 1020 - RNA, 21 nt, synthetic construct
  source 1..21: mol_type = other RNA
tattgatttaaaatgccactc

SEQ ID NO: 1021 - RNA, 21 nt, synthetic construct
  source 1..21: mol_type = other RNA
ttatttcagattgatttaaac

SEQ ID NO: 1022 - RNA, 21 nt, synthetic construct
  source 1..21: mol_type = other RNA
ttttatttcagattgatttac

SEQ ID NO: 1023 - RNA, 21 nt, synthetic construct
  source 1..21: mol_type = other RNA
taaccccaaagaaagatgtgc

SEQ ID NO: 1024 - RNA, 21 nt, synthetic construct
  source 1..21: mol_type = other RNA
ttgaaccccaaagaaagatgc

SEQ ID NO: 1025 - RNA, 21 nt, synthetic construct
  source 1..21: mol_type = other RNA
tttgaaccccaaagaaagatc

SEQ ID NO: 1026 - RNA, 21 nt, synthetic construct
  source 1..21: mol_type = other RNA
ttttcttgaaccccaaagaac

SEQ ID NO: 1027 - RNA, 21 nt, synthetic construct
  source 1..21: mol_type = other RNA
tatttcttgaaccccaaagac

SEQ ID NO: 1028 - RNA, 21 nt, synthetic construct
  source 1..21: mol_type = other RNA
ttatttcttgaaccccaaagc

SEQ ID NO: 1029 - RNA, 21 nt, synthetic construct
  source 1..21: mol_type = other RNA
tgattattatttcttgaaccg

SEQ ID NO: 1030 - RNA, 21 nt, synthetic construct
  source 1..21: mol_type = other RNA
tgatcatggattattatttcc

SEQ ID NO: 1031 - RNA, 21 nt, synthetic construct
  source 1..21: mol_type = other RNA
ttttctgccattttatactgc

SEQ ID NO: 1032 - RNA, 21 nt, synthetic construct
  source 1..21: mol_type = other RNA
tctttctgccattttatactc

SEQ ID NO: 1033 - RNA, 21 nt, synthetic construct
  source 1..21: mol_type = other RNA
taacaaggcaatatcataccg

SEQ ID NO: 1034 - RNA, 21 nt, synthetic construct
  source 1..21: mol_type = other RNA
tgtttcaacaaggcaatatcc

SEQ ID NO: 1035 - RNA, 21 nt, synthetic construct
  source 1..21: mol_type = other RNA
tagtttcaacaaggcaatatg

SEQ ID NO: 1036 - RNA, 21 nt, synthetic construct
  source 1..21: mol_type = other RNA
tccagtttcaacaaggcaatc

SEQ ID NO: 1037 - RNA, 21 nt, synthetic construct
  source 1..21: mol_type = other RNA
tgtttccagtttcaacaaggg

SEQ ID NO: 1038 - RNA, 21 nt, synthetic construct
  source 1..21: mol_type = other RNA
ttgtggtttccagtttcaacc

SEQ ID NO: 1039 - RNA, 21 nt, synthetic construct
  source 1..21: mol_type = other RNA
tgtaattcactgtggtttccc

SEQ ID NO: 1040 - RNA, 21 nt, synthetic construct
  source 1..21: mol_type = other RNA
ttgtgtaattcactgtggttc

SEQ ID NO: 1041 - RNA, 21 nt, synthetic construct
  source 1..21: mol_type = other RNA
ttctgtgtaattcactgtggc

SEQ ID NO: 1042 - RNA, 21 nt, synthetic construct
  source 1..21: mol_type = other RNA
tctttctggagagtattttgc

SEQ ID NO: 1043 - RNA, 21 nt, synthetic construct
  source 1..21: mol_type = other RNA
tatcttggctttctggagagc

SEQ ID NO: 1044 - RNA, 21 nt, synthetic construct
  source 1..21: mol_type = other RNA
tactaagggtatcttggcttc

SEQ ID NO: 1045 - RNA, 21 nt, synthetic construct
  source 1..21: mol_type = other RNA
ttatgtcctctgtatctcttg

SEQ ID NO: 1046 - RNA, 21 nt, synthetic construct
  source 1..21: mol_type = other RNA
tttttatgtcctctgtatctg

SEQ ID NO: 1047 - RNA, 21 nt, synthetic construct
  source 1..21: mol_type = other RNA
tgttattttatgtcctctgtc

SEQ ID NO: 1048 - RNA, 21 nt, synthetic construct
  source 1..21: mol_type = other RNA
tggttattttatgtcctctgc

SEQ ID NO: 1049 - RNA, 21 nt, synthetic construct
  source 1..21: mol_type = other RNA
tatgggttattttatgtcctg

SEQ ID NO: 1050 - RNA, 21 nt, synthetic construct
  source 1..21: mol_type = other RNA
tcttatgggttattttatgtg

SEQ ID NO: 1051 - RNA, 21 nt, synthetic construct
  source 1..21: mol_type = other RNA
ttcttatgggttattttatgc

SEQ ID NO: 1052 - RNA, 21 nt, synthetic construct
  source 1..21: mol_type = other RNA
tcacagatcatcttatgggtc

SEQ ID NO: 1053 - RNA, 21 nt, synthetic construct
  source 1..21: mol_type = other RNA
tttttctccagaatccagtcg

SEQ ID NO: 1054 - RNA, 21 nt, synthetic construct
  source 1..21: mol_type = other RNA
tgttttctccagaatccagtg

SEQ ID NO: 1055 - RNA, 21 nt, synthetic construct
  source 1..21: mol_type = other RNA
tttcatcctcctctgaatgac

SEQ ID NO: 1056 - RNA, 21 nt, synthetic construct
  source 1..21: mol_type = other RNA
tatcatcctgaaaagaccttc

SEQ ID NO: 1057 - RNA, 21 nt, synthetic construct
  source 1..21: mol_type = other RNA
tagaaaatcatcctgaaaagc

SEQ ID NO: 1058 - RNA, 21 nt, synthetic construct
  source 1..21: mol_type = other RNA
ttgatataagaaaatcatccc

SEQ ID NO: 1059 - RNA, 21 nt, synthetic construct
  source 1..21: mol_type = other RNA
taggacacaagtaaaccatcc

SEQ ID NO: 1060 - RNA, 21 nt, synthetic construct
  source 1..21: mol_type = other RNA
ttgtcctattcactcttggcc

SEQ ID NO: 1061 - RNA, 21 nt, synthetic construct
  source 1..21: mol_type = other RNA
tttgaaagaatacccagaaac

SEQ ID NO: 1062 - RNA, 21 nt, synthetic construct
  source 1..21: mol_type = other RNA
tcttgaaagaatacccagaac

SEQ ID NO: 1063 - RNA, 21 nt, synthetic construct
  source 1..21: mol_type = other RNA
ttgttgcaagcgcttatttgc

SEQ ID NO: 1064 - RNA, 21 nt, synthetic construct
  source 1..21: mol_type = other RNA
taaatgtctttgttgcaagcc

SEQ ID NO: 1065 - RNA, 21 nt, synthetic construct
  source 1..21: mol_type = other RNA
tgtccacataaatgtctttgc

SEQ ID NO: 1066 - RNA, 21 nt, synthetic construct
  source 1..21: mol_type = other RNA
ttcatgtctaggtccacatac

SEQ ID NO: 1067 - RNA, 21 nt, synthetic construct
  source 1..21: mol_type = other RNA
ttcttgagcactcttggcaag

SEQ ID NO: 1068 - RNA, 21 nt, synthetic construct
  source 1..21: mol_type = other RNA
tatccgtgcatctttcttggg

SEQ ID NO: 1069 - RNA, 21 nt, synthetic construct
  source 1..21: mol_type = other RNA
tttaaggagacaaagatttcc

SEQ ID NO: 1070 - RNA, 21 nt, synthetic construct
  source 1..21: mol_type = other RNA
ttttaaggagacaaagatttg

SEQ ID NO: 1071 - RNA, 21 nt, synthetic construct
  source 1..21: mol_type = other RNA
tttttaaggagacaaagattc

SEQ ID NO: 1072 - RNA, 21 nt, synthetic construct
  source 1..21: mol_type = other RNA
tatgttttaaggagacaaagc

SEQ ID NO: 1073 - RNA, 21 nt, synthetic construct
  source 1..21: mol_type = other RNA
tagatgttttaaggagacaac

SEQ ID NO: 1074 - RNA, 21 nt, synthetic construct
  source 1..21: mol_type = other RNA
tcagatgttttaaggagacac

SEQ ID NO: 1075 - RNA, 21 nt, synthetic construct
  source 1..21: mol_type = other RNA
ttcagatgttttaaggagacc

SEQ ID NO: 1076 - RNA, 21 nt, synthetic construct
  source 1..21: mol_type = other RNA
tctctcagatgttttaaggac

SEQ ID NO: 1077 - RNA, 21 nt, synthetic construct
  source 1..21: mol_type = other RNA
tccactctcagatgttttaac

SEQ ID NO: 1078 - RNA, 21 nt, synthetic construct
  source 1..21: mol_type = other RNA
tactgaaaccagaaagagctc

SEQ ID NO: 1079 - RNA, 21 nt, synthetic construct
  source 1..21: mol_type = other RNA
tcagcaacaatatccagttcc

SEQ ID NO: 1080 - RNA, 21 nt, synthetic construct
  source 1..21: mol_type = other RNA
tgtatccagagatgcctcctg

SEQ ID NO: 1081 - RNA, 21 nt, synthetic construct
  source 1..21: mol_type = other RNA
taatgtgtatccagagatgcg

SEQ ID NO: 1082 - RNA, 21 nt, synthetic construct
  source 1..21: mol_type = other RNA
tcattatccattttacacaag

SEQ ID NO: 1083 - RNA, 21 nt, synthetic construct
  source 1..21: mol_type = other RNA
tctcattatccattttacacc

SEQ ID NO: 1084 - RNA, 21 nt, synthetic construct
  source 1..21: mol_type = other RNA
tgcttgattttggtggtacag

SEQ ID NO: 1085 - RNA, 21 nt, synthetic construct
  source 1..21: mol_type = other RNA
tgttcctccaacgatcctggc

SEQ ID NO: 1086 - RNA, 21 nt, synthetic construct
  source 1..21: mol_type = other RNA
taaaatcttaggtgactctag

SEQ ID NO: 1087 - RNA, 21 nt, synthetic construct
  source 1..21: mol_type = other RNA
tcagattgatttaaaatgccc

SEQ ID NO: 1088 - RNA, 21 nt, synthetic construct
  source 1..21: mol_type = other RNA
ttcagattgatttaaaatgcg

SEQ ID NO: 1089 - RNA, 21 nt, synthetic construct
  source 1..21: mol_type = other RNA
tttcagattgatttaaaatgg

SEQ ID NO: 1090 - RNA, 21 nt, synthetic construct
  source 1..21: mol_type = other RNA
tccccaaagaaagatgtgtcg

SEQ ID NO: 1091 - RNA, 21 nt, synthetic construct
  source 1..21: mol_type = other RNA
ttcttgaaccccaaagaaagc

SEQ ID NO: 1092 - RNA, 21 nt, synthetic construct
  source 1..21: mol_type = other RNA
tttatttcttgaaccccaaac

SEQ ID NO: 1093 - RNA, 21 nt, synthetic construct
  source 1..21: mol_type = other RNA
ttggattattatttcttgaag

SEQ ID NO: 1094 - RNA, 21 nt, synthetic construct
  source 1..21: mol_type = other RNA
tatggattattatttcttgac

SEQ ID NO: 1095 - RNA, 21 nt, synthetic construct
  source 1..21: mol_type = other RNA
tacaaggcaatatcatacccc

SEQ ID NO: 1096 - RNA, 21 nt, synthetic construct
  source 1..21: mol_type = other RNA
tcagtttcaacaaggcaatac

SEQ ID NO: 1097 - RNA, 21 nt, synthetic construct
  source 1..21: mol_type = other RNA
tggtttccagtttcaacaagc

SEQ ID NO: 1098 - RNA, 21 nt, synthetic construct
  source 1..21: mol_type = other RNA
ttgtaattcactgtggtttcg

SEQ ID NO: 1099 - RNA, 21 nt, synthetic construct
  source 1..21: mol_type = other RNA
ttatcttggctttctggagac

SEQ ID NO: 1100 - RNA, 21 nt, synthetic construct
  source 1..21: mol_type = other RNA
tggtatcttggctttctggac

SEQ ID NO: 1101 - RNA, 21 nt, synthetic construct
  source 1..21: mol_type = other RNA
tctaagggtatcttggctttg

SEQ ID NO: 1102 - RNA, 21 nt, synthetic construct
  source 1..21: mol_type = other RNA
ttcactaagggtatcttggcc

SEQ ID NO: 1103 - RNA, 21 nt, synthetic construct
  source 1..21: mol_type = other RNA
tggtcactaagggtatcttgc

SEQ ID NO: 1104 - RNA, 21 nt, synthetic construct
  source 1..21: mol_type = other RNA
ttggtcactaagggtatcttc

SEQ ID NO: 1105 - RNA, 21 nt, synthetic construct
  source 1..21: mol_type = other RNA
tatctcttctggcactcttcc

SEQ ID NO: 1106 - RNA, 21 nt, synthetic construct
  source 1..21: mol_type = other RNA
tctctgtatctcttctggcag

SEQ ID NO: 1107 - RNA, 21 nt, synthetic construct
  source 1..21: mol_type = other RNA
tatgtcctctgtatctcttcc

SEQ ID NO: 1108 - RNA, 21 nt, synthetic construct
  source 1..21: mol_type = other RNA
tttatgtcctctgtatctctc

SEQ ID NO: 1109 - RNA, 21 nt, synthetic construct
  source 1..21: mol_type = other RNA
tattttatgtcctctgtatcc

SEQ ID NO: 1110 - RNA, 21 nt, synthetic construct
  source 1..21: mol_type = other RNA
ttattttatgtcctctgtatg

SEQ ID NO: 1111 - RNA, 21 nt, synthetic construct
  source 1..21: mol_type = other RNA
ttatgggttattttatgtccc

SEQ ID NO: 1112 - RNA, 21 nt, synthetic construct
  source 1..21: mol_type = other RNA
ttcatcttatgggttattttc

SEQ ID NO: 1113 - RNA, 21 nt, synthetic construct
  source 1..21: mol_type = other RNA
tatcatcttatgggttatttc

SEQ ID NO: 1114 - RNA, 21 nt, synthetic construct
  source 1..21: mol_type = other RNA
tcagatcatcttatgggttac

SEQ ID NO: 1115 - RNA, 21 nt, synthetic construct
  source 1..21: mol_type = other RNA
tacagatcatcttatgggttc

SEQ ID NO: 1116 - RNA, 21 nt, synthetic construct
  source 1..21: mol_type = other RNA
tcagaatccagtccacgtacc

SEQ ID NO: 1117 - RNA, 21 nt, synthetic construct
  source 1..21: mol_type = other RNA
tttctccagaatccagtccag

SEQ ID NO: 1118 - RNA, 21 nt, synthetic construct
  source 1..21: mol_type = other RNA
ttgagttttctccagaatccc

SEQ ID NO: 1119 - RNA, 21 nt, synthetic construct
  source 1..21: mol_type = other RNA
tttgagttttctccagaatcg

SEQ ID NO: 1120 - RNA, 21 nt, synthetic construct
  source 1..21: mol_type = other RNA
tcttgagttttctccagaatg

SEQ ID NO: 1121 - RNA, 21 nt, synthetic construct
  source 1..21: mol_type = other RNA
ttactgtggaggaagcatgcc

SEQ ID NO: 1122 - RNA, 21 nt, synthetic construct
  source 1..21: mol_type = other RNA
tcagtttgttttcttctagcc

SEQ ID NO: 1123 - RNA, 21 nt, synthetic construct
  source 1..21: mol_type = other RNA
tacagtttgttttcttctagg

SEQ ID NO: 1124 - RNA, 21 nt, synthetic construct
  source 1..21: mol_type = other RNA
tgacagtttgttttcttctac

SEQ ID NO: 1125 - RNA, 21 nt, synthetic construct
  source 1..21: mol_type = other RNA
ttgacagtttgttttcttctc

SEQ ID NO: 1126 - RNA, 21 nt, synthetic construct
  source 1..21: mol_type = other RNA
ttgtgacagtttgttttcttg

SEQ ID NO: 1127 - RNA, 21 nt, synthetic construct
  source 1..21: mol_type = other RNA
tttgttttcttctagcatttt

SEQ ID NO: 1128 - RNA, 21 nt, synthetic construct
  source 1..21: mol_type = other RNA
ttttgttttcttctagcattt

SEQ ID NO: 1129 - RNA, 21 nt, synthetic construct
  source 1..21: mol_type = other RNA
tgtttgttttcttctagcatt

SEQ ID NO: 1130 - RNA, 21 nt, synthetic construct
  source 1..21: mol_type = other RNA
tagtttgttttcttctagcat

SEQ ID NO: 1131 - RNA, 21 nt, synthetic construct
  source 1..21: mol_type = other RNA
tcagtttgttttcttctagca

SEQ ID NO: 1132 - RNA, 21 nt, synthetic construct
  source 1..21: mol_type = other RNA
tacagtttgttttcttctagc

SEQ ID NO: 1133 - RNA, 21 nt, synthetic construct
  source 1..21: mol_type = other RNA
tgacagtttgttttcttctag

SEQ ID NO: 1134 - RNA, 21 nt, synthetic construct
  source 1..21: mol_type = other RNA
ttgacagtttgttttcttcta

SEQ ID NO: 1135 - RNA, 21 nt, synthetic construct
  source 1..21: mol_type = other RNA
tgtgacagtttgttttcttct

SEQ ID NO: 1136 - RNA, 21 nt, synthetic construct
  source 1..21: mol_type = other RNA
ttgtgacagtttgttttcttc

SEQ ID NO: 1137 - RNA, 21 nt, synthetic construct
  source 1..21: mol_type = other RNA
tttgtgacagtttgttttctt

SEQ ID NO: 1138 - RNA, 21 nt, synthetic construct
  source 1..21: mol_type = other RNA
tcttgtgacagtttgttttct

SEQ ID NO: 1139 - RNA, 21 nt, synthetic construct
  source 1..21: mol_type = other RNA
tacttgtgacagtttgttttc

SEQ ID NO: 1140 - RNA, 21 nt, synthetic construct
  source 1..21: mol_type = other RNA
taacttgtgacagtttgtttt

SEQ ID NO: 1141 - RNA, 21 nt, synthetic construct
  source 1..21: mol_type = other RNA
tcaacttgtgacagtttgttt

SEQ ID NO: 1142 - RNA, 21 nt, synthetic construct
  source 1..21: mol_type = other RNA
tcattcaccagaaactgacgc

SEQ ID NO: 1143 - RNA, 21 nt, synthetic construct
  source 1..21: mol_type = other RNA
tgaatacccagaaatcgctcg

SEQ ID NO: 1144 - RNA, 19 nt, synthetic construct
  source 1..19: mol_type = other RNA
tataagaaaatcatcctga

SEQ ID NO: 1145 - RNA, 19 nt, synthetic construct
  source 1..19: mol_type = other RNA
ttataagaaaatcatcctg

SEQ ID NO: 1146 - RNA, 19 nt, synthetic construct
  source 1..19: mol_type = other RNA
tcacacattcaccagaaac

SEQ ID NO: 1147 - RNA, 19 nt, synthetic construct
  source 1..19: mol_type = other RNA
tcattgcttgaaagaatac

SEQ ID NO: 1148 - RNA, 19 nt, synthetic construct
  source 1..19: mol_type = other RNA
tttggtgtgagcattgctt

SEQ ID NO: 1149 - RNA, 19 nt, synthetic construct
  source 1..19: mol_type = other RNA
ttttggtgtgagcattgct

SEQ ID NO: 1150 - RNA, 19 nt, synthetic construct
  source 1..19: mol_type = other RNA
tcataaatgtctttgttgc

SEQ ID NO: 1151 - RNA, 19 nt, synthetic construct
  source 1..19: mol_type = other RNA
ttagtttatgcccttcatg

SEQ ID NO: 1152 - RNA, 19 nt, synthetic construct
  source 1..19: mol_type = other RNA
tgctgttatagtttatgcc

SEQ ID NO: 1153 - RNA, 19 nt, synthetic construct
  source 1..19: mol_type = other RNA
tgattttggtggtacactc

SEQ ID NO: 1154 - RNA, 19 nt, synthetic construct
  source 1..19: mol_type = other RNA
ttgattttggtggtacact

SEQ ID NO: 1155 - RNA, 19 nt, synthetic construct
  source 1..19: mol_type = other RNA
tttgattttggtggtacac

SEQ ID NO: 1156 - RNA, 19 nt, synthetic construct
  source 1..19: mol_type = other RNA
taatcttaggtgactctac

SEQ ID NO: 1157 - RNA, 19 nt, synthetic construct
  source 1..19: mol_type = other RNA
tcacgcaaaatcttaggtg

SEQ ID NO: 1158 - RNA, 19 nt, synthetic construct
  source 1..19: mol_type = other RNA
tacacgcaaaatcttaggt

SEQ ID NO: 1159 - RNA, 19 nt, synthetic construct
  source 1..19: mol_type = other RNA
tagacacgcaaaatcttag

SEQ ID NO: 1160 - RNA, 19 nt, synthetic construct
  source 1..19: mol_type = other RNA
ttagacacgcaaaatctta

SEQ ID NO: 1161 - RNA, 19 nt, synthetic construct
  source 1..19: mol_type = other RNA
ttgtagacacgcaaaatct

SEQ ID NO: 1162 - RNA, 19 nt, synthetic construct
  source 1..19: mol_type = other RNA
tttgatttaaaatgccact

SEQ ID NO: 1163 - RNA, 19 nt, synthetic construct
  source 1..19: mol_type = other RNA
tattgatttaaaatgccac

SEQ ID NO: 1164 - RNA, 19 nt, synthetic construct
  source 1..19: mol_type = other RNA
ttatttcagattgatttaa

SEQ ID NO: 1165 - RNA, 19 nt, synthetic construct
  source 1..19: mol_type = other RNA
ttttatttcagattgattt

SEQ ID NO: 1166 - RNA, 19 nt, synthetic construct
  source 1..19: mol_type = other RNA
taaccccaaagaaagatgt

SEQ ID NO: 1167 - RNA, 19 nt, synthetic construct
  source 1..19: mol_type = other RNA
ttgaaccccaaagaaagat

SEQ ID NO: 1168 - RNA, 19 nt, synthetic construct
  source 1..19: mol_type = other RNA
tttgaaccccaaagaaaga

SEQ ID NO: 1169 - RNA, 19 nt, synthetic construct
  source 1..19: mol_type = other RNA
ttttcttgaaccccaaaga

SEQ ID NO: 1170 - RNA, 19 nt, synthetic construct
  source 1..19: mol_type = other RNA
tatttcttgaaccccaaag

SEQ ID NO: 1171 - RNA, 19 nt, synthetic construct
  source 1..19: mol_type = other RNA
ttatttcttgaaccccaaa

SEQ ID NO: 1172 - RNA, 19 nt, synthetic construct
  source 1..19: mol_type = other RNA
tgattattatttcttgaac

SEQ ID NO: 1173 - RNA, 19 nt, synthetic construct
  source 1..19: mol_type = other RNA
tgatcatggattattattt

SEQ ID NO: 1174 - RNA, 19 nt, synthetic construct
  source 1..19: mol_type = other RNA
ttttctgccattttatact

SEQ ID NO: 1175 - RNA, 19 nt, synthetic construct
  source 1..19: mol_type = other RNA
tctttctgccattttatac

SEQ ID NO: 1176 - RNA, 19 nt, synthetic construct
  source 1..19: mol_type = other RNA
taacaaggcaatatcatac

SEQ ID NO: 1177 - RNA, 19 nt, synthetic construct
  source 1..19: mol_type = other RNA
tgtttcaacaaggcaatat

SEQ ID NO: 1178 - RNA, 19 nt, synthetic construct
  source 1..19: mol_type = other RNA
tagtttcaacaaggcaata

SEQ ID NO: 1179 - RNA, 19 nt, synthetic construct
  source 1..19: mol_type = other RNA
tccagtttcaacaaggcaa

SEQ ID NO: 1180 - RNA, 19 nt, synthetic construct
  source 1..19: mol_type = other RNA
tgtttccagtttcaacaag

SEQ ID NO: 1181 - RNA, 19 nt, synthetic construct
  source 1..19: mol_type = other RNA
ttgtggtttccagtttcaa

SEQ ID NO: 1182 - RNA, 19 nt, synthetic construct
  source 1..19: mol_type = other RNA
tgtaattcactgtggtttc

SEQ ID NO: 1183 - RNA, 19 nt, synthetic construct
  source 1..19: mol_type = other RNA
ttgtgtaattcactgtggt

SEQ ID NO: 1184 - RNA, 19 nt, synthetic construct
  source 1..19: mol_type = other RNA
ttctgtgtaattcactgtg

SEQ ID NO: 1185 - RNA, 19 nt, synthetic construct
  source 1..19: mol_type = other RNA
tctttctggagagtatttt

SEQ ID NO: 1186 - RNA, 19 nt, synthetic construct
  source 1..19: mol_type = other RNA
tatcttggctttctggaga

SEQ ID NO: 1187 - RNA, 19 nt, synthetic construct
  source 1..19: mol_type = other RNA
tactaagggtatcttggct

SEQ ID NO: 1188 - RNA, 19 nt, synthetic construct
  source 1..19: mol_type = other RNA
ttatgtcctctgtatctct

SEQ ID NO: 1189 - RNA, 19 nt, synthetic construct
  source 1..19: mol_type = other RNA
tttttatgtcctctgtatc

SEQ ID NO: 1190 - RNA, 19 nt, synthetic construct
  source 1..19: mol_type = other RNA
tgttattttatgtcctctg

SEQ ID NO: 1191 - RNA, 19 nt, synthetic construct
  source 1..19: mol_type = other RNA
tggttattttatgtcctct

SEQ ID NO: 1192 - RNA, 19 nt, synthetic construct
  source 1..19: mol_type = other RNA
tatgggttattttatgtcc

SEQ ID NO: 1193 - RNA, 19 nt, synthetic construct
  source 1..19: mol_type = other RNA
tcttatgggttattttatg

SEQ ID NO: 1194 - RNA, 19 nt, synthetic construct
  source 1..19: mol_type = other RNA
ttcttatgggttattttat

SEQ ID NO: 1195 - RNA, 19 nt, synthetic construct
  source 1..19: mol_type = other RNA
tcacagatcatcttatggg

SEQ ID NO: 1196 - RNA, 19 nt, synthetic construct
  source 1..19: mol_type = other RNA
tttttctccagaatccagt

SEQ ID NO: 1197 - RNA, 19 nt, synthetic construct
  source 1..19: mol_type = other RNA
tgttttctccagaatccag

SEQ ID NO: 1198 - RNA, 19 nt, synthetic construct
  source 1..19: mol_type = other RNA
tttcatcctcctctgaatg

SEQ ID NO: 1199 - RNA, 19 nt, synthetic construct
  source 1..19: mol_type = other RNA
tatcatcctgaaaagacct

SEQ ID NO: 1200 - RNA, 19 nt, synthetic construct
  source 1..19: mol_type = other RNA
tagaaaatcatcctgaaaa

SEQ ID NO: 1201 - RNA, 19 nt, synthetic construct
  source 1..19: mol_type = other RNA
ttgatataagaaaatcatc

SEQ ID NO: 1202 - RNA, 19 nt, synthetic construct
  source 1..19: mol_type = other RNA
taggacacaagtaaaccat

SEQ ID NO: 1203 - RNA, 19 nt, synthetic construct
  source 1..19: mol_type = other RNA
ttgtcctattcactcttgg

SEQ ID NO: 1204 - RNA, 19 nt, synthetic construct
  source 1..19: mol_type = other RNA
tttgaaagaatacccagaa

SEQ ID NO: 1205 - RNA, 19 nt, synthetic construct
  source 1..19: mol_type = other RNA
tcttgaaagaatacccaga

SEQ ID NO: 1206 - RNA, 19 nt, synthetic construct
  source 1..19: mol_type = other RNA
ttgttgcaagcgcttattt

SEQ ID NO: 1207 - RNA, 19 nt, synthetic construct
  source 1..19: mol_type = other RNA
taaatgtctttgttgcaag

SEQ ID NO: 1208 - RNA, 19 nt, synthetic construct
  source 1..19: mol_type = other RNA
tgtccacataaatgtcttt

SEQ ID NO: 1209 - RNA, 19 nt, synthetic construct
  source 1..19: mol_type = other RNA
ttcatgtctaggtccacat

SEQ ID NO: 1210 - RNA, 19 nt, synthetic construct
  source 1..19: mol_type = other RNA
ttcttgagcactcttggca

SEQ ID NO: 1211 - RNA, 19 nt, synthetic construct
  source 1..19: mol_type = other RNA
tatccgtgcatctttcttg

SEQ ID NO: 1212 - RNA, 19 nt, synthetic construct
  source 1..19: mol_type = other RNA
tttaaggagacaaagattt

SEQ ID NO: 1213 - RNA, 19 nt, synthetic construct
  source 1..19: mol_type = other RNA
ttttaaggagacaaagatt

SEQ ID NO: 1214 - RNA, 19 nt, synthetic construct
  source 1..19: mol_type = other RNA
tttttaaggagacaaagat

SEQ ID NO: 1215 - RNA, 19 nt, synthetic construct
  source 1..19: mol_type = other RNA
tatgttttaaggagacaaa

SEQ ID NO: 1216 - RNA, 19 nt, synthetic construct
  source 1..19: mol_type = other RNA
tagatgttttaaggagaca

SEQ ID NO: 1217 - RNA, 19 nt, synthetic construct
  source 1..19: mol_type = other RNA
tcagatgttttaaggagac

SEQ ID NO: 1218 - RNA, 19 nt, synthetic construct
  source 1..19: mol_type = other RNA
ttcagatgttttaaggaga

SEQ ID NO: 1219 - RNA, 19 nt, synthetic construct
  source 1..19: mol_type = other RNA
tctctcagatgttttaagg

SEQ ID NO: 1220 - RNA, 19 nt, synthetic construct
  source 1..19: mol_type = other RNA
tccactctcagatgtttta

SEQ ID NO: 1221 - RNA, 19 nt, synthetic construct
  source 1..19: mol_type = other RNA
tactgaaaccagaaagagc

SEQ ID NO: 1222 - RNA, 19 nt, synthetic construct
  source 1..19: mol_type = other RNA
tcagcaacaatatccagtt

SEQ ID NO: 1223 - RNA, 19 nt, synthetic construct
  source 1..19: mol_type = other RNA
tgtatccagagatgcctcc

SEQ ID NO: 1224 - RNA, 19 nt, synthetic construct
  source 1..19: mol_type = other RNA
taatgtgtatccagagatg

SEQ ID NO: 1225 - RNA, 19 nt, synthetic construct
  source 1..19: mol_type = other RNA
tcattatccattttacaca

SEQ ID NO: 1226 - RNA, 19 nt, synthetic construct
  source 1..19: mol_type = other RNA
tctcattatccattttaca

SEQ ID NO: 1227 - RNA, 19 nt, synthetic construct
  source 1..19: mol_type = other RNA
tgcttgattttggtggtac

SEQ ID NO: 1228 - RNA, 19 nt, synthetic construct
  source 1..19: mol_type = other RNA
tgttcctccaacgatcctg

SEQ ID NO: 1229 - RNA, 19 nt, synthetic construct
  source 1..19: mol_type = other RNA
taaaatcttaggtgactct

SEQ ID NO: 1230 - RNA, 19 nt, synthetic construct
  source 1..19: mol_type = other RNA
tcagattgatttaaaatgc

SEQ ID NO: 1231 - RNA, 19 nt, synthetic construct
  source 1..19: mol_type = other RNA
ttcagattgatttaaaatg

SEQ ID NO: 1232 - RNA, 19 nt, synthetic construct
  source 1..19: mol_type = other RNA
tttcagattgatttaaaat

SEQ ID NO: 1233 - RNA, 19 nt, synthetic construct
  source 1..19: mol_type = other RNA
tccccaaagaaagatgtgt

SEQ ID NO: 1234 - RNA, 19 nt, synthetic construct
  source 1..19: mol_type = other RNA
ttcttgaaccccaaagaaa

SEQ ID NO: 1235 - RNA, 19 nt, synthetic construct
  source 1..19: mol_type = other RNA
tttatttcttgaaccccaa

SEQ ID NO: 1236 - RNA, 19 nt, synthetic construct
  source 1..19: mol_type = other RNA
ttggattattatttcttga

SEQ ID NO: 1237 - RNA, 19 nt, synthetic construct
  source 1..19: mol_type = other RNA
tatggattattatttcttg

SEQ ID NO: 1238 - RNA, 19 nt, synthetic construct
  source 1..19: mol_type = other RNA
tacaaggcaatatcatacc

SEQ ID NO: 1239 - RNA, 19 nt, synthetic construct
  source 1..19: mol_type = other RNA
tcagtttcaacaaggcaat

SEQ ID NO: 1240 - RNA, 19 nt, synthetic construct
  source 1..19: mol_type = other RNA
tggtttccagtttcaacaa

SEQ ID NO: 1241 - RNA, 19 nt, synthetic construct
  source 1..19: mol_type = other RNA
ttgtaattcactgtggttt

SEQ ID NO: 1242 - RNA, 19 nt, synthetic construct
  source 1..19: mol_type = other RNA
ttatcttggctttctggag

SEQ ID NO: 1243 - RNA, 19 nt, synthetic construct
  source 1..19: mol_type = other RNA
tggtatcttggctttctgg

SEQ ID NO: 1244 - RNA, 19 nt, synthetic construct
  source 1..19: mol_type = other RNA
tctaagggtatcttggctt

SEQ ID NO: 1245 - RNA, 19 nt, synthetic construct
  source 1..19: mol_type = other RNA
ttcactaagggtatcttgg

SEQ ID NO: 1246 - RNA, 19 nt, synthetic construct
  source 1..19: mol_type = other RNA
tggtcactaagggtatctt

SEQ ID NO: 1247 - RNA, 19 nt, synthetic construct
  source 1..19: mol_type = other RNA
ttggtcactaagggtatct

SEQ ID NO: 1248 - RNA, 19 nt, synthetic construct
  source 1..19: mol_type = other RNA
tatctcttctggcactctt

SEQ ID NO: 1249 - RNA, 19 nt, synthetic construct
  source 1..19: mol_type = other RNA
tctctgtatctcttctggc

SEQ ID NO: 1250 - RNA, 19 nt, synthetic construct
  source 1..19: mol_type = other RNA
tatgtcctctgtatctctt

SEQ ID NO: 1251 - RNA, 19 nt, synthetic construct
  source 1..19: mol_type = other RNA
tttatgtcctctgtatctc

SEQ ID NO: 1252 - RNA, 19 nt, synthetic construct
  source 1..19: mol_type = other RNA
tattttatgtcctctgtat

SEQ ID NO: 1253 - RNA, 19 nt, synthetic construct
  source 1..19: mol_type = other RNA
ttattttatgtcctctgta

SEQ ID NO: 1254 - RNA, 19 nt, synthetic construct
  source 1..19: mol_type = other RNA
ttatgggttattttatgtc

SEQ ID NO: 1255 - RNA, 19 nt, synthetic construct
  source 1..19: mol_type = other RNA
ttcatcttatgggttattt

SEQ ID NO: 1256 - RNA, 19 nt, synthetic construct
  source 1..19: mol_type = other RNA
tatcatcttatgggttatt

SEQ ID NO: 1257 - RNA, 19 nt, synthetic construct
  source 1..19: mol_type = other RNA
tcagatcatcttatgggtt

SEQ ID NO: 1258 - RNA, 19 nt, synthetic construct
  source 1..19: mol_type = other RNA
tacagatcatcttatgggt

SEQ ID NO: 1259 - RNA, 19 nt, synthetic construct
  source 1..19: mol_type = other RNA
tcagaatccagtccacgta

SEQ ID NO: 1260 - RNA, 19 nt, synthetic construct
  source 1..19: mol_type = other RNA
tttctccagaatccagtcc

SEQ ID NO: 1261 - RNA, 19 nt, synthetic construct
  source 1..19: mol_type = other RNA
ttgagttttctccagaatc

SEQ ID NO: 1262 - RNA, 19 nt, synthetic construct
  source 1..19: mol_type = other RNA
tttgagttttctccagaat

SEQ ID NO: 1263 - RNA, 19 nt, synthetic construct
  source 1..19: mol_type = other RNA
tcttgagttttctccagaa

SEQ ID NO: 1264 - RNA, 19 nt, synthetic construct
  source 1..19: mol_type = other RNA
ttactgtggaggaagcatg

SEQ ID NO: 1265 - RNA, 19 nt, synthetic construct
  source 1..19: mol_type = other RNA
tcagtttgttttcttctag

SEQ ID NO: 1266 - RNA, 19 nt, synthetic construct
  source 1..19: mol_type = other RNA
tacagtttgttttcttcta

SEQ ID NO: 1267 - RNA, 19 nt, synthetic construct
  source 1..19: mol_type = other RNA
tgacagtttgttttcttct

SEQ ID NO: 1268 - RNA, 19 nt, synthetic construct
  source 1..19: mol_type = other RNA
ttgacagtttgttttcttc

SEQ ID NO: 1269 - RNA, 19 nt, synthetic construct
  source 1..19: mol_type = other RNA
ttgtgacagtttgttttct

SEQ ID NO: 1270 - RNA, 19 nt, synthetic construct
  source 1..19: mol_type = other RNA
tttgttttcttctagcatt

SEQ ID NO: 1271 - RNA, 19 nt, synthetic construct
  source 1..19: mol_type = other RNA
ttttgttttcttctagcat

SEQ ID NO: 1272 - RNA, 19 nt, synthetic construct
  source 1..19: mol_type = other RNA
tgtttgttttcttctagca

SEQ ID NO: 1273 - RNA, 19 nt, synthetic construct
  source 1..19: mol_type = other RNA
tagtttgttttcttctagc

SEQ ID NO: 1274 - RNA, 19 nt, synthetic construct
  source 1..19: mol_type = other RNA
tcagtttgttttcttctag

SEQ ID NO: 1275 - RNA, 19 nt, synthetic construct
  source 1..19: mol_type = other RNA
tacagtttgttttcttcta

SEQ ID NO: 1276 - RNA, 19 nt, synthetic construct
  source 1..19: mol_type = other RNA
tgacagtttgttttcttct

SEQ ID NO: 1277 - RNA, 19 nt, synthetic construct
  source 1..19: mol_type = other RNA
ttgacagtttgttttcttc

SEQ ID NO: 1278 - RNA, 19 nt, synthetic construct
  source 1..19: mol_type = other RNA
tgtgacagtttgttttctt

SEQ ID NO: 1279 - RNA, 19 nt, synthetic construct
  source 1..19: mol_type = other RNA
ttgtgacagtttgttttct

SEQ ID NO: 1280 - RNA, 19 nt, synthetic construct
  source 1..19: mol_type = other RNA
tttgtgacagtttgttttc

SEQ ID NO: 1281 - RNA, 19 nt, synthetic construct
  source 1..19: mol_type = other RNA
tcttgtgacagtttgtttt

SEQ ID NO: 1282 - RNA, 19 nt, synthetic construct
  source 1..19: mol_type = other RNA
tacttgtgacagtttgttt

SEQ ID NO: 1283 - RNA, 19 nt, synthetic construct
  source 1..19: mol_type = other RNA
taacttgtgacagtttgtt

SEQ ID NO: 1284 - RNA, 19 nt, synthetic construct
  source 1..19: mol_type = other RNA
tcaacttgtgacagtttgt

SEQ ID NO: 1285 - RNA, 19 nt, synthetic construct
  source 1..19: mol_type = other RNA
tcattcaccagaaactgac

SEQ ID NO: 1286 - RNA, 19 nt, synthetic construct
  source 1..19: mol_type = other RNA
tgaatacccagaaatcgct

SEQ ID NO: 1287 - RNA, 21 nt, synthetic construct
  source 1..21: mol_type = other RNA
gttcaggatgattttcttata

SEQ ID NO: 1288 - RNA, 21 nt, synthetic construct
  source 1..21: mol_type = other RNA
gtcaggatgattttcttataa

SEQ ID NO: 1289 - RNA, 21 nt, synthetic construct
  source 1..21: mol_type = other RNA
gagtttctggtgaatgtgtga

SEQ ID NO: 1290 - RNA, 21 nt, synthetic construct
  source 1..21: mol_type = other RNA
cggtattctttcaagcaatga

SEQ ID NO: 1291 - RNA, 21 nt, synthetic construct
  source 1..21: mol_type = other RNA
gcaagcaatgctcacaccaaa

SEQ ID NO: 1292 - RNA, 21 nt, synthetic construct
  source 1..21: mol_type = other RNA
gaagcaatgctcacaccaaaa

SEQ ID NO: 1293 - RNA, 21 nt, synthetic construct
  source 1..21: mol_type = other RNA
gtgcaacaaagacatttatga

SEQ ID NO: 1294 - RNA, 21 nt, synthetic construct
  source 1..21: mol_type = other RNA
cacatgaagggcataaactaa

SEQ ID NO: 1295 - RNA, 21 nt, synthetic construct
  source 1..21: mol_type = other RNA
ggggcataaactataacagca

SEQ ID NO: 1296 - RNA, 21 nt, synthetic construct
  source 1..21: mol_type = other RNA
gtgagtgtaccaccaaaatca

SEQ ID NO: 1297 - RNA, 21 nt, synthetic construct
  source 1..21: mol_type = other RNA
ggagtgtaccaccaaaatcaa

SEQ ID NO: 1298 - RNA, 21 nt, synthetic construct
  source 1..21: mol_type = other RNA
cagtgtaccaccaaaatcaaa

SEQ ID NO: 1299 - RNA, 21 nt, synthetic construct
  source 1..21: mol_type = other RNA
cggtagagtcacctaagatta

SEQ ID NO: 1300 - RNA, 21 nt, synthetic construct
  source 1..21: mol_type = other RNA
ctcacctaagattttgcgtga

SEQ ID NO: 1301 - RNA, 21 nt, synthetic construct
  source 1..21: mol_type = other RNA
gcacctaagattttgcgtgta

SEQ ID NO: 1302 - RNA, 21 nt, synthetic construct
  source 1..21: mol_type = other RNA
gcctaagattttgcgtgtcta

SEQ ID NO: 1303 - RNA, 21 nt, synthetic construct
  source 1..21: mol_type = other RNA
gctaagattttgcgtgtctaa

SEQ ID NO: 1304 - RNA, 21 nt, synthetic construct
  source 1..21: mol_type = other RNA
gaagattttgcgtgtctacaa

SEQ ID NO: 1305 - RNA, 21 nt, synthetic construct
  source 1..21: mol_type = other RNA
gcagtggcattttaaatcaaa

SEQ ID NO: 1306 - RNA, 21 nt, synthetic construct
  source 1..21: mol_type = other RNA
gagtggcattttaaatcaata

SEQ ID NO: 1307 - RNA, 21 nt, synthetic construct
  source 1..21: mol_type = other RNA
gtttaaatcaatctgaaataa

SEQ ID NO: 1308 - RNA, 21 nt, synthetic construct
  source 1..21: mol_type = other RNA
gtaaatcaatctgaaataaaa

SEQ ID NO: 1309 - RNA, 21 nt, synthetic construct
  source 1..21: mol_type = other RNA
gcacatctttctttggggtta

SEQ ID NO: 1310 - RNA, 21 nt, synthetic construct
  source 1..21: mol_type = other RNA
gcatctttctttggggttcaa

SEQ ID NO: 1311 - RNA, 21 nt, synthetic construct
  source 1..21: mol_type = other RNA
gatctttctttggggttcaaa

SEQ ID NO: 1312 - RNA, 21 nt, synthetic construct
  source 1..21: mol_type = other RNA
gttctttggggttcaagaaaa

SEQ ID NO: 1313 - RNA, 21 nt, synthetic construct
  source 1..21: mol_type = other RNA
gtctttggggttcaagaaata

SEQ ID NO: 1314 - RNA, 21 nt, synthetic construct
  source 1..21: mol_type = other RNA
gctttggggttcaagaaataa

SEQ ID NO: 1315 - RNA, 21 nt, synthetic construct
  source 1..21: mol_type = other RNA
cggttcaagaaataataatca

SEQ ID NO: 1316 - RNA, 21 nt, synthetic construct
  source 1..21: mol_type = other RNA
ggaaataataatccatgatca

SEQ ID NO: 1317 - RNA, 21 nt, synthetic construct
  source 1..21: mol_type = other RNA
gcagtataaaatggcagaaaa

SEQ ID NO: 1318 - RNA, 21 nt, synthetic construct
  source 1..21: mol_type = other RNA
gagtataaaatggcagaaaga

SEQ ID NO: 1319 - RNA, 21 nt, synthetic construct
  source 1..21: mol_type = other RNA
cggtatgatattgccttgtta

SEQ ID NO: 1320 - RNA, 21 nt, synthetic construct
  source 1..21: mol_type = other RNA
ggatattgccttgttgaaaca

SEQ ID NO: 1321 - RNA, 21 nt, synthetic construct
  source 1..21: mol_type = other RNA
catattgccttgttgaaacta

SEQ ID NO: 1322 - RNA, 21 nt, synthetic construct
  source 1..21: mol_type = other RNA
gattgccttgttgaaactgga

SEQ ID NO: 1323 - RNA, 21 nt, synthetic construct
  source 1..21: mol_type = other RNA
cccttgttgaaactggaaaca

SEQ ID NO: 1324 - RNA, 21 nt, synthetic construct
  source 1..21: mol_type = other RNA
ggttgaaactggaaaccacaa

SEQ ID NO: 1325 - RNA, 21 nt, synthetic construct
  source 1..21: mol_type = other RNA
gggaaaccacagtgaattaca

SEQ ID NO: 1326 - RNA, 21 nt, synthetic construct
  source 1..21: mol_type = other RNA
gaaccacagtgaattacacaa

SEQ ID NO: 1327 - RNA, 21 nt, synthetic construct
  source 1..21: mol_type = other RNA
gccacagtgaattacacagaa

SEQ ID NO: 1328 - RNA, 21 nt, synthetic construct
  source 1..21: mol_type = other RNA
gcaaaatactctccagaaaga

SEQ ID NO: 1329 - RNA, 21 nt, synthetic construct
  source 1..21: mol_type = other RNA
gctctccagaaagccaagata

SEQ ID NO: 1330 - RNA, 21 nt, synthetic construct
  source 1..21: mol_type = other RNA
gaagccaagatacccttagta

SEQ ID NO: 1331 - RNA, 21 nt, synthetic construct
  source 1..21: mol_type = other RNA
caagagatacagaggacataa

SEQ ID NO: 1332 - RNA, 21 nt, synthetic construct
  source 1..21: mol_type = other RNA
cagatacagaggacataaaaa

SEQ ID NO: 1333 - RNA, 21 nt, synthetic construct
  source 1..21: mol_type = other RNA
gacagaggacataaaataaca

SEQ ID NO: 1334 - RNA, 21 nt, synthetic construct
  source 1..21: mol_type = other RNA
gcagaggacataaaataacca

SEQ ID NO: 1335 - RNA, 21 nt, synthetic construct
  source 1..21: mol_type = other RNA
caggacataaaataacccata

SEQ ID NO: 1336 - RNA, 21 nt, synthetic construct
  source 1..21: mol_type = other RNA
cacataaaataacccataaga

SEQ ID NO: 1337 - RNA, 21 nt, synthetic construct
  source 1..21: mol_type = other RNA
gcataaaataacccataagaa

SEQ ID NO: 1338 - RNA, 21 nt, synthetic construct
  source 1..21: mol_type = other RNA
gacccataagatgatctgtga

SEQ ID NO: 1339 - RNA, 21 nt, synthetic construct
  source 1..21: mol_type = other RNA
cgactggattctggagaaaaa

SEQ ID NO: 1340 - RNA, 21 nt, synthetic construct
  source 1..21: mol_type = other RNA
cactggattctggagaaaaca

SEQ ID NO: 1341 - RNA, 21 nt, synthetic construct
  source 1..21: mol_type = other RNA
gtcattcagaggaggatgaaa

SEQ ID NO: 1342 - RNA, 21 nt, synthetic construct
  source 1..21: mol_type = other RNA
gaaggtcttttcaggatgata

SEQ ID NO: 1343 - RNA, 21 nt, synthetic construct
  source 1..21: mol_type = other RNA
gcttttcaggatgattttcta

SEQ ID NO: 1344 - RNA, 21 nt, synthetic construct
  source 1..21: mol_type = other RNA
gggatgattttcttatatcaa

SEQ ID NO: 1345 - RNA, 21 nt, synthetic construct
  source 1..21: mol_type = other RNA
ggatggtttacttgtgtccta

SEQ ID NO: 1346 - RNA, 21 nt, synthetic construct
  source 1..21: mol_type = other RNA
ggccaagagtgaataggacaa

SEQ ID NO: 1347 - RNA, 21 nt, synthetic construct
  source 1..21: mol_type = other RNA
gtttctgggtattctttcaaa

SEQ ID NO: 1348 - RNA, 21 nt, synthetic construct
  source 1..21: mol_type = other RNA
gttctgggtattctttcaaga

SEQ ID NO: 1349 - RNA, 21 nt, synthetic construct
  source 1..21: mol_type = other RNA
gcaaataagcgcttgcaacaa

SEQ ID NO: 1350 - RNA, 21 nt, synthetic construct
  source 1..21: mol_type = other RNA
ggcttgcaacaaagacattta

SEQ ID NO: 1351 - RNA, 21 nt, synthetic construct
  source 1..21: mol_type = other RNA
gcaaagacatttatgtggaca

SEQ ID NO: 1352 - RNA, 21 nt, synthetic construct
  source 1..21: mol_type = other RNA
gtatgtggacctagacatgaa

SEQ ID NO: 1353 - RNA, 21 nt, synthetic construct
  source 1..21: mol_type = other RNA
cttgccaagagtgctcaagaa

SEQ ID NO: 1354 - RNA, 21 nt, synthetic construct
  source 1..21: mol_type = other RNA
cccaagaaagatgcacggata

SEQ ID NO: 1355 - RNA, 21 nt, synthetic construct
  source 1..21: mol_type = other RNA
ggaaatctttgtctccttaaa

SEQ ID NO: 1356 - RNA, 21 nt, synthetic construct
  source 1..21: mol_type = other RNA
caaatctttgtctccttaaaa

SEQ ID NO: 1357 - RNA, 21 nt, synthetic construct
  source 1..21: mol_type = other RNA
gaatctttgtctccttaaaaa

SEQ ID NO: 1358 - RNA, 21 nt, synthetic construct
  source 1..21: mol_type = other RNA
gctttgtctccttaaaacata

SEQ ID NO: 1359 - RNA, 21 nt, synthetic construct
  source 1..21: mol_type = other RNA
gttgtctccttaaaacatcta

SEQ ID NO: 1360 - RNA, 21 nt, synthetic construct
  source 1..21: mol_type = other RNA
gtgtctccttaaaacatctga

SEQ ID NO: 1361 - RNA, 21 nt, synthetic construct
  source 1..21: mol_type = other RNA
ggtctccttaaaacatctgaa

SEQ ID NO: 1362 - RNA, 21 nt, synthetic construct
  source 1..21: mol_type = other RNA
gtccttaaaacatctgagaga

SEQ ID NO: 1363 - RNA, 21 nt, synthetic construct
  source 1..21: mol_type = other RNA
gttaaaacatctgagagtgga

SEQ ID NO: 1364 - RNA, 21 nt, synthetic construct
  source 1..21: mol_type = other RNA
gagctctttctggtttcagta

SEQ ID NO: 1365 - RNA, 21 nt, synthetic construct
  source 1..21: mol_type = other RNA
ggaactggatattgttgctga

SEQ ID NO: 1366 - RNA, 21 nt, synthetic construct
  source 1..21: mol_type = other RNA
caggaggcatctctggataca

SEQ ID NO: 1367 - RNA, 21 nt, synthetic construct
  source 1..21: mol_type = other RNA
cgcatctctggatacacatta

SEQ ID NO: 1368 - RNA, 21 nt, synthetic construct
  source 1..21: mol_type = other RNA
cttgtgtaaaatggataatga

SEQ ID NO: 1369 - RNA, 21 nt, synthetic construct
  source 1..21: mol_type = other RNA
ggtgtaaaatggataatgaga

SEQ ID NO: 1370 - RNA, 21 nt, synthetic construct
  source 1..21: mol_type = other RNA
ctgtaccaccaaaatcaagca

SEQ ID NO: 1371 - RNA, 21 nt, synthetic construct
  source 1..21: mol_type = other RNA
gccaggatcgttggaggaaca

SEQ ID NO: 1372 - RNA, 21 nt, synthetic construct
  source 1..21: mol_type = other RNA
ctagagtcacctaagatttta

SEQ ID NO: 1373 - RNA, 21 nt, synthetic construct
  source 1..21: mol_type = other RNA
gggcattttaaatcaatctga

SEQ ID NO: 1374 - RNA, 21 nt, synthetic construct
  source 1..21: mol_type = other RNA
cgcattttaaatcaatctgaa

SEQ ID NO: 1375 - RNA, 21 nt, synthetic construct
  source 1..21: mol_type = other RNA
ccattttaaatcaatctgaaa

SEQ ID NO: 1376 - RNA, 21 nt, synthetic construct
  source 1..21: mol_type = other RNA
cgacacatctttctttgggga

SEQ ID NO: 1377 - RNA, 21 nt, synthetic construct
  source 1..21: mol_type = other RNA
gctttctttggggttcaagaa

SEQ ID NO: 1378 - RNA, 21 nt, synthetic construct
  source 1..21: mol_type = other RNA
gtttggggttcaagaaataaa

SEQ ID NO: 1379 - RNA, 21 nt, synthetic construct
  source 1..21: mol_type = other RNA
cttcaagaaataataatccaa

SEQ ID NO: 1380 - RNA, 21 nt, synthetic construct
  source 1..21: mol_type = other RNA
gtcaagaaataataatccata

SEQ ID NO: 1381 - RNA, 21 nt, synthetic construct
  source 1..21: mol_type = other RNA
ggggtatgatattgccttgta

SEQ ID NO: 1382 - RNA, 21 nt, synthetic construct
  source 1..21: mol_type = other RNA
gtattgccttgttgaaactga

SEQ ID NO: 1383 - RNA, 21 nt, synthetic construct
  source 1..21: mol_type = other RNA
gcttgttgaaactggaaacca

SEQ ID NO: 1384 - RNA, 21 nt, synthetic construct
  source 1..21: mol_type = other RNA
cgaaaccacagtgaattacaa

SEQ ID NO: 1385 - RNA, 21 nt, synthetic construct
  source 1..21: mol_type = other RNA
gtctccagaaagccaagataa

SEQ ID NO: 1386 - RNA, 21 nt, synthetic construct
  source 1..21: mol_type = other RNA
gtccagaaagccaagatacca

SEQ ID NO: 1387 - RNA, 21 nt, synthetic construct
  source 1..21: mol_type = other RNA
caaagccaagatacccttaga

SEQ ID NO: 1388 - RNA, 21 nt, synthetic construct
  source 1..21: mol_type = other RNA
ggccaagatacccttagtgaa

SEQ ID NO: 1389 - RNA, 21 nt, synthetic construct
  source 1..21: mol_type = other RNA
gcaagatacccttagtgacca

SEQ ID NO: 1390 - RNA, 21 nt, synthetic construct
  source 1..21: mol_type = other RNA
gaagatacccttagtgaccaa

SEQ ID NO: 1391 - RNA, 21 nt, synthetic construct
  source 1..21: mol_type = other RNA
ggaagagtgccagaagagata

SEQ ID NO: 1392 - RNA, 21 nt, synthetic construct
  source 1..21: mol_type = other RNA
ctgccagaagagatacagaga

SEQ ID NO: 1393 - RNA, 21 nt, synthetic construct
  source 1..21: mol_type = other RNA
ggaagagatacagaggacata

SEQ ID NO: 1394 - RNA, 21 nt, synthetic construct
  source 1..21: mol_type = other RNA
gagagatacagaggacataaa

SEQ ID NO: 1395 - RNA, 21 nt, synthetic construct
  source 1..21: mol_type = other RNA
ggatacagaggacataaaata

SEQ ID NO: 1396 - RNA, 21 nt, synthetic construct
  source 1..21: mol_type = other RNA
catacagaggacataaaataa

SEQ ID NO: 1397 - RNA, 21 nt, synthetic construct
  source 1..21: mol_type = other RNA
gggacataaaataacccataa

SEQ ID NO: 1398 - RNA, 21 nt, synthetic construct
  source 1..21: mol_type = other RNA
gaaaataacccataagatgaa

SEQ ID NO: 1399 - RNA, 21 nt, synthetic construct
  source 1..21: mol_type = other RNA
gaaataacccataagatgata

SEQ ID NO: 1400 - RNA, 21 nt, synthetic construct
  source 1..21: mol_type = other RNA
gtaacccataagatgatctga

SEQ ID NO: 1401 - RNA, 21 nt, synthetic construct
  source 1..21: mol_type = other RNA
gaacccataagatgatctgta

SEQ ID NO: 1402 - RNA, 21 nt, synthetic construct
  source 1..21: mol_type = other RNA
ggtacgtggactggattctga

SEQ ID NO: 1403 - RNA, 21 nt, synthetic construct
  source 1..21: mol_type = other RNA
ctggactggattctggagaaa

SEQ ID NO: 1404 - RNA, 21 nt, synthetic construct
  source 1..21: mol_type = other RNA
gggattctggagaaaactcaa

SEQ ID NO: 1405 - RNA, 21 nt, synthetic construct
  source 1..21: mol_type = other RNA
cgattctggagaaaactcaaa

SEQ ID NO: 1406 - RNA, 21 nt, synthetic construct
  source 1..21: mol_type = other RNA
cattctggagaaaactcaaga

SEQ ID NO: 1407 - RNA, 21 nt, synthetic construct
  source 1..21: mol_type = other RNA
ggcatgcttcctccacagtaa

SEQ ID NO: 1408 - RNA, 21 nt, synthetic construct
  source 1..21: mol_type = other RNA
ggctagaagaaaacaaactga

SEQ ID NO: 1409 - RNA, 21 nt, synthetic construct
  source 1..21: mol_type = other RNA
cctagaagaaaacaaactgta

SEQ ID NO: 1410 - RNA, 21 nt, synthetic construct
  source 1..21: mol_type = other RNA
gtagaagaaaacaaactgtca

SEQ ID NO: 1411 - RNA, 21 nt, synthetic construct
  source 1..21: mol_type = other RNA
gagaagaaaacaaactgtcaa

SEQ ID NO: 1412 - RNA, 21 nt, synthetic construct
  source 1..21: mol_type = other RNA
caagaaaacaaactgtcacaa

SEQ ID NO: 1413 - RNA, 21 nt, synthetic construct
  source 1..21: mol_type = other RNA
aaaatgctagaagaaaacaaa

SEQ ID NO: 1414 - RNA, 21 nt, synthetic construct
  source 1..21: mol_type = other RNA
aaatgctagaagaaaacaaaa

SEQ ID NO: 1415 - RNA, 21 nt, synthetic construct
  source 1..21: mol_type = other RNA
aatgctagaagaaaacaaaca

SEQ ID NO: 1416 - RNA, 21 nt, synthetic construct
  source 1..21: mol_type = other RNA
atgctagaagaaaacaaacta

SEQ ID NO: 1417 - RNA, 21 nt, synthetic construct
  source 1..21: mol_type = other RNA
tgctagaagaaaacaaactga

SEQ ID NO: 1418 - RNA, 21 nt, synthetic construct
  source 1..21: mol_type = other RNA
gctagaagaaaacaaactgta

SEQ ID NO: 1419 - RNA, 21 nt, synthetic construct
  source 1..21: mol_type = other RNA
ctagaagaaaacaaactgtca

SEQ ID NO: 1420 - RNA, 21 nt, synthetic construct
  source 1..21: mol_type = other RNA
tagaagaaaacaaactgtcaa

SEQ ID NO: 1421 - RNA, 21 nt, synthetic construct
  source 1..21: mol_type = other RNA
agaagaaaacaaactgtcaca

SEQ ID NO: 1422 - RNA, 21 nt, synthetic construct
  source 1..21: mol_type = other RNA
gaagaaaacaaactgtcacaa

SEQ ID NO: 1423 - RNA, 21 nt, synthetic construct
  source 1..21: mol_type = other RNA
aagaaaacaaactgtcacaaa

SEQ ID NO: 1424 - RNA, 21 nt, synthetic construct
  source 1..21: mol_type = other RNA
agaaaacaaactgtcacaaga

SEQ ID NO: 1425 - RNA, 21 nt, synthetic construct
  source 1..21: mol_type = other RNA
gaaaacaaactgtcacaagta

SEQ ID NO: 1426 - RNA, 21 nt, synthetic construct
  source 1..21: mol_type = other RNA
aaaacaaactgtcacaagtta

SEQ ID NO: 1427 - RNA, 21 nt, synthetic construct
  source 1..21: mol_type = other RNA
aaacaaactgtcacaagttga

SEQ ID NO: 1428 - RNA, 21 nt, synthetic construct
  source 1..21: mol_type = other RNA
tataagaaaatcatcctgaac

SEQ ID NO: 1429 - RNA, 21 nt, synthetic construct
  source 1..21: mol_type = other RNA
ttataagaaaatcatcctgac

SEQ ID NO: 1430 - RNA, 21 nt, synthetic construct
  source 1..21: mol_type = other RNA
tcacacattcaccagaaactc

SEQ ID NO: 1431 - RNA, 21 nt, synthetic construct
  source 1..21: mol_type = other RNA
tcattgcttgaaagaataccg

SEQ ID NO: 1432 - RNA, 21 nt, synthetic construct
  source 1..21: mol_type = other RNA
tttggtgtgagcattgcttgc

SEQ ID NO: 1433 - RNA, 21 nt, synthetic construct
  source 1..21: mol_type = other RNA
ttttggtgtgagcattgcttc

SEQ ID NO: 1434 - RNA, 21 nt, synthetic construct
  source 1..21: mol_type = other RNA
tcataaatgtctttgttgcac

SEQ ID NO: 1435 - RNA, 21 nt, synthetic construct
  source 1..21: mol_type = other RNA
ttagtttatgcccttcatgtg

SEQ ID NO: 1436 - RNA, 21 nt, synthetic construct
  source 1..21: mol_type = other RNA
tgctgttatagtttatgcccc

SEQ ID NO: 1437 - RNA, 21 nt, synthetic construct
  source 1..21: mol_type = other RNA
tgattttggtggtacactcac

SEQ ID NO: 1438 - RNA, 21 nt, synthetic construct
  source 1..21: mol_type = other RNA
ttgattttggtggtacactcc

SEQ ID NO: 1439 - RNA, 21 nt, synthetic construct
  source 1..21: mol_type = other RNA
tttgattttggtggtacactg

SEQ ID NO: 1440 - RNA, 21 nt, synthetic construct
  source 1..21: mol_type = other RNA
taatcttaggtgactctaccg

SEQ ID NO: 1441 - RNA, 21 nt, synthetic construct
  source 1..21: mol_type = other RNA
tcacgcaaaatcttaggtgag

SEQ ID NO: 1442 - RNA, 21 nt, synthetic construct
  source 1..21: mol_type = other RNA
tacacgcaaaatcttaggtgc

SEQ ID NO: 1443 - RNA, 21 nt, synthetic construct
  source 1..21: mol_type = other RNA
tagacacgcaaaatcttaggc

SEQ ID NO: 1444 - RNA, 21 nt, synthetic construct
  source 1..21: mol_type = other RNA
ttagacacgcaaaatcttagc

SEQ ID NO: 1445 - RNA, 21 nt, synthetic construct
  source 1..21: mol_type = other RNA
ttgtagacacgcaaaatcttc

SEQ ID NO: 1446 - RNA, 21 nt, synthetic construct
  source 1..21: mol_type = other RNA
tttgatttaaaatgccactgc

SEQ ID NO: 1447 - RNA, 21 nt, synthetic construct
  source 1..21: mol_type = other RNA
tattgatttaaaatgccactc

SEQ ID NO: 1448 - RNA, 21 nt, synthetic construct
  source 1..21: mol_type = other RNA
ttatttcagattgatttaaac

SEQ ID NO: 1449 - RNA, 21 nt, synthetic construct
  source 1..21: mol_type = other RNA
ttttatttcagattgatttac

SEQ ID NO: 1450 - RNA, 21 nt, synthetic construct
  source 1..21: mol_type = other RNA
taaccccaaagaaagatgtgc

SEQ ID NO: 1451 - RNA, 21 nt, synthetic construct
  source 1..21: mol_type = other RNA
ttgaaccccaaagaaagatgc

SEQ ID NO: 1452 - RNA, 21 nt, synthetic construct
  source 1..21: mol_type = other RNA
tttgaaccccaaagaaagatc

SEQ ID NO: 1453 - RNA, 21 nt, synthetic construct
  source 1..21: mol_type = other RNA
ttttcttgaaccccaaagaac

SEQ ID NO: 1454 - RNA, 21 nt, synthetic construct
  source 1..21: mol_type = other RNA
tatttcttgaaccccaaagac

SEQ ID NO: 1455 - RNA, 21 nt, synthetic construct
  source 1..21: mol_type = other RNA
ttatttcttgaaccccaaagc

SEQ ID NO: 1456 - RNA, 21 nt, synthetic construct
  source 1..21: mol_type = other RNA
tgattattatttcttgaaccg

SEQ ID NO: 1457 - RNA, 21 nt, synthetic construct
  source 1..21: mol_type = other RNA
tgatcatggattattatttcc

SEQ ID NO: 1458 - RNA, 21 nt, synthetic construct
  source 1..21: mol_type = other RNA
ttttctgccattttatactgc

SEQ ID NO: 1459 - RNA, 21 nt, synthetic construct
  source 1..21: mol_type = other RNA
tctttctgccattttatactc

SEQ ID NO: 1460 - RNA, 21 nt, synthetic construct
  source 1..21: mol_type = other RNA
taacaaggcaatatcataccg

SEQ ID NO: 1461 - RNA, 21 nt, synthetic construct
  source 1..21: mol_type = other RNA
tgtttcaacaaggcaatatcc

SEQ ID NO: 1462 - RNA, 21 nt, synthetic construct
  source 1..21: mol_type = other RNA
tagtttcaacaaggcaatatg

SEQ ID NO: 1463 - RNA, 21 nt, synthetic construct
  source 1..21: mol_type = other RNA
tccagtttcaacaaggcaatc

SEQ ID NO: 1464 - RNA, 21 nt, synthetic construct
  source 1..21: mol_type = other RNA
tgtttccagtttcaacaaggg

SEQ ID NO: 1465 - RNA, 21 nt, synthetic construct
  source 1..21: mol_type = other RNA
ttgtggtttccagtttcaacc

SEQ ID NO: 1466 - RNA, 21 nt, synthetic construct
  source 1..21: mol_type = other RNA
tgtaattcactgtggtttccc

SEQ ID NO: 1467 - RNA, 21 nt, synthetic construct
  source 1..21: mol_type = other RNA
ttgtgtaattcactgtggttc

SEQ ID NO: 1468 - RNA, 21 nt, synthetic construct
  source 1..21: mol_type = other RNA
ttctgtgtaattcactgtggc

SEQ ID NO: 1469 - RNA, 21 nt, synthetic construct
  source 1..21: mol_type = other RNA
tctttctggagagtattttgc

SEQ ID NO: 1470 - RNA, 21 nt, synthetic construct
  source 1..21: mol_type = other RNA
tatcttggctttctggagagc

SEQ ID NO: 1471 - RNA, 21 nt, synthetic construct
  source 1..21: mol_type = other RNA
tactaagggtatcttggcttc

SEQ ID NO: 1472 - RNA, 21 nt, synthetic construct
  source 1..21: mol_type = other RNA
ttatgtcctctgtatctcttg

SEQ ID NO: 1473 - RNA, 21 nt, synthetic construct
  source 1..21: mol_type = other RNA
tttttatgtcctctgtatctg

SEQ ID NO: 1474 - RNA, 21 nt, synthetic construct
  source 1..21: mol_type = other RNA
tgttattttatgtcctctgtc

SEQ ID NO: 1475 - RNA, 21 nt, synthetic construct
  source 1..21: mol_type = other RNA
tggttattttatgtcctctgc

SEQ ID NO: 1476 - RNA, 21 nt, synthetic construct
  source 1..21: mol_type = other RNA
tatgggttattttatgtcctg

SEQ ID NO: 1477 - RNA, 21 nt, synthetic construct
  source 1..21: mol_type = other RNA
tcttatgggttattttatgtg

SEQ ID NO: 1478 - RNA, 21 nt, synthetic construct
  source 1..21: mol_type = other RNA
ttcttatgggttattttatgc

SEQ ID NO: 1479 - RNA, 21 nt, synthetic construct
  source 1..21: mol_type = other RNA
tcacagatcatcttatgggtc

SEQ ID NO: 1480 - RNA, 21 nt, synthetic construct
  source 1..21: mol_type = other RNA
tttttctccagaatccagtcg

SEQ ID NO: 1481 - RNA, 21 nt, synthetic construct
  source 1..21: mol_type = other RNA
tgttttctccagaatccagtg

SEQ ID NO: 1482 - RNA, 21 nt, synthetic construct
  source 1..21: mol_type = other RNA
tttcatcctcctctgaatgac

SEQ ID NO: 1483 - RNA, 21 nt, synthetic construct
  source 1..21: mol_type = other RNA
tatcatcctgaaaagaccttc

SEQ ID NO: 1484 - RNA, 21 nt, synthetic construct
  source 1..21: mol_type = other RNA
tagaaaatcatcctgaaaagc

SEQ ID NO: 1485 - RNA, 21 nt, synthetic construct
  source 1..21: mol_type = other RNA
ttgatataagaaaatcatccc

SEQ ID NO: 1486 - RNA, 21 nt, synthetic construct
  source 1..21: mol_type = other RNA
taggacacaagtaaaccatcc

SEQ ID NO: 1487 - RNA, 21 nt, synthetic construct
  source 1..21: mol_type = other RNA
ttgtcctattcactcttggcc

SEQ ID NO: 1488 - RNA, 21 nt, synthetic construct
  source 1..21: mol_type = other RNA
tttgaaagaatacccagaaac

SEQ ID NO: 1489 - RNA, 21 nt, synthetic construct
  source 1..21: mol_type = other RNA
tcttgaaagaatacccagaac

SEQ ID NO: 1490 - RNA, 21 nt, synthetic construct
  source 1..21: mol_type = other RNA
ttgttgcaagcgcttatttgc

SEQ ID NO: 1491 - RNA, 21 nt, synthetic construct
  source 1..21: mol_type = other RNA
taaatgtctttgttgcaagcc

SEQ ID NO: 1492 - RNA, 21 nt, synthetic construct
  source 1..21: mol_type = other RNA
tgtccacataaatgtctttgc

SEQ ID NO: 1493 - RNA, 21 nt, synthetic construct
  source 1..21: mol_type = other RNA
ttcatgtctaggtccacatac

SEQ ID NO: 1494 - RNA, 21 nt, synthetic construct
  source 1..21: mol_type = other RNA
ttcttgagcactcttggcaag

SEQ ID NO: 1495 - RNA, 21 nt, synthetic construct
  source 1..21: mol_type = other RNA
tatccgtgcatctttcttggg

SEQ ID NO: 1496 - RNA, 21 nt, synthetic construct
  source 1..21: mol_type = other RNA
tttaaggagacaaagatttcc

SEQ ID NO: 1497 - RNA, 21 nt, synthetic construct
  source 1..21: mol_type = other RNA
ttttaaggagacaaagatttg

SEQ ID NO: 1498 - RNA, 21 nt, synthetic construct
  source 1..21: mol_type = other RNA
tttttaaggagacaaagattc

SEQ ID NO: 1499 - RNA, 21 nt, synthetic construct
  source 1..21: mol_type = other RNA
tatgttttaaggagacaaagc

SEQ ID NO: 1500 - RNA, 21 nt, synthetic construct
  source 1..21: mol_type = other RNA
tagatgttttaaggagacaac

SEQ ID NO: 1501 - RNA, 21 nt, synthetic construct
  source 1..21: mol_type = other RNA
tcagatgttttaaggagacac

SEQ ID NO: 1502 - RNA, 21 nt, synthetic construct
  source 1..21: mol_type = other RNA
ttcagatgttttaaggagacc

SEQ ID NO: 1503 - RNA, 21 nt, synthetic construct
  source 1..21: mol_type = other RNA
tctctcagatgttttaaggac

SEQ ID NO: 1504 - RNA, 21 nt, synthetic construct
  source 1..21: mol_type = other RNA
tccactctcagatgttttaac

SEQ ID NO: 1505 - RNA, 21 nt, synthetic construct
  source 1..21: mol_type = other RNA
tactgaaaccagaaagagctc

SEQ ID NO: 1506 - RNA, 21 nt, synthetic construct
  source 1..21: mol_type = other RNA
tcagcaacaatatccagttcc

SEQ ID NO: 1507 - RNA, 21 nt, synthetic construct
  source 1..21: mol_type = other RNA
tgtatccagagatgcctcctg

SEQ ID NO: 1508 - RNA, 21 nt, synthetic construct
  source 1..21: mol_type = other RNA
taatgtgtatccagagatgcg

SEQ ID NO: 1509 - RNA, 21 nt, synthetic construct
  source 1..21: mol_type = other RNA
tcattatccattttacacaag

SEQ ID NO: 1510 - RNA, 21 nt, synthetic construct
  source 1..21: mol_type = other RNA
tctcattatccattttacacc

SEQ ID NO: 1511 - RNA, 21 nt, synthetic construct
  source 1..21: mol_type = other RNA
tgcttgattttggtggtacag

SEQ ID NO: 1512 - RNA, 21 nt, synthetic construct
  source 1..21: mol_type = other RNA
tgttcctccaacgatcctggc

SEQ ID NO: 1513 - RNA, 21 nt, synthetic construct
  source 1..21: mol_type = other RNA
taaaatcttaggtgactctag

SEQ ID NO: 1514 - RNA, 21 nt, synthetic construct
  source 1..21: mol_type = other RNA
tcagattgatttaaaatgccc

SEQ ID NO: 1515 - RNA, 21 nt, synthetic construct
  source 1..21: mol_type = other RNA
ttcagattgatttaaaatgcg

SEQ ID NO: 1516 - RNA, 21 nt, synthetic construct
  source 1..21: mol_type = other RNA
tttcagattgatttaaaatgg

SEQ ID NO: 1517 - RNA, 21 nt, synthetic construct
  source 1..21: mol_type = other RNA
tccccaaagaaagatgtgtcg

SEQ ID NO: 1518 - RNA, 21 nt, synthetic construct
  source 1..21: mol_type = other RNA
ttcttgaaccccaaagaaagc

SEQ ID NO: 1519 - RNA, 21 nt, synthetic construct
  source 1..21: mol_type = other RNA
tttatttcttgaaccccaaac

SEQ ID NO: 1520 - RNA, 21 nt, synthetic construct
  source 1..21: mol_type = other RNA
ttggattattatttcttgaag

SEQ ID NO: 1521 - RNA, 21 nt, synthetic construct
  source 1..21: mol_type = other RNA
tatggattattatttcttgac

SEQ ID NO: 1522 - RNA, 21 nt, synthetic construct
  source 1..21: mol_type = other RNA
tacaaggcaatatcatacccc

SEQ ID NO: 1523 - RNA, 21 nt, synthetic construct
  source 1..21: mol_type = other RNA
tcagtttcaacaaggcaatac

SEQ ID NO: 1524 - RNA, 21 nt, synthetic construct
  source 1..21: mol_type = other RNA
tggtttccagtttcaacaagc

SEQ ID NO: 1525 - RNA, 21 nt, synthetic construct
  source 1..21: mol_type = other RNA
ttgtaattcactgtggtttcg

SEQ ID NO: 1526 - RNA, 21 nt, synthetic construct
  source 1..21: mol_type = other RNA
ttatcttggctttctggagac

SEQ ID NO: 1527 - RNA, 21 nt, synthetic construct
  source 1..21: mol_type = other RNA
tggtatcttggctttctggac

SEQ ID NO: 1528 - RNA, 21 nt, synthetic construct
  source 1..21: mol_type = other RNA
tctaagggtatcttggctttg

SEQ ID NO: 1529 - RNA, 21 nt, synthetic construct
  source 1..21: mol_type = other RNA
ttcactaagggtatcttggcc

SEQ ID NO: 1530 - RNA, 21 nt, synthetic construct
  source 1..21: mol_type = other RNA
tggtcactaagggtatcttgc

SEQ ID NO: 1531 - RNA, 21 nt, synthetic construct
  source 1..21: mol_type = other RNA
ttggtcactaagggtatcttc

SEQ ID NO: 1532 - RNA, 21 nt, synthetic construct
  source 1..21: mol_type = other RNA
tatctcttctggcactcttcc

SEQ ID NO: 1533 - RNA, 21 nt, synthetic construct
  source 1..21: mol_type = other RNA
tctctgtatctcttctggcag

SEQ ID NO: 1534 - RNA, 21 nt, synthetic construct
  source 1..21: mol_type = other RNA
tatgtcctctgtatctcttcc

SEQ ID NO: 1535 - RNA, 21 nt, synthetic construct
  source 1..21: mol_type = other RNA
tttatgtcctctgtatctctc

SEQ ID NO: 1536 - RNA, 21 nt, synthetic construct
  source 1..21: mol_type = other RNA
tattttatgtcctctgtatcc

SEQ ID NO: 1537 - RNA, 21 nt, synthetic construct
  source 1..21: mol_type = other RNA
ttattttatgtcctctgtatg

SEQ ID NO: 1538 - RNA, 21 nt, synthetic construct
  source 1..21: mol_type = other RNA
ttatgggttattttatgtccc

SEQ ID NO: 1539 - RNA, 21 nt, synthetic construct
  source 1..21: mol_type = other RNA
ttcatcttatgggttattttc

SEQ ID NO: 1540 - RNA, 21 nt, synthetic construct
  source 1..21: mol_type = other RNA
tatcatcttatgggttatttc

SEQ ID NO: 1541 - RNA, 21 nt, synthetic construct
  source 1..21: mol_type = other RNA
tcagatcatcttatgggttac

SEQ ID NO: 1542 - RNA, 21 nt, synthetic construct
  source 1..21: mol_type = other RNA
tacagatcatcttatgggttc

SEQ ID NO: 1543 - RNA, 21 nt, synthetic construct
  source 1..21: mol_type = other RNA
tcagaatccagtccacgtacc

SEQ ID NO: 1544 - RNA, 21 nt, synthetic construct
  source 1..21: mol_type = other RNA
tttctccagaatccagtccag

SEQ ID NO: 1545 - RNA, 21 nt, synthetic construct
  source 1..21: mol_type = other RNA
ttgagttttctccagaatccc

SEQ ID NO: 1546 - RNA, 21 nt, synthetic construct
  source 1..21: mol_type = other RNA
tttgagttttctccagaatcg

SEQ ID NO: 1547 - RNA, 21 nt, synthetic construct
  source 1..21: mol_type = other RNA
tcttgagttttctccagaatg

SEQ ID NO: 1548 - RNA, 21 nt, synthetic construct
  source 1..21: mol_type = other RNA
ttactgtggaggaagcatgcc

SEQ ID NO: 1549 - RNA, 21 nt, synthetic construct
  source 1..21: mol_type = other RNA
tcagtttgttttcttctagcc

SEQ ID NO: 1550 - RNA, 21 nt, synthetic construct
  source 1..21: mol_type = other RNA
tacagtttgttttcttctagg

SEQ ID NO: 1551 - RNA, 21 nt, synthetic construct
  source 1..21: mol_type = other RNA
tgacagtttgttttcttctac

SEQ ID NO: 1552 - RNA, 21 nt, synthetic construct
  source 1..21: mol_type = other RNA
ttgacagtttgttttcttctc

SEQ ID NO: 1553 - RNA, 21 nt, synthetic construct
  source 1..21: mol_type = other RNA
ttgtgacagtttgttttcttg

SEQ ID NO: 1554 - RNA, 21 nt, synthetic construct
  source 1..21: mol_type = other RNA
tttgttttcttctagcatttt

SEQ ID NO: 1555 - RNA, 21 nt, synthetic construct
  source 1..21: mol_type = other RNA
ttttgttttcttctagcattt

SEQ ID NO: 1556 - RNA, 21 nt, synthetic construct
  source 1..21: mol_type = other RNA
tgtttgttttcttctagcatt

SEQ ID NO: 1557 - RNA, 21 nt, synthetic construct
  source 1..21: mol_type = other RNA
tagtttgttttcttctagcat

SEQ ID NO: 1558 - RNA, 21 nt, synthetic construct
  source 1..21: mol_type = other RNA
tcagtttgttttcttctagca

SEQ ID NO: 1559 - RNA, 21 nt, synthetic construct
  source 1..21: mol_type = other RNA
tacagtttgttttcttctagc

SEQ ID NO: 1560 - RNA, 21 nt, synthetic construct
  source 1..21: mol_type = other RNA
tgacagtttgttttcttctag

SEQ ID NO: 1561 - RNA, 21 nt, synthetic construct
  source 1..21: mol_type = other RNA
ttgacagtttgttttcttcta

SEQ ID NO: 1562 - RNA, 21 nt, synthetic construct
  source 1..21: mol_type = other RNA
tgtgacagtttgttttcttct

SEQ ID NO: 1563 - RNA, 21 nt, synthetic construct
  source 1..21: mol_type = other RNA
ttgtgacagtttgttttcttc

SEQ ID NO: 1564 - RNA, 21 nt, synthetic construct
  source 1..21: mol_type = other RNA
tttgtgacagtttgttttctt

SEQ ID NO: 1565 - RNA, 21 nt, synthetic construct
  source 1..21: mol_type = other RNA
tcttgtgacagtttgttttct

SEQ ID NO: 1566 - RNA, 21 nt, synthetic construct
  source 1..21: mol_type = other RNA
tacttgtgacagtttgttttc

SEQ ID NO: 1567 - RNA, 21 nt, synthetic construct
  source 1..21: mol_type = other RNA
taacttgtgacagtttgtttt

SEQ ID NO: 1568 - RNA, 21 nt, synthetic construct
  source 1..21: mol_type = other RNA
tcaacttgtgacagtttgttt

SEQ ID NO: 1569 - RNA, 21 nt, synthetic construct
  source 1..21: mol_type = other RNA
gagtttctggtgaatgtgtga

SEQ ID NO: 1570 - RNA, 21 nt, synthetic construct
  source 1..21: mol_type = other RNA
gcaagcaatgctcacaccaaa

SEQ ID NO: 1571 - RNA, 21 nt, synthetic construct
  source 1..21: mol_type = other RNA
gaagcaatgctcacaccaaaa

SEQ ID NO: 1572 - RNA, 21 nt, synthetic construct
  source 1..21: mol_type = other RNA
cacatgaagggcataaactaa

SEQ ID NO: 1573 - RNA, 21 nt, synthetic construct
  source 1..21: mol_type = other RNA
ggggcataaactataacagca

SEQ ID NO: 1574 - RNA, 21 nt, synthetic construct
  source 1..21: mol_type = other RNA
cggtagagtcacctaagatta

SEQ ID NO: 1575 - RNA, 21 nt, synthetic construct
  source 1..21: mol_type = other RNA
gaagattttgcgtgtctacaa

SEQ ID NO: 1576 - RNA, 21 nt, synthetic construct
  source 1..21: mol_type = other RNA
gcagtggcattttaaatcaaa

SEQ ID NO: 1577 - RNA, 21 nt, synthetic construct
  source 1..21: mol_type = other RNA
gagtggcattttaaatcaata

SEQ ID NO: 1578 - RNA, 21 nt, synthetic construct
  source 1..21: mol_type = other RNA
gtaaatcaatctgaaataaaa

SEQ ID NO: 1579 - RNA, 21 nt, synthetic construct
  source 1..21: mol_type = other RNA
gtctttggggttcaagaaata

SEQ ID NO: 1580 - RNA, 21 nt, synthetic construct
  source 1..21: mol_type = other RNA
cggttcaagaaataataatca

SEQ ID NO: 1581 - RNA, 21 nt, synthetic construct
  source 1..21: mol_type = other RNA
gcagtataaaatggcagaaaa

SEQ ID NO: 1582 - RNA, 21 nt, synthetic construct
  source 1..21: mol_type = other RNA
gaaccacagtgaattacacaa

SEQ ID NO: 1583 - RNA, 21 nt, synthetic construct
  source 1..21: mol_type = other RNA
gccacagtgaattacacagaa

SEQ ID NO: 1584 - RNA, 21 nt, synthetic construct
  source 1..21: mol_type = other RNA
caggacataaaataacccata

SEQ ID NO: 1585 - RNA, 21 nt, synthetic construct
  source 1..21: mol_type = other RNA
gaaggtcttttcaggatgata

SEQ ID NO: 1586 - RNA, 21 nt, synthetic construct
  source 1..21: mol_type = other RNA
gcttttcaggatgattttcta

SEQ ID NO: 1587 - RNA, 21 nt, synthetic construct
  source 1..21: mol_type = other RNA
gggatgattttcttatatcaa

SEQ ID NO: 1588 - RNA, 21 nt, synthetic construct
  source 1..21: mol_type = other RNA
ggatggtttacttgtgtccta

SEQ ID NO: 1589 - RNA, 21 nt, synthetic construct
  source 1..21: mol_type = other RNA
gactgccaagagtgaatagga

SEQ ID NO: 1590 - RNA, 21 nt, synthetic construct
  source 1..21: mol_type = other RNA
ggccaagagtgaataggacaa

SEQ ID NO: 1591 - RNA, 21 nt, synthetic construct
  source 1..21: mol_type = other RNA
ggatttctgggtattctttca

SEQ ID NO: 1592 - RNA, 21 nt, synthetic construct
  source 1..21: mol_type = other RNA
gtttctgggtattctttcaaa

SEQ ID NO: 1593 - RNA, 21 nt, synthetic construct
  source 1..21: mol_type = other RNA
gttctgggtattctttcaaga

SEQ ID NO: 1594 - RNA, 21 nt, synthetic construct
  source 1..21: mol_type = other RNA
cgtattctttcaagcaatgca

SEQ ID NO: 1595 - RNA, 21 nt, synthetic construct
  source 1..21: mol_type = other RNA
gcaaataagcgcttgcaacaa

SEQ ID NO: 1596 - RNA, 21 nt, synthetic construct
  source 1..21: mol_type = other RNA
ggcttgcaacaaagacattta

SEQ ID NO: 1597 - RNA, 21 nt, synthetic construct
  source 1..21: mol_type = other RNA
gcaaagacatttatgtggaca

SEQ ID NO: 1598 - RNA, 21 nt, synthetic construct
  source 1..21: mol_type = other RNA
gtatgtggacctagacatgaa

SEQ ID NO: 1599 - RNA, 21 nt, synthetic construct
  source 1..21: mol_type = other RNA
cttgccaagagtgctcaagaa

SEQ ID NO: 1600 - RNA, 21 nt, synthetic construct
  source 1..21: mol_type = other RNA
cccaagaaagatgcacggata

SEQ ID NO: 1601 - RNA, 21 nt, synthetic construct
  source 1..21: mol_type = other RNA
ggaaatctttgtctccttaaa

SEQ ID NO: 1602 - RNA, 21 nt, synthetic construct
  source 1..21: mol_type = other RNA
cagcaaagctctttctggtta

SEQ ID NO: 1603 - RNA, 21 nt, synthetic construct
  source 1..21: mol_type = other RNA
cgatctccaactaaaatacta

SEQ ID NO: 1604 - RNA, 21 nt, synthetic construct
  source 1..21: mol_type = other RNA
cttgtgtaaaatggataatga

SEQ ID NO: 1605 - RNA, 21 nt, synthetic construct
  source 1..21: mol_type = other RNA
ctcacctaagattttgcgtga

SEQ ID NO: 1606 - RNA, 21 nt, synthetic construct
  source 1..21: mol_type = other RNA
gcacctaagattttgcgtgta

SEQ ID NO: 1607 - RNA, 21 nt, synthetic construct
  source 1..21: mol_type = other RNA
gcctaagattttgcgtgtcta

SEQ ID NO: 1608 - RNA, 21 nt, synthetic construct
  source 1..21: mol_type = other RNA
cgcattttaaatcaatctgaa

SEQ ID NO: 1609 - RNA, 21 nt, synthetic construct
  source 1..21: mol_type = other RNA
gcacatctttctttggggtta

SEQ ID NO: 1610 - RNA, 21 nt, synthetic construct
  source 1..21: mol_type = other RNA
gcatctttctttggggttcaa

SEQ ID NO: 1611 - RNA, 21 nt, synthetic construct
  source 1..21: mol_type = other RNA
gttctttggggttcaagaaaa

SEQ ID NO: 1612 - RNA, 21 nt, synthetic construct
  source 1..21: mol_type = other RNA
gagtataaaatggcagaaaga

SEQ ID NO: 1613 - RNA, 21 nt, synthetic construct
  source 1..21: mol_type = other RNA
cggtatgatattgccttgtta

SEQ ID NO: 1614 - RNA, 21 nt, synthetic construct
  source 1..21: mol_type = other RNA
ggatattgccttgttgaaaca

SEQ ID NO: 1615 - RNA, 21 nt, synthetic construct
  source 1..21: mol_type = other RNA
cgaaaccacagtgaattacaa

SEQ ID NO: 1616 - RNA, 21 nt, synthetic construct
  source 1..21: mol_type = other RNA
gcaaaatactctccagaaaga

SEQ ID NO: 1617 - RNA, 21 nt, synthetic construct
  source 1..21: mol_type = other RNA
caaagccaagatacccttaga

SEQ ID NO: 1618 - RNA, 21 nt, synthetic construct
  source 1..21: mol_type = other RNA
gacagaggacataaaataaca

SEQ ID NO: 1619 - RNA, 21 nt, synthetic construct
  source 1..21: mol_type = other RNA
gacccataagatgatctgtga

SEQ ID NO: 1620 - RNA, 21 nt, synthetic construct
  source 1..21: mol_type = other RNA
cgactggattctggagaaaaa

SEQ ID NO: 1621 - RNA, 21 nt, synthetic construct
  source 1..21: mol_type = other RNA
gcgtcagtttctggtgaatga

SEQ ID NO: 1622 - RNA, 21 nt, synthetic construct
  source 1..21: mol_type = other RNA
gcttcagtttctggtgaatga

SEQ ID NO: 1623 - RNA, 21 nt, synthetic construct
  source 1..21: mol_type = other RNA
gttctggtgaatgtgtgacta

SEQ ID NO: 1624 - RNA, 21 nt, synthetic construct
  source 1..21: mol_type = other RNA
cgagcgatttctgggtattca

SEQ ID NO: 1625 - RNA, 21 nt, synthetic construct
  source 1..21: mol_type = other RNA
ccagcgatttctgggtattca

SEQ ID NO: 1626 - RNA, 21 nt, synthetic construct
  source 1..21: mol_type = other RNA
gaatctttgtctccttaaaaa

SEQ ID NO: 1627 - RNA, 21 nt, synthetic construct
  source 1..21: mol_type = other RNA
gctttgtctccttaaaacata

SEQ ID NO: 1628 - RNA, 21 nt, synthetic construct
  source 1..21: mol_type = other RNA
ggtctccttaaaacatctgaa

SEQ ID NO: 1629 - RNA, 21 nt, synthetic construct
  source 1..21: mol_type = other RNA
gccaggatcgttggaggaaca

SEQ ID NO: 1630 - RNA, 21 nt, synthetic construct
  source 1..21: mol_type = other RNA
gctttctttggggttcaagaa

SEQ ID NO: 1631 - RNA, 21 nt, synthetic construct
  source 1..21: mol_type = other RNA
gtcaagaaataataatccata

SEQ ID NO: 1632 - RNA, 21 nt, synthetic construct
  source 1..21: mol_type = other RNA
ggggtatgatattgccttgta

SEQ ID NO: 1633 - RNA, 21 nt, synthetic construct
  source 1..21: mol_type = other RNA
ggatacagaggacataaaata

SEQ ID NO: 1634 - RNA, 21 nt, synthetic construct
  source 1..21: mol_type = other RNA
catacagaggacataaaataa

SEQ ID NO: 1635 - RNA, 21 nt, synthetic construct
  source 1..21: mol_type = other RNA
gggacataaaataacccataa

SEQ ID NO: 1636 - RNA, 21 nt, synthetic construct
  source 1..21: mol_type = other RNA
ctggactggattctggagaaa

SEQ ID NO: 1637 - RNA, 21 nt, synthetic construct
  source 1..21: mol_type = other RNA
ggctagaagaaaacaaactga

SEQ ID NO: 1638 - RNA, 21 nt, synthetic construct
  source 1..21: mol_type = other RNA
cctagaagaaaacaaactgta

SEQ ID NO: 1639 - RNA, 21 nt, synthetic construct
  source 1..21: mol_type = other RNA
gtagaagaaaacaaactgtca

SEQ ID NO: 1640 - RNA, 21 nt, synthetic construct
  source 1..21: mol_type = other RNA
gagaagaaaacaaactgtcaa

SEQ ID NO: 1641 - RNA, 21 nt, synthetic construct
  source 1..21: mol_type = other RNA
caagaaaacaaactgtcacaa

SEQ ID NO: 1642 - RNA, 21 nt, synthetic construct
  source 1..21: mol_type = other RNA
ggatttctgggtattctttct

SEQ ID NO: 1643 - RNA, 19 nt, synthetic construct
  source 1..19: mol_type = other RNA
gggtattctttcaagcaat

SEQ ID NO: 1644 - RNA, 19 nt, synthetic construct
  source 1..19: mol_type = other RNA
atttctgggtattctttca

SEQ ID NO: 1645 - RNA, 19 nt, synthetic construct
  source 1..19: mol_type = other RNA
tcaggatgattttcttatt

SEQ ID NO: 1646 - RNA, 19 nt, synthetic construct
  source 1..19: mol_type = other RNA
gggtattctttcaagcaat

SEQ ID NO: 1647 - RNA, 19 nt, synthetic construct
  source 1..19: mol_type = other RNA
atttctgggtattctttca

SEQ ID NO: 1648 - RNA, 21 nt, synthetic construct
  source 1..21: mol_type = other RNA
tataagaaaatcatcctgaac

SEQ ID NO: 1649 - RNA, 21 nt, synthetic construct
  source 1..21: mol_type = other RNA
ttataagaaaatcatcctgac

SEQ ID NO: 1650 - RNA, 21 nt, synthetic construct
  source 1..21: mol_type = other RNA
tcacacattcaccagaaactc

SEQ ID NO: 1651 - RNA, 21 nt, synthetic construct
  source 1..21: mol_type = other RNA
tttggtgtgagcattgcttgc

SEQ ID NO: 1652 - RNA, 21 nt, synthetic construct
  source 1..21: mol_type = other RNA
ttttggtgtgagcattgcttc

SEQ ID NO: 1653 - RNA, 21 nt, synthetic construct
  source 1..21: mol_type = other RNA
ttagtttatgcccttcatgtg

SEQ ID NO: 1654 - RNA, 21 nt, synthetic construct
  source 1..21: mol_type = other RNA
tgctgttatagtttatgcccc

SEQ ID NO: 1655 - RNA, 21 nt, synthetic construct
  source 1..21: mol_type = other RNA
taatcttaggtgactctaccg

SEQ ID NO: 1656 - RNA, 21 nt, synthetic construct
  source 1..21: mol_type = other RNA
ttgtagacacgcaaaatcttc

SEQ ID NO: 1657 - RNA, 21 nt, synthetic construct
  source 1..21: mol_type = other RNA
tttgatttaaaatgccactgc

SEQ ID NO: 1658 - RNA, 21 nt, synthetic construct
  source 1..21: mol_type = other RNA
tattgatttaaaatgccactc

SEQ ID NO: 1659 - RNA, 21 nt, synthetic construct
  source 1..21: mol_type = other RNA
ttttatttcagattgatttac

SEQ ID NO: 1660 - RNA, 21 nt, synthetic construct
  source 1..21: mol_type = other RNA
tatttcttgaaccccaaagac

SEQ ID NO: 1661 - RNA, 21 nt, synthetic construct
  source 1..21: mol_type = other RNA
tgattattatttcttgaaccg

SEQ ID NO: 1662 - RNA, 21 nt, synthetic construct
  source 1..21: mol_type = other RNA
ttttctgccattttatactgc

SEQ ID NO: 1663 - RNA, 21 nt, synthetic construct
  source 1..21: mol_type = other RNA
ttgtgtaattcactgtggttc

SEQ ID NO: 1664 - RNA, 21 nt, synthetic construct
  source 1..21: mol_type = other RNA
ttctgtgtaattcactgtggc

SEQ ID NO: 1665 - RNA, 21 nt, synthetic construct
  source 1..21: mol_type = other RNA
tatgggttattttatgtcctg

SEQ ID NO: 1666 - RNA, 21 nt, synthetic construct
  source 1..21: mol_type = other RNA
tatcatcctgaaaagaccttc

SEQ ID NO: 1667 - RNA, 21 nt, synthetic construct
  source 1..21: mol_type = other RNA
tagaaaatcatcctgaaaagc

SEQ ID NO: 1668 - RNA, 21 nt, synthetic construct
  source 1..21: mol_type = other RNA
ttgatataagaaaatcatccc

SEQ ID NO: 1669 - RNA, 21 nt, synthetic construct
  source 1..21: mol_type = other RNA
taggacacaagtaaaccatcc

SEQ ID NO: 1670 - RNA, 21 nt, synthetic construct
  source 1..21: mol_type = other RNA
tcctattcactcttggcagtc

SEQ ID NO: 1671 - RNA, 21 nt, synthetic construct
  source 1..21: mol_type = other RNA
ttgtcctattcactcttggcc

SEQ ID NO: 1672 - RNA, 21 nt, synthetic construct
  source 1..21: mol_type = other RNA
tgaaagaatacccagaaatcc

SEQ ID NO: 1673 - RNA, 21 nt, synthetic construct
  source 1..21: mol_type = other RNA
tttgaaagaatacccagaaac

SEQ ID NO: 1674 - RNA, 21 nt, synthetic construct
  source 1..21: mol_type = other RNA
tcttgaaagaatacccagaac

SEQ ID NO: 1675 - RNA, 21 nt, synthetic construct
  source 1..21: mol_type = other RNA
tgcattgcttgaaagaatacg

SEQ ID NO: 1676 - RNA, 21 nt, synthetic construct
  source 1..21: mol_type = other RNA
ttgttgcaagcgcttatttgc

SEQ ID NO: 1677 - RNA, 21 nt, synthetic construct
  source 1..21: mol_type = other RNA
taaatgtctttgttgcaagcc

SEQ ID NO: 1678 - RNA, 21 nt, synthetic construct
  source 1..21: mol_type = other RNA
tgtccacataaatgtctttgc

SEQ ID NO: 1679 - RNA, 21 nt, synthetic construct
  source 1..21: mol_type = other RNA
ttcatgtctaggtccacatac

SEQ ID NO: 1680 - RNA, 21 nt, synthetic construct
  source 1..21: mol_type = other RNA
ttcttgagcactcttggcaag

SEQ ID NO: 1681 - RNA, 21 nt, synthetic construct
  source 1..21: mol_type = other RNA
tatccgtgcatctttcttggg

SEQ ID NO: 1682 - RNA, 21 nt, synthetic construct
  source 1..21: mol_type = other RNA
tttaaggagacaaagatttcc

SEQ ID NO: 1683 - RNA, 21 nt, synthetic construct
  source 1..21: mol_type = other RNA
taaccagaaagagctttgctg

SEQ ID NO: 1684 - RNA, 21 nt, synthetic construct
  source 1..21: mol_type = other RNA
tagtattttagttggagatcg

SEQ ID NO: 1685 - RNA, 21 nt, synthetic construct
  source 1..21: mol_type = other RNA
tcattatccattttacacaag

SEQ ID NO: 1686 - RNA, 21 nt, synthetic construct
  source 1..21: mol_type = other RNA
tcacgcaaaatcttaggtgag

SEQ ID NO: 1687 - RNA, 21 nt, synthetic construct
  source 1..21: mol_type = other RNA
tacacgcaaaatcttaggtgc

SEQ ID NO: 1688 - RNA, 21 nt, synthetic construct
  source 1..21: mol_type = other RNA
tagacacgcaaaatcttaggc

SEQ ID NO: 1689 - RNA, 21 nt, synthetic construct
  source 1..21: mol_type = other RNA
ttcagattgatttaaaatgcg

SEQ ID NO: 1690 - RNA, 21 nt, synthetic construct
  source 1..21: mol_type = other RNA
taaccccaaagaaagatgtgc

SEQ ID NO: 1691 - RNA, 21 nt, synthetic construct
  source 1..21: mol_type = other RNA
ttgaaccccaaagaaagatgc

SEQ ID NO: 1692 - RNA, 21 nt, synthetic construct
  source 1..21: mol_type = other RNA
ttttcttgaaccccaaagaac

SEQ ID NO: 1693 - RNA, 21 nt, synthetic construct
  source 1..21: mol_type = other RNA
tctttctgccattttatactc

SEQ ID NO: 1694 - RNA, 21 nt, synthetic construct
  source 1..21: mol_type = other RNA
taacaaggcaatatcataccg

SEQ ID NO: 1695 - RNA, 21 nt, synthetic construct
  source 1..21: mol_type = other RNA
tgtttcaacaaggcaatatcc

SEQ ID NO: 1696 - RNA, 21 nt, synthetic construct
  source 1..21: mol_type = other RNA
ttgtaattcactgtggtttcg

SEQ ID NO: 1697 - RNA, 21 nt, synthetic construct
  source 1..21: mol_type = other RNA
tctttctggagagtattttgc

SEQ ID NO: 1698 - RNA, 21 nt, synthetic construct
  source 1..21: mol_type = other RNA
tctaagggtatcttggctttg

SEQ ID NO: 1699 - RNA, 21 nt, synthetic construct
  source 1..21: mol_type = other RNA
tgttattttatgtcctctgtc

SEQ ID NO: 1700 - RNA, 21 nt, synthetic construct
  source 1..21: mol_type = other RNA
tcacagatcatcttatgggtc

SEQ ID NO: 1701 - RNA, 21 nt, synthetic construct
  source 1..21: mol_type = other RNA
tttttctccagaatccagtcg

SEQ ID NO: 1702 - RNA, 21 nt, synthetic construct
  source 1..21: mol_type = other RNA
tcattcaccagaaactgacgc

SEQ ID NO: 1703 - RNA, 21 nt, synthetic construct
  source 1..21: mol_type = other RNA
tcattcaccagaaactgaagc

SEQ ID NO: 1704 - RNA, 21 nt, synthetic construct
  source 1..21: mol_type = other RNA
tagtcacacattcaccagaac

SEQ ID NO: 1705 - RNA, 21 nt, synthetic construct
  source 1..21: mol_type = other RNA
tgaatacccagaaatcgctcg

SEQ ID NO: 1706 - RNA, 21 nt, synthetic construct
  source 1..21: mol_type = other RNA
tgaatacccagaaatcgctgg

SEQ ID NO: 1707 - RNA, 21 nt, synthetic construct
  source 1..21: mol_type = other RNA
tttttaaggagacaaagattc

SEQ ID NO: 1708 - RNA, 21 nt, synthetic construct
  source 1..21: mol_type = other RNA
tatgttttaaggagacaaagc

SEQ ID NO: 1709 - RNA, 21 nt, synthetic construct
  source 1..21: mol_type = other RNA
ttcagatgttttaaggagacc

SEQ ID NO: 1710 - RNA, 21 nt, synthetic construct
  source 1..21: mol_type = other RNA
tgttcctccaacgatcctggc

SEQ ID NO: 1711 - RNA, 21 nt, synthetic construct
  source 1..21: mol_type = other RNA
ttcttgaaccccaaagaaagc

SEQ ID NO: 1712 - RNA, 21 nt, synthetic construct
  source 1..21: mol_type = other RNA
tatggattattatttcttgac

SEQ ID NO: 1713 - RNA, 21 nt, synthetic construct
  source 1..21: mol_type = other RNA
tacaaggcaatatcatacccc

SEQ ID NO: 1714 - RNA, 21 nt, synthetic construct
  source 1..21: mol_type = other RNA
tattttatgtcctctgtatcc

SEQ ID NO: 1715 - RNA, 21 nt, synthetic construct
  source 1..21: mol_type = other RNA
ttattttatgtcctctgtatg

SEQ ID NO: 1716 - RNA, 21 nt, synthetic construct
  source 1..21: mol_type = other RNA
ttatgggttattttatgtccc

SEQ ID NO: 1717 - RNA, 21 nt, synthetic construct
  source 1..21: mol_type = other RNA
tttctccagaatccagtccag

SEQ ID NO: 1718 - RNA, 21 nt, synthetic construct
  source 1..21: mol_type = other RNA
tcagtttgttttcttctagcc

SEQ ID NO: 1719 - RNA, 21 nt, synthetic construct
  source 1..21: mol_type = other RNA
tacagtttgttttcttctagg

SEQ ID NO: 1720 - RNA, 21 nt, synthetic construct
  source 1..21: mol_type = other RNA
tgacagtttgttttcttctac

SEQ ID NO: 1721 - RNA, 21 nt, synthetic construct
  source 1..21: mol_type = other RNA
ttgacagtttgttttcttctc

SEQ ID NO: 1722 - RNA, 21 nt, synthetic construct
  source 1..21: mol_type = other RNA
ttgtgacagtttgttttcttg

SEQ ID NO: 1723 - RNA, 21 nt, synthetic construct
  source 1..21: mol_type = other RNA
agaaagaatacccagaaatcc

SEQ ID NO: 1724 - RNA, 21 nt, synthetic construct
  source 1..21: mol_type = other RNA
attgcttgaaagaatacccag

SEQ ID NO: 1725 - RNA, 21 nt, synthetic construct
  source 1..21: mol_type = other RNA
tgaaagaatacccagaaatcg

SEQ ID NO: 1726 - RNA, 21 nt, synthetic construct
  source 1..21: mol_type = other RNA
tataagaaaatcatcctgaaa

SEQ ID NO: 1727 - RNA, 21 nt, synthetic construct
  source 1..21: mol_type = other RNA
attgcttgaaagaatacccag

SEQ ID NO: 1728 - RNA, 21 nt, synthetic construct
  source 1..21: mol_type = other RNA
tgaaagaatacccagaaatcg

SEQ ID NO: 1729 - RNA, 21 nt, synthetic construct
  source 1..21: mol_type = other RNA
ggatttctgggtattctttct

SEQ ID NO: 1730 - RNA, 21 nt, synthetic construct
  source 1..21: mol_type = other RNA
agaaagaatacccagaaatcc

SEQ ID NO: 1731 - RNA, 21 nt, synthetic construct
  source 1..21: mol_type = other RNA
gttcaggatgattttcttata

SEQ ID NO: 1732 - RNA, 21 nt, synthetic construct
  source 1..21: mol_type = other RNA
gtcaggatgattttcttataa

SEQ ID NO: 1733 - RNA, 19 nt, synthetic construct
  source 1..19: mol_type = other RNA
atttctgggtattctttct

SEQ ID NO: 1734 - RNA, 19 nt, synthetic construct
  source 1..19: mol_type = other RNA
agaaagaatacccagaaat